(19) United States
(12) Patent Application Publication
(10) Pub. No.: US 2022/0402977 A1
(43) Pub. Date: Dec. 22, 2022

(54) SELF-ASSEMBLING VIRAL SPIKE-EABR NANOPARTICLES

(71) Applicant: California Institute of Technology, Pasadena, CA (US)

(72) Inventors: Magnus AG. Hoffmann, Pasadena, CA (US); Pamela J. Bjorkman, Altadena, CA (US)

(21) Appl. No.: 17/835,751

(22) Filed: Jun. 8, 2022

Related U.S. Application Data
(60) Provisional application No. 63/208,889, filed on Jun. 9, 2021.

(51) Int. Cl. C07K 14/005; C12N 15/62; A61P 31/14; A61K 39/215; C07K 14/735

(52) U.S. Cl. CPC C07K 14/005; C12N 15/62; A61P 31/14; A61K 39/215; C07K 14/70535; C07K 2319/06; A61K 2039/53

(57) ABSTRACT

Disclosed herein include methods, compositions, and kits suitable for use in vaccination. There are provided, in some embodiments, nucleic acid compositions (e.g., mRNA vaccine, DNA vaccine) comprising a polynucleotide encoding a fusion protein. The fusion protein can comprise an antigenic polypeptide (AP) and an endosomal sorting complex required for transport (ESCRT)-recruiting domain (ERD). A plurality of fusion proteins can be capable of self-assembling into an enveloped nanoparticle (ENP) secreted from a cell in which the fusion proteins are expressed. There are provided, in some embodiments, populations of ENPs.

### RELATED APPLICATIONS

This application claims the benefit under 35 U.S.C. § 119(e) of U.S. Provisional Patent Application No. 63/208,889, filed Jun. 9, 2021, the content of this related application is incorporated herein by reference in its entirety for all purposes.

### REFERENCE TO SEQUENCE LISTING

The present application is being filed along with a Sequence Listing in electronic format. The Sequence Listing is provided as a file entitled 30KJ 302443 US Sequence Listing, created Jun. 5, 2022, which is 28 kilobytes in size. The information in the electronic format of the Sequence Listing is incorporated herein by reference in its entirety.

### BACKGROUND

### Field

The present disclosure relates generally to the field of immune medicine, in particular, to self-assembling nanoparticles as a vaccine platform.

### Description of the Related Art

The ongoing COVID-19 pandemic has caused over 500 million infections and 6 million deaths worldwide and represents the third outbreak triggered by zoonotic transmission of a beta-coronavirus (beta-CoV) in the last two decades. The causative agent of COVID-19 is Severe Acute Respiratory Syndrome coronavirus 2 (SARS-CoV-2), which is related to SARS-CoV and Middle East Respiratory Syndrome coronavirus (MERS-CoV), the CoVs responsible for the outbreaks in 2002 and 2012, respectively. While the origin of SARS-CoV-2 is still being investigated, bats have been identified as the natural reservoir for SARS-CoV and MERS-CoV, and human transmission required intermediate hosts such as civets and camels. Various CoV strains are known to cause disease in other animal species and a small number of mutations might be sufficient to enable human transmission posing a risk for future CoV outbreaks. For instance, two recent reports documented human transmission of an alpha-CoV that normally infects dogs and a delta-CoV known to infect pigs. Once a zoonotic CoV strain has spilled over to humans, with enough human hosts (as is definitely the case for SARS-CoV-2), the virus will continue to evolve leading to the emergence of new variants that can become resistant to vaccines and therapeutics, a major concern for a number of recently-identified SARS-CoV-2 variants of concern (VOCs).

Two types of vaccine technologies are needed to help prevent future CoV pandemics and protect against emerging SARS-CoV-2 VOCs: i) Rapid-response vaccines, and ii) universal CoV vaccines. Rapid-response vaccines can be developed immediately once human transmission of a particular CoV has been detected and would be specific to the newly-identified CoV strain or variant. This type of vaccine requires a technology that enables fast, scalable, and adaptable production to ensure rapid global distribution. During the COVID-19 pandemic, mRNA vaccines have emerged as an ideal platform for the development of rapid-response vaccines. The mRNA vaccines produced by Pfizer and Moderna encode the SARS-CoV-2 spike (S) protein, the main target of antibody responses during natural infections. Clinical studies have demonstrated that mRNA vaccines have been highly effective, preventing >90% of symptomatic and severe SARS-CoV-2 infections. However, pre-clinical and clinical studies have shown that neutralizing antibody titers elicited by mRNA vaccines are ˜10-fold lower than titers elicited by protein nanoparticle (NP)-based vaccines. This is concerning with regards to the emergence of VOCs such as Delta, Omicron, and its BA.2 subvariant that are up to 10-fold less sensitive to antibodies elicited by mRNA vaccines. Thus rapid-response vaccine technologies need to be developed that achieve robust neutralizing antibody responses to prevent viral escape and ensure lasting protection during the ongoing and future CoV pandemics.

In contrast to rapid-response vaccines, the aim of a universal CoV vaccine is to confer broad immunity to a wide range of CoV strains and their potential variants before human transmission even occurred. Broad immunity can be achieved by guiding the immune response to parts of the virus that are conserved among CoVs and their potential variants. Even weak immune responses elicited by a universal CoV vaccine can be sufficient to prevent severe infections and rapid spread following future zoonotic transmission events and/or emergence of new variants. Mosaic protein NPs that presented receptor-binding domains (RBDs), the part of the S protein that interacts with host receptors to enter cells, from eight different SARS-like beta-CoV strains have been shown to elicit heterologous antibody responses against beta-CoV strains that were not displayed on the mosaic NP. However, this strategy is likely limited to closely-related strains as the RBD is not widely conserved among CoV families. There is a need for universal vaccines that present the entire S protein of various CoV strains and be more effective, as other parts of the S protein, including the N-terminal domain and the S2 domain, are more conserved than the RBD. Hence universal vaccine technologies are needed that generate densely-coated NPs for a wide range of CoV S proteins without the need for extensive protein engineering.

### SUMMARY

Disclosed herein include compositions (e.g., vaccine compositions). In some embodiments, the composition comprises: a nucleic acid composition comprising a polynucleotide encoding a fusion protein, wherein the fusion protein comprises an antigenic polypeptide (AP) and an endosomal sorting complex required for transport (ESCRT)-recruiting domain (ERD), and wherein a plurality of fusion proteins are capable of self-assembling into an enveloped nanoparticle (ENP) secreted from a cell in which the fusion proteins are expressed, thereby generating a population of ENPs.

Disclosed herein include compositions (e.g., vaccine compositions). In some embodiments, the composition comprises: a nucleic acid composition comprising n polynucleotides each encoding an nth fusion protein, wherein n is an integer from 2 to 500, wherein each fusion protein comprises an AP and an endosomal sorting complex required for transport (ESCRT)-recruiting domain (ERD), wherein at least two of the fusion proteins differ with respect to the AP, and wherein a plurality of fusion proteins are capable of self-assembling into an ENP secreted from a cell in which the fusion proteins are expressed, thereby generating a population of ENPs.

In some embodiments, fusion proteins are capable of being presented on the surface of a cell in which the fusion proteins are expressed. In some embodiments, the self-assembly of an ENP: does not require an exogenous nucleic acid other than the nucleic acid composition, does not require any exogenous components other than the plurality of fusion proteins; and/or only requires a single component (e.g., the fusion protein). The cell can be, e.g., a cell of a subject; an in vivo cell, an ex vivo cell, or an in situ cell; and/or an adherent cell or a suspension cell.

In some embodiments, upon secretion from a cell of a subject, the ENPs are capable of distributing within one or more tissues of a subject (e.g., adrenal gland tissue, appendix tissue, bladder tissue, bone, bowel tissue, brain tissue, breast tissue, bronchi, coronal tissue, ear tissue, esophagus tissue, eye tissue, gall bladder tissue, genital tissue, heart tissue, hypothalamus tissue, kidney tissue, large intestine tissue, intestinal tissue, larynx tissue, liver tissue, lung tissue, lymph nodes, mouth tissue, nose tissue, pancreatic tissue, parathyroid gland tissue, pituitary gland tissue, prostate tissue, rectal tissue, salivary gland tissue, skeletal muscle tissue, skin tissue, small intestine tissue, spinal cord, spleen tissue, stomach tissue, thymus gland tissue, trachea tissue, thyroid tissue, ureter tissue, urethra tissue, soft and connective tissue, peritoneal tissue, blood vessel tissue, fat tissue, or any combination thereof). In some embodiments, said ENPs engage a plurality of immune cells in said one or more tissues, thereby mimicking a natural infection.

Disclosed herein include compositions (e.g., vaccine compositions). In some embodiments, the composition comprises: a population of enveloped nanoparticles (ENPs), wherein each of the ENPs comprises a plurality of fusion proteins each comprising an antigenic polypeptide (AP) and an endosomal sorting complex required for transport (ESCRT)-recruiting domain (ERD).

In some embodiments, the ENPs are derived from expression of a nucleic acid composition provided herein. In some embodiments, the ENPs comprise a lipid bilayer (e.g., a lipid bilayer derived from the cell from which the ENP was secreted). In some embodiments, the ERD recruits one or more ESCRT proteins to the cytoplasmic tail of the fusion protein. In some embodiments, the recruitment of ESCRT proteins via the ERD induces the self-assembly and budding of ENPs. In some embodiments, the cytoplasmic portion of the fusion protein comprises the ERD. In some embodiments, the cytoplasmic tail of the fusion protein comprises the ERD. In some embodiments, the ERD interacts with the ESCRT proteins TSG101, NEDD4, and/or ALIX.

In some embodiments, the ERD comprises or is derived from a nonhuman protein, optionally a nonmammalian protein, further optionally a chicken protein, a mouse protein, a lizard protein, a reptile protein, a hamster protein, or a goldfish protein. In some embodiments, the ERD comprises or is derived from the ESCRT and ALIX binding region (EABR) of the human CEP55 protein, optionally residues 170-213. In some embodiments, the ERD comprises or is derived from Syntenin-1, rat Galectin-3 (rGalectin-3), Hrs, and/or CD2AP. In some embodiments, the ERD comprises or is derived from a viral protein, optionally a fragment of a viral protein, further optionally a retroviral protein, herpes simplex viral protein, vaccinia viral protein, hepadnaviral protein, togaviral protein, flaviviral protein, arenaviral protein, coronaviral protein, orthomyxoviral protein, paramyxoviral protein, bunyaviral protein, bornaviral protein, rhabdoviral protein or filoviral protein, optionally a Gag protein, further optionally derived from EIAV, HTLV-1, MLV, or MPMV, optionally EIAV p9 and/or HIV-1 p6. In some embodiments, the ERD comprises or is derived from: an Ebola protein, optionally EBOV VP40. In some embodiments, the ERD comprises an amino acid sequence having at least 65%, 70%, 75%, 80%, 85%, 90%, 95%, 98%, or 99% sequence identity to an amino acid sequence selected from the group consisting of SEQ ID NOs: 1-20 and 23.

The fusion protein can comprise an endocytosis-preventing motif (EPM) capable of preventing endocytosis of the fusion protein. In some embodiments, the EPM: tethers the fusion protein to the cytoskeleton, thereby preventing localization to coated pits and endocytosis; enhances ENP assembly, ENP production, and/or ENP secretion; and/or prevents endocytosis of the fusion protein, thereby extending the time a fusion protein remains at the plasma membrane to interact with ESCRT proteins. In some embodiments, the EPM: increases the abundance and/or density of fusion proteins on and/or in the ENP by at least about 2-fold as compared to a ENP comprising a fusion protein that does not comprise the EPM; and/or increases the number of ENPs secreted by a cell by at least about 2-fold as compared to a cell expressing a fusion protein that does not comprise the EPM. In some embodiments, the EPM comprises or is derived from a portion of murine low-affinity gamma Fc region receptor II isoform FcRII-B1. In some embodiments, the EPM comprises all or a portion of the cytoplasmic tail of FcRII-B1. In some embodiments, the EPM comprises an amino acid sequence having at least 65%, 70%, 75%, 80%, 85%, 90%, 95%, 98%, or 99% sequence identity to the amino acid sequence of SEQ ID NO: 21.

In some embodiments, the AP, or a portion thereof, is displayed in and/or on the surface of the ENP. In some embodiments, the AP is about 1 amino acid to about 10000 amino acids in length. In some embodiments, the AP comprises or is derived from an antigenic protein associated with a disease or disorder, optionally an immunogenic variant and/or an immunogenic fragment of said antigenic protein. In some embodiments, the AP comprises or is derived from a conserved portion of said antigenic protein. In some embodiments, the AP is present on and/or in the ENP in its natural membrane-associated conformation.

In some embodiments, the AP comprises or is derived from at least about 5 percent of the full length of said antigenic protein, optionally the AP comprises or is derived from the full-length surface protein of an infectious agent.

In some embodiments, the disease or disorder is an infectious disease or disorder caused by an infectious agent, wherein the AP comprises or is derived from an antigenic protein of said infectious agent, and wherein the antigenic protein of said infectious agent is a pathogenic antigen. In some embodiments, the disease or disorder is a disease associated with expression of a tumor-associated antigen, and the antigenic protein is a tumor-associated antigen. In some embodiments, the disease or disorder is an autoimmune disease or disorder, and the antigenic protein is an autoimmune antigen. In some embodiments, the disease or disorder is an allergic disease or disorder, and the antigenic protein is an allergenic antigen.

The infectious agent can be a bacterium, a fungus, a virus, or a protist. In some embodiments, the infectious agent is a coronavirus (CoV) (e.g., an alphacoronavirus, a betacoronavirus, a gammacoronavirus, or a deltacoronavirus). In some embodiments, the infectious agent is selected from the group comprising Acinetobacter baumannii, Anaplasma genus, Anaplasma phagocytophilum, Ancylostoma braziliense, Ancylostoma duodenale, Arcanobacterium haemolyticum, Ascaris lumbricoides, Aspergillus genus, Astroviridae, Babesia genus, Bacillus anthracis, Bacillus cereus, Bartonella henselae, BK virus, Blastocystis hominis, Blastomyces dermatitidis, Bordetella pertussis, Borrelia burgdorferi, Borrelia genus, Borrelia spp, Brucella genus, Brugia malayi, Bunyaviridae family, Burkholderia cepacia and other Burkholderia species, Burkholderia mallei, Burkholderia pseudomallei, Caliciviridae family, Campylobacter genus, Candida albicans, Candida spp, Chlamydia trachomatis, Chlamydophila pneumoniae, Chlamydophila psittaci, CJD prion, Clonorchis sinensis, Clostridium botulinum, Clostridium difficile, Clostridium perfringens, Clostridium perfringens, Clostridium spp, Clostridium tetani, Coccidioides spp, coronaviruses, Corynebacterium diphtherias, Coxiella burnetii, Crimean-Congo hemorrhagic fever virus, Cryptococcus neoformans, Cryptosporidium genus, Cytomegalovirus (CMV), Dengue v iruses (DEN-1, DEN-2, DEN-3 and DEN-4), Dientamoeba fragilis, Ebolavirus (EBOV), Echinococcus genus, Ehrlichia chaffeensis, Ehrlichia ewingii, Ehrlichia genus, Entamoeba histolytica, Enterococcus genus, Enterovirus genus, Enteroviruses, mainly Coxsackie A virus and Enterovirus 71 (EV71), Epidermophyton spp, Epstein-Barr Virus (EBV), Escherichia coli O157:H7, O111 and O104:H4, Fasciola hepatica and Fasciola gigantica, FFI prion, Filarioidea superfamily, Filoviruses, Flaviviruses, Francisella tularensis, Fusobacterium genus, Geotrichum candidum, Giardia intestinalis, Gnathostoma spp, GSS prion, Guanarito virus, Haemophilus ducreyi, Haemophilus influenzae, Helicobacter pylori, Henipavirus (Hendra virus Nipah virus), Hepatitis A Virus, Hepatitis B Virus (HBV), Hepatitis C Virus (HCV), Hepatitis D Virus, Hepatitis E Virus, Herpes simplex virus 1 and 2 (HSV-1 and HSV-2), Histoplasma capsulatum, HIV (Human immunodeficiency virus), Hortaea werneckii, Human bocavirus (HBoV), Human herpesvirus 6 (HHV-6) and Human herpesvirus 7 (HHV-7), Human metapneumovirus (hMPV), Human papillomavirus (HPV), Human parainfluenza viruses (HPIV), Japanese encephalitis virus, JC virus, Junin virus, Kingella kingae, Klebsiella granulomatis, Kuru prion, Lassa virus, Legionella pneumophila, Leishmania genus, Leptospira genus, Listeria monocytogenes, Lymphocytic choriomeningitis virus (LCMV), Machupo virus, Malassezia spp, Marburg virus, Measles virus, Metagonimus yokagawai, Microsporidia phylum, Molluscum contagiosum virus (MCV), Mumps virus, Mycobacterium leprae and Mycobacterium lepromatosis, Mycobacterium tuberculosis, Mycobacterium ulcerans, Mycoplasma pneumoniae, Naegleria fowleri, Necator americanus, Neisseria gonorrhoeae, Neisseria meningitidis, Nocardia asteroides, Nocardia spp, Onchocerca volvulus, Orientia tsutsugamushi, Orthomyxoviridae family (Influenza), Paracoccidioides brasiliensis, Paragonimus spp, Paragonimus westermani, Parvovirus B19, Pasteurella genus, Plasmodium genus, Pneumocystis jirovecii, Poliovirus, Rabies virus, Respiratory syncytial virus (RSV), Rhinovirus, rhinoviruses, Rickettsia akari, Rickettsia genus, Rickettsia prowazekii, Rickettsia rickettsii, Rickettsia typhi, Rift Valley fever virus, Rotavirus, Rubella virus, Sabia virus, Salmonella genus, Sarcoptes scabiei, SARS coronavirus, Schistosoma genus, Shigella genus, Sin Nombre virus, Hantavirus, Sporothrix schenckii, Staphylococcus genus, Staphylococcus genus, Streptococcus agalactiae, Streptococcus pneumoniae, Streptococcus pyogenes, Strongyloides stercoralis, Taenia genus, Taenia solium, Tick-borne encephalitis virus (TBEV), Toxocara canis or Toxocara cati, Toxoplasma gondii, Treponema pallidum, Trichinella spiralis, Trichomonas vaginalis, Trichophyton spp, Trichuris trichiura, Trypanosoma brucei, Trypanosoma cruzi, Ureaplasma urealyticum, Varicella zoster virus (VZV), Variola major or Variola minor, vCJD prion, Venezuelan equine encephalitis virus, Vibrio cholerae, West Nile virus, Western equine encephalitis virus, Wuchereria bancrofti, Yellow fever virus, Yersinia enterocolitica, Yersinia pestis, and Yersinia pseudotuberculosis.

In some embodiments, the AP comprises a membrane protein (e.g., a multi-span transmembrane protein). In some embodiments, the AP is not configured to be a soluble protein. In some embodiments, the AP does not comprise one or more mutations configured to enhance its solubility and/or stability. In some embodiments, the AP does not comprise a transmembrane domain and/or is a soluble protein, and wherein the fusion protein comprises a transmembrane domain (TD). In some embodiments, the transmembrane domain comprises or is derived from a nonhuman transmembrane protein (e.g., a nonmammalian transmembrane protein). In some embodiments, the TD comprises or is derived from a natural protein, a recombinant protein, and/or synthetic protein (e.g., a synthetic protein comprising predominantly hydrophobic residues).

The fusion protein can comprise one or more linkers. In some embodiments, the one or more linkers comprise one or more flexible amino acid residues, e.g., about 1 to about 18 flexible amino acid residues, further optionally the flexible amino acid residues comprise glycine, serine, or a combination thereof. The one or more linkers can be a glycine-serine (GS) linker, optionally 1-15 amino acids in length. In some embodiments, the one or more linkers is situated between the ERD and the AP, between the ERD and the EPM, between the ERD and the TD, between the EPM and the TD, between the AP and the EPM, and/or between the AP and the TD.

In some embodiments, at least two of the fusion proteins of the plurality of fusion proteins are different from each other with respect to the AP, and wherein the population of ENPs thereby display a plurality of disparate AP.

In some embodiments, the population of ENPs comprise one or more homotypic ENPs, wherein the plurality of fusion proteins of a homotypic ENP are the same as each other with respect to the AP, and wherein a homotypic ENP thereby does not display a plurality of disparate AP. In some embodiments, the population of ENPs comprise one or more heterotypic ENPs, wherein at least two of the fusion proteins of a heterotypic ENP are different from each other with respect to the AP, and wherein a heterotypic ENP thereby displays a plurality of disparate AP. In some embodiments, the population of ENPs comprise a mixture of two or more homotypic ENPs that differ from each other with respect to the AP of the plurality of fusion proteins present in said two or more homotypic ENPs, and wherein the population of ENPs thereby displays a plurality of disparate AP. In some embodiments, the population of ENPs comprise a mixture of two or more heterotypic ENPs that differ from each other with respect to the AP of the plurality of fusion proteins of said two or more heterotypic ENPs. In some embodiments, heterotypic ENPs are capable of eliciting heterologous antibody responses against an additional infectious agent, and wherein said heterotypic ENPs do not display AP derived from said additional infectious agent.

In some embodiments, the plurality of disparate AP comprises: between about 2 and about 500 antigenic polypeptides that differ from each other; AP of a same protein type; and/or AP of different protein types. In some embodiments, the same ENP comprises the AP derived from two or more strains of the same family, same genus, and/or same species, of infectious agent. In some embodiments, the plurality of disparate AP has a sequence identity of about, at least, or at least about 65%, 70%, 75%, 80%, 85%, 90%, 95%, 98%, or 99% with one another.

In some embodiments, the plurality of disparate AP comprise a plurality of coronavirus (CoV) antigens, wherein the plurality of CoV antigens comprises a first CoV antigen of a first CoV and a second CoV antigen of a second CoV that is different from the first CoV. In some embodiments, the plurality of CoV antigens comprise a CoV spike protein (S protein) or a portion thereof, a CoV envelope protein (E protein) or a portion thereof, a CoV nucleocapsid protein (N protein) or a portion thereof, a CoV hemagglutinin-esterase protein (HE protein) or a portion thereof, a CoV papain-like protease or a portion thereof, a CoV 3CL protease or a portion thereof, a CoV membrane protein (M protein) or a portion thereof, or a combination thereof. In some embodiments, the plurality of CoV antigens comprise a CoV S protein or a portion thereof. In some embodiments, the first CoV antigen, the second CoV antigen, or both comprise a CoV S protein or a portion thereof. In some embodiments, the number of the first CoV antigen molecules and the number of the second CoV antigen molecules are in a ratio from 1:100 to 100:1. In some embodiments, the plurality of CoV antigens comprise three, four, five, size seven, or eight CoV antigens, each of a CoV different from one another. The plurality of CoV antigens can comprise at least a third CoV antigen of a third CoV and a fourth CoV antigen of a fourth CoV, and wherein the first, second, third and fourth CoVs are different from one another.

In some embodiments, the plurality of disparate AP comprise at least m pathogenic antigens of an mth infectious agent, wherein m is an integer greater than 2, and wherein each mth pathogenic antigen is different from one another, optionally m is an integer greater than 50. In some embodiments, the plurality of disparate AP comprise two or more of a 1st pathogenic antigen (PA) of a 1st infectious agent (IA), a 2nd PA of a 2nd IA, a 3rd PA of a 3rd IA, a 4th PA of a 4th IA, a 5th PA of a 5th IA, a 6th PA of a 6th IA, a 7th PA of a 7th IA, a 8th PA of a 8th IA, a 9th PA of a 9th IA, a 10th PA of a 10th IA, a 11th PA of a 11th IA, a 12th PA of a 12th IA, a 13th PA of a 13th IA, a 14th PA of a 14th IA, a 15th PA of a 15th IA, a 16th PA of a 16th IA, a 17th PA of a 17th IA, a 18th PA of a 18th IA, a 19th PA of a 19th IA, a 20th PA of a 20th IA, a 21st PA of a 21st IA, a 22nd PA of a 22nd IA, a 23rd PA of a 23rd IA, a 24th PA of a 24th IA, a 25th PA of a 25th IA, a 26th PA of a 26th IA, a 27th PA of a 27th IA, a 28th PA of a 28th IA, a 29th PA of a 29th IA, a 30th PA of a 30th IA, a 31st PA of a 31st IA, a 32nd PA of a 32nd IA, a 33rd PA of a 33rd IA, a 34th PA of a 34th IA, a 35th PA of a 35th IA, a 36th PA of a 36th IA, a 37th PA of a 37th IA, a 38th PA of a 38th IA, a 39th PA of a 39th IA, a 40th PA of a 40th IA, a 41st PA of a 41st IA, a 42nd PA of a 42nd IA, a 43rd PA of a 43rd IA, a 44th PA of a 44th IA, a 45th PA of a 45th IA, a 46th PA of a 46th IA, a 47th PA of a 47th IA, a 48th PA of a 48th IA, a 49th PA of a 49th IA, and a 50th PA of a 50th IA, wherein the 1st, 2nd, 3rd, 4th, 5th, 6th, 7th, 8th, 9th, 10th, 11th, 12th, 13th, 14th, 15th, 16th, 17th, 18th, 19th, 20th, 21st, 22nd, 23rd, 24th, 25th, 26th, 27th, 28th, 29th, 30th, 31st, 32nd, 33rd, 34th, 35th, 36th, 37th, 38th, 39th, 40th, 41st, 42nd, 43rd, 44th, 45th, 46th, 47th, 48th, 49th, and 50th pathogenic antigens are different from one another. In some embodiments, the 1st, 2nd, 3rd, 4th, 5th, 6th, 7th, 8th, 9th, 10th, 11th, 12th, 13th, 14th, 15th, 16th, 17th, 18th, 19th, 20th, 21st, 22nd, 23rd, 24th, 25th, 26th, 27th, 28th, 29th, 30th, 31st, 32nd, 33rd, 34th, 35th, 36th, 37th, 38th, 39th, 40th, 41st, 42nd, 43rd, 44th, 45th, 46th, 47th, 48th, 49th, and 50th infectious agents are different from one another. In some embodiments, the plurality of disparate AP comprise a plurality of CoV antigens, a plurality of influenza antigens, and/or a plurality of HIV antigens.

The one or more of the plurality of CoV antigens can be of CoVs in the genus of Alpha-CoV and/or Beta-CoV, and optionally wherein each of the plurality of CoV antigens are of CoVs in the genus of Beta-CoV. In some embodiments, the plurality of CoV antigens are of CoVs in the subgenus of Sarbecovirus. In some embodiments, the first CoV and the second CoV are in the genus of Beta-CoV, optionally in the subgenus of Sarbecovirus. In some embodiments, the plurality of CoV antigens are of CoVs selected from the group consisting of: SARS-CoV, SARS-CoV-2, WIV1, SHC014, Rf1, RmYN02, pang17, RaTG13, Rs4081, LYRa11, HKU3, Yunnan2011, BtKY72, BM48-31, WIV16, Khosta-1, and Khosta-2. In some embodiments, the first CoV, the second CoV, or both are selected from the group consisting of: SARS-CoV, SARS-CoV-2, WIV1, SHC014, Rf1, RmYN02, pang17, RaTG13, Rs4081, LYRa11, HKU3, Yunnan2011, BtKY72, BM48-31, WIV16, Khosta-1, and Khosta-2. In some embodiments, the CoV is selected from a species or subspecies of SARS-CoV, SARS-CoV-1, SARS-CoV-2, MERS-CoV, SL-CoV-WIV1, HKU4, HKU5, HCoV-OC43, HCoV-HKU1, HKU9, HKU3, HKU8, HKU24, NL63, SHC014, 229E and/or SARS-CoV-2 variants B.1.351, B.1.1.7, P.1, B.1.617.2, B.1.1.529, BA.1, BA.1.1, BA.2, BA.3, BA.4, BA.5 and other descendent lineages. In some embodiments, the CoV is selected from a species or subspecies of Embecovirus, Sarbecovirus, Merbecovirus, Nobevovirus, Hibecovirus, SARSr-CoV, MERS-CoV, or any combination thereof. In some embodiments, the CoV is selected from a beta-CoV from the sarbe-, embeco-, merbeco-, and/or nobecovirus lineages. In some embodiments, the CoV is selected from a sarbecovirus strain, optionally, SARS, LYRa11, Rf1, Rs4081, BtKY72, and/or BM48-31; In some embodiments, the CoV is selected from a merbecovirus strain, optionally HKU4, HKU5, HKU25, BtCoV-Vs-CoV1, MERS-related NL13845, MERS-related NL140422. In some embodiments, the CoV is selected from an embecovirus strain, optionally HKU1, Rat CoV Parker, PHEV, Equine CoV, Rodent CoV, Longquan Rat CoV.

In some embodiments, the ENPs comprise at least about 2-fold more of the AP and/or are at least as immunogenic as compared to a multi-component nanoparticle approach, optionally as compared to a SpyCatcher-based nanoparticle approach or a lentiviral Gag-based approach, further optionally the multi-component nanoparticle approach comprises two or more separate polypeptides. In some embodiments, the ENPs comprise at least about 2-fold more of the AP, an at least about 2-fold higher density of the AP, and/or are at least as immunogenic, as compared to a nanoparticle approach that does not comprise the ERD, optionally as compared to a SpyCatcher-based or Gag-based nanoparticle approach.

In some embodiments, the nucleic acid composition does not comprise a polynucleotide encoding SpyTag or lentiviral Gag. In some embodiments, the ENPs do not comprise SpyTag or lentiviral Gag.

In some embodiments, the ENPs have one or more dimensions of a eukaryotic virus. In some embodiments, less than about 10% of the ENPs of the population of ENPs have a particle size smaller than about 10 nm. In some embodiments, less than about 10% of the ENPs of the population of ENPs have a particle size exceeding about 80 nm. In some embodiments, the average diameter of the ENPs of the population of ENPs range from about 5 nm to about 80 nm, from about 15 nm to about 50 nm, or from about 20 nm to about 40 nm. In some embodiments, the average diameter of the ENPs of the population of ENPs is about 10 nm, about 12 nm, about 14 nm, about 16 nm, about 18 nm, about 20 nm, about 22 nm, about 24 nm, about 26 nm, about 28 nm, about 30 nm, about 32 nm, about 34 nm, about 36 nm, about 38 nm, about 40 nm, about 42 nm, about 44 nm, about 46 nm, about 48 nm, or about 50 nm, optionally the average is the mean, median or mode, optionally the mean is the arithmetic mean, geometric mean, and/or harmonic mean. In some embodiments, the ENPs have a minimum diameter of about 10 nm, about 12 nm, about 14 nm, about 16 nm, about 18 nm, about 20 nm, about 22 nm, about 24 nm, about 26 nm, about 28 nm, about 30 nm, about 32 nm, about 34 nm, about 36 nm, about 38 nm, about 40 nm, about 42 nm, about 44 nm, about 46 nm, about 48 nm, or about 50 nm. In some embodiments, the ENPs have a maximum diameter of about 10 nm, about 12 nm, about 14 nm, about 16 nm, about 18 nm, about 20 nm, about 22 nm, about 24 nm, about 26 nm, about 28 nm, about 30 nm, about 32 nm, about 34 nm, about 36 nm, about 38 nm, about 40 nm, about 42 nm, about 44 nm, about 46 nm, about 48 nm, about 50 nm, about 52 nm, about 54 nm, about 56 nm, about 58 nm, about 60 nm, about 62 nm, about 64 nm, about 66 nm, about 68 nm, about 70 nm, about 72 nm, about 74 nm, about 76 nm, about 78 nm, or about 80 nm.

In some embodiments, the ENPs are derived from cell cultures transiently transfected with the nucleic acid composition, optionally derived via ultracentrifugation and/or size exclusion chromatography, further optionally ultracentrifugation on a 20% sucrose cushion, optionally transfected via calcium phosphate transfection, DEAE-dextran mediated transfection, cationic lipid-mediated transfection, electroporation, electrical nuclear transport, chemical transduction, electrotransduction, Lipofectamine-mediated transfection, Effectene-mediated transfection, lipid nanoparticle (LNP)-mediated transfection, or any combination thereof. In some embodiments, storage of the ENPs at 4° C. for at least three months reduces immunogenicity less than about 50 percent. In some embodiments, the composition is stable for at least about 2 weeks, about 1 month, about 2 months, about 3 months, about 4 months, about 5 months, about 6 months, or about 1 year, after storage as a liquid at a temperature of about 4° C. In some embodiments, at least about 70%, 75%, 80%, 85%, 90% or 95% of the ENPs are immunogenic at least 1 month after storage as a liquid at a temperature of about 5° C.

In some embodiments, the nucleic acid composition is complexed or associated with one or more lipids or lipid-based carriers, thereby forming liposomes, LNPs, lipoplexes, and/or nanoliposomes, optionally encapsulating the nucleic acid composition. In some embodiments, the nucleic acid composition is, comprises, or further comprises, one or more vectors. In some embodiments, at least one of the one or more vectors is a viral vector, a plasmid, a transposable element, a naked DNA vector, a LNP, or any combination thereof. In some embodiments, the viral vector is an AAV vector, a lentivirus vector, a retrovirus vector, an adenovirus vector, a herpesvirus vector, a herpes simplex virus vector, a cytomegalovirus vector, a vaccinia virus vector, a MVA vector, a baculovirus vector, a vesicular stomatitis virus vector, a human papillomavirus vector, an avipox virus vector, a Sindbis virus vector, a VEE vector, a Measles virus vector, an influenza virus vector, a hepatitis B virus vector, an integration-deficient lentivirus (IDLV) vector, or any combination thereof. In some embodiments, the transposable element is piggybac transposon or sleeping beauty transposon.

The polynucleotide(s) encoding fusion protein(s) can be comprised in the one or more vectors. In some embodiments, the polynucleotide(s) encoding fusion protein(s) are comprised in the same vector and/or different vectors. In some embodiments, the polynucleotide(s) encoding fusion protein(s) are situated on the same nucleic acid and/or different nucleic acids.

In some embodiments, the one or more vectors is a DNA vaccine. In some embodiments, the polynucleotide(s) encoding fusion protein(s) are operably linked to one or more promoters capable of inducing transcription of said polynucleotide(s). In some embodiments, the DNA vaccine is a plasmid-based DNA vaccine, a minicircle-based DNA vaccine, a bacmid-based DNA vaccine, a minigene-based DNA vaccine, a ministring DNA (linear covalently closed DNA vector) vaccine, a closed-ended linear duplex DNA (CELiD or ceDNA) vaccine, a doggybone™ DNA vaccine, a dumbbell shaped DNA vaccine, or a minimalistic immunological-defined gene expression (MIDGE)-vector DNA vaccine. In some embodiments, the DNA vaccine elicits at least 2-fold higher neutralizing antibody responses against an infectious agent as compared to a DNA vaccine that encodes the AP but not the ERD.

In some embodiments, the promoter comprises a ubiquitous promoter, an inducible promoter, a tissue-specific promoter and/or a lineage-specific promoter. In some embodiments, the ubiquitous promoter is selected from the group comprising a cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EFIa) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus, a Ubiquitin C promoter (UBC), a phosphoglycerate kinase-1 (PGK) promoter, 3-phosphoglycerate kinase promoter, a cytomegalovirus enhancer, human β-actin (HBA) promoter, chicken β-actin (CBA) promoter, a CAG promoter, a CASI promoter, a CBH promoter, or any combination thereof.

In some embodiments, the polynucleotide(s) encoding fusion protein(s) are operably linked to a tandem gene expression element (e.g., an internal ribosomal entry site (IRES), foot-and-mouth disease virus 2A peptide (F2A), equine rhinitis A virus 2A peptide (E2A), porcine teschovirus 2A peptide (P2A) or Thosea asigna virus 2A peptide (T2A), or any combination thereof). In some embodiments, the polynucleotide(s) encoding fusion protein(s) comprises a transcript stabilization element (e.g., woodchuck hepatitis post-translational regulatory element (WPRE), bovine growth hormone polyadenylation (bGH-polyA) signal sequence, human growth hormone polyadenylation (hGH-polyA) signal sequence, or any combination thereof).

The nucleic acid composition can be or can comprise mRNA. In some embodiments, the mRNA is formulated in a LNP. In some embodiments, the mRNA comprises a 5′ untranslated region (UTR), a 3′ UTR, and/or a cap. The mRNA can comprise one or more modified nucleotides selected from the group comprising pseudouridine, N-1-methyl-pseudouridine, 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine. In some embodiments, the mRNA comprises a modified nucleotide in place of one or more uridines. In some embodiments, the modified nucleoside is selected from pseudouridine (ψ), N 1-methyl-pseudouridine (m PF), and 5-methyl-uridine (m5U).

In some embodiments, the LNP comprises one or more of an ionizable cationic lipid, a non-cationic lipid, a sterol, and a PEG-modified lipid, optionally the non-cationic lipid is a neutral lipid. In some embodiments, the LNP comprises 0.5-15 mol % PEG-modified lipid, 5-25 mol % non-cationic lipid, 25-55 mol % sterol, and 20-60 mol % ionizable cationic lipid. In some embodiments, the LNP comprises 40-55 mol % ionizable cationic lipid, 5-15 mol % neutral lipid, 35-45 mol % sterol, and 1-5 mol % PEG-modified lipid.

In some embodiments, the LNP comprises: 47 mol % ionizable cationic lipid, 11.5 mol % neutral lipid, 38.5 mol % sterol, and 3.0 mol % PEG-modified lipid; 48 mol % ionizable cationic lipid, 11 mol % neutral lipid, 38.5 mol % sterol, and 2.5 mol % PEG-modified lipid; 49 mol % ionizable cationic lipid, 10.5 mol % neutral lipid, 38.5 mol % sterol, and 2.0 mol % PEG-modified lipid; 50 mol % ionizable cationic lipid, 10 mol % neutral lipid, 38.5 mol % sterol, and 1.5 mol % PEG-modified lipid; or 51 mol % ionizable cationic lipid, 9.5 mol % neutral lipid, 38.5 mol % sterol, and 1.0 mol % PEG-modified lipid.

In some embodiments, the ionizable cationic lipid is heptadecan-9-yl 8 ((2 hydroxyethyl)(6 oxo 6-(undecyloxy)hexyl)amino)octanoate; the neutral lipid is 1,2 distearoyl sn glycero-3 phosphocholine (DSPC); the sterol is cholesterol; and/or the PEG-modified lipid is 1-monomethoxypolyethyleneglycol-2,3-dimyristylglycerol with polyethylene glycol of average molecular weight 2000 (PEG2000 DMG). In some embodiments, the wt/wt ratio of lipid to mRNA is from about 1:100 to about 100:1.

In some embodiments, (i) the nucleic acid composition comprises one or more polynucleotides encoding immunostimulatory agents; and/or (ii) the population of ENPs comprises one or more immunostimulatory agents (e.g., toll-like receptor (TLR) agonists, cytokine receptor agonists, CD40 agonists, Fc receptor agonists, CpG-containing nucleic acids, complement receptor agonists, or any combination thereof). In some embodiments, the TLR agonist is a TLR-1 agonist, TLR-2 agonist, TLR-3 agonist, TLR-4 agonist, TLR-5 agonist, TLR-6 agonist, TLR-7 agonist, TLR-8 agonist, TLR-9 agonist, and/or TLR-10 agonist; the Fc receptor agonist is a Fc-gamma receptor agonist; the complement receptor agonist binds to CD21 or CD35; the complement receptor agonist induces endogenous complement opsonization of the ENP; the cytokine receptor agonist is a cytokine; and/or the cytokine receptor agonist is a small molecule, antibody, fusion protein, or aptamer.

In some embodiments, the composition comprises Tris buffer, sucrose, and/or sodium acetate. In some embodiments, the composition comprises an adjuvant (e.g., aluminum hydroxide, alhydrogel, AddaVax, MF59, AS03, Freund's adjuvant, Montanide ISA51, CpG, Poly I:C, glucopyranosyl lipid A, flagellin, resiquimod, or any combination thereof). In some embodiments, the composition is a lyophilized composition. In some embodiments, the lyophilized composition has a water content of less than about 10%.

In some embodiments, the composition is formulated or is to be formulated: as a liquid, a solid, or a combination thereof; for injection; for intramuscular administration, intranasal administration, transdermal administration, aerosol delivery, nasal delivery, vaginal delivery, rectal delivery, buccal delivery, ocular delivery, local delivery, topical delivery, intracisternal delivery, intraperitoneal delivery, oral delivery, intramuscular injection, intravenous injection, subcutaneous injection, intranodal injection, intratumoral injection, intraperitoneal injection, intradermal injection; and/or as particles (e.g., iron oxide particles, liposomes, micelles, polymer complexes, cationic peptide nanoemulsions, virus-like particles (VLPs), LNPs and/or lipoplex (LPX) particles). In some embodiments, the nucleic acid composition and the LNP-forming components are in separate vials.

In some embodiments, the composition is a pharmaceutical composition, wherein the pharmaceutical composition comprises one or more pharmaceutically acceptable carriers, diluents and/or excipients. In some embodiments, the composition comprises instructions for use of the composition for: stimulating an immune response in a subject in need thereof; treating or preventing a disease or disorder caused by an infectious agent in a subject in need thereof; and/or treating or preventing a CoV infection in a subject in need thereof.

Disclosed herein include kits. In some embodiments, the kit comprises: a composition disclosed herein (e.g., a nucleic acid composition, a population of ENPs). Disclosed herein include cells. In some embodiments, a cell comprises: a nucleic acid composition disclosed herein.

Disclosed herein include methods of stimulating an immune response in a subject in need thereof. In some embodiments, the method comprises: administering to the subject a pharmaceutically effective amount of a composition disclosed herein (e.g., a nucleic acid composition, a population of ENPs), thereby stimulating an immune response in the subject.

Disclosed herein include methods of treating or preventing a disease or disorder in a subject in need thereof. In some embodiments, the method comprises: administering to the subject a pharmaceutically effective amount of a composition disclosed herein (e.g., a nucleic acid composition, a population of ENPs), thereby treating or preventing the disease or disorder in the subject. In some embodiments, the disease or disorder is a disease or disorder caused by an infectious agent. In some embodiments, the disease or disorder caused by an infectious agent is a disease or disorder caused by a coronavirus (CoV) infection.

Disclosed herein include methods for treating or preventing a CoV infection in a subject in need thereof. In some embodiments, the method comprises: administering to the subject a pharmaceutically effective amount of a composition disclosed herein (e.g., a nucleic acid composition, a population of ENPs), thereby treating or preventing the CoV infection in the subject.

In some embodiments, immunogenic levels of the fusion protein and/or ENP are produced in serum of the subject at about 1 hour to about 6 months post administration of the composition. In some embodiments, a neutralizing antibody titer of about 50 to about 100000 half-maximal inhibitory dilutions (ID₅₀s values) is produced in the serum of the subject at about 1 hour to about 6 months post administration of the composition. In some embodiments, the composition elicits at least about 2-fold less off-target immune responses against undesired epitopes as compared to a non-enveloped NP-based composition. In some embodiments, said undesired epitopes comprise the NP scaffold of said non-enveloped NP-based composition. In some embodiments, the method comprises administering to the subject at least two doses of the composition. In some embodiments, the second dose of the composition is administered to the subject at least 14 days after a first dose of the composition is administered to the subject.

In some embodiments, administering the composition induces neutralizing responses against the infectious agent(s) from which the antigenic polypeptide(s) are derived; and/or additional infectious agent(s) from which the antigenic polypeptide(s) are not derived, optionally different from the 1st, 2nd, 3rd, 4th, 5th, 6th, 7th, 8th, 9th, 10th, 11th, 12th, 13th, 14th, 15th, 16th, 17th, 18th, 19th, 20th, 21st, 22nd, 23rd, 24th, 25th, 26th, 27th, 28th, 29th, 30th, 31st, 32nd, 33rd, 34th, 35th, 36th, 37th, 38th, 39th, 40th, 41st, 42nd, 43rd, 44th, 45th, 46th, 47th, 48th, 49th, and/or 50th infectious agent.

In some embodiments, administering the composition induces neutralizing responses against: the coronaviruses the plurality of coronavirus antigens are of; coronaviruses different from the 1st, 2nd, 3rd, 4th, 5th, 6th, 7th, 8th, 9th, 10th, 11th, 12th, 13th, 14th, 15th, 16th, 17th, 18th, 19th, 20th, 21st, 22nd, 23rd, 24th, 25th, 26th, 27th, 28th, 29th, 30th, 31st, 32nd, 33rd, 34th, 35th, 36th, 37th, 38th, 39th, 40th, 41st, 42nd, 43rd, 44th, 45th, 46th, 47th, 48th, 49th, and/or 50th CoV; and/or additional coronaviruses different from the coronaviruses the plurality of coronavirus antigens are of.

In some embodiments, administering the composition results in treating or preventing: infection caused by a coronavirus different from the first coronavirus and the second coronavirus; infection caused by additional coronaviruses different from the coronaviruses the plurality of coronavirus antigens are of infection caused by the coronaviruses the plurality of coronavirus antigens are of the disease or disorder caused by a coronavirus different from the first coronavirus and the second coronavirus; the disease or disorder caused by additional coronaviruses different from the coronaviruses the plurality of coronavirus antigens are of; and/or the disease or disorder caused by the coronaviruses the plurality of coronavirus antigens are of. In some embodiments, the composition elicits an at least 2-fold higher neutralizing antibody titer as compared to an approach comprising administration of (i) a soluble version of the AP, and/or (ii) an mRNA vaccine encoding the AP and not encoding the ERD. In some embodiments, the composition elicits an at least as high neutralizing antibody titer as compared to an approach comprising administration of a protein-based nanoparticle presenting the AP. In some embodiments, administration of the composition elicits protective and long-lasting immunity against the infectious agent(s) and variants thereof.

In some embodiments, an at least as low dose of the composition is needed to generate a comparable immune response as compared to an approach comprising administration of a protein-based nanoparticle presenting the AP. In some embodiments, an at least about 2-fold lower dose of the composition is needed to generate a comparable immune response as compared to an approach comprising administration of (i) a soluble version of the AP, and/or (ii) an mRNA vaccine encoding the AP and not encoding the ERD.

In some embodiments, following administration of a first dose or second dose of the composition, the composition induces: (i) an at least as potent serum neutralizing titers against the infectious agent or variants thereof as compared to an approach comprising administration of a protein-based nanoparticle presenting the AP, optionally the composition comprises a population of ENPs; (ii) an at least 2-fold more potent serum neutralizing titers against the infectious agent or variants thereof as compared to an approach comprising administration of a soluble version of the AP, optionally the composition comprises a population of ENPs; and/or (iii) an at least 2-fold more potent serum neutralizing titers against the infectious agent or variants thereof as compared to an approach comprising administration of an mRNA vaccine encoding the AP and not encoding the ERD, optionally the composition comprises an mRNA vaccine encoding a fusion protein comprising the AP. In some embodiments, potency of serum neutralizing titers are measured by geometric means for serum half-maximal inhibitory dilutions (ID50s values) against the infectious agent or variants thereof, optionally about 1 day to about 6 months after administration of a first dose of the composition or about 1 day to about 6 months after administration of a second dose of the composition.

In some embodiments, the subject: is a human subject; is a newborn or infant of an age of not more than 3 years, of not more than 2 years, of not more than 1.5 years, of not more than 1 year (12 months), of not more than 9 months, 6 months or 3 months, or is between 6 months and 2 years; is immunocompromised, has a pulmonary disease, and/or is 65 years of age or older; has a chronic pulmonary disease, optionally chronic obstructive pulmonary disease (COPD) or asthma; and/or has an underlying comorbid condition, optionally selected from heart disease, diabetes, and lung disease.

In some embodiments, the composition is administered in an effective amount to: (i) induce a robust antibody response against the AP in the subject, optionally a robust antibody response comprises a neutralizing antibody response, further optionally a robust antibody response comprises Fc domain effector functions that recruit immune cells to infected cells, optionally said immune cells are macrophages, neutrophils, and/or natural killer cells, further optionally said recruitment induces antibody-dependent cellular cytotoxicity (ADCC) and/or antibody-dependent cellular phagocytosis (ADCP); (ii) elicit a robust CD4 and/or CD8 T cell response against the AP in the subject; and/or (iii) elicit a balanced Th1/Th2 response against the AP in the subject. In some embodiments, the composition is: (i) co-administered with an adjuvant; or (ii) not co-administered with an adjuvant.

The disease or disorder can be a blood disease, an immune disease, a neurological disease or disorder, a cancer, an infectious disease, a genetic disease, a disorder caused by aberrant mtDNA, a metabolic disease, a disorder caused by aberrant cell cycle, a disorder caused by aberrant angiogenesis, a solid tumor, a disorder cause by aberrant DNA damage repair, or any combination thereof.

The disease or disorder can be an infectious disease selected from Acute Flaccid Myelitis (AFM), Anaplasmosis, Anthrax, Babesiosis, Botulism, Brucellosis, Campylobacteriosis, Carbapenem-resistant Infection, Chancroid, Chikungunya Virus Infection, Chlamydia, Ciguatera, Difficile Infection, Perfringens, Coccidioidomycosis fungal infection, coronavirus infection, Covid-(SARS-CoV-2), Creutzfeldt-Jacob Disease/transmissible spongiform encephalopathy, Cryptosporidiosis (Crypto), Cyclosporiasis, Dengue 1,2,3 or 4, Diphtheria, E. coli infection/Shiga toxin-producing (STEC), Eastern Equine Encephalitis, Hemorrhagic Fever (Ebola), Ehrlichiosis, Encephalitis, Arboviral or parainfectious, Non-Polio Enterovirus, D68 Enteroviru (EV-D68), Giardiasis, Glanders, Gonococcal Infection, Granuloma inguinale, Haemophilus Influenza disease Type B (Hib or H-flu), Hantavirus Pulmonary Syndrome (HPS), Hemolytic Uremic Syndrome (HUS), Hepatitis A (Hep A), Hepatitis B (Hep B), Hepatitis C (Hep C), Hepatitis D (Hep D), Hepatitis E (Hep E), Herpes, Herpes Zoster (Shingles), Histoplasmosis infection, Human Immunodeficiency Virus/AIDS (HIV/AIDS), Human Papillomavirus (HPV), Influenza (Flu), Legionellosis (Legionnaires Disease), Leprosy (Hansens Disease), Leptospirosis, Listeriosis (Listeria), Lyme Disease, Lymphogranuloma venereum infection (LGV), Malaria, Measles, Melioidosis, Meningitis (Viral), Meningococcal Disease (Meningitis (Bacterial)), Middle East Respiratory Syndrome Coronavirus (MERS-CoV), Mumps, Norovirus, Pediculosis, Pelvic Inflammatory Disease (PID), Pertussis (Whooping Cough), Plague (Bubonic, Septicemic, Pneumonic), Pneumococcal Disease (Pneumonia), Poliomyelitis (Polio), Powassan, Psittacosis, Pthiriasis, Pustular Rash diseases (Small pox, monkeypox, cowpox), Q-Fever, Rabies, Rickettsiosis (Rocky Mountain Spotted Fever), Rubella (German Measles), Salmonellosis gastroenteritis (Salmonella), Scabies, Scombroid, Sepsis, Severe Acute Respiratory Syndrome (SARS), Shigellosis gastroenteritis (Shigella), Smallpox, Staphyloccal Infection Methicillin-resistant (MRSA), Staphylococcal Food Poisoning Enterotoxin B Poisoning (Staph Food Poisoning), Saphylococcal Infection Vancomycin Intermediate (VISA), Staphylococcal Infection Vancomycin Resistant (VRSA), Streptococcal Disease Group A (invasive) (Strep A (invasive), Streptococcal Disease, Group B (Strep-B), Streptococcal Toxic-Shock Syndrome STSS Toxic Shock, Syphilis (primary, secondary, early latent, late latent, congenital), Tetanus Infection, Trichomoniasis, Trichonosis Infection, Tuberculosis (TB), Tuberculosis Latent (LTBI), Tularemia, Typhoid Fever Group D, Vaginosis, Varicella (Chickenpox), Vibrio cholerae (Cholera), Vibriosis (Vibrio), Ebola Virus Hemorrhagic Fever, Lasa Virus Hemorrhagic Fever, Marburg Virus Hemorrhagic Fever, West Nile Virus, Yellow Fever, Yersenia, and Zika Virus Infection.

The disease can be associated with expression of a tumor-associated antigen (e.g., a proliferative disease, a precancerous condition, a cancer, and a non-cancer related indication associated with expression of the tumor antigen). In some embodiments, the cancer is selected from the group consisting of colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of said cancers, and metastatic lesions of said cancers. In some embodiments, the cancer is a hematologic cancer chosen from one or more of chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CIVIL), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or pre-leukemia.

In some embodiments, administering comprises aerosol delivery, nasal delivery, vaginal delivery, rectal delivery, buccal delivery, ocular delivery, local delivery, topical delivery, intracisternal delivery, intraperitoneal delivery, oral delivery, intramuscular injection, intravenous injection, subcutaneous injection, intranodal injection, intratumoral injection, intraperitoneal injection, intradermal injection, or any combination thereof. In some embodiments, the composition is administered intramuscularly (e.g., into a deltoid region of an arm).

### BRIEF DESCRIPTION OF THE DRAWINGS

FIG. 1A-FIG. 1G depict data related to the generation of self-assembling SARS-CoV-2 S-EABR NPs (e.g., ENPs). FIG. 1A shows a non-limiting exemplary schematic presenting mechanisms of self-assembling S-EABR NP technology. The EABR domain is fused to the cytoplasmic tail of the SARS-CoV-2 S protein and recruits host proteins from the ESCRT pathway, which induces self-assembly and budding of enveloped S-EABR NPs. FIG. 1B shows a simplified map of the SARS-CoV-2 S-p6, S-VP40, S-p9, S-EABR, S-EABRₘᵢₙ₁, and S-EABRₘᵢₙ₂ constructs. FIG. 1C depicts a representative cryo-electron tomogram showing S-EABR NPs that were purified by sucrose ultracentrifugation and size-exclusion chromatography. Scale bar=20 nm. FIG. 1D shows western blot analysis of SARS-CoV-2 S-containing NPs that were generated by transfecting Expi293 cells with S (lanes 1 and 5), S+Gag (lanes 2 and 6), S+SARS-CoV-2 structural proteins M, N, and E (lanes 3 and 7), or S-EABR (lane 8). NPs were purified from culture supernatants by sucrose ultracentrifugation and size-exclusion chromatography. Sample dilutions of 1:20 were loaded for lanes 1-3 (lane 4 is empty), and dilutions of 1:200 were loaded for lanes 5-8. FIG. 1E shows western blot analysis of purified SARS-CoV-2 S-containing NPs that were generated by transfecting Expi293 cells with S-p6 (lane 1), S-VP40 (lane 2), S-p9 protein (lane 3), or S-EABR (lane 4). The samples in lanes 1-3 were diluted 1:40, while the S-EABR sample in lane 4 was diluted 1:400. FIG. 1F shows western blot analysis of purified SARS-CoV-2 S-containing NPs that were generated by transfecting Expi293 cells with S-EABR (lane 1), S fused to two EABR domains (S-2XEABR) (lane 2), or S-EABR containing a Y187A mutation (lanes 3-4). Samples in lanes 1-3 were diluted 1:200, while the sample in lane 4 was diluted 1:20. FIG. 1G shows western blot analysis of SARS-CoV-2 S-containing NPs that were generated by transfection of S-EABR, S-EABRₘᵢₙ₁, and S-EABRₘᵢₙ₂. All samples were diluted 1:50.

FIG. 2 shows western blot analysis of SARS-CoV-2 S-containing NPs that were generated by transfection of S-EABRₘᵢₙ₁, ₘᵤEABRₘᵤₜ₁, _{chic}EABRₘᵤₜ₁, _{chic}EABRₘᵤₜ₂, _{cham}EABRₘᵤₜ₁, and _{gold}EABRₘᵤₜ₁ (Also, See, Table 1, FIG. 15). Samples were diluted 1:50 or 1:200 as indicated.

FIG. 3A-FIG. 3D show results from western blot analysis of SARS-CoV-2 S-containing NPs that were generated by transfection of (FIG. 3A) S-EABRₘᵢₙ₁, S-Syntenin-1₂₋₆₀, S-rGalectin-3; (FIG. 3B) S-EABRₘᵢₙ₁, S-rGalectin-3, S-rGalectin-3ₘᵢₙ₁, S-rGalectin-3ₘᵢₙ₂; (FIG. 3C) S-EABR, S-Hrs, S-CD2APₘᵢₙ₁, CD2APₘᵢₙ₂; (FIG. 3D) S-p6, S-HTLV-1₁₁₁₋₁₃₀, S-MLV₁₀₈₋₁₇₇, S-MPMV₁₉₇₋₂₁₅. Samples were diluted 1:50, 1:200, or 1:20 as indicated. Also, See, Table 2 and FIG. 16.

FIG. 4A-FIG. 4C depict non-limiting exemplary data showing that the FcR domain prevents endocytosis and enhances S-EABR NP assembly. FIG. 4A shows a simplified map of the SARS-CoV-2 S-EABR and S-FcR-EABR constructs. The amino acid sequences for the FcR and EABR domains are presented. FIG. 4B shows western blot analysis comparing S-containing NPs after transfecting Expi293 cells with the S-EABR (lane 1) or S-FcR-EABR (lane 2) constructs. Both samples were diluted 1:200. FIG. 4C shows western blot analysis of CD4-containing NPs after transfecting Expi293 cells with CD4 (lane 1), CD4+Gag (lane 2), CD4-EABR (lane 3), or CD4-FcR-EABR (lane 4). All samples were purified by sucrose ultracentrifugation and diluted 1:100.

FIG. 5A-FIG. 5C depict non-limiting exemplary embodiments showing that EABR NPs can be generated for a wide range of membrane proteins. FIG. 5A shows results from western blot analysis for SARS, HKU1, MERS, and 229E S-EABR NPs that were purified by sucrose ultracentrifugation and size exclusion chromatography. All S-EABR constructs were detected through a C-terminal myc-tag and samples were diluted 1:400. FIG. 5B shows western blot analysis for purified HIV-1 Env-containing NPs generated by transfecting Expi293 cells with HIV-1 Env (lane 1), Env+Gag (lane 2), or Env-EABR (lane 3). All samples were diluted 1:200. FIG. 5C shows results from western blot analysis for purified CCR5-containing NPs generated by transfecting Expi293 cells with CCR5 (lane 1), CCR5+Gag (lane 2), or CCR5-EABR (lane 3). All samples were diluted 1:200.

FIG. 6A-FIG. 6F depicts non-limiting exemplary data showing that SARS-CoV-2 S-EABR NPs elicit potent antibody responses in vivo. FIG. 6A-FIG. 6B show data of SARS-CoV-2 neutralization for serum samples from C57BL/6 mice immunized with soluble S, RBD-mi3 NPs, and S-EABR NPs. Potencies are presented as half-maximal inhibitory dilutions (ID₅₀ values) and are shown for (FIG. 6A) post-prime (day 14) and (FIG. 6B) post-boost (day 42) samples. The dashed horizontal line corresponds to the limit of detection. Data points represent ID₅₀s for individual animals and rectangles represent mean ID₅₀s for 8 animals per group with SDs shown as vertical lines. Statistical significance (p<0.05) between groups linked by horizontal lines is indicated by asterisks. FIG. 6C shows results from PRNT assays to determine the neutralization activity against authentic SARS-CoV-2 virus for post-boost (day 42) serum samples from C57BL/6 mice immunized with S-EABR NPs. PRNT assays were performed against the early pandemic Wuhan strain, as well as the Beta and Delta VOCs. Data points represent ID₅₀s for individual animals and rectangles represent mean ID₅₀s for 8 animals per group with SDs shown as vertical lines. FIG. 6D shows ELISA data for IgG responses against SARS-CoV-2 S for serum samples from mice immunized with S-2P-EABR NPs, S-6P-EABR NPs, and S-6P-EABR NPs that were stored at 4° C. for 2 months. Serum samples were taken 14 days after a single injection of the respective immunogen and results are shown as area under the curve (AUC) for individual animals and rectangles represent mean AUC for 6-8 animals per group with SDs shown as vertical lines. FIG. 6E-FIG. 6F show SARS-CoV-2 neutralization for post-boost (day 42) serum samples from BALB/c mice immunized with an mRNA vaccine encoding SARS-CoV-2 S or S-EABR NPs against the (FIG. 6E) Wuhan and (FIG. 6F) Omicron variants. Potencies are presented as half-maximal inhibitory dilutions (ID₅₀ values). The dashed horizontal line corresponds to the limit of detection. Data points represent ID₅₀s for individual animals and rectangles represent mean ID₅₀s for 10 animals per group with SDs shown as vertical lines. Statistical significance (p<0.05) between groups linked by horizontal lines is indicated by asterisks.

FIG. 7A-FIG. 7E depict non-limiting exemplary embodiments for self-assembling S-EABR NPs as a platform technology for the development of hybrid mRNA vaccines with enhanced potency. Shown in FIG. 7A-FIG. 7B are exemplary schematics of immune activation mechanisms for S (FIG. 7A) and S-EABR (FIG. 7B) mRNA vaccines delivered by lipid nanoparticles (LNPs). FIG. 7C shows data of post-boost (day 36) neutralization against SARS-CoV-2 for serum samples from BALB/c mice immunized with 10 μg S DNA, 10 μg S-EABR DNA, or 1 μg purified S-EABR NPs plus adjuvant. FIG. 7D-FIG. 7E show graphs of post-boost (day 42) neutralization against (FIG. 7D) SARS-CoV-2 Wuhan strain and the (FIG. 7E) Delta VOC for serum samples from C57BL/6 mice immunized with 1 μg S mRNA, 1 μg of the combination of S+S-EABR mRNA (0.5 μg S+0.5 μg S-EABR), or 1 μg purified S-EABR NPs plus adjuvant. Potencies for data shown in FIG. 7C-FIG. 7E are presented as half-maximal inhibitory dilutions (ID₅₀ values). Dashed horizontal lines correspond to the detection limit. Data points represent ID₅₀s for individual animals and rectangles represent geometric mean ID₅₀s for 6 animals per group with SDs shown as vertical lines. Statistical significance (p<0.05) between groups linked by a horizontal line is indicated by an asterisk.

FIG. 8A-FIG. 8I depict exemplary embodiments showing production of S-EABR NPs for various CoV strains. Western blot analysis is shown for purified (FIG. 8A) SARS-CoV-2 RBD-EABR NPs, (FIG. 8B) SARS-CoV-2 S-2P-EABR NPs (B.1.351 variant), (FIG. 8C) SARS-CoV and HKU-1 S-EABR NPs, (FIG. 8D) Rf1 and HKU-4 S-EABR NPs, (FIG. 8E) 229E and BtKY72 S-EABR NPs, (FIG. 8F) MERS-CoV S-EABR NPs, and (FIG. 8G) NL63 S-EABR NPs. FIG. 8H-FIG. 8I show results from western blot analysis for purified mosaic S-EABR NPs consisting of (FIG. 8H) SARS-CoV, Rf1, BtKY72, HKU-1, HKU-4, and 229E S-EABR or (FIG. 8I) SHC014, HKU-3, HKU-5, HKU-8, HKU-24, and BM48-31 S-EABR. All S-EABR NPs were purified by sucrose ultracentrifugation and size-exclusion chromatography.

FIG. 9A-FIG. 9D depict non-limiting exemplary data showing that mosaic S-EABR NPs elicit heterologous antibody responses against SARS-CoV-2, MERS-CoV, and SHC014. FIG. 9A shows a schematic comparing different approaches to designing a universal CoV vaccine. Shown on the left is a schematic of Homotypic S-EABR NPs that present the S protein from a single CoV strain. The middle schematic displays a cocktail of homotypic S-EABR NPs that each present the S protein from a single CoV strain. Shown on the right are heterotypic mosaic S-EABR NPs that display S proteins from multiple CoV strains on the same NP. FIG. 9B-FIG. 9D ELISA show results against (FIG. 9B) SARS-CoV-2 S and (FIG. 9C) MERS-CoV S, and neutralization data against (FIG. 9D) lentivirus-based SHC014 pseudovirus for post-boost (day 42) serum samples from mice immunized with SARS S-EABR NPs, a cocktail of SARS, Rf1, BtKY72, HKU1, HKU4, and 229E S-EABR NPs, or mosaic S-EABR NPs generated by co-transfection of SARS, Rf1, BtKY72, HKU1, HKU4, and 229E S-EABR constructs. Results are shown as area under the curve (AUC) for ELISAs and half-maximal inhibitory dilutions (ID₅₀ values) for neutralization assays for individual animals and rectangles represent mean values for 8 animals per group with SDs shown as vertical lines. Statistical significance (p<0.05) between groups linked by horizontal lines are indicated by asterisks.

FIG. 10 shows a non-limiting schematic presenting the mechanism of the self-assembling S-EABR NP technology compared to a conventional approach that requires co-expression of a structural scaffold protein that assembles the NP, e.g., Gag.

FIG. 11A-FIG. 11B show data related to generation of self-assembling nanoparticle. FIG. 11A shows western blot analysis of SARS-CoV-2 S-containing NPs that were generated by transfecting Expi293 cells with S (lane 1), S+Gag (lane 2), S+SARS-CoV-2 structural proteins M, N, and E (lane 3), S-EABR (lane 4), or S-FcR-EABR (lane 5). NPs were purified from culture supernatants by sucrose ultracentrifugation. Samples in lanes 1-3 were diluted 1:10-fold, while samples in lanes 4-5 were diluted 1:200-fold. FIG. 11B shows western blot analysis of purified SARS-CoV-2 S-containing NPs that were generated by transfecting Expi293 cells with S-EABR (lane 1), S fused to the EIAV p9 protein (lane 2), S fused to the EBOV VP40 protein (lane 3), or S fused to the HIV-1 p6 protein (lane 4). The sample in lane 1 was diluted 1:400-fold, while samples in lanes 2-4 were diluted 1:40-fold.

FIG. 12 shows ELISA data for IgG responses against SARS-CoV-2 S for serum samples from mice immunized with S-2P-EABR NPs using either Sigma adjuvant or AddaVax adjuvant. All analyzed serum samples were taken 14 days after a single injection of the respective immunogen and results are shown as area under the curve (AUC) for individual animals and rectangles represent mean AUC for 6-8 animals per group with SDs shown as vertical lines.

FIG. 13A-FIG. 13B depict non-limiting exemplary data showing that mosaic S-EABR NPs elicit heterologous antibody responses against SARS-CoV-2 and MERS-CoV. Shown is ELISA data for IgG responses against (FIG. 13A) SARS-CoV-2 S and (FIG. 13B) MERS-CoV S for serum samples from mice immunized with SARS-CoV S-EABR NPs, an admixture (admix) of SARS-CoV, Rf1, BtKY72, HKU-1, HKU-4, and 229E S-EABR NPs, or mosaic S-EABR NPs generated by co-transfection of SARS-CoV, Rf1, BtKY72, HKU-1, HKU-4, and 229E S-EABR. All analyzed serum samples were taken 14 days after a single injection of the respective immunogen and results are shown as area under the curve (AUC) for individual animals and rectangles represent mean AUC for 8 animals per group with SDs shown as vertical lines. Statistical significance (p<0.05) between groups linked by horizontal lines are indicated by asterisks.

FIG. 14 depicts an exemplary schematic showing immune activation mechanisms for a potential SARS-CoV-2 S-EABR mRNA vaccine delivered by S-EABR NPs.

FIG. 15 depicts the amino acid sequences for the EABR domain used herein (EABR, SEQ ID NO: 4), as well as ₘᵤEABRₘᵤₜ₁ (SEQ ID NO: 7), _{chic}EABRₘᵤₜ₁ (SEQ ID NO: 8), _{chic}EABRₘᵤₜ₂ (SEQ ID NO: 9), _{cham}EABRₘᵤₜ₁ (SEQ ID NO: 10), and _{gold}EABRₘᵤₜ₁ (SEQ ID NO: 11). Residues that differ from the human EABR sequence are in gray text.

FIG. 16 depicts amino acid sequences for the ESCRT-binding domains Syntenin-1₂₋₆₀ (SEQ ID NO: 12), rGalectin-3 (SEQ ID NO: 13), rGalectin-3ₘᵢₙ₁ (SEQ ID NO: 14), rGalectin-3ₘᵢₙ₂ (SEQ ID NO: 15), CD2APₘᵢₙ₁ (SEQ ID NO: 16), CD2APₘᵢₙ₂ (SEQ ID NO: 17), HTLV-1 Gag 111-130 (SEQ ID NO: 18), MLV Gag₁₀₈₋₁₇₇ (SEQ ID NO: 19), and MPMV Gag 197-215 (SEQ ID NO: 20).

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include compositions (e.g., vaccine compositions). In some embodiments, the composition comprises: a nucleic acid composition comprising a polynucleotide encoding a fusion protein, wherein the fusion protein comprises an antigenic polypeptide (AP) and an endosomal sorting complex required for transport (ESCRT)-recruiting domain (ERD), and wherein a plurality of fusion proteins are capable of self-assembling into an enveloped nanoparticle (ENP) secreted from a cell in which the fusion proteins are expressed, thereby generating a population of ENPs. In some embodiments, the composition comprises: a nucleic acid composition comprising n polynucleotides each encoding an nth fusion protein, wherein n is an integer from 2 to 500, wherein each fusion protein comprises an AP and an endosomal sorting complex required for transport (ESCRT)-recruiting domain (ERD), wherein at least two of the fusion proteins differ with respect to the AP, and wherein a plurality of fusion proteins are capable of self-assembling into an ENP secreted from a cell in which the fusion proteins are expressed, thereby generating a population of ENPs. In some embodiments, the composition comprises: a population of ENPs, wherein each of the ENPs comprises a plurality of fusion proteins each comprising an AP and an endosomal sorting complex required for transport (ESCRT)-recruiting domain (ERD).

Disclosed herein include kits. In some embodiments, the kit comprises: a composition disclosed herein (e.g., a nucleic acid composition, a population of ENPs). Disclosed herein include cells. In some embodiments, a cell comprises: a nucleic acid composition disclosed herein.

Disclosed herein include methods of stimulating an immune response in a subject in need thereof. In some embodiments, the method comprises: administering to the subject a pharmaceutically effective amount of a composition disclosed herein (e.g., a nucleic acid composition, a population of ENPs), thereby stimulating an immune response in the subject.

Disclosed herein include methods of treating or preventing a disease or disorder in a subject in need thereof. In some embodiments, the method comprises: administering to the subject a pharmaceutically effective amount of a composition disclosed herein (e.g., a nucleic acid composition, a population of ENPs), thereby treating or preventing the disease or disorder in the subject. In some embodiments, the disease or disorder is a disease or disorder caused by an infectious agent. In some embodiments, the disease or disorder caused by an infectious agent is a disease or disorder caused by a coronavirus (CoV) infection.

Disclosed herein include methods for treating or preventing a CoV infection in a subject in need thereof. In some embodiments, the method comprises: administering to the subject a pharmaceutically effective amount of a composition disclosed herein (e.g., a nucleic acid composition, a population of ENPs), thereby treating or preventing the CoV infection in the subject.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, N.Y. 1994); Sambrook et al., Molecular Cloning, A Laboratory Manual, Cold Spring Harbor Press (Cold Spring Harbor, N.Y. 1989). For purposes of the present disclosure, the following terms are defined below.

As used herein, the terms “antigen” or “immunogen” are used interchangeably to refer to a substance, typically a protein, which is capable of inducing an immune response in a subject (e.g. a mammal, such as a human). The term also refers to proteins that are immunologically active in the sense that once administered to a subject, either directly or in the form of a nucleotide sequence or vector that encodes the protein, is able to evoke an immune response of the humoral and/or cellular type directed against that protein or a variant thereof.

As used herein, “sequence identity” or “identity” in the context of two nucleic acid or polypeptide sequences makes reference to the nucleotide bases or residues in the two sequences that are the same when aligned for maximum correspondence over a specified comparison window. Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; Pearson et al., Meth. Mol. Bio. 24:307-31, 1994; and Altschul et al., J. Mol. Biol. 215:403-10, 1990 (the content of each of these references is incorporated herein in its entirety).

When percentage of sequence identity or similarity is used in reference to proteins, it is recognized that residue positions which are not identical often differ by conservative amino acid substitutions, where amino acid residues are substituted with a functionally equivalent residue of the amino acid residues with similar physiochemical properties and therefore do not change the functional properties of the molecule. A functionally equivalent residue of an amino acid used herein typically can refer to other amino acid residues having physiochemical and stereochemical characteristics substantially similar to the original amino acid. The physiochemical properties include water solubility (hydrophobicity or hydrophilicity), dielectric and electrochemical properties, physiological pH, partial charge of side chains (positive, negative or neutral) and other properties identifiable to one of skill in the art. The stereochemical characteristics include spatial and conformational arrangement of the amino acids and their chirality. For example, glutamic acid is considered to be a functionally equivalent residue to aspartic acid in the sense of the current disclosure. Tyrosine and tryptophan are considered as functionally equivalent residues to phenylalanine. Arginine and lysine are considered as functionally equivalent residues to histidine.

The term “substantially identical” as used herein in the context of two or more sequences refers to a specified percentage of amino acid residues or nucleotides that are identical or functionally equivalent, such as about, at least or at least about 65% identity, optionally, about, at least or at least about 70%, 75%, 80%, 85%, 90%, 95%, or 99% identity over a specified region or over the entire sequence.

As used herein, the term “variant” refers to a polynucleotide or polypeptide having a sequence substantially similar or identical to a reference (e.g., the parent) polynucleotide or polypeptide. In the case of a polynucleotide, a variant can have deletions, substitutions, additions of one or more nucleotides at the 5′ end, 3′ end, and/or one or more internal sites in comparison to the reference polynucleotide. Similarities and/or differences in sequences between a variant and the reference polynucleotide can be detected using conventional techniques known in the art, for example polymerase chain reaction (PCR) and hybridization techniques. Variant polynucleotides also include synthetically derived polynucleotides, such as those generated, for example, by using site-directed mutagenesis. Generally, a variant of a polynucleotide, including, but not limited to, a DNA, can have at least, or at least about, 50%, 55%, 60%, 65%, 70%, 75%, 80%, 85%, 90%, 91%, 92%, 93%, 94%, 95%, 96%, 97%, 98%, 99%, or more sequence identity to the reference polynucleotide as determined by sequence alignment programs known in the art. In the case of a polypeptide, a variant can have deletions, substitutions, additions of one or more amino acids in comparison to the reference polypeptide. Similarities and/or differences in sequences between a variant and the reference polypeptide can be detected using conventional techniques known in the art, for example Western blot. A variant of a polypeptide can have, for example, at least, or at least about, 60%, 65%, 70%, 75%, 80%, 85%, 90%, 91%, 92%, 93%, 94%, 95%, 96%, 97%, 98%, 99%, or more sequence identity to the reference polypeptide as determined by sequence alignment programs known in the art.

Standard techniques can be used for recombinant DNA, oligonucleotide synthesis, and tissue culture and transformation (e.g., electroporation, lipofection). Enzymatic reactions and purification techniques can be performed according to manufacturer's specifications or as commonly accomplished in the art or as described herein. The foregoing techniques and procedures can be generally performed according to conventional methods well known in the art and as described in various general and more specific references that are cited and discussed throughout the present specification. See, e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose. Unless specific definitions are provided, the nomenclatures utilized in connection with, and the laboratory procedures and techniques of, analytical chemistry, synthetic organic chemistry, and medicinal and pharmaceutical chemistry described herein are those commonly known and used in the art. Standard techniques can be used for chemical syntheses, chemical analyses, pharmaceutical preparation, formulation, and delivery, and treatment of patients.

The term “construct,” as used herein, refers to a recombinant nucleic acid that has been generated for the purpose of the expression of a specific nucleotide sequence(s), or that is to be used in the construction of other recombinant nucleotide sequences.

As used herein, the terms “nucleic acid” and “polynucleotide” are interchangeable and refer to any nucleic acid, whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sultone linkages, and combinations of such linkages. The terms “nucleic acid” and “polynucleotide” also specifically include nucleic acids composed of bases other than the five biologically occurring bases (adenine, guanine, thymine, cytosine and uracil).

### Self-Assembling ENPs as a Vaccine Platform Technology

There are provided, in some embodiments, compositions (e.g., nucleic acid compositions, population(s) of ENPs). In some embodiments, the composition is a vaccine composition. In some embodiments, the composition comprises a nucleic acid composition (e.g., mRNA vaccine, DNA vaccine, a construct) comprising a polynucleotide encoding a fusion protein, wherein the fusion protein comprises an antigenic polypeptide (AP) and an endosomal sorting complex required for transport (ESCRT)-recruiting domain (ERD), and wherein a plurality of fusion proteins are capable of self-assembling into an enveloped nanoparticle (ENP) secreted from a cell in which the fusion proteins are expressed, thereby generating a population of ENPs. There are provided, in some embodiments, ENP-generating cells. In some embodiments, an ENP-generating cell comprises a nucleic acid composition disclosed herein.

There are provided, in some embodiments, compositions (e.g., nucleic acid compositions, population(s) of ENPs). In some embodiments, the composition comprises a nucleic acid composition (e.g., mRNA vaccine, DNA vaccine) comprising n polynucleotides each encoding an nth fusion protein. In some embodiments, n is an integer from 2 to 500 (e.g., 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 128, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, or a number or a range between any two of these values). Each fusion protein can comprise an antigenic polypeptide (AP) and an endosomal sorting complex required for transport (ESCRT)-recruiting domain (ERD). At least two of the nth fusion proteins can differ with respect to the AP (e.g., heterologous antigens, disparate AP). In some embodiments, some or all of the nth fusion proteins differ with respect to the AP presented (e.g., heterologous antigens, disparate AP), thereby the population of ENPs thereby displays a plurality of disparate AP (heterologous antigens). The plurality of fusion proteins can be capable of self-assembling into an enveloped nanoparticle (ENP) secreted from a cell in which the fusion proteins are expressed, thereby generating a population of ENPs.

In some embodiments, fusion proteins are capable of being presented on the surface of a cell in which the fusion proteins are expressed. In some embodiments, the self-assembly of an ENP: does not require an exogenous nucleic acid other than the nucleic acid composition, does not require any exogenous components other than the plurality of fusion proteins; and/or only requires a single component (e.g., the fusion protein). In some embodiments, the cell is: a cell of a subject; an in vivo cell, an ex vivo cell, or an in situ cell; and/or an adherent cell or a suspension cell.

In some embodiments, upon secretion from a cell of a subject, the ENPs are capable of distributing within one or more tissues of a subject (e.g., adrenal gland tissue, appendix tissue, bladder tissue, bone, bowel tissue, brain tissue, breast tissue, bronchi, coronal tissue, ear tissue, esophagus tissue, eye tissue, gall bladder tissue, genital tissue, heart tissue, hypothalamus tissue, kidney tissue, large intestine tissue, intestinal tissue, larynx tissue, liver tissue, lung tissue, lymph nodes, mouth tissue, nose tissue, pancreatic tissue, parathyroid gland tissue, pituitary gland tissue, prostate tissue, rectal tissue, salivary gland tissue, skeletal muscle tissue, skin tissue, small intestine tissue, spinal cord, spleen tissue, stomach tissue, thymus gland tissue, trachea tissue, thyroid tissue, ureter tissue, urethra tissue, soft and connective tissue, peritoneal tissue, blood vessel tissue, fat tissue, or any combination thereof). In some embodiments, said ENPs engage a plurality of immune cells in said one or more tissues, thereby mimicking a natural infection.

In some embodiments, the composition comprises: a population of enveloped nanoparticles (ENPs), wherein each of the ENPs comprises a plurality of fusion proteins each comprising an antigenic polypeptide (AP) and an endosomal sorting complex required for transport (ESCRT)-recruiting domain (ERD). The ENPs can be derived from expression of a nucleic acid composition provided herein. At least two of the fusion proteins differ with respect to the AP, and wherein the population of ENPs thereby display a plurality of disparate AP (e.g., heterologous antigens).

The ENPs can comprise a lipid bilayer (e.g., a lipid bilayer derived from the cell from which the ENP was secreted). In some embodiments, the ERD recruits one or more ESCRT proteins to the cytoplasmic tail of the fusion protein. In some embodiments, the recruitment of ESCRT proteins via the ERD induces the self-assembly and budding of ENPs. The cytoplasmic portion of the fusion protein can comprise the ERD. The cytoplasmic tail of the fusion protein can comprise the ERD. In some embodiments, the ERD interacts with the ESCRT proteins TSG101, NEDD4, and/or ALIX.

The ERD can comprise or can be derived from a nonhuman protein, optionally a nonmammalian protein, further optionally a chicken protein, a mouse protein, a lizard protein, a reptile protein, a hamster protein, or a goldfish protein. The ERD can comprise or can be derived from the ESCRT and ALIX binding region (EABR) of the human CEP55 protein, optionally residues 170-213. The ERD can comprise or can be derived from Syntenin-1, rat Galectin-3 (rGalectin-3), Hrs, and/or CD2AP. The ERD can comprise or can be derived from a viral protein, optionally a fragment of a viral protein, further optionally a retroviral protein, herpes simplex viral protein, vaccinia viral protein, hepadnaviral protein, togaviral protein, flaviviral protein, arenaviral protein, coronaviral protein, orthomyxoviral protein, paramyxoviral protein, bunyaviral protein, bornaviral protein, rhabdoviral protein or filoviral protein, optionally a Gag protein, further optionally derived from EIAV, HTLV-1, MLV, or MPMV, optionally EIAV p9 and/or HIV-1 p6. The ERD can comprise or can be derived from: an Ebola protein, optionally EBOV VP40. The ERD can comprise an amino acid sequence at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values, identical to SEQ ID NO: 1-20 and 23. Tables 1 and 2 depict non-limiting exemplary ERD provided herein.

In some embodiments, the nucleic acid composition does not comprise a polynucleotide encoding SpyTag or lentiviral Gag. In some embodiments, the ENPs do not comprise SpyTag or lentiviral Gag.

The fusion protein can comprise an endocytosis-preventing motif (EPM) capable of preventing endocytosis of the fusion protein. In some embodiments, the EPM: tethers the fusion protein to the cytoskeleton, thereby preventing localization to coated pits and endocytosis; enhances ENP assembly, ENP production, and/or ENP secretion; and/or prevents endocytosis of the fusion protein, thereby extending the time a fusion protein remains at the plasma membrane to interact with ESCRT proteins. In some embodiments, an endocytosis-preventing motif (EPM) disclosed herein is employed to generate fusion proteins with T-cell receptors (TCRs) or chimeric antigen receptors (CARs), which can, in some embodiments, enhance the activity and/or potency of T-cells and/or CAR-T-cells against diseases or disorders (e.g., cancer, infectious diseases), optionally at least about 1.1-fold. There are provided, in some embodiments, an EPM-TCR fusion protein wherein an EPM is fused with a TCR. There are provided, in some embodiments, an EPM-CAR fusion protein wherein an EPM is fused with a CAR. In some embodiments, without being bound by any particular theory, the TCRs or CARs with EPM fusions stay on the T-cell surface for a longer period of time (e.g., at least about 1.1-longer), and, in some embodiments, can be more effective at interacting with target antigens on cancer or infected cells, and consequently kill those cells more effectively. There are provided, in some embodiments, nucleic acid compositions encoding one or more EPM-TCR fusion proteins and/or one or more EPM-CAR fusion proteins. There are provided, in some embodiments, cells (e.g., T-cells and/or CAR-T-cells) comprising one or more EPM-TCR fusion proteins and/or one or more EPM-CAR fusion proteins. There are provided, in some embodiments, methods of treating a disease or disorder (e.g., cancer, an infectious disease) comprising administering an effective amount of said nucleic acid compositions and/or cells (e.g., nucleic acid compositions encoding one or more EPM-TCR fusion proteins and/or one or more EPM-CAR fusion proteins, T-cells and/or CAR-T-cells comprising one or more EPM-TCR fusion proteins and/or one or more EPM-CAR fusion proteins) to a subject.

The EPM can be capable of: increasing the abundance and/or density of fusion proteins on and/or in the ENP by at least about 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) as compared to a ENP comprising a fusion protein that does not comprise the EPM; and/or increasing the number of ENPs secreted by a cell by at least about 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) as compared to a cell expressing a fusion protein that does not comprise the EPM.

The EPM can comprise or can be derived from a portion of murine low-affinity gamma Fc region receptor II isoform FcRII-B1. The EPM can comprise all or a portion of the cytoplasmic tail of FcRII-B1. The EPM can comprise an amino acid sequence at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values, identical to SEQ ID NO: 21.

The fusion protein can comprise one or more linkers. The one or more linkers can comprise one or more flexible amino acid residues, optionally about 1, 2, 3, 4, 5, 6, 7, 8, 9, 10, 11, 12, 13, 14, 15, 16, 17, 18, 19, 20, 25, 30, 35, 40, or a number or a range between any two of these values, flexible amino acid residues, further optionally the flexible amino acid residues comprise glycine, serine, or a combination thereof. The one or more linkers can be a glycine-serine (GS) linker, optionally 1, 2, 3, 4, 5, 6, 7, 8, 9, 10, 11, 12, 13, 14, 15, 16, 17, 18, 19, 20, 25, 30, 35, 40, or a number or a range between any two of these values, amino acids in length. The one or more linkers can be situated between the ERD and the AP, between the ERD and the EPM, between the ERD and the TD, between the EPM and the TD, between the AP and the EPM, and/or between the AP and the TD.

The APs presented on the ENPs herein described can be displayed on its surface. Alternatively, the AP presented on the ENPs herein described can be partially encapsulated or embedded such that at least an immunogenic portion of the AP is exposed and accessible by a host cell receptor so as to induce an immune response. The AP, or a portion thereof, can be displayed in and/or on the surface of the ENP. The AP can be about 1 to about 10000 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, 2000, 3000, 4000, 5000, 6000, 7000, 8000, 9000, 10000, or a number or a range between any of these values) amino acids in length.

The AP can comprise or can be derived from an antigenic protein associated with a disease or disorder, optionally an immunogenic variant and/or an immunogenic fragment of said antigenic protein. The AP can comprise or be derived from at least a portion of an antigenic protein. The AP can comprise or can be derived from a conserved portion of said antigenic protein. The AP can be present on and/or in the ENP in its natural membrane-associated conformation. The AP can comprise or can be derived from at least about 5 percent (e.g., 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) of the full length of said antigenic protein. The AP can comprise a membrane protein (e.g., a multi-span transmembrane protein). In some embodiments, the AP is not configured to be a soluble protein. In some embodiments, the AP does not comprise one or more mutations configured to enhance its solubility and/or stability.

Accordingly, the APs can contain amino acid substitutions relative to the antigenic proteins disclosed herein. Any amino acid substitution is permissible so long as the immunogenic activity of the protein is not significantly altered (e.g., at most 10%, 20%, 30%, 40% or 50% decrease relative to the coronavirus protein antigens disclosed herein) and the variants retain the desired activity. Preferred variants typically contains substitutions with one or more amino acids substituted with their functional equivalents.

In some embodiments, the AP does not comprise a transmembrane domain and/or is a soluble protein, and wherein the fusion protein comprises a transmembrane domain (TD). The transmembrane domain can comprise or can be derived from a nonhuman transmembrane protein (e.g., a nonmammalian transmembrane protein). The TD can comprise or can be derived from a natural protein, a recombinant protein, and/or synthetic protein (e.g., a synthetic protein comprising predominantly hydrophobic residues).

The compositions herein described (e.g., a nucleic acid composition, a population of ENPs) can be prepared using any standard molecular biology procedures known to the person skilled in the art as well as the protocols exemplified herein (see e.g., Examples).

Production of the nucleic acid compositions can use recombinant DNA technology well known in the art. For example, an ENP can be produced using an expression vector comprising a nucleic acid molecule encoding fusion protein(s) described herein. The nucleic acid molecule can be operably linked to appropriate regulatory elements including, but not limited to, a promoter, enhancer, transcription initiation site, termination site, and translation initiation site. The vector may comprise one or more selectable marker genes such as gene providing ampicillin resistance or kanamycin resistance. Methods for the construction of nucleic acid constructs are well known. See, for example, Molecular Cloning: a Laboratory Manual, 3^{rd}. edition, Sambrook et al. 2001 Cold Spring Harbor Laboratory Press, and Current Protocols in Molecular Biology, Ausubel et al. eds., John Wiley & Sons, 1994. In vitro Transcription of RNA and/or cDNA encoding the polynucleotides described herein may be transcribed using an in vitro transcription (IVT) system. In vitro transcription of RNA is known in the art and is described in International Publication WO 2014/152027, which is incorporated by reference herein in its entirety. In some embodiments, the RNA of the present disclosure is prepared in accordance with any one or more of the methods described in WO 2018/053209, US 2021/0251898, and WO 2019/036682, each of which is incorporated by reference herein. ENPs can be produced (and isolated) from cells comprising nucleic acid compositions provided herein. For example, ENPs can be derived from cell cultures transiently transfected with the nucleic acid composition, optionally derived via ultracentrifugation and/or size exclusion chromatography, further optionally ultracentrifugation on a 20% sucrose cushion, optionally transfected via calcium phosphate transfection, DEAE-dextran mediated transfection, cationic lipid-mediated transfection, electroporation, electrical nuclear transport, chemical transduction, electrotransduction, Lipofectamine-mediated transfection, Effectene-mediated transfection, lipid nanoparticle (LNP)-mediated transfection, or any combination thereof. The systems, methods, compositions, and kits provided herein can, in some embodiments, be employed in concert with the systems, methods, compositions, and kits described in PCT Application Publication No. WO2022103891A1, entitled, “Multivalent carriers and related vaccine compositions,” filed Nov. 10, 2021, the content of which is incorporated herein by reference in its entirety.

Pathogenic Antigens

The AP can comprise or can be derived from the full-length surface protein of an infectious agent. The disease or disorder can be an infectious disease or disorder caused by an infectious agent, the AP can comprise or can be derived from an antigenic protein of said infectious agent, and the antigenic protein of said infectious agent can be a pathogenic antigen. In some embodiments, the pathogenic antigen is selected from the group comprising: Outer membrane protein A OmpA, biofilm associated protein Bap, transport protein MucK (Acinetobacter baumannii, Acinetobacter infections)); variable surface glycoprotein VSG, microtubule-associated protein MAPP15, trans-sialidase TSA (Trypanosoma brucei, African sleeping sickness (African trypanosomiasis)); HIV p24 antigen, HIV envelope proteins (Gp120, Gp41, Gp160), polyprotein GAG, negative factor protein Nef, trans-activator of transcription Tat (HIV (Human immunodeficiency virus), AIDS (Acquired immunodeficiency syndrome)); galactose-inhibitable adherence protein GIAP, 29 kDa antigen Eh29, Gal/GaINAc lectin, protein CRT, 125 kDa immunodominant antigen, protein M17, adhesin ADH112, protein STIRP (Entamoeba histolytica, Amoebiasis); Major surface proteins 1-5 (MSP1a, MSP1b, MSP2, MSP3, MSP4, MSP5), type IV secreotion system proteins (VirB2, VirB7, VirB11, VirD4) (Anaplasma genus, Anaplasmosis); protective Antigen PA, edema factor EF, lethal facotor LF, the S-layer homology proteins SLH (Bacillus anthracis, Anthrax); acranolysin, phospholipase D, collagen-binding protein CbpA (Arcanobacterium haemolyticum, Arcanobacterium haemolyticum infection); nucleocapsid protein NP, glycoprotein precursor GPC, glycoprotein GP1, glycoprotein GP2 (Junin virus, Argentine hemorrhagic fever); chitin-protein layer proteins, 14 kDa suarface antigen A14, major sperm protein MSP, MSP polymerization-organizing protein MPOP, MSP fiber protein 2 MFP2, MSP polymerization-activating kinase MPAK, ABA-1-like protein ALB, protein ABA-1, cuticulin CUT-1 (Ascaris lumbricoides, Ascariasis); 41 kDa allergen Asp v13, allergen Asp f3, major conidial surface protein rodlet A, protease Pep1p, GPI-anchored protein Gel1p, GPI-anchored protein Crap (Aspergillus genus, Aspergillosis); family VP26 protein, VP29 protein (Astroviridae, Astrovirus infection); Rhoptry-associated protein 1 RAP-1, merozoite surface antigens MSA-1, MSA-2 (a1, a2, c), 12D3, 1105, 21134, P29, variant erythrocyte surface antigen VESA1, Apical Membrane Antigen 1 AMA-1 (Babesia genus, Babesiosis); hemolysin, enterotoxin C, PX01-51, glycolate oxidase, ABC-transporter, penicillin-bingdn protein, zinc transporter family protein, pseudouridine synthase Rsu, plasmid replication protein RepX, oligoendopeptidase F, prophage membrane protein, protein HemK, flagellar antigen H, 28.5-kDa cell surface antigen (Bacillus cereus, Bacillus cereus infection); large T antigen LT, small T antigen, capsid protein VP1, capsid protein VP2 (BK virus, BK virus infection); 29 kDa-protein, caspase-3-like antigens, glycoproteins (Blastocystis hominis, Blastocystis hominis infection); yeast surface adhesin WI-1 (Blastomyces dermatitidis, Blastomycosis); nucleoprotein N, polymerase L, matrix protein Z, glycoprotein GP (Machupo virus, Bolivian hemorrhagic fever); outer surface protein A OspA, outer surface protein OspB, outer surface protein OspC, decorin binding protein A DbpA, decorin binding protein B DbpB, flagellar filament 41 kDa core protein Fla, basic membrane protein A precursor BmpA (Immunodominant antigen P39), outer surface 22 kDa lipoprotein precursor (antigen IPLA7), variable surface lipoprotein vlsE (Borrelia genus, Borrelia infection); Botulinum neurotoxins BoNT/A1, BoNT/A2, BoNT/A3, BoNT/B, BoNT/C, BoNT/D, BoNT/E, BoNT/F, BoNT/G, recombinant botulinum toxin F Hc domain FHc (Clostridium botulinum, Botulism (and Infant botulism)); nucleocapsid, glycoprotein precursor (Sabia virus, Brazilian hemorrhagic fever); copper/Zinc superoxide dismutase SodC, bacterioferritin Bfr, 50S ribosomal protein Rp1L, OmpA-like transmembrane domain-containing protein Omp31, immunogenic 39-kDa protein M5 P39, zinc ABC transporter periplasmic zinc-bnding protein znuA, periplasmic immunogenic protein Bp26, 30S ribosomal protein S12 RpsL, glyceraldehyde-3-phosphate dehydrogenase Gap, 25 kDa outer-membrane immunogenic protein precursor Omp25, invasion protein B1aIB, trigger factor Tig, molecular chaperone DnaK, putative peptidyl-prolyl cis-trans isomerase SurA, lipoprotein Omp19, outer membrane protein MotY Omp16, conserved outer membrane protein D15, malate dehydrogenase Mdh, component of the Type-IV secretion system (TOSS) VirJ, lipoprotein of unknown function BAB1_0187 (Brucella genus, Brucellosis); members of the ABC transporter family (LoIC, OppA, and PotF), putative lipoprotein releasing system transmembrane protein LoIC/E, flagellin FliC, Burkholderia intracellular motility A BimA, bacterial Elongation factor-Tu EF-Tu, 17 kDa OmpA-like protein, boaA coding protein, boaB coding protein (Burkholderia cepacia and other Burkholderia species, Burkholderia infection); mycolyl-transferase Ag85A, heat-shock protein Hsp65, protein TB10.4, 19 kDa antigen, protein PstS3, heat-shock protein Hsp70 (Mycobacterium ulcerans, Buruli ulcer); norovirus major and minor viral capsid proteins VP1 and VP2, genome polyprotein, Sapoviurus capsid protein VP1, protein Vp3, geome polyprotein (Caliciviridae family, Calicivirus infection (Norovirus and Sapovirus)); major outer membrane protein PorA, flagellin FlaA, surface antigen CjaA, fibronectin binding protein CadF, aspartate/glutamate-binding ABC transporter protein PeblA, protein FspA1, protein FspA2 (Campylobacter genus, Campylobacteriosis); glycolytic enzyme enolase, secreted aspartyl proteinases SAP1-10, glycophosphatidylinositol (GPI)-linked cell wall protein, protein Hyrl, complement receptor 3-related protein CR3-RP, adhesin Als3p, heat shock protein 90 kDa hsp90, cell surface hydrophobicity protein CSH (usually Candida albicans and other Candida species, Candidiasis); 17-kDa antigen, protein P26, trimeric autotransporter adhesins TAAs, Bartonella adhesin A BadA, variably expressed outer-membrane proteins Vomps, protein Pap3, protein HbpA, envelope-associated protease HtrA, protein OMP89, protein GroEL, protein LaIB, protein OMP43, dihydrolipoamide succinyltransferase SucB (Bartonella henselae, Cat-scratch disease); amastigote surface protein-2, amastigote-specific surface protein SSP4, cruzipain, trans-sialidase TS, trypomastigote surface glycoprotein TSA-1, complement regulatory protein CRP-10, protein G4, protein G2, paraxonemal rod protein PAR2, paraflagellar rod component Part, mucin-Associated Surface Proteins MPSP (Trypanosoma cruzi, Chagas Disease (American trypanosomiasis)); envelope glycoproteins (gB, gC, gE, gH, gI, gK, gL) (Varicella zoster virus (VZV), Chickenpox); major outer membrane protein MOMP, probable outer membrane protein PMPC, outer membrane complex protein B OmcB, heat shock proteins Hsp60 HSP10, protein IncA, proteins from the type III secretion system, ribonucleotide reductase small chain protein NrdB, plasmid protein Pgp3, chlamydial outer protein N CopN, antigen CT521, antigen CT425, antigen CT043, antigen TC0052, antigen TC0189, antigen TC0582, antigen TC0660, antigen TC0726, antigen TC0816, antigen TC0828 (Chlamydia trachomatis, Chlamydia); low calcium response protein E LCrE, chlamydial outer protein N CopN, serine/threonine-protein kinase PknD, acyl-carrier-protein S-malonyltransferase FabD, single-stranded DNA-binding protein Ssb, major outer membrane protein MOMP, outer membrane protein 2 Omp2, polymorphic membrane protein family (Pmp1, Pmp2, Pmp3, Pmp4, Pmp5, Pmp6, Pmp7, Pmp8, Pmp9, Pmp10, Pmp11, Pmp12, Pmp13, Pmp14, Pmp15, Pmp16, Pmp17, Pmp18, Pmp19, Pmp20, Pmp21) (Chlamydophila pneumoniae, Chlamydophila pneumoniae infection); cholera toxin B CTB, toxin coregulated pilin A TcpA, toxin coregulated pilin TcpF, toxin co-regulated pilus biosynthesis ptrotein F TcpF, cholera enterotoxin subunit A, cholera enterotoxin subunit B, Heat-stable enterotoxin ST, mannose-sensitive hemagglutinin MSHA, outer membrane protein U Porin ompU, Poring B protein, polymorphic membrane protein-D (Vibrio cholerae, Cholera); propionyl-CoA carboxylase PCC, 14-3-3 protein, prohibitin, cysteine proteases, glutathione transferases, gelsolin, cathepsin L proteinase CatL, Tegumental Protein 20.8 kDa TP20.8, tegumental protein 31.8 kDa TP31.8, lysophosphatidic acid phosphatase LPAP, (Clonorchis sinensis, Clonorchiasis); surface layer proteins SLPs, glutamate dehydrogenase antigen GDH, toxin A, toxin B, cysteine protease Cwp84, cysteine protease Cwp13, cysteine protease Cwp19, Cell Wall Protein CwpV, flagellar protein FliC, flagellar protein FliD (Clostridium difficile, Clostridium difficile infection); rhinoviruses: capsid proteins VP1, VP2, VP3, VP4; coronaviruses: sprike proteins S, envelope proteins E, membrane proteins M, nucleocapsid proteins N (usually rhinoviruses and coronaviruses, Common cold (Acute viral rhinopharyngitis; Acute coryza)); prion protein Prp (CJD prion, Creutzfeldt-Jakob disease (CJD)); envelope protein Gc, envelope protein Gn, nucleocapsid proteins (Crimean-Congo hemorrhagic fever virus, Crimean-Congo hemorrhagic fever (CCHF)); virulence-associated DEAD-box RNA helicase VAD1, galactoxylomannan-protein GaIXM, glucuronoxylomannan GXM, mannoprotein MP (Cryptococcus neoformans, Cryptococcosis); acidic ribosomal protein P2 CpP2, mucin antigens Muc1, Muc2, Muc3 Muc4, Muc5, Much, Muc7, surface adherence protein CP20, surface adherence protein CP23, surface protein CP12, surface protein CP21, surface protein CP40, surface protein CP60, surface protein CP15, surface-associated glycopeptides gp40, surface-associated glycopeptides gp15, oocyst wall protein AB, profilin PRF, apyrase (Cryptosporidium genus, Cryptosporidiosis); fatty acid and retinol binding protein-1 FAR-1, tissue inhibitor of metalloproteinase TIMP (TMP), cysteine proteinase ACEY-1, cysteine proteinase ACCP-1, surface antigen Ac-16, secreted protein 2 ASP-2, metalloprotease 1 MTP-1, aspartyl protease inhibitor API-1, surface-associated antigen SAA-1, adult-specific secreted factor Xa serine protease inhibitor anticoagulant AP, cathepsin D-like aspartic protease ARR-1 (usually Ancylostoma braziliense; multiple other parasites, Cutaneous larva migrans (CLM)); cathepsin L-like proteases, 53/25-kDa antigen, 8 kDa family members, cysticercus protein with a marginal trypsin-like activity TsAg5, oncosphere protein TSOL18, oncosphere protein TSOL45-1A, lactate dehydrogenase A LDHA, lactate dehydrogenase B LDHB (Taenia solium, Cysticercosis); pp65 antigen, membrane protein pp15, capsid-proximal tegument protein pp150, protein M45, DNA polymerase UL54, helicase UL105, glycoprotein gM, glycoprotein gN, glcoprotein H, glycoprotein B gB, protein UL83, protein UL94, protein UL99 (Cytomegalovirus (CMV), Cytomegalovirus infection); capsid protein C, premembrane protein prM, membrane protein M, envelope protein E (domain I, domain II, domain II), protein NS1, protein NS2A, protein NS2B, protein NS3, protein NS4A, protein 2K, protein NS4B, protein NS5 (Dengue viruses (DEN-1, DEN-2, DEN-3 and DEN-4)-Flaviviruses, Dengue fever); 39 kDa protein (Dientamoeba fragilis, Dientamoebiasis); diphtheria toxin precursor Tox, diphteria toxin DT, pilin-specific sortase SrtA, shaft pilin protein SpaA, tip pilin protein SpaC, minor pilin protein SpaB, surface-associated protein D1P1281 (Corynebacterium diphtherias, Diphtheria); glycoprotein GP, nucleoprotein NP, minor matrix protein VP24, major matrix protein VP40, transcription activator VP30, polymerase cofactor VP35, RNA polymerase L (Ebolavirus (EBOV), Ebola hemorrhagic fever); prion protein (vCJD prion, Variant Creutzfeldt-Jakob disease (vCJD, nvCJD)); UvrABC system protein B, protein Flp1, protein Flp2, protein Flp3, protein TadA, hemoglobin receptor HgbA, outer membrane protein TdhA, protein CpsRA, regulator CpxR, protein SapA, 18 kDa antigen, outer membrane protein NcaA, protein LspA, protein LspA1, protein LspA2, protein LspB, outer membrane component DsrA, lectin DItA, lipoprotein Hip, major outer membrane protein OMP, outer membrane protein OmpA2 (Haemophilus ducreyi, Chancroid); aspartyl protease 1 Pep 1, phospholipase B PLB, alpha-mannosidase 1 AMN1, glucanosyltransferase GEL1, urease URE, peroxisomal matrix protein Pmp1, proline-rich antigen Pra, humal T-cell reative protein TcrP (Coccidioides immitis and Coccidioides posadasii, Coccidioidomycosis); allergen Tri r 2, heat shock protein 60 Hsp60, fungal actin Act, antigen Tri r2, antigen Tri r4, antigen Tri t1, protein IV, glycerol-3-phosphate dehydrogenase Gpd1, osmosensor HwSho1A, osmosensor HwSho1B, histidine kinase HwHhk7B, allergen Mala s 1, allergen Mala s 11, thioredoxin Trx Mala s 13, allergen Mala f, allergen Mala s (usually Trichophyton spp, Epidermophyton spp., Malassezia spp., Hortaea werneckii, Dermatophytosis); protein EG95, protein EG10, protein EG18, protein EgA31, protein EM18, antigen EPC1, antigen B, antigen 5, protein P29, protein 14-3-3, 8-kDa protein, myophilin, heat shock protein 20 HSP20, glycoprotein GP-89, fatty acid binding protein FAPB (Echinococcus genus, Echinococcosis); major surface protein 2 MSP2, major surface protein 4 MSP4, MSP variant SGV1, MSP variant SGV2, outer membrane protein OMP, outer membrande protein 19 OMP-19, major antigenic protein MAP1, major antigenic protein MAP1-2, major antigenic protein MAP1B, major antigenic protein MAP1-3, Erum2510 coding protein, protein GroEL, protein GroES, 30-kDA major outer membrane proteins, GE 100-kDa protein, GE 130-kDa protein, GE 160-kDa protein (Ehrlichia genus, Ehrlichiosis); secreted antigen SagA, sagA-like proteins SalA and SaIB, collagen adhesin Scm, surface proteins Fms1 (EbpA(fm), Fms5 (EbpB(fm), Fms9 (EpbC(fm) and Fms10, protein EbpC(fm), 96 kDa immunoprotective glycoprotein G1 (Enterococcus genus, Enterococcus infection); genome polyprotein, polymerase 3D, viral capsid protein VP1, viral capsid protein VP2, viral capsid protein VP3, viral capsid protein VP4, protease 2A, protease 3C (Enterovirus genus, Enterovirus infection); outer membrane proteins OM, 60 kDa outer membrane protein, cell surface antigen OmpA, cell surface antigen OmpB (sca5), 134 kDa outer membrane protein, 31 kDa outer membrane protein, 29.5 kDa outer membrane protein, cell surface protein SCA4, cell surface protein Adr1 (RP827), cell surface protein Adr2 (RP828), cell surface protein SCA1, Invasion protein invA, cell division protein fts, secretion proteins sec Ofamily, virulence proteins virB, tlyA, tlyC, parvulin-like protein Plp, preprotein translocase SecA, 120-kDa surface protein antigen SPA, 138 kD complex antigen, major 100-kD protein (protein I), intracytoplasmic protein D, protective surface protein antigen SPA (Rickettsia prowazekii, Epidemic typhus); Epstein-Barr nuclear antigens (EBNA-1, EBNA-2, EBNA-3A, EBNA-3B, EBNA-3C, EBNA-leader protein (EBNA-LP)), latent membrane proteins (LMP-1, LMP-2A, LMP-2B), early antigen EBV-EA, membrane antigen EBV-MA, viral capsid antigen EBV-VCA, alkaline nuclease EBV-AN, glycoprotein glycoprotein gp350, glycoprotein gp110, glycoprotein gp42, glycoprotein gHgL, glycoprotein gB (Epstein-Barr Virus (EBV), Epstein-Barr Virus Infectious Mononucleosis); capsid protein VP2, capsid protein VP1, major protein NS1 (Parvovirus B19, Erythema infectiosum (Fifth disease)); pp65 antigen, glycoprotein 105, major capsid protein, envelope glycoprotein H, protein U51 (Human herpesvirus 6 (HHV-6) and Human herpesvirus 7 (HHV-7), Exanthem subitum); thioredoxin-glutathione reductase TGR, cathepsins L1 and L2, Kunitz-type protein KTM, leucine aminopeptidase LAP, cysteine proteinase Fast, saposin-like protein-2 SAP-2, thioredoxin peroxidases TPx, Prx-1, Prx-2, cathepsin I cysteine proteinase CL3, protease cathepsin L CL1, phosphoglycerate kinase PGK, 27-kDa secretory protein, 60 kDa protein HSP35alpha, glutathione transferase GST, 28.5 kDa tegumental antigen 28.5 kDa TA, cathepsin B3 protease CatB3, Type I cystatin stefin-1, cathepsin L5, cathepsin L1g and cathepsin B, fatty acid binding protein FABP, leucine aminopeptidases LAP (Fasciola hepatica and Fasciola gigantica, Fasciolosis); prion protein (FFI prion, Fatal familial insomnia (FFI)); venom allergen homolog-like protein VAL-1, abundant larval transcript ALT-1, abundant larval transcript ALT-2, thioredoxin peroxidase TPX, vespid allergen homologue VAH, thiordoxin peroxidase 2 TPX-2, antigenic protein SXP (peptides N, N1, N2, and N3), activation associated protein-1 ASP-1, Thioredoxin TRX, transglutaminase BmTGA, glutathione-S-transferases GST, myosin, vespid allergen homologue VAH, 175 kDa collagenase, glyceraldehyde-3-phosphate dehydrogenase GAPDH, cuticular collagen Col-4, secreted larval acidic proteins SLAPs, chitinase CHI-1, maltose binding protein MBP, glycolytic enzyme fructose-1,6-bisphosphate aldolase Fba, tropomyosin TMY-1, nematode specific gene product OvB20, onchocystatin CPI-2, Cox-2 (Filarioidea superfamily, Filariasis); phospholipase C PLC, heat-labile enterotoxin B, Iota toxin component Ib, protein CPE1281 pyruvate ferredoxin oxidoreductase, elongation factor G EF-G, perfringolysin 0 Pfo, glyceraldehyde-3-phosphate dehydrogenase GapC, Fructose-bisphosphate aldolase Alf2, Clostridium perfringens enterotoxin CPE, alpha toxin AT, alpha toxoid ATd, epsilon-toxoid ETd, protein HP, large cytotoxin TpeL, endo-beta-N-acetylglucosaminidase Naglu, phosphoglyceromutase Pgm (Clostridium perfringens, Food poisoning by Clostridium perfringens); leukotoxin IktA, adhesion FadA, outer membrane protein RadD, high-molecular weight arginine-binding protein (Fusobacterium genus, Fusobacterium infection); phospholipase C PLC, heat-labile enterotoxin B, Iota toxin component Ib, protein CPE1281, pyruvate ferredoxin oxidoreductase, elongation factor G EF-G, perfringolysin 0 Pfo, glyceraldehyde-3-phosphate dehydrogenase GapC, fructose-bisphosphate aldolase Alf2, Clostridium perfringens enterotoxin CPE, alpha toxin AT, alpha toxoid ATd, epsilon-toxoid ETd, protein HP, large cytotoxin TpeL, endo-beta-N-acetylglucosaminidase Naglu, phosphoglyceromutase Pgm (usually Clostridium perfringens; other Clostridium species, Gas gangrene (Clostridial myonecrosis)); lipase A, lipase B, peroxidase Dec1 (Geotrichum candidum, Geotrichosis); prion protein (GSS prion, Gerstmann-Straussler-Scheinker syndrome (GSS)); cyst wall proteins CWP1, CWP2, CWP3, variant surface protein VSP, VSP1, VSP2, VSP3, VSP4, VSP5, VSP6, 56 kDa antigen, pyruvate ferredoxin oxidoreductase PFOR, alcohol dehydrogenase E ADHE, alpha-giardin, alpha8-giardin, alphal-guiardin, beta-giardin, cystein proteases, glutathione-S-transferase GST, arginine deiminase ADI, fructose-1,6-bisphosphat aldolase FBA, Giardia trophozoite antigens GTA (GTA1, GTA2), ornithine carboxyl transferase OCT, striated fiber-asseblin-like protein SALP, uridine phosphoryl-like protein UPL, alpha-tubulin, beta-tubulin (Giardia intestinalis, Giardiasis); members of the ABC transporter family (LoIC, OppA, and PotF), putative lipoprotein releasing system transmembrane protein LoIC/E, flagellin FliC, Burkholderia intracellular motility A BimA, bacterial Elongation factor-Tu EF-Tu, 17 kDa OmpA-like protein, boaA coding protein (Burkholderia mallei, Glanders); cyclophilin CyP, 24 kDa third-stage larvae protien GS24, excretion-secretion products ESPs (40, 80, 120 and 208 kDa) (Gnathostoma spinigerum and Gnathostoma hispidum, Gnathostomiasis); pilin proteins, minor pilin-associated subunit pilC, major pilin subunit and variants pilE, pilS, phase variation protein porA, Porin B PorB, protein TraD, Neisserial outer membrane antigen H.8, 70 kDa antigen, major outer membrane protein PI, outer membrane proteins PIA and PIB, W antigen, surface protein A NspA, transferrin binding protein TbpA, transferrin binding protein TbpB PBP2, mtrR coding protein, ponA coding protein, membrane permease FbpBC, FbpABC protein system, LbpAB proteins, outer membrane protein Opa, outer membrane transporter FetA, iron-repressed regulator MpeR (Neisseria gonorrhoeae, Gonorrhea); outer membrane protein A OmpA, outer membrane protein C OmpC, outer membrane protein K17 OmpK17 (Klebsiella granulomatis, Granuloma inguinale (Donovanosis)); fibronectin-binding protein Sfb, fibronectin/fibrinogen-binding protein FBP54, fibronectin-binding protein FbaA, M protein type 1 Emm1, M protein type 6 Emm6, immunoglobulin-binding protein 35 Sib35, Surface protein R28 Spr28, superoxide dismutase SOD, C5a peptidase ScpA, antigen I/II AgI/II, adhesin AspA, G-related alpha2-macroglobulin-binding protein GRAB, surface fibrillar protein M5 (Streptococcus pyogenes, Group A streptococcal infection); C protein f3 antigen, arginine deiminase proteins, adhesin BibA, 105 kDA protein BPS, surface antigens c, surface antigens R, surface antigens X, trypsin-resistant protein R1, trypsin-resistant protein R3, trypsin-resistant protein R4, surface immunogenic protein Sip, surface protein Rib, Leucine-rich repeats protein LrrG, serine-rich repeat protein Srr-2, C protein alpha-antigen Bca, Beta antigen Bag, surface antigen Epsilon, alpha-like protein ALP1, alpha-like protein ALP5 surface antigen delta, alpha-like protein ALP2, alpha-like protein ALP3, alpha-like protein ALP4, Cbeta protein Bac (Streptococcus agalactiae, Group B streptococcal infection); transferrin-binding protein 2 Tbp2, phosphatase P4, outer membrane protein P6, peptidoglycan-associated lipoprotein Pal, protein D, protein E, adherence and penetration protein Hap, outer membrane protein 26 Omp26, outer membrane protein P5 (Fimbrin), outer membrane protein D15, outer membrane protein OmpP2, 5′-nucleotidase NucA, outer membrane protein P1, outer membrane protein P2, outer membrane lipoprotein Pcp, Lipoprotein E, outer membrane protein P4, fuculokinase FucK, [Cu,Zn]-superoxide dismutase SodC, protease HtrA, protein 0145, alpha-galactosylceramide (Haemophilus influenzae, Haemophilus influenzae infection); polymerase 3D, viral capsid protein VP1, viral capsid protein VP2, viral capsid protein VP3, viral capsid protein VP4, protease 2A, protease 3C (Enteroviruses, mainly Coxsackie A virus and Enterovirus 71 (EV71), Hand, foot and mouth disease (HFMD)); RNA polymerase L, protein L, glycoprotein Gn, glycoprotein Gc, nucleocapsid protein 5, envelope glycoprotein G1, nucleoprotein NP, protein N, polyprotein M (Sin Nombre virus, Hantavirus, Hantavirus Pulmonary Syndrome (HPS)); heat shock protein HspA, heat shock protein HspB, citrate synthase GItA, protein UreB, heat shock protein Hsp60, neutrophil-activating protein NAP, catalase KatA, vacuolating cytotoxin VacA, urease alpha UreA, urease beta Ureb, protein Cpn10, protein groES, heat shock protein Hsp10, protein MopB, cytotoxicity-associated 10 kDa protein CAG, 36 kDa antigen, beta-lactamase HcpA, Beta-lactamase HcpB (Helicobacter pylori, Helicobacter pylori infection); integral membrane proteins, aggregation-prone proteins, 0-antigen, toxin-antigens Stx2B, toxin-antigen Stx1B, adhesion-antigen fragment Int28, protein EspA, protein EspB, Intimin, protein Tir, protein IntC300, protein Eae (Escherichia coli O157:H7, O111 and O104:H4, Hemolytic-uremic syndrome (HUS)); RNA polymerase L, protein L, glycoprotein Gn, glycoprotein Gc, nucleocapsid protein 5, envelope glycoprotein G1, nucleoprotein NP, protein N, polyprotein M (Bunyaviridae family, Hemorrhagic fever with renal syndrome (HFRS)); glycoprotein G, matrix protein M, nucleoprotein N, fusion protein F, polymerase L, protein W, proteinC, phosphoprotein p, non-structural protein V (Henipavirus (Hendra virus Nipah virus), Henipavirus infections); polyprotein, glycoproten Gp2, hepatitis A surface antigen HBAg, protein 2A, virus protein VP1, virus protein VP2, virus protein VP3, virus protein VP4, protein P1B, protein P2A, protein P3AB, protein P3D (Hepatitis A Virus, Hepatitis A); hepatitis B surface antigen HBsAg, Hepatitis B core antigen HbcAg, polymerase, protein Hbx, preS2 middle surface protein, surface protein L, large S protein, virus protein VP1, virus protein VP2, virus protein VP3, virus protein VP4 (Hepatitis B Virus (HBV), Hepatitis B); envelope glycoprotein E1 gp32 gp35 envelope glycoprotein E2 NS1 gp68 gp70, capsid protein C core protein Core, polyprotein, virus protein VP1, virus protein VP2, virus protein VP3, virus protein VP4, antigen G, protein NS3, protein NSSA, (Hepatitis C Virus, Hepatitis C); virus protein VP1, virus protein VP2, virus protein VP3, virus protein VP4, large hepaptitis delta antigen, small hepaptitis delta antigen (Hepatitis D Virus, Hepatitis D); virus protein VP1, virus protein VP2, virus protein VP3, virus protein VP4, capsid protein E2 (Hepatitis E Virus, Hepatitis E); glycoprotein L UL1, uracil-DNA glycosylase UL2, protein UL3, protein UL4, DNA replication protein UL5, portal protein UL6, virion maturation protein UL7, DNA helicase ULB, replication origin-binding protein UL9, glycoprotein M UL10, protein UL11, alkaline exonuclease UL12, serine-threonine protein kinase UL13, tegument protein UL14, terminase UL15, tegument protein UL16, protein UL17, capsid protein VP23 UL18, major capsid protein VP5 UL19, membrane protein UL20, tegument protein UL21, Glycoprotein H (UL22), Thymidine Kinase UL23, protein UL24, protein UL25, capsid protein P40 (UL26, VP24, VP22A), glycoprotein B (UL27), ICP18.5 protein (UL28), major DNA-binding protein ICP8 (UL29), DNA polymerase UL30, nuclear matrix protein UL31, envelope glycoprotein UL32, protein UL33, inner nuclear membrane protein UL34, capsid protein VP26 (UL35), large tegument protein UL36, capsid assembly protein UL37, VP19C protein (UL38), ribonucleotide reductase (Large subunit) UL39, ribonucleotide reductase (Small subunit) UL40, tegument protein/virion host shutoff VHS protein (UL41), DNA polymerase processivity factor UL42, membrane protein UL43, glycoprotein C (UL44), membrane protein UL45, tegument proteins VP11/12 (UL46), tegument protein VP13/14 (UL47), virion maturation protein VP16 (UL48, Alpha-TIF), envelope protein UL49, dUTP diphosphatase UL50, tegument protein UL51, DNA helicase/primase complex protein UL52, glycoprotein K (UL53), transcriptional regulation protein 1E63 (ICP27, UL54), protein UL55, protein UL56, viral replication protein ICP22 (1E68, US1), protein U52, serine/threonine-protein kinase U53, glycoprotein G (U54), glycoprotein J (U55), glycoprotein D (U56), glycoprotein I (U57), glycoprotein E (U58), tegument protein U59, capsid/tegument protein US10, Vmw21 protein (US11), ICP47 protein (IE12, US12), major transcriptional activator ICP4 (1E175, RS1), E3 ubiquitin ligase ICPO (IE110), latency-related protein 1 LRP1, latency-related protein 2 LRP2, neurovirulence factor RL1 (ICP34.5), latency-associated transcript LAT (Herpes simplex virus 1 and 2 (HSV-1 and HSV-2), Herpes simplex); heat shock protein Hsp60, cell surface protein H1C, dipeptidyl peptidase type IV DppIV, M antigen, 70 kDa protein, 17 kDa histone-like protein (Histoplasma capsulatum, Histoplasmosis); fatty acid and retinol binding protein-1 FAR-1, tissue inhibitor of metalloproteinase TIMP (TMP), cysteine proteinase ACEY-1, cysteine proteinase ACCP-1, surface antigen Ac-16, secreted protein 2 ASP-2, metalloprotease 1 MTP-1, aspartyl protease inhibitor API-1, surface-associated antigen SAA-1, surface-associated antigen SAA-2, adult-specific secreted factor Xa, serine protease inhibitor anticoagulant AP, cathepsin D-like aspartic protease ARR-1, 5-transferase GST, aspartic protease APR-1, acetylcholinesterase AChE (Ancylostoma duodena le and Necator americanus, Hookworm infection); protein NS1, protein NP1, protein VP1, protein VP2, protein VP3 (Human bocavirus (HBoV), Human bocavirus infection); major surface protein 2 MSP2, major surface protein 4 MSP4, MSP variant SGV1, MSP variant SGV2, outer membrane protein OMP, outer membrande protein 19 OMP-19, major antigenic protein MAP1, major antigenic protein MAP1-2, major antigenic protein MAP1B, major antigenic protein MAP1-3, Erum2510 coding protein, protein GroEL, protein GroES, 30-kDA major outer membrane proteins, GE 100-kDa protein, GE 130-kDa protein, GE 160-kDa protein (Ehrlichia ewingii, Human ewingii ehrlichiosis); major surface proteins 1-5 (MSP1a, MSP1b, MSP2, MSP3, MSP4, MSP5), type IV secreotion system proteins VirB2, VirB7, VirB11, VirD4 (Anaplasma phagocytophilum, Human granulocytic anaplasmosis (HGA)); protein NS1, small hydrophobic protein N52, SH protein, fusion protein F, glycoprotein G, matrix protein M, matrix protein M2-1, matrix protein M2-2, phosphoprotein P, nucleoprotein N, polymerase L (Human metapneumovirus (hMPV), Human metapneumovirus infection); major surface protein 2 MSP2, major surface protein 4 MSP4, MSP variant SGV1, MSP variant SGV2, outer membrane protein OMP, outer membrande protein 19 OMP-19, major antigenic protein MAP1, major antigenic protein MAP1-2, major antigenic protein MAP1B, major antigenic protein MAP1-3, Erum2510 coding protein, protein GroEL, protein GroES, 30-kDA major outer membrane proteins, GE 100-kDa protein, GE 130-kDa protein, GE 160-kDa protein (Ehrlichia chaffeensis, Human monocytic ehrlichiosis); replication protein E1, regulatory protein E2, protein E3, protein E4, protein ES, protein E6, protein E7, protein E8, major capsid protein L1, minor capsid protein L2 (Human papillomavirus (HPV), Human papillomavirus (HPV) infection); fusion protein F, hemagglutinin-neuramidase HN, glycoprotein G, matrix protein M, phosphoprotein P, nucleoprotein N, polymerase L (Human parainfluenza viruses (HPIV), Human parainfluenza virus infection); Hemagglutinin (HA), Neuraminidase (NA), Nucleoprotein (NP), M1 protein, M2 protein, NS1 protein, NS2 protein (NEP protein: nuclear export protein), PA protein, PB1 protein (polymerase basic 1 protein), PB1-F2 protein and PB2 protein (Orthomyxoviridae family, Influenza virus (flu)); genome polyprotein, protein E, protein M, capsid protein C (Japanese encephalitis virus, Japanese encephalitis); RTX toxin, type IV pili, major pilus subunit PilA, regulatory transcription factors PilS and PilR, protein sigma54, outer membrane proteins (Kingella kingae, Kingella kingae infection); prion protein (Kuru prion, Kuru); nucleoprotein N, polymerase L, matrix protein Z, glycoprotein GP (Lassa virus, Lassa fever); peptidoglycan-associated lipoprotein PAL, 60 kDa chaperonin Cpn60 (groEL, HspB), type IV pilin PilE, outer membrane protein MIP, major outer membrane protein MompS, zinc metalloproteinase MSP (Legionella pneumophila, Legionellosis (Legionnaires' disease, Pontiac fever)); P4 nuclease, protein WD, ribonucleotide reductase M2, surface membrane glycoprotein Pg46, cysteine proteinase CP, glucose-regulated protein 78 GRP-78, stage-specific S antigen-like protein A2, ATPase F1, beta-tubulin, heat shock protein 70 Hsp70, KMP-11, glycoprotein GP63, protein BT1, nucleoside hydrolase NH, cell surface protein B 1, ribosomal protein P1-like protein P1, sterol 24-c-methyltransferase SMT, LACK protein, histone H1, SPB1 protein, thiol specific antioxidant TSA, protein antigen STI1, signal peptidase SP, histone H2B, surface antigen PSA-2, cystein proteinase b Cpb (Leishmania genus, Leishmaniasis); major membrane protein I, serine-rich antigen-45 kDa, 10 kDa caperonin GroES, HSP kDa antigen, amino-oxononanoate synthase AONS, protein recombinase A RecA, Acetyl-/propionyl-coenzyme A carboxylase alpha, alanine racemase, 60 kDa chaperonin 2, ESAT-6-like protein EcxB (L-ESAT-6), protein Lsr2, protein ML0276, Heparin-binding hemagglutinin HBHA, heat-shock protein 65 Hsp65, mycP1 or ML0041 coding protein htrA2 or ML0176 coding protein htrA4 or ML2659 coding protein, gcp or ML0379 coding protein, clpC or ML0235 coding protein (Mycobacterium leprae and Mycobacterium lepromatosis, Leprosy); outer membrane protein LipL32, membrane protein LIC10258, membrane protein LP30, membrane protein LIC12238, Ompa-like protein Lsa66, surface protein LigA, surface protein LigB, major outer membrane protein OmpL1, outer membrane protein LipL41, protein LigAni, surface protein LcpA, adhesion protein LipL53, outer membrane protein UpL32, surface protein Lsa63, flagellin FlaB1, membrane lipoprotein LipL21, membrane protein pL40, leptospiral surface adhesin Lsa27, outer membrane protein OmpL36, outer membrane protein OmpL37, outer membrane protein OmpL47, outer membrane protein OmpL54, acyltransferase LpxA (Leptospira genus, Leptospirosis); listeriolysin 0 precursor Hly (LL0), invasion-associated protein Iap (P60), Listeriolysin regulatory protein PrfA, Zinc metalloproteinase Mpl, Phosphatidylinositol-specific phospholipase C PLC (PIcA, PlcB), 0-acetyltransferase Oat, ABC-transporter permease Im.G_1771, adhesion protein LAP, LAP receptor Hsp60, adhesin LapB, haemolysin listeriolysin 0 LLO, protein ActA, Internalin A InIA, protein InIB (Listeria monocytogenes, Listeriosis); outer surface protein A OspA, outer surface protein OspB, outer surface protein OspC, decorin binding protein A DbpA, decorin binding protein B DbpB, flagellar filament 41 kDa core protein Fla, basic membrane protein A BmpA (Immunodominant antigen P39), outer surface 22 kDa lipoprotein precursor (antigen IPLA7), variable surface lipoprotein vlsE (usually Borrelia burgdorferi and other Borrelia species, Lyme disease (Lyme borreliosis)); venom allergen homolog-like protein VAL-1, abundant larval transcript ALT-1, abundant larval transcript ALT-2, thioredoxin peroxidase TPX, vespid allergen homologue VAH, thiordoxin peroxidase 2 TPX-2, antigenic protein SXP (peptides N, N1, N2, and N3), activation associated protein-1 ASP-1, thioredoxin TRX, transglutaminase BmTGA, glutathione-S-transferases GST, myosin, vespid allergen homologue VAH, 175 kDa collagenase, glyceraldehyde-3-phosphate dehydrogenase GAPDH, cuticular collagen Col-4, Secreted Larval Acidic Proteins SLAPs, chitinase CHI-1, maltose binding protein MBP, glycolytic enzyme fructose-1,6-bisphosphate aldolase Fba, tropomyosin TMY-1, nematode specific gene product OvB20, onchocystatin CPI-2, protein Cox-2 (Wuchereria bancrofti and Brugia malayi, Lymphatic filariasis (Elephantiasis)); glycoprotein GP, matrix protein polymerase L, nucleoprotein N (Lymphocytic choriomeningitis virus (LCMV), Lymphocytic choriomeningitis); thrombospondin-related anonymous protein TRAP, SSP2 Sporozoite surface protein 2, apical membrane antigen 1 AMA1, rhoptry membrane antigen RMA1, acidic basic repeat antigen ABRA, cell-traversal protein PF, protein Pvs25, merozoite surface protein 1 MSP-1, merozoite surface protein 2 MSP-2, ring-infected erythrocyte surface antigen RESALiver stage antigen 3 LSA-3, protein Eba-175, serine repeat antigen 5 SERA-5, circumsporozoite protein CS, merozoite surface protein 3 MSP3, merozoite surface protein 8 MSP5, enolase PF10, hepatocyte erythrocyte protein 17 kDa HEP17, erythrocyte membrane protein 1 EMP1, protein Kbetamerozoite surface protein 4/5 MSP 4/5, heat shock protein Hsp90, glutamate-rich protein GLURP, merozoite surface protein 4 MSP-4, protein STARP, circumsporozoite protein-related antigen precursor CRA (Plasmodium genus, Malaria); nucleoprotein N, membrane-associated protein VP24, minor nucleoprotein VP30, polymerase cofactor VP35, polymerase L, matrix protein VP40, envelope glycoprotein GP (Marburg virus, Marburg hemorrhagic fever (MHF)); protein C, matrix protein M, phosphoprotein P, non-structural protein V, hemagglutinin glycoprotein H, polymerase L, nucleoprotein N, fusion protein F (Measles virus, Measles); members of the ABC transporter family (LoIC, OppA, and PotF), putative lipoprotein releasing system transmembrane protein LoIC/E, flagellin FliC, Burkholderia intracellular motility A BimA, bacterial Elongation factor-Tu EF-Tu, 17 kDa OmpA-like protein, boaA coding protein, boaB coding protein (Burkholderia pseudomallei, Melioidosis (Whitmore's disease)); pilin proteins, minor pilin-associated subunit pilC, major pilin subunit and variants pilE, pilS, phase variation protein porA, Porin B PorB, protein TraD, Neisserial outer membrane antigen H.8, 70 kDa antigen, major outer membrane protein PI, outer membrane proteins PIA and PIB, W antigen, surface protein A NspA, transferrin binding protein TbpA, transferrin binding protein TbpB PBP2, mtrR coding protein, ponA coding protein, membrane permease FbpBC, FbpABC protein system, LbpAB proteins, outer membrane protein Opa, outer membrane transporter FetA, iron-repressed regulator MpeR, factor H-binding protein fHbp, adhesin NadA, protein NhbA, repressor FarR (Neisseria meningitidis, Meningococcal disease); 66 kDa protein, 22 kDa protein (usually Metagonimus yokagawai, Metagonimiasis); polar tube proteins (34, 75, and 170 kDa in Glugea, 35, 55 and 150 kDa in Encephalitozoon), kinesin-related protein, RNA polymerase II largest subunit, similar of integral membrane protein YIPA, anti-silencing protein 1, heat shock transcription factor HSF, protein kinase, thymidine kinase, NOP-2 like nucleolar protein (Microsporidia phylum, Microsporidiosis); CASP8 and FADD-like apoptosis regulator, Glutathione peroxidase GPX1, RNA helicase NPH-II NPH2, Poly(A) polymerase catalytic subunit PAPL, Major envelope protein P43K, early transcription factor 70 kDa subunit VETFS, early transcription factor 82 kDa subunit VETFL, metalloendopeptidase G1-type, nucleoside triphosphatase I NPH1, replication protein A28-like MC134L, RNA polymease 7 kDa subunit RPO7 (Molluscum contagiosum virus (MCV), Molluscum contagiosum (MC)); matrix protein M, phosphoprotein P/V, small hydrophobic protein SH, nucleoprotein N, protein V, fusion glycoprotein hemagglutinin-neuraminidase HN, RNA polymerase L (Mumps virus, Mumps); Outer membrane proteins OM, cell surface antigen OmpA, cell surface antigen OmpB (sca5), cell surface protein SCA4, cell surface protein SCA1, intracytoplasmic protein D, crystalline surface layer protein SLP, protective surface protein antigen SPA (Rickettsia typhi, Murine typhus (Endemic typhus)); adhesin P1, adhesion P30, protein p116, protein P40, cytoskeletal protein HMW1, cytoskeletal protein HMW2, cytoskeletal protein HMW3, MPN152 coding protein, MPN426 coding protein, MPN456 coding protein, MPN-500coding protein (Mycoplasma pneumoniae, Mycoplasma pneumonia); NocA, Iron dependent regulatory protein, VapA, VapD, VapF, VapG, caseinolytic protease, filament tip-associated 43-kDa protein, protein P24, protein P61, 15-kDa protein, 56-kDa protein (usually Nocardia asteroides and other Nocardia species, Nocardiosis); venom allergen homolog-like protein VAL-1, abundant larval transcript ALT-1, abundant larval transcript ALT-2, thioredoxin peroxidase TPX, vespid allergen homologue VAH, thiordoxin peroxidase 2 TPX-2, antigenic protein SXP (peptides N, N1, N2, and N3), activation associated protein-1 ASP-1, Thioredoxin TRX, transglutaminase BmTGA, glutathione-S-transferases GST, myosin, vespid allergen homologue VAH, 175 kDa collagenase, glyceraldehyde-3-phosphate dehydrogenase GAPDH, cuticular collagen Col-4, Secreted Larval Acidic Proteins SLAPs, chitinase CHI-1, maltose binding protein MBP, glycolytic enzyme fructose-1,6-bisphosphate aldolase Fba, tropomyosin TMY-1, nematode specific gene product OvB20, onchocystatin CPI-2, Cox-2 (Onchocerca volvulus, Onchocerciasis (River blindness)); 43 kDa secreted glycoprotein, glycoprotein gp0, glycoprotein gp75, antigen Pb27, antigen Pb40, heat shock protein Hsp65, heat shock protein Hsp70, heat shock protein Hsp90, protein P10, triosephosphate isomerase TPI, N-acetyl-glucosamine-binding lectin Paracoccin, 28 kDa protein Pb28 (Paracoccidioides brasiliensis, Paracoccidioidomycosis (South American blastomycosis)); 28-kDa cruzipain-like cystein protease Pw28CCP (usually Paragonimus westermani and other Paragonimus species, Paragonimiasis); outer membrane protein OmpH, outer membrane protein Omp28, protein PM1539, protein PM0355, protein PM1417, repair protein MutL, protein BcbC, prtein PM0305, formate dehydrogenase-N, protein PM0698, protein PM1422, DNA gyrase, lipoprotein PIpE, adhesive protein Cp39, heme aquisition system receptor HasR, 39 kDa capsular protein, iron-regulated OMP IROMP, outer membrane protein OmpA87, fimbrial protein Ptf, fimbrial subunit protein PtfA, transferrin binding protein Tbpl, esterase enzyme MesA, Pasteurella multocida toxin PMT, adhesive protein Cp39 (Pasteurella genus, Pasteurellosis); “filamentous hemagglutinin FhaB, adenylate cyclase CyaA, pertussis toxin subunit 4 precursor PtxD, pertactin precursor Prn, toxin subunit 1 PtxA, protein Cpn60, protein brkA, pertussis toxin subunit 2 precursor PtxB, pertussis toxin subunit 3 precursor PtxC, pertussis toxin subunit 5 precursor PtxE, pertactin Pm, protein Fim2, protein Fim3;” (Bordetella pertussis, Pertussis (Whooping cough)); “F1 capsule antigen, virulence-associated V antigen, secreted effector protein LcrV, V antigen, outer membrane protease Pla, secreted effector protein YopD, putative secreted protein-tyrosine phosphatase YopH, needle complex major subunit YscF, protein kinase YopO, putative autotransporter protein YapF, inner membrane ABC-transporter YbtQ (Irp7), putative sugar binding protein YP00612, heat shock protein 90 HtpG, putative sulfatase protein YdeN, outer-membrane lipoprotein carrier protein LoIA, secretion chaperone YerA, putative lipoprotein YP00420, hemolysin activator protein HpmB, pesticin/yersiniabactin outer membrane receptor Psn, secreted effector protein YopE, secreted effector protein YopF, secreted effector protein YopK, outer membrane protein YopN outer membrane protein YopM, Coagulase/fibrinolysin precursor Pla;” (Yersinia pestis, Plague); protein PhpA, surface adhesin PsaA, pneumolysin Ply, ATP-dependent protease CIp, lipoate-protein ligase LpIA, cell wall surface anchored protein psrP, sortase SrtA, glutamyl-tRNA synthetase GItX, choline binding protein A CbpA, pneumococcal surface protein A PspA, pneumococcal surface protein C PspC, 6-phosphogluconate dehydrogenase Gnd, iron-binding protein PiaA, Murein hydrolase LytB, proteon LytC, protease A1 (Streptococcus pneumoniae, Pneumococcal infection); major surface protein B, kexin-like protease KEX1, protein A12, 55 kDa antigen P55, major surface glycoprotein Msg (Pneumocystis jirovecii, Pneumocystis pneumonia (PCP)); genome polyprotein, polymerase 3D, viral capsid protein VP1, viral capsid protein VP2, viral capsid protein VP3, viral capsid protein VP4, protease 2A, protease 3C (Poliovirus, Poliomyelitis); protein Nfa1, exendin-3, secretory lipase, cathepsin B-like protease, cysteine protease, cathepsin, peroxiredoxin, protein Cry1Ac (usually Naegleria fowleri, Primary amoebic meningoencephalitis (PAM)); agnoprotein, large T antigen, small T antigen, major capsid protein VP1, minor capsid protein Vp2 (JC virus, Progressive multifocal leukoencephalopathy); low calcium response protein E LCrE, chlamydial outer protein N CopN, serine/threonine-protein kinase PknD, acyl-carrier-protein S-malonyltransferase FabD, single-stranded DNA-binding protein Ssb, major outer membrane protein MOMP, outer membrane protein 2 Omp2, polymorphic membrane protein family (Pmp1, Pmp2, Pmp3, Pmp4, Pmp5, Pmp6, Pmp7, Pmp8, Pmp9, Pmp10, Pmp11, Pmp12, Pmp13, Pmp14, Pmp15, Pmp16, Pmp17, Pmp18, Pmp19, Pmp20, Pmp21) (Chlamydophila psittaci, Psittacosis); outer membrane protein P1, heat shock protein B HspB, peptide ABC transporter, GTP-binding protein, protein IcmB, ribonuclease R, phosphatas SixA, protein DsbD, outer membrane protein ToIC, DNA-binding protein PhoB, ATPase DotB, heat shock protein B HspB, membrane protein Coml, 28 kDa protein, DNA-3-methyladenine glycosidase I, pouter membrane protein OmpH, outer membrane protein AdaA, glycine cleavage system T-protein (Coxiella burnetii, Q fever); nucleoprotein N, large structural protein L, phophoprotein P, matrix protein M, glycoprotein G (Rabies virus, Rabies); fusionprotein F, nucleoprotein N, matrix protein M, matrix protein M2-1, matrix protein M2-2, phophoprotein P, small hydrophobic protein SH, major surface glycoprotein G, polymerase L, non-structural protein 1 NS1, non-structural protein 2 NS2 (Respiratory syncytial virus (RSV), Respiratory syncytial virus infection); genome polyprotein, polymerase 3D, viral capsid protein VP1, viral capsid protein VP2, viral capsid protein VP3, viral capsid protein VP4, protease 2A, protease 3C (Rhinovirus, Rhinovirus infection); outer membrane proteins OM, cell surface antigen OmpA, cell surface antigen OmpB (sca5), cell surface protein SCA4, cell surface protein SCA1, protein PS120, intracytoplasmic protein D, protective surface protein antigen SPA (Rickettsia genus, Rickettsial infection); outer membrane proteins OM, cell surface antigen OmpA, cell surface antigen OmpB (sca5), cell surface protein SCA4, cell surface protein SCA1, intracytoplasmic protein D (Rickettsia akari, Rickettsialpox); envelope glycoprotein GP, polymerase L, nucleoprotein N, non-structural protein NSS (Rift Valley fever virus, Rift Valley fever (RVF)); outer membrane proteins OM, cell surface antigen OmpA, cell surface antigen OmpB (sca5), cell surface protein SCA4, cell surface protein SCA1, intracytoplasmic protein D (Rickettsia rickettsii, Rocky mountain spotted fever (RMSF)); non-structural protein 6 N56, non-structural protein 2 N52, intermediate capsid protein VP6, inner capsid protein VP2, non-structural protein 3 NS3, RNA-directed RNA polymerase L, protein VP3, non-structural protein 1 NS1, non-structural protein 5 N55, outer capsid glycoprotein VP7, non-structural glycoprotein 4 N54, outer capsid protein VP4; (Rotavirus, Rotavirus infection); polyprotein P200, glycoprotein E1, glycoprotein E2, protein N52, capsid protein C (Rubella virus, Rubella); chaperonin GroEL (MopA), inositol phosphate phosphatase SopB, heat shock protein HslU, chaperone protein DnaJ, protein TviB, protein IroN, flagellin FliC, invasion protein SipC, glycoprotein gp43, outer membrane protein LamB, outer membrane protein PagC, outer membrane protein ToIC, outer membrane protein NmpC, outer membrane protein FadL, transport protein SadA, transferase WgaP, effector proteins SifA, SteC, SseL, SseJ and SseF (Salmonella genus, Salmonellosis); “protein 14, non-structural protein NS7b, non-structural protein NS8a, protein 9b, protein 3a, nucleoprotein N, non-structural protein NS3b, non-structural protein N56, protein 7a, non-structural protein NS8b, membrane protein M, envelope small membrane protein EsM, replicase polyprotein la, spike glycoprotein S, replicase polyprotein lab; SARS coronavirus, SARS (Severe Acute Respiratory Syndrome)); serin protease, Atypical Sarcoptes Antigen 1 ASAI, glutathione 5-transferases GST, cystein protease, serine protease, apolipoprotein (Sarcoptes scabiei, Scabies); glutathione 5-transferases GST, paramyosin, hemoglbinase SM32, major egg antigen, 14 kDa fatty acid-binding protein Sm14, major larval surface antigen P37, 22.6 kDa tegumental antigen, calpain CANP, triphospate isomerase Tim, surface protein 9B, outer capsid protein VP2, 23 kDa integral membrane protein Sm23, Cu/Zn-superoxide dismutase, glycoprotein Gp, myosin (Schistosoma genus, Schistosomiasis (Bilharziosis)); 60 kDa chaperonin, 56 kDa type-specific antigen, pyruvate phosphate dikinase, 4-hydroxybenzoate octaprenyltransferase (Orientia tsutsugamushi, Scrub typhus); dehydrogenase GuaB, invasion protein Spa32, invasin IpaA, invasin IpaB, invasin IpaC, invasin IpaD, invasin IpaH, invasin IpaJ (Shigella genus, Shigellosis (Bacillary dysentery)); protein P53, virion protein US10 homolog, transcriptional regulator 1E63, transcriptional transactivator 1E62, protease P33, alpha trans-inducing factor 74 kDa protein, deoxyuridine 5′-triphosphate nucleotidohydrolase, transcriptional transactivator 1E4, membrane protein UL43 homolog, nuclear phosphoprotein UL3 homolog, nuclear protein UL4 homolog, replication origin-binding protein, membrane protein 2, phosphoprotein 32, protein 57, DNA polymerase processivity factor, portal protein 54, DNA primase, tegument protein UL14 homolog, tegument protein UL21 homolog, tegument protein UL55 homolog, tripartite terminase subunit UL33 homolog, tripartite terminase subunit UL15 homolog, capsid-binding protein 44, virion-packaging protein 43 (Varicella zoster virus (VZV), Shingles (Herpes zoster)); truncated 3-beta hydroxy-5-ene steroid dehydrogenase homolog, virion membrane protein A13, protein A19, protein A31, truncated protein A35 homolog, protein A37.5 homolog, protein A47, protein A49, protein A51, semaphorin-like protein A43, serine proteinase inhibitor 1, serine proteinase inhibitor 2, serine proteinase inhibitor 3, protein A6, protein B15, protein C1, protein C5, protein C6, protein F7, protein F8, protein F9, protein F11, protein F14, protein F15, protein F16 (Variola major or Variola minor, Smallpox (Variola)); adhesin/glycoprotein gp70, proteases (Sporothrix schenckii, Sporotrichosis); heme-iron binding protein IsdB, collagen adhesin Cna, clumping factor A CIfA, protein MecA, fibronectin-binding protein A FnbA, enterotoxin type A EntA, enterotoxin type B EntB, enterotoxin type C EntC1, enterotoxin type C EntC2, enterotoxin type D EntD, enterotoxin type E EntE, Toxic shock syndrome toxin-1 TSST-1, Staphylokinase, Penicillin binding protein 2a PBP2a (MecA), secretory antigen SssA (Staphylococcus genus, Staphylococcal food poisoning); heme-iron binding protein IsdB, collagen adhesin Cna, clumping factor A CIfA, protein MecA, fibronectin-binding protein A FnbA, enterotoxin type A EntA, enterotoxin type B EntB, enterotoxin type C EntC1, enterotoxin type C EntC2, enterotoxin type D EntD, enterotoxin type E EntE, Toxic shock syndrome toxin-1 TSST-1, Staphylokinase, Penicillin binding protein 2a PBP2a (MecA), secretory antigen SssA (Staphylococcus genus e.g. aureus, Staphylococcal infection); antigen Ss-IR, antigen NIE, strongylastacin, Na+-K+ ATPase Sseat-6, tropomysin SsTmy-1, protein LEC-5, 41 kDa aantigen P5, 41-kDa larval protein, 31-kDa larval protein, 28-kDa larval protein (Strongyloides stercoralis, Strongyloidiasis); glycerophosphodiester phosphodiesterase GlpQ (Gpd), outer membrane protein TmpB, protein Tp92, antigen TpF 1, repeat protein Tpr, repeat protein F TprF, repeat protein G TprG, repeat protein I Tprl, repeat protein J TprJ, repeat protein KTprK, treponemal membrane protein A TmpA, lipoprotein, 15 kDa Tpp15, 47 kDa membrane antigen, miniferritin TpF1, adhesin Tp0751, lipoprotein TP0136, protein TpN17, protein TpN47, outer membrane protein TP0136, outer membrane protein TP0155, outer membrane protein TP0326, outer membrane protein TP0483, outer membrane protein TP0956 (Treponema pallidum, Syphilis); Cathepsin L-like proteases, 53/25-kDa antigen, 8 kDa family members, cysticercus protein with a marginal trypsin-like activity TsAg5, oncosphere protein TSOL18, oncosphere protein TSOL45-1A, lactate dehydrogenase A LDHA, lactate dehydrogenase B LDHB (Taenia genus, Taeniasis); tetanus toxin TetX, tetanus toxin C TTC, 140 kDa S layer protein, flavoprotein beta-subunit CT3, phospholipase (lecithinase), phosphocarrier protein HPr (Clostridium tetani, Tetanus (Lockjaw)); genome polyprotein, protein E, protein M, capsid protein C (Tick-borne encephalitis virus (TBEV), Tick-borne encephalitis); 58-kDa antigen, 68-kDa antigens, Toxocara larvae excretory-secretory antigen TES, 32-kDa glycoprotein, glycoprotein TES-70, glycoprotein GP31, excretory-secretory antigen TcES-57, perienteric fluid antigen Pe, soluble extract antigens Ex, excretory/secretory larval antigens ES, antigen TES-120, polyprotein allergen TBA-1, cathepsin L-like cysteine protease c-cp1-1, 26-kDa protein (Toxocara canis or Toxocara cati, Toxocariasis (Ocular Larva Migrans (OLM) and Visceral Larva Migrans (VLM))); microneme proteins (MIC1, MIC2, MIC3, MIC4, MIC5, MICE, MIC7, MICE), rhoptry protein Rop2, rhoptry proteins (Rop1, Rop2, Rop3, Rop4, Rop5, Rop6, Rop7, Rop16, Rjop17), protein SR1, surface antigen P22, major antigen p24, major surface antigen p30, dense granule proteins (GRA1, GRA2, GRA3, GRA4, GRA5, GRA6, GRA7, GRAB, GRA9, GRA10), 28 kDa antigen, surface antigen SAG1, SAG2 related antigen, nucleoside-triphosphatase 1, nucleoside-triphosphatase 2, protein Stt3, HesB-like domain-containing protein, rhomboid-like protease 5, toxomepsin 1 (Toxoplasma gondii, Toxoplasmosis); 43 kDa secreted glycoprotein, 53 kDa secreted glycoprotein, paramyosin, antigen Ts21, antigen Ts87, antigen p46000, TSL-1 antigens, caveolin-1 CAV-1, 49 kDa newborn larva antigen, prosaposin homologue, serine protease, serine proteinase inhibitor, 45-kDa glycoprotein Gp45 (Trichinella spiralis, Trichinellosis); Myb-like transcriptional factors (Myb1, Myb2, Myb3), adhesion protein AP23, adhesion protein AP33, adhesin protein AP33-3, adhesins AP51, adhesin AP65, adhesion protein AP65-1, alpha-actinin, kinesin-associated protein, teneurin, 62 kDa proteinase, subtilisin-like serine protease SUB1, cysteine proteinase gene 3 CP3, alpha-enolase Enol, cysteine proteinase CP30, heat shock proteins (Hsp70, Hsp60) immunogenic protein P270, (Trichomonas vaginalis, Trichomoniasis); beta-tubulin, 47-kDa protein, secretory leucocyte-like proteinase-1 SLP-1, 50-kDa protein TT50, 17 kDa antigen, 43/47 kDa protein (Trichuris trichiura, Trichuriasis (Whipworm infection)); protein ESAT-6 (EsxA), 10 kDa filtrate antigen EsxB, secreted antigen 85-B FBPB, fibronectin-binding protein A FbpA (Ag85A), serine protease PepA, PPE family protein PPE18, fibronectin-binding protein D FbpD, immunogenic protein MPT64, secreted protein MPT51, catalase-peroxidase-peroxynitritase T KATG, periplasmic phosphate-binding lipoprotein PSTS3 (PBP-3, Phos-1), iron-regulated heparin binding hemagglutinin Hbha, PPE family protein PPE14, PPE family protein PPE68, protein Mtb72F, protein Apa, immunogenic protein MPT63, periplasmic phosphate-binding lipoprotein PSTS1 (PBP-1), molecular chaperone DnaK, cell surface lipoprotein Mpt83, lipoprotein P23, phosphate transport system permease protein pstA, 14 kDa antigen, fibronectin-binding protein C FbpC1, Alanine dehydrogenase TB43, Glutamine synthetase 1, ESX-1 protein, protein CFP10, TB10.4 protein, protein MPT83, protein MTB12, protein MTBE, Rpf-like proteins, protein MTB32, protein MTB39, crystallin, heat-shock protein HSP65, protein PST-S (usually Mycobacterium tuberculosis, Tuberculosis); outer membrane protein FobA, outer membrane protein FobB, intracellular growth locus IgIC1, intracellular growth locus IgIC2, aminotransferase Wbt1, chaperonin GroEL, 17 kDa major membrane protein TUL4, lipoprotein LpnA, chitinase family 18 protein, isocitrate dehydrogenase, Nif3 family protein, type IV pili glycosylation protein, outer membrane protein tolC, FAD binding family protein, type IV pilin multimeric outer membrane protein, two component sensor protein KdpD, chaperone protein DnaK, protein TolQ (Francisella tularensis, Tularemia); “MB antigen, urease, protein GyrA, protein GyrB, protein ParC, protein ParE, lipid associated membrane proteins LAMP, thymidine kinase TK, phospholipase PL-A1, phospholipase PL-A2, phospholipase PL-C, surface-expressed 96-kDa antigen;” (Ureaplasma urealyticum, Ureaplasma urealyticum infection); non-structural polyprotein, structural polyprotein, capsid protein CP, protein E1, protein E2, protein E3, protease Pb, protease P2, protease P3 (Venezuelan equine encephalitis virus, Venezuelan equine encephalitis); glycoprotein GP, matrix protein Z, polymerase L, nucleoprotein N (Guanarito virus, Venezuelan hemorrhagic fever); polyprotein, protein E, protein M, capsid protein C, protease NS3, protein NS1, protein NS2A, protein AS2B, brotein NS4A, protein NS4B, protein NS5 (West Nile virus, West Nile Fever); cpasid protein CP, protein E1, protein E2, protein E3, protease P2 (Western equine encephalitis virus, Western equine encephalitis); genome polyprotein, protein E, protein M, capsid protein C, protease NS3, protein NS1, protein NS2A, protein AS2B, protein NS4A, protein NS4B, protein NS5 (Yellow fever virus, Yellow fever); putative Yop targeting protein YobB, effector protein YopD, effector protein YopE, protein YopH, effector protein YopJ, protein translocation protein YopK, effector protein YopT, protein YpkA, flagellar biosyntheses protein FIhA, peptidase M48, potassium efflux system KefA, transcriptional regulatoer RovA, adhesin Ifp, translocator portein LcrV, protein PcrV, invasin Inv, outer membrane protein OmpF-like porin, adhesin YadA, protein kinase C, phospholipase C1, protein PsaA, mannosyltransferase-like protein WbyK, protein YscU, antigen YPMa (Yersinia pseudotuberculosis, Yersinia pseudotuberculosis infection); and effector protein YopB, 60 kDa chaperonin, protein WbcP, tyrosin-protein phosphatase YopH, protein YopQ, enterotoxin, Galactoside permease, reductaase NrdE, protein YasN, Invasin Inv, adhesin YadA, outer membrane porin F OmpF, protein UspA1, protein EibA, protein Hia, cell surface protein Ail, chaperone SycD, protein LcrD, protein LcrG, protein LcrV, protein SycE, protein YopE, regulator protein TyeA, protein YopM, protein YopN, protein YopO, protein YopT, protein YopD, protease CIpP, protein MyfA, protein FilA, and protein PsaA (Yersinia enterocolitica, Yersiniosis).

The infectious agent can be a bacterium, a fungus, a virus, or a protist. The infectious agent can be a coronavirus (CoV) (e.g., an alphacoronavirus, a betacoronavirus, a gammacoronavirus, or a deltacoronavirus). The infectious agent can be selected from the group comprising Acinetobacter baumannii, Anaplasma genus, Anaplasma phagocytophilum, Ancylostoma braziliense, Ancylostoma duodenale, Arcanobacterium haemolyticum, Ascaris lumbricoides, Aspergillus genus, Astroviridae, Babesia genus, Bacillus anthracis, Bacillus cereus, Bartonella henselae, BK virus, Blastocystis hominis, Blastomyces dermatitidis, Bordetella pertussis, Borrelia burgdorferi, Borrelia genus, Borrelia spp, Brucella genus, Brugia malayi, Bunyaviridae family, Burkholderia cepacia and other Burkholderia species, Burkholderia mallei, Burkholderia pseudomallei, Caliciviridae family, Campylobacter genus, Candida albicans, Candida spp, Chlamydia trachomatis, Chlamydophila pneumoniae, Chlamydophila psittaci, CJD prion, Clonorchis sinensis, Clostridium botulinum, Clostridium difficile, Clostridium perfringens, Clostridium perfringens, Clostridium spp, Clostridium tetani, Coccidioides spp, coronaviruses, Corynebacterium diphtherias, Coxiella burnetii, Crimean-Congo hemorrhagic fever virus, Cryptococcus neoformans, Cryptosporidium genus, Cytomegalovirus (CMV), Dengue viruses (DEN-1, DEN-2, DEN-3 and DEN-4), Dientamoeba fragilis, Ebolavirus (EBOV), Echinococcus genus, Ehrlichia chaffeensis, Ehrlichia ewingii, Ehrlichia genus, Entamoeba histolytica, Enterococcus genus, Enterovirus genus, Enteroviruses, mainly Coxsackie A virus and Enterovirus 71 (EV71), Epidermophyton spp, Epstein-Barr Virus (EBV), Escherichia coli O157:H7, O111 and O104:H4, Fasciola hepatica and Fasciola gigantica, FFI prion, Filarioidea superfamily, Filoviruses, Flaviviruses, Francisella tularensis, Fusobacterium genus, Geotrichum candidum, Giardia intestinalis, Gnathostoma spp, GSS prion, Guanarito virus, Haemophilus ducreyi, Haemophilus influenzae, Helicobacter pylori, Henipavirus (Hendra virus Nipah virus), Hepatitis A Virus, Hepatitis B Virus (HBV), Hepatitis C Virus (HCV), Hepatitis D Virus, Hepatitis E Virus, Herpes simplex virus 1 and 2 (HSV-1 and HSV-2), Histoplasma capsulatum, HIV (Human immunodeficiency virus), Hortaea werneckii, Human bocavirus (HBoV), Human herpesvirus 6 (HHV-6) and Human herpesvirus 7 (HHV-7), Human metapneumovirus (hMPV), Human papillomavirus (HPV), Human parainfluenza viruses (HPIV), Japanese encephalitis virus, JC virus, Junin virus, Kingella kingae, Klebsiella granulomatis, Kuru prion, Lassa virus, Legionella pneumophila, Leishmania genus, Leptospira genus, Listeria monocytogenes, Lymphocytic choriomeningitis virus (LCMV), Machupo virus, Malassezia spp, Marburg virus, Measles virus, Metagonimus yokagawai, Microsporidia phylum, Molluscum contagiosum virus (MCV), Mumps virus, Mycobacterium leprae and Mycobacterium lepromatosis, Mycobacterium tuberculosis, Mycobacterium ulcerans, Mycoplasma pneumoniae, Naegleria fowleri, Necator americanus, Neisseria gonorrhoeae, Neisseria meningitidis, Nocardia asteroides, Nocardia spp, Onchocerca volvulus, Orientia tsutsugamushi, Orthomyxoviridae family (Influenza), Paracoccidioides brasiliensis, Paragonimus spp, Paragonimus westermani, Parvovirus B19, Pasteurella genus, Plasmodium genus, Pneumocystis jirovecii, Poliovirus, Rabies virus, Respiratory syncytial virus (RSV), Rhinovirus, rhinoviruses, Rickettsia akari, Rickettsia genus, Rickettsia prowazekii, Rickettsia rickettsii, Rickettsia typhi, Rift Valley fever virus, Rotavirus, Rubella virus, Sabia virus, Salmonella genus, Sarcoptes scabiei, SARS coronavirus, Schistosoma genus, Shigella genus, Sin Nombre virus, Hantavirus, Sporothrix schenckii, Staphylococcus genus, Staphylococcus genus, Streptococcus agalactiae, Streptococcus pneumoniae, Streptococcus pyogenes, Strongyloides stercoralis, Taenia genus, Taenia solium, Tick-borne encephalitis virus (TBEV), Toxocara canis or Toxocara cati, Toxoplasma gondii, Treponema pallidum, Trichinella spiralis, Trichomonas vaginalis, Trichophyton spp, Trichuris trichiura, Trypanosoma brucei, Trypanosoma cruzi, Ureaplasma urealyticum, Varicella zoster virus (VZV), Variola major or Variola minor, vCJD prion, Venezuelan equine encephalitis virus, Vibrio cholerae, West Nile virus, Western equine encephalitis virus, Wuchereria bancrofti, Yellow fever virus, Yersinia enterocolitica, Yersinia pestis, and Yersinia pseudotuberculosis.

The AP can comprise or can be derived from the full-length surface protein of a coronavirus. The disease or disorder can be an infectious disease or disorder caused by a coronavirus, and the AP can comprise or can be derived from an antigenic protein of a coronavirus. The term “coronavirus” as used herein refers to a virus in the family Coronaviridae, which is in turn classified within the order Nidovirales. The coronaviruses are large, enveloped, positive-stranded RNA viruses. The coronaviruses have the largest genomes of the RNA viruses known in the art and replicate by a unique mechanism that results in a high frequency of recombination. The coronaviruses include antigenic groups I, II, and III. Nonlimiting examples of coronaviruses include SARS coronavirus (e.g., SARS-CoV and SARS-CoV-2), MERS coronavirus, transmissible gastroenteritis virus (TGEV), human respiratory coronavirus, porcine respiratory coronavirus, canine coronavirus, feline enteric coronavirus, feline infectious peritonitis virus, rabbit coronavirus, murine hepatitis virus, sialodacryoadenitis virus, porcine hemagglutinating encephalomyelitis virus, bovine coronavirus, avian infectious bronchitis virus, and turkey coronavirus, as well as chimeras thereof. Additional information related to coronavirus including classification, virion structure, genome structure, genetics and pathology is described, for example, in KV Holmes, Encyclopedia of Virology, 1999: 291-298, the content of which is incorporated herein by reference.

In some embodiments, a coronavirus described herein is in the genus of Alpha-coronavirus and the coronavirus antigens can be of or derived from any species or strains in the genus of Alpha-coronavirus. In some embodiments, a coronavirus described herein is in the genus of Beta-coronavirus and the coronavirus antigens can be of or derived from any species or strains in the genus of Beta-coronavirus. Member viruses in the genus of Alpha-coronavirus and Beta-coronavirus are enveloped, positive-strand RNA viruses that can infect mammals.

A coronavirus described herein can be of any subgenus of Alpha-coronavirus genus, including but not limited to Colacovirus (e.g. Bat coronavirus CDPHEJ5), Decacovirus (e.g. Bat coronavirus HKU10 and Rhinolophus ferrumequinum alphacoronavirus HuB-2013), Duvinacovirus (Human coronavirus 229E), Luchacovirus (e.g. Lucheng Rn rat coronavirus), Minacovirus (e.g. Mink coronavirus 1), Minunacovirus (e.g. Miniopterus bat coronavirus 1 and Miniopterus bat coronavirus HKU8), Myotacovirus (e.g. Myotis ricketti alphacoronavirus Sax-2011), Nyctacovirus (e.g. Nyctalus velutinus alphacoronavirus SC-2013 and Pipistrellus kuhlii coronavirus 3398), Pedacovirus (e.g. Porcine epidemic diarrhea virus and Scotophilus bat coronavirus 512), Rhinacovirus (e.g. Rhinolophus bat coronavirus HKU2), Setracovirus (e.g. Human coronavirus NL63 and NL63-related bat coronavirus strain BtKYNL63-9b), Soracovirus (e.g. Sorex araneus coronavirus T14), Sunacovirus (e.g. Suncus murinus coronavirus X74), and Tegacovirus (e.g. Alphacoronavirus 1).

Within the genus Beta-coronavirus, five subgenera or lineages have been recognized, including Embecovirus (lineage A), Sarbecovirus (lineage B), Merbecovirus (lineage C), Nobecovirus (lineage D), and Hibecovirus. Accordingly, in some embodiments, a coronavirus described herein can be any strain or species in any of the subgenera or lineages of Beta-coronavirus.

For example, a coronavirus antigen can be of or derived from any species or strains in the subgenus of Embecovirus, including but not limited to Beta-coronavirus 1 (e.g. Bovine coronavirus and human coronavirus 0C43), China Rattus coronavirus HKU24, Human coronavirus HKU1, Murine coronavirus (e.g. mouse hepatitis virus), and Myodes coronavirus 2JL14. The coronavirus antigen can be of or derived from any species or strains in the subgenus of Sarbecovirus, including but not limited to SARS-CoV, SARS-CoV2, 16BO133, Bat SARS CoV Rf1, Bat coronavirus HKU3 (BtCoV HKU3), LYRa11, Bat SARS-CoV/Rp3, Bat SL-CoV YNLF 31C, Bat SL-CoV YNLF 34C, SHC014-CoV, WIV1, WIV16, Civet SARS-CoV, Rc-o319, SL-ZXC21, SL-ZC45, Pangolin SARSr-COV-GX, Pangolin SARSr-COV-GD, RshSTT182, RshSTT200, RacCS203, RmYN02, RpYN06, RaTG13, Bat CoV BtKY72, and Bat CoV BM48-31. The coronavirus antigen can be of any species or strains in the subgenus of Merbecovirus, including but not limited to Hedgehog coronavirus 1, MERS-CoV, Pipistrellus bat coronavirus HKU5, and Tylonycteris bat coronavirus HKU4. The coronavirus antigen can be of any species or strains in the subgenus of Nobecovirus, including but not limited to Eidolon bat coronavirus C704, Rousettus bat coronavirus GCCDC1, and Rousettus bat coronavirus HKU9. The coronavirus antigen can be of any species or strains in the subgenus of Hibecovirus, including but not limited to Bat Hp-betacoronavirus Zhejiang 2013.

The coronaviruses described herein can be, for example, phylogenetically clustered in functionally distinct clades. For example, the coronaviruses of lineage B Beta-coronavirus (Sarbecovirus) can be clustered into clade 1, clade 2, clade 1/2, or clade 3 using the nucleotide sequences of nonstructural protein gene ORF1a and ORF1b (see, for example, Hu et al., PLoS Pathog 13(11): e1006698). Accordingly, the coronavirus antigens can be of or derived from any species or strain in any one of these clades. For example, the coronavirus antigens can be of any species or strain in clade 1, including but not limited to SARS-CoV, WIV1, LYRa11, Rs7327, Rs4231, Rs4084, and SHC014. The coronavirus antigens can be of any species or strain in clade 2, including but not limited to As6526, Yunnan 2011, Shaanxi 2011, 279-2005, Rs4237, Rs4081, Rp3, Rs4247, HKU3-8, HKU3-13, GX2013, Longquan-140, YN2013, Rf4092, ZXC21, ZC45, JL2012, HuB2013, Rf1, HeB2013, and 273-2005. The coronavirus antigens can be of any species or strain in clade 1/2, including but not limited to SARS-CoV2. The coronavirus antigens can be of any species or strain in clade 3, including but not limited to BM48-31. The coronavirus antigen described herein can be of a coronavirus, for example, SARS, SARS-2, WIV1, SHC014, Rf1, RmYN02, pang17, RaTG13, and Rs4081.

As exemplified herein, SARS virus (e.g., SARS-CoV and SARS-CoV-2) is an enveloped coronavirus carrying a single-stranded positive-sense RNA genome (˜30 kb), belonging to the genus Betacoronavirus from the Coronaviridae family. The virus RNA encodes four structural proteins including spike (S), envelope (E), membrane (M), and nucleocapsid (N) proteins, 16 non-structural proteins, and nine accessory proteins. The S glycoprotein contains an ectodomain that can be processed into S1 and S2 subunits, a transmembrane domain, and an intracellular domain. Both SARS-CoV and SARS-CoV-2 bind the human ACE2 via the receptor binding domain within the S1 subunit to facilitate entry into host cells, followed by membrane fusion mediated by the S2 subunit.

A coronavirus antigen of a coronavirus herein described can be any of a variety of coronavirus proteins capable of inducing an immune response against a coronavirus. Suitable coronavirus antigens are those that can elicit a protective immune response, such as producing broadly neutralizing antibodies. For example, the coronavirus antigen can comprise a coronavirus spike (S) protein, spike receptor binding domain (RBD), S1 subunit, S2 subunit, spike full ectodomain proteins, papain-like proteases, 3CL proteases, nucleocapsid proteins, envelope proteins, membrane proteins, or any of the structural, non-structural or accessory proteins that form a coronavirus.

In some embodiments, a coronavirus antigen used herein comprises a spike (S) protein or a portion thereof. A S protein is one of four major structural proteins covering the surface of each virion. The S protein, comprising a S1 subunit and a S2 subunit, is a highly glycosylated, type I transmembrane protein capable of binding to a host-cell receptor and mediates viral entry. The S protein comprises a domain referred to as the RBD that mediates the interaction with the host-cell receptor to enter the host cell after one or more RBDs adopts an “up” position to bind the host receptor. It is believed that after binding the receptor, a nearby host protease cleaves the spike, which releases the spike fusion peptide, facilitating virus entry. Known host receptors for coronaviruses (e.g., Beta-coronaviruses) include antiotensin-converting enzyme 2 (ACE2), dipeptidyl peptidase-4 (DPP4) or sialic acids. For example, the RBDs of human coronaviruses SARS-CoV-2, SARS-CoV, HCoV-NL63, and related animal coronaviruses (WIV1 and SCH014) use ACE2 as their host receptor, while MERS-CoV uses DPP4 as its host receptor.

The coronavirus antigen used herein can, for example, comprise a coronavirus nucleocapsid protein (N protein) or a portion thereof. The N protein is a multifunctional RNA-binding protein required for viral RNA transcription, replication, and packaging. The N protein consists of three domains, an N-terminal RNA-binding domain, a central intrinsically disordered region, followed by a C-terminal dimerization domain. The RNA-binding domain contains multiple positively charged binding surfaces that form charged interactions with RNA promoting its helical arrangement. The coronavirus antigen used herein can comprise any of these N protein domains or a portion thereof.

In some embodiments, the coronavirus antigen used herein comprises a coronavirus membrane protein (M protein) or a portion thereof. The M protein is the most abundant structural protein and defines the shape of the viral envelope. The M protein is regarded as the central organizer of the viral assembly, interacting with other major coronaviral structural proteins.

In some embodiments, the coronavirus antigen used herein comprises a coronavirus envelope protein (E protein) or a portion thereof. The E protein is a small membrane protein and minor component of the virus particles. Without being bound to any theory, it is believed that the E protein plays roles in virion assembly and morphogenesis, alteration of the membrane of host cells and virus-host cell interaction.

In some embodiments, the coronavirus antigen used herein comprises a coronavirus hemagglutinin-esterase protein (HE protein) or a portion thereof. The HE protein, which is another envelope protein, mediates reversible attachment to 0-acetylated sialic acids by acting both as lectins and receptor-destroying enzymes.

In some embodiments, the coronavirus antigen used herein comprises a coronavirus papain-like protease or a portion thereof. The coronavirus papain-like protease is one of several nonstructural proteins, and is responsible for processing of viral proteins into functional, mature subunits during maturation. For example, the coronavirus papain-like protease can cleave a site at the amino-terminal end of the viral replicase region. In addition to its role in viral protein maturation, papain-like protease exhibits both a deubiquitinating and deISG15ylating activity. In vivo, this protease antagonizes innate immunity by acting on IFN beta and NF-kappa B signaling pathways.

In some embodiments, the coronavirus antigen used herein comprises a coronavirus 3CL protease or a portion thereof. The 3CL protease is another main protease in addition to the papain-like protease and is required for processing of viral polypeptides into distinct, functional proteins. In some embodiments, the 3CL protease is a SARS-CoV-2 3CL Protease, which is a C30-type cysteine protease located within the non-structural proteins 3 (NS3) region of the viral polypeptide. Analysis of the Coronavirus genome reveals at least 11 sites of cleavage for the 3CL protease, many containing the amino acid sequence LQ[S/A/G].

The coronavirus antigen disclosed herein can, in some embodiments, comprise a S protein or a portion thereof, a N protein or a portion thereof, a HE protein or a portion thereof, a papain-like protease or a portion thereof, a coronavirus 3CL protease or a portion thereof, a M protein or a portion thereof, or a combination thereof.

In some embodiments, the coronavirus antigen can be an immunogenic portion of a coronavirus protein herein described. It will be appreciated by those skilled in the art that an immunogenic portion of a coronavirus antigen can be fragments of the S protein (e.g., spike protein RBD), N protein, HE protein, papain-like protease, 3CL protease, or M protein capable of eliciting an immune response against one or more coronaviruses. The immunogenic portion can comprise about, at least or at least about, at most or at most about, 50, 60, 70, 80, 90, 100, 110, 120, 130, 140, 150, 160, 170, 180, 190, 200, 210, 220, 230, 240, 250, or a number or a range between any two of these values, contiguous amino acid residues from the coronavirus proteins. In some embodiments, the immunogenic portion comprises a S protein RBD or a portion thereof. The portion of the S protein RBD can comprise the receptor binding motif of the S protein RBD.

One or more of the plurality of CoV antigens can be of CoVs in the genus of Alpha-CoV and/or Beta-CoV, and optionally each of the plurality of CoV antigens are of CoVs in the genus of Beta-CoV. The plurality of CoV antigens can be of CoVs in the subgenus of Sarbecovirus. The first CoV and the second CoV can be in the genus of Beta-CoV, optionally in the subgenus of Sarbecovirus. The plurality of CoV antigens can be of CoVs selected from the group consisting of: SARS-CoV, SARS-CoV-2, WIV1, SHC014, Rf1, RmYN02, pang17, RaTG13, Rs4081, LYRa11, HKU3, Yunnan2011, BtKY72, BM48-31, WIV16, Khosta-1, and Khosta-2. The first CoV, the second CoV, or both can be selected from the group consisting of: SARS-CoV, SARS-CoV-2, WIV1, SHC014, Rf1, RmYN02, pang17, RaTG13, Rs4081, LYRa11, HKU3, Yunnan2011, BtKY72, BM48-31, WIV16, Khosta-1, and Khosta-2. The CoV can be selected from a species or subspecies of SARS-CoV, SARS-CoV-1, SARS-CoV-2, MERS-CoV, SL-CoV-WIV1, HKU4, HKU5, HCoV-OC43, HCoV-HKU1, HKU9, HKU3, HKU8, HKU24, NL63, SHC014, 229E and/or SARS-CoV-2 variants B.1.351, B.1.1.7, P.1, B.1.617.2, B.1.1.529, BA.1, BA.1.1, BA.2, BA.3, BA.4, BA.5 and other descendent lineages.

The CoV can be selected from a species or subspecies of Embecovirus, Sarbecovirus, Merbecovirus, Nobevovirus, Hibecovirus, SARSr-CoV, MERS-CoV, or any combination thereof. The CoV can be selected from a beta-CoV from the sarbe-, embeco-, merbeco-, and/or nobecovirus lineages. The CoV can be selected from a sarbecovirus strain, optionally, SARS, LYRa11, Rf1, Rs4081, BtKY72, and/or BM48-31. The CoV can be selected from a merbecovirus strain, optionally HKU4, HKU5, HKU25, BtCoV-Vs-CoV1, MERS-related NL13845, MERS-related NL140422. The CoV can be selected from an embecovirus strain, optionally HKU1, Rat CoV Parker, PHEV, Equine CoV, Rodent CoV, Longquan Rat CoV.

Tumor-Associated Antigens, Autoimmune Antigens, and Allergenic Antigens

The disease or disorder can be a disease associated with expression of a tumor-associated antigen, and the antigenic protein can be a tumor-associated antigen. A tumor-associated antigen can be a tumor-specific antigen. In some embodiments, the tumor-associated antigen is selected from the group comprising: 1A01_HLA-A/m (UniProtKB: P30443); 1A02 (UniProtKB: P01892); 5T4 (UniProtKB: Q13641); ACRBP (UniProtKB: Q8NEB7); AFP (UniProtKB: P02771); AKAP4 (UniProtKB: Q5JQC9); alpha-actinin-_4/m (UniProtKB: B4DSX0); alpha-actinin-_4/m (UniProtKB: B4E337); alpha-actinin-_4/m (UniProtKB: 043707); alpha-methylacyl-coenzyme_A_racemase (UniProtKB: A0A024RE16); alpha-methylacyl-coenzyme_A_racemase (UniProtKB: A8KAC3); ANDR (UniProtKB: P10275); ART-4 (UniProtKB: Q9ULX3); ARTC1/m (UniProtKB: P52961); AURKB (UniProtKB: Q96GD4); B2MG (UniProtKB: P61769); B3GN5 (UniProtKB: Q9BYGO); B4GN1 (UniProtKB: Q00973); B7H4 (UniProtKB: Q7Z7D3); BAGE-1 (UniProtKB: Q13072); BASI (UniProtKB: P35613); BCL-2 (UniProtKB: A9QXG9); bcr/abl (UniProtKB: A9UEZ4); bcr/abl (UniProtKB: A9UEZ7); bcr/abl (UniProtKB: A9UEZ8); bcr/abl (UniProtKB: A9UEZ9); bcr/abl (UniProtKB: A9UF00); bcr/abl (UniProtKB: A9UF01); bcr/abl (UniProtKB: A9UF03); bcr/abl (UniProtKB: A9UF04); bcr/abl (UniProtKB: A9UF05); bcr/abl (UniProtKB: A9UF06); bcr/abl (UniProtKB: A9UF08); beta-catenin/m (UniProtKB: P35222); beta-catenin/m (UniProtKB: Q8WYA6); BING-4 (UniProtKB: 015213); BIRC7 (UniProtKB: Q96CA5); BRCA1/m (UniProtKB: A0A024R1V0); BRCA1/m (UniProtKB: A0A024R1V7); BRCA1/m (UniProtKB: A0A024R1Z8); BRCA1/m (UniProtKB: A0A068BFX7); BRCA1/m (UniProtKB: C6YB45); BRCA1/m (UniProtKB: C6YB47); BRCA1/m (UniProtKB: G3XAC3); BY55 (UniProtKB: 095971); calreticulin (UniProtKB: B4DHR1); calreticulin (UniProtKB: B4E2Y9); calreticulin (UniProtKB: P27797); calreticulin (UniProtKB: Q96L12); CAMEL (UniProtKB: 095987); CASP-8/m (UniProtKB: Q14790); CASPA (UniProtKB: Q92851-4); cathepsin_B (UniProtKB: A0A024R374); cathepsin_B (UniProtKB: P07858); cathepsin_L (UniProtKB: A0A024R276); cathepsin_L (UniProtKB: P07711); cathepsin_L (UniProtKB: Q9HBQ7); CD1A (UniProtKB: P06126); CD1B (UniProtKB: P29016); CD1C (UniProtKB: P29017); CD1D (UniProtKB: P15813); CD1E (UniProtKB: P15812); CD20 (UniProtKB: P11836); CD22 (UniProtKB: 060926); CD22 (UniProtKB: P20273); CD22 (UniProtKB: QOEAF5); CD276 (UniProtKB: Q5ZPR3); CD33 (UniProtKB: B4DF51); CD33 (UniProtKB: P20138); CD33 (UniProtKB: Q546G0); CD3E (UniProtKB: P07766); CD3Z (UniProtKB: P20963); CD44_Isoform_1 (UniProtKB: P16070); CD44_Isoform_6 (UniProtKB: P16070-6); CD4 (UniProtKB: P01730); CD52 (UniProtKB: P31358); CD52 (UniProtKB: Q6IBDO); CD52 (UniProtKB: V9HWN9); CD55 (UniProtKB: B1AP15); CD55 (UniProtKB: D3DT85); CD55 (UniProtKB: D3DT86); CD55 (UniProtKB: P08174); CD56 (UniProtKB: P13591); CD80 (UniProtKB: AONOP2); CD80 (UniProtKB: P33681); CD86 (UniProtKB: P42081); CD8A (UniProtKB: P01732); CDCl27/m (UniProtKB: G5EA36); CDCl27/m (UniProtKB: P30260); CDE30 (UniProtKB: P28908); CDK4/m (UniProtKB: A0A024RBB6); CDK4/m (UniProtKB: P11802); CDK4/m (UniProtKB: Q6LC83); CDK4/m (UniProtKB: Q96BE9); CDKN2A/m (UniProtKB: D1LYX3); CDKN2A/m (UniProtKB: G3XAG3); CDKN2A/m (UniProtKB: K7PML8); CDKN2A/m (UniProtKB: L8E941); CDKN2A/m (UniProtKB: Q8N726); CEA (RefSeq: NP_004354); CEAM6 (UniProtKB: P40199); CH3L2 (UniProtKB: Q15782); CLCA2 (UniProtKB: Q9UQC9); CML28 (UniProtKB: Q9NQT4); CML66 (UniProtKB: Q96RS6); COA-1/m (UniProtKB: Q5T124); coactosin-like_protein (UniProtKB: Q14019); collagen_XXIII (UniProtKB: L8EAS4); collagen_XXIII (UniProtKB: Q86Y22); COX-2 (UniProtKB: Q6ZYK7); CP1B1 (UniProtKB: Q16678); CSAG2 (UniProtKB: Q9Y5P2-2); CSAG2 (UniProtKB: Q9Y5P2); CT45A1 (UniProtKB: Q5HYN5); CT55 (UniProtKB: Q8WUE5); CT-_9/BRD6 (UniProtKB: Q58F21); CTAG2_Isoform_LAGE-1A (UniProtKB: 075638-2); CTAG2_Isoform_LAGE-1B (UniProtKB: 075638); CTCFL (UniProtKB: Q8NI51); Cten (UniProtKB: Q8IZW8); cyclin_B1 (UniProtKB: P14635); cyclin_D1 (UniProtKB: P24385); cyp-B (UniProtKB: P23284); DAM-10 (UniProtKB: P43366); DEP1A (UniProtKB: Q5TB30); E7 (UniProtKB: P03129); E7 (UniProtKB: P06788); E7 (UniProtKB: P17387); E7 (UniProtKB: P06429); E7 (UniProtKB: P27230); E7 (UniProtKB: P24837); E7 (UniProtKB: P21736); E7 (UniProtKB: P26558); E7 (UniProtKB: P36831); E7 (UniProtKB: P36833); E7 (UniProtKB: Q9QCZ1); E7 (UniProtKB: Q81965); E7 (UniProtKB: Q80956); EF1A2 (UniProtKB: Q05639); EFTUD2/m (UniProtKB: Q15029); EGFR (UniProtKB: A0A0B4J1Y5); EGFR (UniProtKB: E7BSVO); EGFR (UniProtKB: LOR6G1); EGFR (UniProtKB: P00533-2); EGFR (UniProtKB: P00533); EGFR (UniProtKB: Q147T7); EGFR (UniProtKB: Q504U8); EGFR (UniProtKB: Q8NDU8); EGLN3 (UniProtKB: Q9H6Z9); ELF2/m (UniProtKB: B7Z720); EMMPRIN (UniProtKB: Q54A51); EpCam (UniProtKB: P16422); EphA2 (UniProtKB: P29317); EphA3 (UniProtKB: P29320); EphA3 (UniProtKB: Q6P4R6); ErbB3 (UniProtKB: B3KWG5); ErbB3 (UniProtKB: B4DGQ7); ERBB4 (UniProtKB: Q15303); ERG (UniProtKB: P11308); ETV6 (UniProtKB: P41212); EWS (UniProtKB: Q01844); EZH2 (UniProtKB: F2YMM1); EZH2 (UniProtKB: G3XAL2); EZH2 (UniProtKB: LOR855); EZH2 (UniProtKB: Q15910); EZH2 (UniProtKB: S4S3R8); FABP7 (UniProtKB: 015540); FCGR3A_Version_1 (UniProtKB: P08637); FCGR3A_Version_2 (CCDS: CCDS1232.1); FGFS (UniProtKB: P12034); FGFS (UniProtKB: Q60518); FGFR2 (UniProtKB: P21802); fibronectin (UniProtKB: A0A024R5I6); fibronectin (UniProtKB: A0A024RB01); fibronectin (UniProtKB: A0A024RDT9); fibronectin (UniProtKB: A0A024RDV5); fibronectin (UniProtKB: A6NH44); fibronectin (UniProtKB: A8K6A5); fibronectin (UniProtKB: B2R627); fibronectin (UniProtKB: B3KXM5); fibronectin (UniProtKB: B4DIC5); fibronectin (UniProtKB: B4DN21); fibronectin (UniProtKB: B4DS98); fibronectin (UniProtKB: B4DTH2); fibronectin (UniProtKB: B4DTK1); fibronectin (UniProtKB: B4DU16); fibronectin (UniProtKB: B7Z3W5); fibronectin (UniProtKB: B7Z939); fibronectin (UniProtKB: G5E9X3); fibronectin (UniProtKB: Q9H382); FOS (UniProtKB: P01100); FOXP3 (UniProtKB: Q9BZS1); FUT1 (UniProtKB: P19526); G250 (UniProtKB: Q16790); GAGE-1 (Genbank: AAA82744); GAGE-2 (UniProtKB: Q6NT46); GAGE-3 (UniProtKB: Q13067); GAGE-4 (UniProtKB: Q13068); GAGE-5 (UniProtKB: Q13069); GAGE-6 (UniProtKB: Q13070); GAGE7b (UniProtKB: 076087); GAGE-8_(GAGE-2D) (UniProtKB: Q9UEU5); GASR (UniProtKB: P32239); GnT-V (UniProtKB: Q09328); GPC3 (UniProtKB: I6QTG3); GPC3 (UniProtKB: P51654); GPC3 (UniProtKB: Q8IYG2); GPNMB/m (UniProtKB: A0A024RA55); GPNMB/m (UniProtKB: Q14956); GPNMB/m (UniProtKB: Q8IXJ5); GPNMB/m (UniProtKB: Q96F58); GRM3 (UniProtKB: Q14832); HAGE (UniProtKB: Q9NXZ2); hepsin (UniProtKB: B2ZDQ2); hepsin (UniProtKB: P05981); Her2/neu (UniProtKB: B4DTR1); Her2/neu (UniProtKB: L8E8G2); Her2/neu (UniProtKB: P04626); Her2/neu (UniProtKB: Q9UK79); HLA-A2/m (UniProtKB: Q95387); HLA-A2/m (UniProtKB: Q9MYF8); homeobox NKX3.1 (UniProtKB: Q99801); HOM-TES-85 (UniProtKB: B2RBQ6); HOM-TES-85 (UniProtKB: Q9P127); HPG1 (Pubmed: 12543784); HS71A (UniProtKB: PODMV8); HS71B (UniProtKB: PODMV9); HST-2 (UniProtKB: P10767); hTERT (UniProtKB: 094807); iCE (UniProtKB: 000748); IF2B3 (UniProtKB: 000425); IL10 (UniProtKB: P22301); IL-13Ra2 (UniProtKB: Q14627); IL2-RA (UniProtKB: P01589); IL2-RB (UniProtKB: P14784); IL2-RG (UniProtKB: P31785); IL-5 (UniProtKB: P05113); IMP3 (UniProtKB: Q9NV31); ITA5 (UniProtKB: P08648); ITB1 (UniProtKB: P05556); ITB6 (UniProtKB: P18564); kallikrein-2 (UniProtKB: A0A024R4J4); kallikrein-2 (UniProtKB: A0A024R4N3); kallikrein-2 (UniProtKB: BOAZU9); kallikrein-2 (UniProtKB: B4DU77); kallikrein-2 (UniProtKB: P20151); kallikrein-2 (UniProtKB: Q6T774); kallikrein-2 (UniProtKB: Q6T775); kallikrein-4 (UniProtKB: A0A0C4DFQ5); kallikrein-4 (UniProtKB: Q5BQA0); kallikrein-4 (UniProtKB: Q96PTO); kallikrein-4 (UniProtKB: Q96PT1); kallikrein-4 (UniProtKB: Q9Y5K2); KI20A (UniProtKB: 095235); KIAA0205 (UniProtKB: Q92604); KIF2C (UniProtKB: Q99661); KK-LC-1 (UniProtKB: Q5H943); LDLR (UniProtKB: P01130); LGMN (UniProtKB: Q99538); LIRB2 (UniProtKB: Q8N423); LY6K (UniProtKB: Q17RY6); MAGAS (UniProtKB: P43359); MAGA8 (UniProtKB: P43361); MAGAB (UniProtKB: P43364); MAGE-A10 (UniProtKB: A0A024RC14); MAGE-A12 (UniProtKB: P43365); MAGE-A1 (UniProtKB: P43355); MAGE-A2 (UniProtKB: P43356); MAGE-A3 (UniProtKB: P43357); MAGE-A4 (UniProtKB: A0A024RC12); MAGE-A4 (UniProtKB: P43358); MAGE-A4 (UniProtKB: Q1RN33); MAGE-A6 (UniProtKB: A8K072); MAGE-A6 (UniProtKB: P43360); MAGE-A6 (UniProtKB: Q6FHI5); MAGE-A9 (UniProtKB: P43362); MAGE-B10 (UniProtKB: Q96LZ2); MAGE-B 16 (UniProtKB: A2A368); MAGE-B 17 (UniProtKB: A8MXT2); MAGE-_B1 (UniProtKB: Q96TG1); MAGE-B2 (UniProtKB: 015479); MAGE-B3 (UniProtKB: 015480); MAGE-B4 (UniProtKB: 015481); MAGE-B5 (UniProtKB: Q9BZ81); MAGE-B6 (UniProtKB: Q8N7X4); MAGE-C1 (UniProtKB: 060732); MAGE-C2 (UniProtKB: Q9UBF1); MAGE-C3 (UniProtKB: Q8TD91); MAGE-D1 (UniProtKB: Q9Y5V3); MAGE-D2 (UniProtKB: Q9UNF1); MAGE-D4 (UniProtKB: Q96JG8); MAGE-_E1 (UniProtKB: Q6IAI7); MAGE-E1_(MAGE1) (UniProtKB: Q9HCI5); MAGE-E2 (UniProtKB: Q8TD90); MAGE-F1 (UniProtKB: Q9HAY2); MAGE-H1 (UniProtKB: Q9H213); MAGEL2 (UniProtKB: Q9U355); mammaglobin_A (UniProtKB: Q13296); mammaglobin_A (UniProtKB: Q6NX70); MART-1/melan-A (UniProtKB: Q16655); MART-2 (UniProtKB: Q5VTY9); MC1_R (UniProtKB: Q01726); MC1_R (UniProtKB: Q1JUL4); MC1_R (UniProtKB: Q1JUL6); MC1_R (UniProtKB: Q1JUL8); MC1_R (UniProtKB: Q1JUL9); MC1_R (UniProtKB: Q1JUM0); MC1_R (UniProtKB: Q1JUM2); MC1_R (UniProtKB: Q1JUM3); MC1_R (UniProtKB: Q1JUM4); MC1_R (UniProtKB: Q1JUM5); MC1_R (UniProtKB: Q6UR92); MC1_R (UniProtKB: Q6UR94); MC1_R (UniProtKB: Q6UR95); MC1_R (UniProtKB: Q6UR96); MC1_R (UniProtKB: Q6UR97); MC1_R (UniProtKB: Q6UR98); MC1_R (UniProtKB: Q6UR99); MC1_R (UniProtKB: Q6URA0); MC1_R (UniProtKB: Q86YW1); MC1_R (UniProtKB: V9Q5S2); MC1_R (UniProtKB: V9Q671); MC1_R (UniProtKB: V9Q783); MC1_R (UniProtKB: V9Q7F1); MC1_R (UniProtKB: V9Q8N1); MC1_R (UniProtKB: V9Q977); MC1_R (UniProtKB: V9Q9P5); MC1_R (UniProtKB: V9Q9R8); MC1_R (UniProtKB: V9QAE0); MC1_R (UniProtKB: V9QAR2); MC1_R (UniProtKB: V9QAW3); MC1_R (UniProtKB: V9QB02); MC1_R (UniProtKB: V9QB58); MC1_R (UniProtKB: V9QBY6); MC1_R (UniProtKB: V9QC17); MC1_R (UniProtKB: V9QC66); MC1_R (UniProtKB: V9QCQ4); MC1_R (UniProtKB: V9QDF4); MC1_R (UniProtKB: V9QDN7); MC1_R (UniProtKB: V9QDQ6); M-CSF (UniProtKB: P09603); mesothelin (UniProtKB: Q13421); MITF (UniProtKB: 075030-8); MITF (UniProtKB: 075030-9); MITF (UniProtKB: 075030); MMP1_1 (UniProtKB: B3KQS8); MMP7 (UniProtKB: P09237); MUC-1 (Genbank: AAA60019); MUM-1/m (RefSeq: NP_116242); MUM-2/m (UniProtKB: Q9Y5R8); MYCN (UniProtKB: P04198); MY01A (UniProtKB: Q9UBC5); MY01B (UniProtKB: 043795); MY01C (UniProtKB: 000159); MYO1D (UniProtKB: 094832); MY01E (UniProtKB: Q12965); MY01F (UniProtKB: 000160); MY01G (UniProtKB: B0I1T2); MY01H (RefSeq: NP_001094891); NA17 (UniProtKB: Q3V5L5); NA88-A (Pubmed: 10790436); Neo-PAP (UniProtKB: Q9BWT3); NFYC/m (UniProtKB: Q13952); NGEP (UniProtKB: Q6IWH7); NPM (UniProtKB: P06748); NRCAM (UniProtKB: Q92823); NSE (UniProtKB: P09104); NUF2 (UniProtKB: Q9BZD4); NY-ESO-1 (UniProtKB: P78358); 0A1 (UniProtKB: P51810); OGT (UniProtKB: 015294); OS-9 (UniProtKB: B4DH11); OS-9 (UniProtKB: B4E321); OS-9 (UniProtKB: B7Z8E7); OS-9 (UniProtKB: Q13438); osteocalcin (UniProtKB: P02818); osteopontin (UniProtKB: A0A024RDE2); osteopontin (UniProtKB: A0A024RDE6); osteopontin (UniProtKB: A0A024RDJ0); osteopontin (UniProtKB: B7Z351); osteopontin (UniProtKB: F2YQ21); osteopontin (UniProtKB: P10451); p53 (UniProtKB: P04637); PAGE-4 (UniProtKB: 060829); PAI-1 (UniProtKB: P05121); PAI-2 (UniProtKB: P05120); PAP (UniProtKB: Q06141); PAP (UniProtKB: Q53S56); PATE (UniProtKB: Q8WXA2); PAX3 (UniProtKB: P23760); PAXS (UniProtKB: Q02548); PD1L1 (UniProtKB: Q9NZQ7); PDCD1 (UniProtKB: Q15116); PDEF (UniProtKB: 095238); PECA1 (UniProtKB: P16284); PGCB (UniProtKB: Q96GW7); PGFRB (UniProtKB: P09619); Pim-1-Kinase (UniProtKB: A0A024RD25); Pin-1 (UniProtKB: 015428); Pin-1 (UniProtKB: Q13526); Pin-1 (UniProtKB: Q49AR7); PLAC1 (UniProtKB: Q9HBJ0); PMEL (UniProtKB: P40967); PML (UniProtKB: P29590); POTEF (UniProtKB: A5A3E0); POTE (UniProtKB: Q86YR6); PRAME (UniProtKB: A0A024R1E6); PRAME (UniProtKB: P78395); PRDXS/m (UniProtKB: P30044); PRM2 (UniProtKB: P04554); prostein (UniProtKB: Q96JT2); proteinase-3 (UniProtKB: D6CHE9); proteinase-3 (UniProtKB: P24158); PSA (UniProtKB: P55786); PSB9 (UniProtKB: P28065); PSCA (UniProtKB: D3DWI6); PSCA (UniProtKB: 043653); PSGR (UniProtKB: Q9H255); PSM (UniProtKB: Q04609); PTPRC (RefSeq: NP_002829); RAB8A (UniProtKB: P61006); RAGE-1 (UniProtKB: Q9UQ07); RARA (UniProtKB: P10276); RASH (UniProtKB: P01112); RASK (UniProtKB: P01116); RASN (UniProtKB: P01111); RGSS (UniProtKB: 015539); RHAMM/CD168 (UniProtKB: 075330); RHOC (UniProtKB: P08134); RSSA (UniProtKB: P08865); RU1 (UniProtKB: Q9UHJ3); RU2 (UniProtKB: Q9UHG0); RUNX1 (UniProtKB: Q01196); S-100 (UniProtKB: V9HW39); SAGE (UniProtKB: Q9NXZ1); SART-_1 (UniProtKB: 043290); SART-2 (UniProtKB: Q9UL01); SART-3 (UniProtKB: Q15020); SEPR (UniProtKB: Q12884); SERPINBS (UniProtKB: P36952); SIA7F (UniProtKB: Q969X2); SIA8A (UniProtKB: Q92185); SIAT9 (UniProtKB: Q9UNP4); SIRT2/m (UniProtKB: A0A024ROG8); SIRT2/m (UniProtKB: Q8IXJ6); SOX10 (UniProtKB: P56693); SP17 (UniProtKB: Q15506); SPNXA (UniProtKB: Q9NS26); SPXN3 (UniProtKB: Q5MJ09); SSX-1 (UniProtKB: Q16384); SSX-2 (UniProtKB: Q16385); SSX3 (UniProtKB: Q99909); SSX-4 (UniProtKB: 060224); ST1A1 (UniProtKB: P50225); STAG2 (UniProtKB: Q8N3U4-2); STAMP-1 (UniProtKB: Q8NFT2); STEAP-1 (UniProtKB: A0A024RA63); STEAP-1 (UniProtKB: Q9UHE8); Survivin-2B (UniProtKB: 015392-2); survivin (UniProtKB: 015392); SYCP1 (UniProtKB: A0A024R0I2); SYCP1 (UniProtKB: B7ZLS9); SYCP1 (UniProtKB: Q15431); SYCP1 (UniProtKB: Q3MHC4); SYT-SSX-1 (UniProtKB: A4PIV7); SYT-SSX-1 (UniProtKB: A4PIV8); SYT-SSX-2 (UniProtKB: A4PIV9); SYT-SSX-2 (UniProtKB: A4PIWO); TARP (UniProtKB: QOVGM3); TCRg (UniProtKB: A2JGV3); TF2AA (UniProtKB: P52655); TGFB1 (UniProtKB: P01137); TGFR2 (UniProtKB: P37173); TGM-4 (UniProtKB: B2R7D1); TIE2 (UniProtKB: Q02763); TKIL1 (UniProtKB: P51854); TPI/m (UniProtKB: P60174); TRGV11 (UniProtKB: Q99601); TRGV9 (UniProtKB: A4D1X2); TRGV9 (UniProtKB: Q99603); TRGV9 (UniProtKB: Q99604); TRPC1 (UniProtKB: P48995); TRP-p8 (UniProtKB: Q7Z2W7); TSG10 (UniProtKB: Q9BZW7); TSPY1 (UniProtKB: Q01534); TVC_(TRGV3) (Genbank: M13231.1); TX101 (UniProtKB: Q9BY14-2); tyrosinase (UniProtKB: A0A024DBG7); tyrosinase (UniProtKB: L8B082); tyrosinase (UniProtKB: L8B086); tyrosinase (UniProtKB: L8B0B9); tyrosinase (UniProtKB: 075767); tyrosinase (UniProtKB: P14679); tyrosinase (UniProtKB: U3M8N0); tyrosinase (UniProtKB: U3M9D5); tyrosinase (UniProtKB: U3M9J2); TYRP1 (UniProtKB: P17643); TYRP2 (UniProtKB: P40126); UPA (UniProtKB: Q96NZ9); VEGFR1 (UniProtKB: B5A924); WT1 (UniProtKB: A0A0H5AUY0); WT1 (UniProtKB: P19544); WT1 (UniProtKB: Q06250); and XAGE1 (UniProtKB: Q9HD64).

The disease or disorder can be an autoimmune disease or disorder, and the antigenic protein can be an autoimmune antigen. The autoimmune antigen can comprise: myelin basic protein (MBP), proteolipid protein (PLP), and myelin oligodendrocyte glycoprotein (MOG), in each case associated with multiple sclerosis (MS); CD44, preproinsulin, proinsulin, insulin, glutamic acid decaroxylase (GAD65), tyrosine phosphatase-like insulinoma antigen 2 (IA2), zinc transporter ((ZnT8), and heat shock protein 60 (HSP60), in each case associated with diabetes Typ I; interphotoreceptor retinoid-binding protein (IRBP) associated with autoimmune uveitis; acetylcholine receptor AchR, and insulin-like growth factor-1 receptor (IGF-1R), in each case associated with Myasthenia gravis; M-protein from beta-hemolytic streptocci (pseudo-autoantigen) associated with Rheumatic Fever, or any combination thereof. Autoimmune antigens (antigens associated with autoimmune disease or autoantigens) are selected from autoantigens associated with autoimmune diseases selected from Addison disease (autoimmune adrenalitis, Morbus Addison), alopecia areata, Addison's anemia (Morbus Biermer), autoimmune hemolytic anemia (AIHA), autoimmune hemolytic anemia (AIHA) of the cold type (cold hemagglutinine disease, cold autoimmune hemolytic anemia (AIHA) (cold agglutinin disease), (CHAD)), autoimmune hemolytic anemia (AIHA) of the warm type (warm AIHA, warm autoimmune haemolytic anemia (AIHA)), autoimmune hemolytic Donath-Landsteiner anemia (paroxysmal cold hemoglobinuria), antiphospholipid syndrome (APS), atherosclerosis, autoimmune arthritis, arteriitis temporalis, Takayasu arteriitis (Takayasu's disease, aortic arch disease), temporal arteriitis/giant cell arteriitis, autoimmune chronic gastritis, autoimmune infertility, autoimmune inner ear disease (AIED), Basedow's disease (Morbus Basedow), Bechterew's disease (Morbus Bechterew, ankylosing spondylitis, spondylitis ankylosans), Behcet's syndrome (Morbus Behcet), bowel disease including autoimmune inflammatory bowel disease (including colitis ulcerosa (Morbus Crohn, Crohn's disease), cardiomyopathy, particularly autoimmune cardiomyopathy, idiopathic dilated cardiomyopathy (DCM), celiac sprue dermatitis (gluten mediated enteropathia), chronic fatigue immune dysfunction syndrome (CFIDS), chronic inflammatory demyelinating polyneuropathy (CIDP), chronic polyarthritis, Churg-Strauss syndrome, cicatricial pemphigoid, Cogan syndrome, CREST syndrome (syndrom with Calcinosis cutis, Raynaud phenomenon, motility disorders of the esophagus, sklerodaktylia and teleangiectasia), Crohn's disease (Morbus Crohn, colitis ulcerosa), dermatitis herpetiformis during, dermatologic autoimmune diseases, dermatomyositis, Diabetes, Diabetes mellitus Type 1 (type I diabetes, insuline dependent Diabetes mellitus), Diabetes mellitus Type 2 (type II diabetes), essential mixed cryoglobulinemia, essential mixed cryoglobulinemia, fibromyalgia, fibromyositis, Goodpasture syndrome (anti-GBM mediated glomerulonephritis), graft versus host disease, Guillain-Barré syndrome (GBM, Polyradikuloneuritis), haematologic autoimmune diseases, Hashimoto thyroiditis, hemophilia, acquired hemophilia, hepatitis, autoimmune hepatitis, particularly autoimmune forms of chronic hepatitis, idiopathic pulmonary fibrosis (IPF), idiopathic thrombocytopenic purpura, Immuno-thrombocytopenic purpura (Morbus Werlhof; ITP), IgA nephropathy, infertility, autoimmune infertility, juvenile rheumatoid arthritis (Morbus Still, Still syndrome), Lambert-Eaton syndrome, lichen planus, lichen sclerosus, lupus erythematosus, systemic lupus erythematosus (SLE), lupus erythematosus (discoid form), Lyme arthritis (Lyme disease, borrelia arthritis), Meniere's disease (Morbus Meniere); mixed connective tissue disease (MCTD) multiple sclerosis (MS, encephalomyelitis disseminate, Charcot's disease), Myasthenia gravis (myasthenia, MG), myosits, polymyositis, neural autoimmune diseases, neurodermitis, pemphigus vulgaris, bullous pemphigoid, scar forming pemphigoid; polyarteriitis nodosa (periarteiitis nodosa), polychondritis (panchondritis), polyglandular (autoimmune) syndrome (PGA syndrome, Schmidt's syndrome), Polymyalgia rheumatica, primary agammaglobulinemia, primary biliary cirrhosis PBC, primary autoimmune cholangitis), progressive systemic sclerosis (PSS), Psoriasis, Psoriasis vulgaris, Raynaud's phenomena, Reiter's syndrome (Morbus Reiter, urethral conjunctive synovial syndrome)), rheumatoid arthritis (RA, chronic polyarthritis, rheumatic disease of the joints, rheumatic fever), sarcoidosis (Morbus Boeck, Besnier-Boeck-Schaumann disease), stiff-man syndrome, Sclerodermia, Scleroderma, Sjögren's syndrome, sympathetic ophtalmia; Transient gluten intolerance, transplanted organ rejection, uveitis, autoimmune uveiitis, Vasculitis, Vitiligo, (leucoderma, piebold skin), and Wegner's disease (Morbus Wegner, Wegner's granulomatosis).

The disease or disorder can be an allergic disease or disorder, and the antigenic protein can be an allergenic antigen. In some embodiments, the allergenic antigen is selected from the group comprising: Acarus spp (Aca s 1, Aca s 10, Aca s 10.0101, Aca s 13, Aca s 13.0101, Aca s 2, Aca s 3, Aca s 7, Aca s 8), Acanthocybium spp (Aca so 1), Acanthocheilonema spp (Aca v 3, Aca v 3.0101), Acetes spp (Ace ja 1), Actinidia spp (Act a 1, Act c 1, Act c 10, Act c 10.0101, Act c 2, Act c 4, Act c 5, Act c 5.0101, Act c 8, Act c 8.0101, Act c Chitinase, Act d 1, Act d 1.0101, Act d 10, Act d 10.0101, Act d 10.0201, Act d 11, Act d 11.0101, Act d 2, Act d 2.0101, Act d 3, Act d 3.0101, Act d 3.02, Act d 4, Act d 4.0101, Act d 5, Act d 5.0101, Act d 6, Act d 6.0101, Act d 7, Act d 7.0101, Act d 8, Act d 8.0101, Act d 9, Act d 9.0101, Act d Chitinase, Act e 1, Act e 5), Acyrthosiphon spp (Acy pi 7, Acy pi 7.0101, Acy pi 7.0102), Adenia spp (Ade v RIP), Aedes spp (Aed a 1, Aed a 1.0101, Aed a 2, Aed a 2.0101, Aed a 3, Aed a 3.0101, Aed a 4, Aed a 7, Aed a 7.0101, Aed a 7.0102, Aed a 7.0103, Aed a 7.0104, Aed a 7.0105, Aed a 7.0106, Aed a 7.0107, Aed a 7.0108, Aed a 7.0109, Aed a 7.0110, Aed a 7.0111, Aed al 1, Aed al 3, Aed al 37 kD, Aed v 37 kD, Aed v 63 kD), Aegilops spp (Aeg ta 28, Aeg ta alpha_Gliadin, Aeg um 28, Aeg un 28), Aethaloperca spp (Aet ro 1), Agropyron spp (Agr c 7), Agrostis spp (Agr ca 1, Agr ca 5, Agr g 1, Agr g 4, Agr s 5), Agrobacterium spp (Agr sp CP4 EPSPS), Ailuropoda spp (Ail me Phosvitin, Ail me TCTP), Aix spp (Aix ga 1, Aix sp 1), Aleuroglyphus spp (Ale o 1, Ale o 10, Ale o 10.0101, Ale o 10.0102, Ale o 13, Ale o 14, Ale o 2, Ale o 20, Ale o 3, Ale o 5, Ale o 7, Ale o 8, Ale o 9), Allium spp (All a 3, All a Alliin lyase, All c 3, All c 30 kD, All c 4, All c Alliin lyase, All p Alliin lyase, All s Alliin lyase), Alnus spp (Aln g 1, Aln g 1.0101, Aln g 1/Bet v 1/Cor a 1 TPC7, Aln g 1/Bet v 1/Cor a 1 TPC9, Aln g 2, Aln g 4, Aln g 4.0101), Alopochen spp (Alo ae 1), Alopecurus spp (Alo p 1, Alo p 5), Alternaria spp (Alt a 1, Alt a 1.0101, Alt a 1.0102, Alt a 10, Alt a 10.0101, Alt a 12, Alt a 12.0101, Alt a 13, Alt a 13.0101, Alt a 2, Alt a 3, Alt a 3.0101, Alt a 4, Alt a 4.0101, Alt a 5, Alt a 5.0101, Alt a 6, Alt a 6.0101, Alt a 7, Alt a 7.0101, Alt a 70 kD, Alt a 8, Alt a 8.0101, Alt a 9, Alt a MnSOD, Alt a NTF2, Alt a TCTP, Alt ar 1, Alt arg 1, Alt b 1, Alt bl 1, Alt br 1, Alt c 1, Alt ca 1, Alt ce 1, Alt ch 1, Alt ci 1, Alt co 1, Alt cr 1, Alt ct 1, Alt cu 1, Alt cy 1, Alt d 1, Alt du 1, Alt e 1, Alt et 1, Alt eu 1, Alt ga 1, Alt gr 1, Alt j 1, Alt l 1, Alt lo 1, Alt m 1, Alt me 1, Alt mi 1, Alt mo 1, Alto 1, Alt p 1, Alt ph 1, Alt po 1, Alt ps 1, Alt r 1, Alt s 1, Alt se 1, Alt sm 1, Alt so 1, Alt su 1, Alt t 1, Alt to 1, Alt to 1), Amaranthus spp (Ama r 2, Ama r 2.0101, Ama v 2, Ama v 2.0101, Ama v 2.0201), Ambrosia spp (Amb a 1, Amb a 1.0101, Amb a 1.0201, Amb a 1.0202, Amb a 1.0301, Amb a 1.0302, Amb a 1.0303, Amb a 1.0304, Amb a 1.0305, Amb a 1.0401, Amb a 1.0402, Amb a 1.0501, Amb a 1.0502, Amb a 10, Amb a 10.0101, Amb a 3, Amb a 3.0101, Amb a 4, Amb a 4.0101, Amb a 5, Amb a 5.0101, Amb a 6, Amb a 6.0101, Amb a 7, Amb a 7.0101, Amb a 8, Amb a 8.0101, Amb a 8.0102, Amb a 9, Amb a 9.0101, Amb a 9.0102, Amb a CPI, Amb p 1, Amb p 5, Amb p 5.0101, Amb p 5.0201, Amb t 5, Amb t 5.0101, Amb t 8), Ammothea spp (Amm h 7, Amm h 7.0101), Anadara spp (Ana br 1), Ananas spp (Ana c 1, Ana c 1.0101, Ana c 2, Ana c 2.0101, Ana c 2.0101 (MUXF3)), Anas spp (Ana ca 1), Anarhichas spp (Ana I 1), Anacardium spp (Ana o 1, Ana o 1.0101, Ana o 1.0102, Ana o 2, Ana o 2.0101, Ana o 3, Ana o 3.0101), Anas spp (Ana p 1, Ana p 2, Ana p 3), Anguilla spp (Ang a 1, Ang j 1), Anisakis spp (Ani s 1, Ani s 1.0101, Ani s 10, Ani s 10.0101, Ani s 11, Ani s 11.0101, Ani s 12, Ani s 12.0101, Ani s 2, Ani s 2.0101, Ani s 24 kD, Ani s 3, Ani s 3.0101, Ani s 4, Ani s 4.0101, Ani s 5, Ani s 5.0101, Ani s 6, Ani s 6.0101, Ani s 7, Ani s 7.0101, Ani s 8, Ani s 8.0101, Ani s 9, Ani s 9.0101, Ani s CCOS3, Ani s Cytochrome B, Ani s FBPP, Ani s NADHDS4L, Ani s NARaS, Ani s PEPB, Ani s Troponin), Annona spp (Ann c Chitinase), Anopheles spp (Ano da 17, Ano da 17.0101, Ano da 27, Ano da 27.0101, Ano da 7, Ano da 7.0101, Ano g 7, Ano g 7.0101), Anser spp (Ansa 1, Ans a 2, Ans a 3, Ans in 1), Anthoxanthum spp (Ant o 1, Ant o 1.0101, Ant o 12, Ant o 13, Ant o 2, Ant o 4, Ant o 5, Ant o 6, Ant o 7), Apis spp (Api c 1, Api c 1.0101, Api c 10, Api c 2, Api c 4, Api d 1, Api d 1.0101, Api d 4, Api fl 4), Apium spp (Api g 1, Api g 1.0101, Api g 1.0201, Api g 2, Api g 2.0101, Api g 3, Api g 3.0101, Api g 4, Api g 4.0101, Api g 5, Api g 5.0101, Api g 6, Api g 6.0101), Apis spp (Api m 1, Api m 1.0101, Api m 10, Api m 10.0101, Api m 11, Api m 11.0101, Api m 11.0201, Api m 13 kD, Api m 2, Api m 2.0101, Api m 3, Api m 3.0101, Api m 4, Api m 4.0101, Api m 5, Api m 5.0101, Api m 6, Api m 6.0101, Api m 7, Api m 7.0101, Api m 8, Api m 8.0101, Api m 9, Api m 9.0101, Api m A1-A2, Api m A1-A2-A3, Api m Apalbumin 1, Api m Apalbumin 2, Api me 1, Api me 4), Arachis spp (Ara d 2, Ara d 6, Ara f 3, Ara f 4, Ara h 1, Ara h 1.0101, Ara h 10, Ara h 10.0101, Ara h 10.0102, Ara h 11, Ara h 11.0101, Ara h 2, Ara h 2.0101, Ara h 2.0102, Ara h 2.0201, Ara h 2.0202, Ara h 3, Ara h 3.0101, Ara h 4, Ara h 4.0101, Ara h 5, Ara h 5.0101, Ara h 6, Ara h 6.0101, Ara h 7, Ara h 7.0101, Ara h 7.0201, Ara h 7.0202, Ara h 8, Ara h 8.0101, Ara h 8.0201, Ara h 9, Ara h 9.0101, Ara h 9.0201, Ara h Agglutinin, Ara h Oleosin 18 kD, Ara i 2, Ara i 6), Arabidopsis spp (Ara t 3, Ara t 8, Ara t GLP), Archosargus spp (Arc pr 1), Archaeopotamobius spp (Arc s 8, Arc s 8.0101), Aequipecten spp (Arg i 1), Argas spp (Arg r 1, Arg r 1.0101), Ariopsis spp (Ari fe 1), Armoracia spp (Arm r HRP), Arrhenatherum spp (Arr e 1, Arr e 5), Artemisia spp (Art a 1, Art ap 1), Artemia spp (Art fr 1, Art fr 1.0101, Art fr 5, Art fr 5.0101), Arthrobacter spp (Art gl CO), Achorion spp (Art gy 7), Artocarpus spp (Art h 17 kD, Art h 4), Arthrospira spp (Art pl beta_Phycocyanin), Artemisia spp (Art v 1, Art v 1.0101, Art v 1.0102, Art v 1.0103, Art v 1.0104, Art v 1.0105, Art v 1.0106, Art v 1.0107, Art v 2, Art v 2.0101, Art v 3, Art v 3.0101, Art v 3.0201, Art v 3.0202, Art v 3.0301, Art v 4, Art v 4.0101, Art v 4.0201, Art v 47 kD, Art v 5, Art v 5.0101, Art v 6, Art v 6.0101, Art v 60 kD), Arthroderma spp (Art va 4), Ascaris spp (Asc 13, Asc 1 3.0101, Asc 1 3.0102, Asc 1 34 kD, Asc s 1, Asc s 1.0101, Asc s 3, Asc s 3.0101, Asc s GST), Aspergillus spp (Asp aw Glucoamylase, Asp c 22, Asp f 1, Asp f 1.0101, Asp f 10, Asp f 10.0101, Asp f 11, Asp f 11.0101, Asp f 12, Asp f 12.0101, Asp f 13, Asp f 13.0101, Asp f 15, Asp f 15.0101, Asp f 16, Asp f 16.0101, Asp f 17, Asp f 17.0101, Asp f 18, Asp f 18.0101, Asp f 2, Asp f 2.0101, Asp f 22, Asp f 22.0101, Asp f 23, Asp f 23.0101, Asp f 27, Asp f 27.0101, Asp f 28, Asp f 28.0101, Asp f 29, Asp f 29.0101, Asp f 3, Asp f 3.0101, Asp f 34, Asp f 34.0101, Asp f 4, Asp f 4.0101, Asp f 5, Asp f 5.0101, Asp f 56 kD, Asp f 6, Asp f 6.0101, Asp f 7, Asp f 7.0101, Asp f 8, Asp f 8.0101, Asp f 9, Asp f 9.0101, Asp f AfCalAp, Asp f AT_V, Asp f Catalase, Asp f Chitosanase, Asp f CP, Asp f DPPV, Asp f FDH, Asp f gamma_Actin, Asp f Glucosidase, Asp f GPI, Asp f GST, Asp f GT, Asp f IAO, Asp f IPMI, Asp f LPL1, Asp f LPL3, Asp f Mannosidase, Asp f MDH, Asp f PL, Asp f PUP, Asp f RPS3, Asp f SXR, Asp fl 13, Asp fl 13.0101, Asp fl 18, Asp fl 2, Asp fl 21, Asp fl 3, Asp fl 4, Asp fl 7, Asp fl 8, Asp fl 9, Asp me Sea prose, Asp n 14, Asp n 14.0101, Asp n 18, Asp n 18.0101, Asp n 25, Asp n 25.0101, Asp n 30, Asp n Glucoamylase, Asp n Hemicellulase, Asp n Pectinase, Asp o 13, Asp o 13.0101, Asp o 21, Asp o 21.0101, Asp o 3, Asp o 4, Asp o 7, Asp o 8, Asp o Lactase, Asp o Lipase, Asp oc 13, Asp r 1, Asp sa AP, Asp sp Glucoamylase, Asp sp Glucoseoxidase, Asp sp PL, Asp sp PME, Asp sy 13, Asp v 13, Asp v 13.0101, Asp v Catalase A, Asp v Enolase, Asp v GAPDH, Asp v MDH, Asp v SXR), Asparagus spp (Aspa o 1, Aspa o 1.01, Aspa o 1.02, Aspa o 17 kD, Aspa o 4), Aspergillus spp (Aspe ni 2, Aspe ni 3, Aspe ni 4, Aspe ni 7, Aspe ni 8, Aspe ni 9), Avena spp (Ave s 1, Ave s 12, Ave s 13, Ave s 2, Ave s 4, Ave s 5, Ave s 7), Babylonia spp (Bab ja 1), Bacillus spp (Bac al Subtilisin, Bac cl Subtilisin, Bac I Subtilisin, Bac Ii aA, Bac Ii Subtilisin), Bactrocera spp (Bac ol 27, Bac ol 27.0101), Bacillus spp (Bac sp aA1, Bac sp aA3, Bac sp Decarboxylase, Bac st amyM, Bac su Subtilisin, Bac t Cry1Ab, Bac t Cry1Fa, Bac t Cry3Bb1, Bac t Cry9c), Bagre spp (Bag ma 1), Balistes spp (Bal ca 1), Balanus spp (Bal r 1, Bal r 1.0101), Beauveria spp (Bea b AId, Bea b Enol, Bea b f2, Bea b Hex), Bertholletia spp (Ber e 1, Ber e 1.0101, Ber e 2, Ber e 2.0101), Beryx spp (Ber sp 1), Betula spp (Bet ab 1, Bet al 1, Bet ch 1, Bet co 1, Bet da 1, Bet gr 1, Bet hu 1, Bet le 1, Bet me 1, Bet n 1, Bet p 1, Bet pa 1, Bet po 1, Bet pu 1, Bet pu 2, Bet pu 4, Bet pu 6, Bet pu 7, Bet sc 1, Bet ut 1, Bet v 1, Bet v 1 B1-131-131, Bet v 1 fv Mal 4x, Bet v 1.0101, Bet v 1.0102, Bet v 1.0103, Bet v 1.0201, Bet v 1.0301, Bet v 1.0401, Bet v 1.0402, Bet v 1.0501, Bet v 1.0601, Bet v 1.0602, Bet v 1.0701, Bet v 1.0801, Bet v 1.0901, Bet v 1.1001, Bet v 1.1101, Bet v 1.1201, Bet v 1.1301, Bet v 1.1401, Bet v 1.1402, Bet v 1.1501, Bet v 1.1502, Bet v 1.1601, Bet v 1.1701, Bet v 1.1801, Bet v 1.1901, Bet v 1.2001, Bet v 1.2101, Bet v 1.2201, Bet v 1.2301, Bet v 1.2401, Bet v 1.2501, Bet v 1.2601, Bet v 1.2701, Bet v 1.2801, Bet v 1.2901, Bet v 1.3001, Bet v 1.3101, Bet v 2, Bet v 2.0101, Bet v 3, Bet v 3.0101, Bet v 4, Bet v 4.0101, Bet v 6, Bet v 6.0101, Bet v 6.0102, Bet v 7, Bet v 7.0101, Bet v 8, Bet v Glucanase), Beta spp (Beta v 1, Beta v 1.0101, Beta v 2, Beta v 2.0101), Blattella spp (Bla g 1, Bla g 1.0101, Bla g 1.0102, Bla g 1.0103, Bla g 1.0201, Bla g 1.0202, Bla g 2, Bla g 2.0101, Bla g 2.0201, Bla g 36 kD, Bla g 4, Bla g 4.0101, Bla g 4.0201, Bla g 5, Bla g 5.0101, Bla g 5.0201, Bla g 6, Bla g 6.0101, Bla g 6.0201, Bla g 6.0301, Bla g 7, Bla g 7.0101, Bla g 8, Bla g 8.0101, Bla g 9, Bla g Enolase, Bla g GSTD1, Bla g RACK1, Bla g TPI, Bla g Trypsin, Bla g Vitellogenin), Blatta spp (Bla o 1, Bla o 7), Blomia spp (Blo t 1, Blo t 1.0101, Blo t 1.0201, Blo t 10, Blot 10.0101, Blot 10.0102, Blot 11, Blot 11.0101, Blot 12, Blot 12.0101, Blot 12.0102, Blot 13, Blot 13.0101, Blot 14, Blot 15, Blot 18, Blot 19, Blot 19.0101, Blot 2, Blot 2.0101, Blo t 2.0102, Blo t 2.0103, Blo t 20, Blo t 21, Blo t 21.0101, Blo t 3, Blo t 3.0101, Blo t 4, Blo t 4.0101, Blo t 5, Blo t 5.0101, Blo t 6, Blo t 6.0101, Blo t 7, Blo t 8, Blo t 9, Blo t HSP70), Bombus spp (Bom ar 4, Bom by 4, Bom p 1, Bom p 1.0101, Bom p 2, Bom p 3, Bom p 4, Bom p 4.0101, Bom t 1, Bom t 1.0101, Bom t 4, Bom t 4.0101), Bombyx spp (Bomb m 1, Bomb m 1.0101, Bomb m 7, Bomb m 7.0101, Bomb m 7.0102, Bomb m 7.0103, Bomb m 7.0104, Bomb m 7.0105, Bomb m 7.0106), Boophilus spp (Boo m 1, Boo m 7, Boo m 7.0101), Bos spp (Bos d 2, Bos d 2.0101, Bos d 2.0102, Bos d 2.0103, Bos d 3, Bos d 3.0101, Bos d 4, Bos d 4.0101, Bos d 5, Bos d 5.0101, Bos d 5.0102, Bos d 6, Bos d 6 (MDA), Bos d 6.0101, Bos d 7, Bos d 7.0101, Bos d 8, Bos d 8 alphaS1, Bos d 8 alphaS2, Bos d 8 beta, Bos d 8 kappa, Bos d alpha2l, Bos d alpha2I.0101, Bos d Chymosin, Bos d Fibrin, Bos d Gelatin, Bos d HG, Bos d Insulin, Bos d Lactoferrin, Bos d Lactoperoxidase, Bos d Myoglobin, Bos d OBP, Bos d OSCP, Bos d Phosvitin, Bos d PLA2, Bos d PRVB, Bos d Thrombin, Bos d TI, Bos gr ALA, Bos gr Myoglobin), Bothrops spp (Bot as 1, Bot at 1), Bouteloua spp (Bou g 1), Biting spp (Boy ov 1), Brama spp (Bra du 1), Brassica spp (Bra j 1, Bra j 1.0101, Bran 1, Bran 1.0101, Bran 4, Bran 7, Bran 8, Bran PG, Bra ni 8, Bra o 3, Bra o 3.0101, Bra r 1, Bra r 1.0101, Bra r 2, Bra r 2.0101, Bra r 3, Bra r 4, Bra r 7), Bromus spp (Bro a 1, Bro a 4), Brosme spp (Bro br 1), Bromus spp (Bro i 1, Bro i 5, Bro i 7), Brugia spp (Bru m 3, Bru m 3.0101, Bru m Bm33), Bubalus spp (Bub b ALA, Bub b BLG, Bub b Casein, Bub b Casein alphaS1, Bub b Casein alphaS2, Bub b Casein beta, Bub b Casein kappa), Caenorhabditis spp (Cae b 3, Cae b 3.0101, Cae br 3, Cae br 3.0101, Cae e 3, Cae e 3.0101, Cae e 3.0102, Cae re 13, Cae re 13.0101), Cajanus spp (Caj c 1), Caligus spp (Cal cl 1, Cal cl 1.0101, Cal cl 1.0102), Calamus spp (Cal le 1), Callinectes spp (Cal s 2), Camelus spp (Cam d ALA, Cam d Casein, Cam d Casein alphaS1, Cam d Casein alphaS2, Cam d Casein beta, Cam d Casein kappa), Camponotus spp (Cam fl 7, Cam fl 7.0101), Canis spp (Can f 1, Can f 1.0101, Can f 2, Can f 2.0101, Can f 3, Can f 3.0101, Can f 4, Can f 4.0101, Can f 5, Can f 5.0101, Can f 6, Can f 6.0101, Can f Feld1-like, Can f Homs2-like, Can f Phosvitin, Can f TCTP), Canthidermis spp (Can ma 1), Cancer spp (Can mg 2, Can p 1), Cannabis spp (Can s 3), Candida spp (Cand a 1, Cand a 1.0101, Cand a 3, Cand a 3.0101, Cand a CAAP, Cand a CyP, Cand a Enolase, Cand a FPA, Cand a MnSOD, Cand a PGK, Cand b 2, Cand b 2.0101, Cand b FDH, Cand r Lipase), Capsicum spp (Cap a 1, Cap a 1.0101, Cap a 17 kD, Cap a 2, Cap a 2.0101, Cap a 30 kD, Cap a Glucanase, Cap ch 17 kD), Caprella spp (Cap e 1), Capra spp (Cap h ALA, Cap h BLG, Cap h Casein, Cap h Casein alphaS1, Cap h Casein alphaS2, Cap h Casein beta, Cap h Casein kappa, Cap h GSA), Capitulum spp (Cap m 1), Carassius spp (Car au 1), Carpinus spp (Car b 1, Car b 1.0101, Car b 1.0102, Car b 1.0103, Car b 1.0104, Car b 1.0105, Car b 1.0106, Car b 1.0107, Car b 1.0108, Car b 1.0109, Car b 1.0110, Car b 1.0111, Car b 1.0112, Car b 1.0113, Car b 1.0201, Car b 1.0301, Car b 1.0302, Car b 2, Car b 4), Caranx spp (Car cr 1), Carya spp (Car i 1, Car i 1.0101, Car i 2, Car i 4, Car i 4.0101), Carcinus spp (Car ma 2), Caryota spp (Car mi 2), Carica spp (Car p 1, Car p Chitinase, Car p Chymopapain, Car p Endoproteinase), Castanea spp (Cas c 24 kD, Cas s 1, Cas s 1.0101, Cas s 1.0102, Cas s 1.0103, Cas s 2, Cas s 5, Cas s 5.0101, Cas s 8, Cas s 8.0101, Cas s 9, Cas s 9.0101), Catharanthus spp (Cat r 1, Cat r 1.0101, Cat r 17 kD, Cat r 2), Caulolatilus spp (Cau ch 1), Cavia spp (Cav p 1, Cav p 1.0101, Cav p 2, Cav p 2.0101, Cav p 3, Cav p 3.0101, Cav p Gelatin, Cav p GSA), Centropristis spp (Cen s 1), Cephalopholis spp (Cep so 1), Charybdis spp (Cha f 1, Cha f 1.0101), Chaetodipterus spp (Cha fa 1), Chamaecyparis spp (Cha o 1, Cha o 1.0101, Cha o 2, Cha o 2.0101), Chenopodium spp (Che a 1, Che a 1.0101, Che a 2, Che a 2.0101, Che a 3, Che a 3.0101), Chironomus spp (Chi k 1, Chi k 10, Chi k 10.0101), Chinchilla spp (Chi I 21 kD_a, Chi I 21 kD_b), Chionoecetes spp (Chi o 1, Chi o 1.0101, Chi o 2, Chi o 4, Chi o 6, Chi o alpha_Actin, Chi o SERCA), Chironomus spp (Chi t 1, Chi t 1.0101, Chi t 1.0201, Chi t 2, Chi t 2.0101, Chi t 2.0102, Chi t 3, Chi t 3.0101, Chi t 4, Chi t 4.0101, Chi t 5, Chi t 5.0101, Chi t 6, Chi t 6.0101, Chi t 6.0201, Chi t 7, Chi t 7.0101, Chi t 8, Chi t 8.0101, Chi t 9, Chi t 9.0101), Chlamys spp (Chl n 1), Chloephaga spp (Chl pi 1), Chortoglyphus spp (Cho a 10), Chrysomela spp (Chr tr 7, Chr tr 7.0101), Cicer spp (Cic a 2S Albumin, Cic a Albumin), Cichorium spp (Cic i 1), Cimex spp (Cim I Nitrophorin), Citrus spp (Cit 11, Cit I 3, Cit I 3.0101), Citrullus spp (Cit la 2, Cit la MDH, Cit la TPI), Citrus spp (Cit r 3, Cit r 3.0101, Cit s 1, Cit s 1.0101, Cit s 2, Cit s 2.0101, Cit s 3, Cit s 3.0101, Cit s 3.0102, Cit s IFR), Cladosporium spp (Cla c 14, Cla c 14.0101, Cla c 9, Cla c 9.0101, Cla h 1, Cla h 10, Cla h 10.0101, Cla h 12, Cla h 12.0101, Cla h 2, Cla h 2.0101, Cla h 42 kD, Cla h 5, Cla h 5.0101, Cla h 6, Cla h 6.0101, Cla h 7, Cla h 7.0101, Cla h 8, Cla h 8 CSP, Cla h 8.0101, Cla h 9, Cla h 9.0101, Cla h abH, Cla h GST, Cla h HCh1, Cla h HSP70, Cla h NTF2, Cla h TCTP), Clostridium spp (Clo hi Collagenase, Clot Toxoid), Clupea spp (Clu h 1, Clu h 1.0101, Clu h 1.0201, Clu h 1.0301), Cocos spp (Coc n 2, Coc n 4, Coc n 5), Coccidioides spp (Coc po 8), Coffea spp (Cof a 1, Cof a 1.0101), Columba spp (Col I PSA), Coprinus spp (Cop c 1, Cop c 1.0101, Cop c 2, Cop c 2.0101, Cop c 3, Cop c 3.0101, Cop c 4, Cop c 5, Cop c 5.0101, Cop c 6, Cop c 7, Cop c 7.0101), Corylus spp (Cor a 1, Cor a 1.0101, Cor a 1.0102, Cor a 1.0103, Cor a 1.0104, Cor a 1.0201, Cor a 1.0301, Cor a 1.0401, Cor a 1.0402, Cor a 1.0403, Cor a 1.0404, Cor a 10, Cora 10.0101, Cora 11, Cora 11.0101, Cora 12, Cora 12.0101, Cora 13, Cora 13.0101, Cor a 14, Cor a 14.0101, Cor a 2, Cor a 2.0101, Cor a 2.0102, Cor a 8, Cor a 8.0101, Cor a 9, Cor a 9.0101), Corynebacterium spp (Cor d Toxoid), Corylus spp (Cor he 1), Coryphaena spp (Cor hi 1), Coriandrum spp (Cor s 1, Cor s 11 kD, Cor s 2), Cotoneaster spp (Cot I 3), Crangon spp (Cra c 1, Cra c 1.0101, Cra c 2, Cra c 2.0101, Cra c 4, Cra c 4.0101, Cra c 5, Cra c 5.0101, Cra c 6, Cra c 6.0101, Cra c 8, Cra c 8.0101), Crassostrea spp (Cra g 1), Cricetus spp (Cri c HSA), Crivellia spp (Cri pa 1), Crocus spp (Cro s 1, Cro s 1.0101, Cro s 2, Cro s 2.0101, Cro s 3, Cro s 3.01, Cro s 3.02), Cryptomeria spp (Cry j 1, Cry j 1.0101, Cry j 1.0102, Cry j 1.0103, Cry j 2, Cry j 2.0101, Cry j 2.0102, Cry j 3, Cry j 3.1, Cry j 3.2, Cry j 3.3, Cry j 3.4, Cry j 3.5, Cry j 3.6, Cry j 3.7, Cry j 3.8, Cry j 4, Cry j AP, Cry j Chitinase, Cry j CPA9, Cry j IFR, Cry j LTP, Cry j P1-P2), Cryphonectria spp (Cry p AP), Ctenocephalides spp (Cte f 1, Cte f 1.0101, Cte f 2, Cte f 2.0101, Cte f 3, Cte f 3.0101), Ctenopharyngodon spp (Cte id 1), Cucumis spp (Cuc m 1, Cuc m 1.0101, Cuc m 2, Cuc m 2.0101, Cuc m 3, Cuc m 3.0101, Cuc m Lec17, Cuc m MDH), Cucurbita spp (Cuc ma 18 kD, Cuc ma 2, Cuc p 2, Cuc p AscO), Cucumis spp (Cuc s 2), Culicoides spp (Cul n 1, Cul n 10, Cul n 11, Cul n 2, Cul n 3, Cul n 4, Cul n 5, Cul n 6, Cul n 7, Cul n 8, Cul n 9, Cul n HSP70), Culex spp (Cul q 28 kD, Cul q 35 kD, Cul q 7, Cul q 7.0101, Cul q 7.0102), Culicoides spp (Cul so 1), Cuminum spp (Cum c 1, Cum c 2), Cupressus spp (Cup a 1, Cup a 1.0101, Cup a 1.02, Cup a 2, Cup a 3, Cup a 4, Cup a 4.0101, Cups 1, Cups 1.0101, Cups 1.0102, Cup s 1.0103, Cup s 1.0104, Cup s 1.0105, Cup s 3, Cup s 3.0101, Cup s 3.0102, Cup s 3.0103, Cup s 8), Cochliobolus spp (Cur 11, Cur 11.0101, Cur I 2, Cur I 2.0101, Cur I 3, Cur I 3.0101, Cur I 4, Cur I 4.0101, Cur I ADH, Cur I GST, Cur I MnSOD, Cur I Oryzin, Cur I Trx, Cur I ZPS1), Cyanochen spp (Cya cy 1), Cynoscion spp (Cyn ar 1), Cynosurus spp (Cyn cr 1, Cyn cr 5), Cynodon spp (Cyn d 1, Cyn d 1.0101, Cyn d 1.0102, Cyn d 1.0103, Cyn d 1.0104, Cyn d 1.0105, Cyn d 1.0106, Cyn d 1.0107, Cyn d 1.0201, Cyn d 1.0202, Cyn d 1.0203, Cyn d 1.0204, Cyn d 10, Cyn d 11, Cyn d 12, Cyn d 12.0101, Cyn d 13, Cyn d 15, Cyn d 15.0101, Cyn d 2, Cyn d 22, Cyn d 22.0101, Cyn d 23, Cyn d 23.0101, Cyn d 24, Cyn d 24.0101, Cyn d 4, Cyn d 5, Cyn d 6, Cyn d 7, Cyn d 7.0101), Cynoscion spp (Cyn ne 1), Cynomys spp (Cyn sp Lipocalin), Cyprinus spp (Cyp c 1, Cyp c 1.01, Cyp c 1.02), Daboia spp (Dab ru 1), Dactyhs spp (Dac g 1, Dac g 1.01, Dac g 1.0101, Dac g 1.02, Dac g 12, Dac g 13, Dac g 2, Dac g 2.0101, Dac g 3, Dac g 3.0101, Dac g 4, Dac g 4.0101, Dac g 5, Dac g 5.0101, Dac g 7), Dama spp (Dam d CSA), Danio spp (Dan re 1, Dan re 2, Dan re alpha2l, Dan re CK), Dasyatis spp (Das ak 1, Das am 1, Das sa 1), Daucus spp (Dau c 1, Dau c 1.0101, Dau c 1.0102, Dau c 1.0103, Dau c 1.0104, Dau c 1.0105, Dau c 1.0201, Dau c 1.0301, Dau c 3, Dau c 4, Dau c 4.0101, Dau c CyP), Decapterus spp (Dec ru 1), Dendronephthya spp (Den n 1, Den n 1.0101), Dermatophagoides spp (Der f 1, Der f 1.0101, Der f 1.0102, Der f 1.0103, Der f 1.0104, Der f 1.0105, Der f 1.0106, Der f 1.0107, Der f 1.0108, Der f 1.0109, Der f 1.0110, Der f 10, Der f 10.0101, Der f 10.0102, Der f 11, Der f 11.0101, Der f 13, Der f 13.0101, Der f 14, Der f 14.0101, Der f 15, Der f 15.0101, Der f 16, Der f 16.0101, Der f 17, Der f 17.0101, Der f 18, Der f 18.0101, Der f 2, Der f 2.0101, Der f 2.0102, Der f 2.0103, Der f 2.0104, Der f 2.0105, Der f 2.0106, Der f 2.0107, Der f 2.0108, Der f 2.0109, Der f 2.0110, Der f 2.0111, Der f 2.0112, Der f 2.0113, Der f 2.0114, Der f 2.0115, Der f 2.0116, Der f 2.0117, Der f 20, Der f 21, Der f 22, Der f 22.0101, Der f 3, Der f 3.0101, Der f 4, Der f 5, Der f 6, Der f 6.0101, Der f 7, Der f 7.0101, Der f 8, Der f 9, Der f HSP70), Dermanyssus spp (Der g 10, Der g 10.0101), Dermatophagoides spp (Der m 1, Der m 1.0101, Der p 1, Der p 1.0101, Der p 1.0102, Der p 1.0103, Der p 1.0104, Der p 1.0105, Der p 1.0106, Der p 1.0107, Der p 1.0108, Der p 1.0109, Der p 1.0110, Der p 1.0111, Der p 1.0112, Der p 1.0113, Der p 1.0114, Der p 1.0115, Der p 1.0116, Der p 1.0117, Der p 1.0118, Der p 1.0119, Der p 1.0120, Der p 1.0121, Der p 1.0122, Der p 1.0123, Der p 1.0124, Der p 10, Der p 10.0101, Der p 10.0102, Der p 10.0103, Der p 11, Der p 11.0101, Der p 13, Der p 14, Der p 14.0101, Der p 15, Der p 18, Der p 2, Der p 2.0101, Der p 2.0102, Der p 2.0103, Der p 2.0104, Der p 2.0105, Der p 2.0106, Der p 2.0107, Der p 2.0108, Der p 2.0109, Der p 2.0110, Der p 2.0111, Der p 2.0112, Der p 2.0113, Der p 2.0114, Der p 2.0115, Der p 20, Der p 20.0101, Der p 21, Der p 21.0101, Der p 23, Der p 23.0101, Der p 3, Der p 3.0101, Der p 4, Der p 4.0101, Der p 5, Der p 5.0101, Der p 5.0102, Der p 6, Der p 6.0101, Der p 7, Der p 7.0101, Der p 8, Der p 8.0101, Der p 9, Der p 9.0101, Der p 9.0102, Der p P1-P2, Der p P2-P1, Der s 1, Der s 2, Der s 3), Dianthus spp (Dia c RIP), Dicranopteris spp (Dic I 2S Albumin), Diospyros spp (Dio k 17 kD, Dio k 4, Dio k IFR), Dioscorea spp (Dio p TSP), Diplodus spp (Dip ho 1), Distichlis spp (Dis s 1, Dis s 7), Ditrema spp (Dit to 1), Dolichovespula spp (Dol a 1, Dol a 2, Dol a 5, Dol a 5.0101), Dolichos spp (Dol b Agglutinin), Dolichovespula spp (Dol m 1, Dol m 1.0101, Dol m 1.02, Dol m 2, Dol m 2.0101, Dol m 5, Dol m 5.0101, Dol m 5.02), Drosophila spp (Dro an 7, Dro an 7.0101, Dro er 7, Dro er 7.0101, Dro er 7.0102, Dro gr 7, Dro gr 7.0101, Dro gr 7.0102, Dro m 7, Dro m 7.0101, Dro m 7.0102, Dro m 7.0103, Dro m 7.0104, Dro m 7.0105, Dro m 7.0106, Dro m 7.0107, Dro m 7.0108, Dro m 7.0109, Dro m 7.0110, Dro m 7.0111, Dro m 7.0112, Dro m 7.0113, Dro m 9, Dro m MnSOD, Dro mo 7, Dro mo 7.0101, Dro pp 7, Dro pp 7.0101, Dro se 7, Dro se 7.0101, Dro si 7, Dro si 7.0101, Dro si 7.0102, Dro vi 7, Dro vi 7.0101, Dro wi 7, Dro wi 7.0101, Dro y 7, Dro y 7.0101, Dro y 7.0102, Dro y 7.0103), Echium spp (Ech p Cytochrome C), Elaeis spp (Ela g 2, Ela g Bd31 kD), Elops spp (Elo sa 1), Embellisia spp (Emb a 1, Emb i 1, Emb nz 1, Emb t 1), Engraulis spp (Eng e 1), Enteroctopus spp (Ent d 1), Epinephelus spp (Epi bl 1, Epi co 1, Epi fl 1, Epi mc 1, Epi mo 1), Epicoccum spp (Epi p 1, Epi p 1.0101, Epi p 12 kD, Epi p GST), Epinephelus spp (Epi po 1, Epi un 1), Equisetum spp (Equ a 17 kD), Equus spp (Equ as 4, Equ as DSA, Equ bu 4, Equ c 1, Equ c 1.0101, Equ c 2, Equ c 2.0101, Equ c 2.0102, Equ c 3, Equ c 3.0101, Equ c 4, Equ c 4.0101, Equ c 5, Equ c 5.0101, Equ c ALA, Equ c BLG, Equ c Casein, Equ c Casein beta, Equ c Casein kappa, Equ c PRVB, Equ he 4, Equ z ZSA), Erimacrus spp (En i 1, En i 1.0101, Eri i 1.0102), Eriocheir spp (Eri s 1, Eri s 1.0101, En s 2), Envinia spp (Erw ch Asparaginase), Escherichia spp (Esc c Asparaginase, Esc c beta GAL), Esox spp (Eso 11), Euphausia spp (Eup p 1, Eup p 1.0101), Euphasia spp (Eup s 1, Eup s 1.0101), Euroglyphus spp (Eur m 1, Eur m 1.0101, Eur m 1.0102, Eur m 1.0103, Eur m 10, Eur m 14, Eur m 14.0101, Eur m 2, Eur m 2.0101, Eur m 2.0102, Eur m 3, Eur m 3.0101, Eur m 4, Eur m 4.0101), Evynnis spp (Evy j 1), Fagopyrum spp (Fag e 1, Fag e 1.0101, Fag e 10 kD, Fag e 19 kD, Fag e 2, Fag e 2.0101, Fag e TI), Fagus spp (Fag s 1, Fag s 1.0101, Fag s 2, Fag s 4), Fagopyrum spp (Fag t 1, Fag t 10 kD, Fag t 2, Fag t 2.0101), Felis spp (Fel d 1, Fel d 1.0101, Fel d 2, Fel d 2.0101, Fel d 3, Fel d 3.0101, Fel d 4, Fel d 4.0101, Fel d 5, Fel d 5.0101, Fel d 6, Fel d 6.0101, Fel d 7, Fel d 7.0101, Fel d 8, Fel d 8.0101, Feld IgG), Fenneropenaeus spp (Fen c 1, Fen c 2, Fen me 1, Fen me 1.0101), Festuca spp (Fes e 1, Fes e 13, Fes e 4, Fes e 5, Fes e 7, Fes p 1, Fes p 13, Fes p 4, Fes p 4.0101, Fes p 5, Fes r 1, Fes r 5), Ficus spp (Fic c 17 kD, Fic c 4, Fic c Ficin), Foeniculum spp (Foe v 1, Foe v 2), Forsythia spp (For s 1), Forcipomyia spp (Fort 1, Fort 1.0101, Fort 2, Fort 2.0101, Fort 7, Fort FPA, Fort Myosin, Fort TPI), Fragaria spp (Fra a 1, Fra a 1.0101, Fra a 3, Fra a 3.0101, Fra a 3.0102, Fra a 3.0201, Fra a 3.0202, Fra a 3.0203, Fra a 3.0204, Fra a 3.0301, Fra a 4, Fra a 4.0101, Fra c 1), Fraxinus spp (Fra e 1, Fra e 1.0101, Fra e 1.0102, Fra e 1.0201, Fra e 12, Fra e 2, Fra e 3, Fra e 9), Fragaria spp (Fra v 1), Fusarium spp (Fus c 1, Fus c 1.0101, Fus c 2, Fus c 2.0101, Fus c 3, Fus s 1, Fus s 45 kD, Fus sp Lipase), Gadus spp (Gad c 1, Gad c 1.0101, Gad c APDH, Gad m 1, Gad m 1.0101, Gad m 1.0102, Gad m 1.0201, Gad m 1.0202, Gad m 45 kD, Gad m Gelatin, Gad ma 1), Gallus spp (Gal d 1, Gal d 1.0101, Gal d 2, Gal d 2.0101, Gal d 3, Gal d 3.0101, Gal d 4, Gal d 4.0101, Gal d 5, Gal d 5.0101, Gal d 6, Gal d 6.0101, Gal d Apo I, Gal d Apo VI, Gal d GPI, Gal d HG, Gal d IgY, Gal d L-PGDS, Gal d Ovomucin, Gal d Phosvitin, Gal d PRVB, Gal la 4), Galleria spp (Gal m 18 kD, Gal m 24 kD), Gallus spp (Gal so 4), Gammarus spp (Gam s TM), Gelonium spp (Gel m RIP), Geothelphusa spp (Geo de 1), Glossina spp (Glo m 5, Glo m 5.0101, Glo m 7, Glo m 7.0101, Glo m 7.0102, Glo m 7.0103), Glycine spp (Gly a Bd30K, Gly ar Bd30K, Gly ca Bd30K, Gly cl Bd30K, Gly cu Bd30K, Gly cy Bd30K), Glycyphagus spp (Gly d 10, Gly d 10.0101, Gly d 13, Gly d 2, Gly d 2.0101, Gly d 2.0201, Gly d 2.03, Gly d 2/Lep d 2 L1, Gly d 2/Lep d 2 L2, Gly d 2/Lep d 2 L3, Gly d 2/Lep d 2 L4, Gly d 2/Lep d 2 R1, Gly d 2/Lep d 2 R2, Gly d 2/Lep d 2 R3, Gly d 2/Lep d 2 R4, Gly d 2/Lep d 2 R5, Gly d 20, Gly d 3, Gly d 5, Gly d 5.01, Gly d 5.02, Gly d 7, Gly d 8), Glycine spp (Gly f Bd30K, Gly I Bd30K, Gly m 1, Gly m 1.0101, Gly m 1.0102, Gly m 2, Gly m 2.0101, Gly m 2S Albumin, Gly m 3, Gly m 3.0101, Gly m 3.0102, Gly m 39 kD, Gly m 4, Gly m 4.0101, Gly m 5, Gly m 5.0101, Gly m 5.0201, Gly m 5.0301, Gly m 5.0302, Gly m 50 kD, Gly m 6, Gly m 6.0101, Gly m 6.0201, Gly m 6.0301, Gly m 6.0401, Gly m 6.0501, Gly m 68 kD, Gly m Agglutinin, Gly m Bd28K, Gly m Bd30K, Gly m Bd60K, Gly m CPI, Gly m EAP, Gly m TI, Gly mi Bd30K, Gly s Bd30K, Gly t Bd30K, Gly to Bd30K), Gossypium spp (Gos h Vicilin), Haemophilus spp (Hae in P6), Haemaphysalis spp (Hae 17, Hae 1 7.0101, Hae q 7, Hae q 7.0101), Haliotis spp (Hal a 1, Hal d 1, Hal di 1, Hal di PM, Hal m 1, Hal m 1.0101, Hal r 1, Hal r 49 kD, Hal ru 1), Harmonia spp (Har a 1, Har a 1.0101, Har a 2, Har a 2.0101), Harpegnathos spp (Har sa 7, Har sa 7.0101, Har sa 7.0102), Helianthus spp (Hel a 1, Hel a 1.0101, Hel a 2, Hel a 2.0101, Hel a 2S Albumin, Hel a 3, Hel a 3.0101, Hel a 4), Helix spp (Hel ap 1, Hel as 1, Hel as 1.0101), Heligmosomoides spp (Hel p 3, Hel p 3.0101), Helianthus spp (Hel to 1), Hemanthias spp (Hem le 1), Hemifusus spp (Hem t 1), Heterodera spp (Het g 3, Het g 3.0101), Hevea spp (Hey b 1, Hey b 1.0101, Hey b 10, Hey b 10.0101, Hey b 10.0102, Hey b 10.0103, Hey b 11, Hey b 11.0101, Hey b 11.0102, Hey b 12, Hey b 12.0101, Hey b 13, Hey b 13.0101, Hey b 14, Hey b 14.0101, Hey b 2, Hey b 2.0101, Hey b 3, Hey b 3.0101, Hey b 4, Hey b 4.0101, Hey b 5, Hey b 5.0101, Hey b 6, Hey b 6.01, Hey b 6.02, Hey b 6.0202, Hey b 6.03, Hey b 7, Hey b 7.01, Hey b 7.02, Hey b 7.D2, Hey b 7.S2, Hey b 8, Hey b 8.0101, Hey b 8.0102, Hey b 8.0201, Hey b 8.0202, Hey b 8.0203, Hey b 8.0204, Hey b 9, Hey b 9.0101, Hey b Citrate binding Protein, Hey b GAPDH, Hey b HSP80, Hey b IFR, Hey b Proteasome subunit, Hey b Rotamase, Hey b SPI, Hey b Trx, Hey b UDPGP), Hexagrammos spp (Hex of 1), Hippoglossus spp (Hip h 1), Hippoglossoides spp (Hip pl 1), Hippoglossus spp (Hip st 1), Hirudo spp (Hir me Hirudin), Holcus spp (Hol 1 1, Hol 1 1.0101, Hol 1 1.0102, Hol 1 2, Hol 1 4, Hol 1 5, Hol 1 5.0101, Hol 1 5.0201), Holocnemus spp (Hol pl 9, Hol pl Hemocyanin), Homarus spp (Hom a 1, Hom a 1.0101, Hom a 1.0102, Hom a 1.0103, Hom a 3, Hom a 3.0101, Hom a 4, Hom a 6, Hom a 6.0101, Hom g 1, Hom g 2), Homo spp (Hom s 1, Hom s 1.0101, Hom s 2, Hom s 2.0101, Hom s 3, Hom s 3.0101, Hom s 4, Hom s 4.0101, Hom s 5, Hom s 5.0101, Hom s AAT, Hom s ACTH, Hom s Adalimumab, Hom s ALA, Hom s alpha_Actin, Hom s alpha-Galactosidase, Hom s APDH, Hom s Arylsulfatase B, Hom s Casein, Hom s CyP A, Hom s CyP B, Hom s CyP C, Hom s DSF70, Hom s DSG3, Hom s eIF6, Hom s Etanercept, Hom s Factor IX, Hom s Factor VII, Hom s Factor VIII, Hom s G-CSF, Hom s Glucocerebrosidase, Hom s Glucosidase, Hom s HLA-DR-alpha, Hom s HSA, Hom s Iduronidase, Hom s Idursulfase, Hom s IgA, Hom s Insulin, Hom s Lactoferrin, Hom s Laminin gamma_2, Hom s MnSOD, Hom s Oxytocin, Hom s P2, Hom s Phosvitin, Hom s Profilin, Hom s PSA, Hom s RP1, Hom s TCTP, Hom s TL, Hom s TPA, Hom s TPO, Hom s Transaldolase, Hom s Trx, Hom s Tubulin-alpha, Hom s/Mus m Basiliximab, Hom s/Mus m Cetuximab, Hom s/Mus m Cetuximab (Gal-Gal), Hom s/Mus m Infliximab, Hom s/Mus m Natalizumab, Hom s/Mus m Omalizumab, Hom s/Mus m Palivizumab, Hom s/Mus m Rituximab, Hom s/Mus m Tocilizumab, Hom s/Mus m Trastuzumab), Hoplostethus spp (Hop a 1), Hordeum spp (Hor v 1, Hor v 12, Hor v 12.0101, Hor v 13, Hor v 14, Hor v 15, Hor v 15.0101, Hor v 16, Hor v 16.0101, Hor v 17, Hor v 17.0101, Hor v 18 kD, Hor v 2, Hor v 21, Hor v 21.0101, Hor v 28, Hor v 33, Hor v 4, Hor v 5, Hor v 5.0101, Hor v BDAI, Hor v BTI), Humicola spp (Hum in Cellulose), Humulus spp (Hum j 1, Hum j 1.0101, Hum j 10 kD, Hum j 2), Huso spp (Hus h 1), Hylocereus spp (Hyl un LTP), Hymenocephalus spp (Hym st 1), Hyperoglyphe spp (Hyp by 1), Hypophthalmichthys spp (Hyp mo 1), Hypophthalmichthy spp (Hyp no 1), Ictalurus spp (Ict fu 1, Ict p 1), Imperata spp (Imp c 4, Imp c 5, Imp c VIIIel), Ixodes spp (Ixo r 2, Ixo sc 7, Ixo sc 7.0101), Jasus spp (Jas la 1, Jas la 1.0101, Jas la 1.0102), Juglans spp (Jug ca 1, Jug ca 2, Jug ci 1, Jug ci 2, Jug n 1, Jug n 1.0101, Jug n 2, Jug n 2.0101, Jug r 1, Jug r 1.0101, Jug r 2, Jug r 2.0101, Jug r 3, Jug r 3.0101, Jug r 4, Jug r 4.0101, Jug r 5), Juniperus spp (Jun a 1, Jun a 1.0101, Jun a 1.0102, Jun a 2, Jun a 2.0101, Jun a 3, Jun a 3.0101, Jun c 1, Jun o 1, Jun o 4, Jun o 4.0101, Jun r 3, Jun r 3.1, Jun r 3.2, Jun v 1, Jun v 1.0101, Jun v 1.0102, Jun v 3, Jun v 3.0101, Jun v 3.0102, Jun v 4), Katsuwonus spp (Kat p 1), Kyphosus spp (Kyp se 1), Lachnolaimus spp (Lac ma 1), Lachesis spp (Lac mu 1), Lactuca spp (Lac s 1, Lac s 1.0101), Lagocephalus spp (Lag la 1), Larus spp (Lar a 1, Lar a 2, Lar a 3), Larimichthys spp (Lar po 1), Lates spp (Lat c 1), Lateolabrax spp (Lat ja 1), Lathyrus spp (Lat oc Agglutinin), Leiostomus spp (Lei xa 1), Lens spp (Len c 1, Len c 1.0101, Len c 1.0102, Len c 1.0103, Len c 2, Len c 2.0101, Len c 3, Len c 3.0101, Len c Agglutinin), Leopardus spp (Leo p 1), Lepidoglyphus spp (Lep d 10, Lep d 10.0101, Lep d 12, Lep d 13, Lep d 13.0101, Lep d 2, Lep d 2.0101, Lep d 2.0102, Lep d 2.0201, Lep d 2.0202, Lep d 3, Lep d 39 kD, Lep d 5, Lep d 5.0101, Lep d 5.0102, Lep d 5.0103, Lep d 7, Lep d 7.0101, Lep d 8, Lep d alpha Tubulin), Lepomis spp (Lep gi 1), Leptomelanosoma spp (Lep i 1), Lepomis spp (Lep ma 1), Lepisma spp (Lep s 1, Lep s 1.0101, Lep s 1.0102), Lepeophtheirus spp (Lep sa 1, Lep sa 1.0101, Lep sa 1.0102, Lep sa 1.0103), Leptailurus spp (Lep se 1), Lepidorhombus spp (Lep w 1, Lep w 1.0101), Lethocerus spp (Let in 7, Let in 7.0101, Let in 7.0102), Leuciscus spp (Leu ce 1), Lewia spp (Lew in 1), Ligustrum spp (Lig v 1, Lig v 1.0101, Lig v 1.0102, Lig v 2), Lilium spp (Lil 12, Lil I PG), Limanda spp (Lim fe 1), Limnonectes spp (Lim m 1), Limulus spp (Lim p 1, Lim p 1.0101, Lim p 2, Lim p LPA), Liposcelis spp (Lip b 1, Lip b 1.0101), Litchi spp (Lit c 1, Lit c 1.0101, Lit c IFR, Lit c TPI), Lithobates spp (Lit ca 1), Litopenaeus spp (Lit se 1, Lit v 1, Lit v 1.0101, Lit v 2, Lit v 2.0101, Lit v 3, Lit v 3.0101, Lit v 4, Lit v 4.0101), Filiaria spp (Loa lo 3, Loa lo 3.0101), Lobotes spp (Lob su 1), Locusta spp (Loc m 7, Loc m 7.0101), Loligo spp (Lol b 1, Lol e 1), Lolium spp (Lol m 2, Lol m 5, Lol p 1, Lol p 1.0101, Lol p 1.0102, Lol p 1.0103, Lol p 10, Lol p 11, Lol p 11.0101, Lol p 12, Lol p 13, Lol p 2, Lol p 2.0101, Lol p 3, Lol p 3.0101, Lol p 4, Lol p 4.0101, Lol p 5, Lol p 5.0101, Lol p 5.0102, Lol p 7, Lol p CyP, Lol p FT, Lol p Legumin), Lonomia spp (Lon o 7, Lon o 7.0101), Lophodytes spp (Lop cu 1), Lophonetta spp (Lop sp 1), Lupinus spp (Lup a 1, Lup a alpha_Conglutin, Lup a delta_Conglutin, Lup a gamma_Conglutin, Lup an 1, Lup an 1.0101, Lup an alpha_Conglutin, Lup an delta_Conglutin, Lup an gamma_Conglutin, Lup 117 kD), Lutjanus spp (Lut a 1, Lut c 1, Lut cy 1, Lut gr 1, Lut gu 1, Lut jo 1), Lutraria spp (Lut p 1), Lutjanus spp (Lut pu 1, Lut sy 1), Lycopersicon spp (Lyc e 1, Lyc e 1.0101, Lyc e 11S Globulin, Lyc e 2, Lyc e 2.0101, Lyc e 2.0102, Lyc e 3, Lyc e 3.0101, Lyc e 4, Lyc e 4.0101, Lyc e ARP60S, Lyc e Chitinase, Lyc e Glucanase, Lyc e Peroxidase, Lyc e PG, Lyc e PME, Lyc e PR23, Lyc e Vicilin), Maconellicoccus spp (Mac h 7, Mac h 7.0101), Macruronus spp (Mac ma 1, Mac n 1), Madura spp (Mac po 17 kD), Macrobrachium spp (Mac ro 1, Mac ro 1.0101, Mac ro Hemocyanin), Macropus spp (Marr s Gelatin), Malta spp (Mal d 1, Mal d 1.0101, Mal d 1.0102, Mal d 1.0103, Mal d 1.0104, Mal d 1.0105, Mal d 1.0106, Mal d 1.0107, Mal d 1.0108, Mal d 1.0109, Mal d 1.0201, Mal d 1.0202, Mal d 1.0203, Mal d 1.0204, Mal d 1.0205, Mal d 1.0206, Mal d 1.0207, Mal d 1.0208, Mal d 1.0301, Mal d 1.0302, Mal d 1.0303, Mal d 1.0304, Mal d 1.0401, Mal d 1.0402, Mal d 1.0403, Mal d 2, Mal d 2.0101, Mal d 3, Mal d 3.0101, Mal d 3.0102, Mal d 3.0201, Mal d 3.0202, Mal d 3.0203, Mal d 4, Mal d 4.0101, Mal d 4.0102, Mal d 4.0201, Mal d 4.0202, Mal d 4.0301, Mal d 4.0302), Malpighia spp (Mal g 4, Mal g Hevein), Malta spp (Mal p 1), Malassezia spp (Mala f 2, Mala f 2.0101, Mala f 3, Mala f 3.0101, Mala f 4, Mala f 4.0101, Mala g 10, Malas 1, Malas 1.0101, Malas 10, Malas 10.0101, Malas 11, Malas 11.0101, Mala s 12, Mala s 12.0101, Mala s 13, Mala s 13.0101, Mala s 5, Mala s 5.0101, Mala s 6, Mala s 6.0101, Mala s 7, Mala s 7.0101, Mala s 8, Mala s 8.0101, Mala s 9, Mala s 9.0101), Manihot spp (Mane 5, Man e 5.0101, Mane FPA, Man e GAPDH), Mangifera spp (Man i 1, Man i 14 kD, Man i 2, Man i 3, Man i 3.01, Man i 3.02, Man i Chitinase), Marsupenaeus spp (Mar j 1, Mar j 1.0101, Mar j 2, Mar j 4), Matricaria spp (Mat c 17 kD), Mecopoda spp (Mec e 7), Megalobrama spp (Meg am 2, Meg am CK), Megathura spp (Meg c Hemocyanin), Megalops spp (Meg sp 1), Melanogrammus spp (Mel a 1), Meleagris spp (Mel g 1, Mel g 2, Mel g 3, Mel g PRVB, Mel g TSA), Mehcertus spp (Mel 11), Menticirrhus spp (Men am 1), Mercurialis spp (Mer a 1, Mer a 1.0101), Merluccius spp (Mer ap 1, Mer au 1, Mer bi 1, Mer ca 1, Mer ga 1, Mer hu 1), Merlangius spp (Mer me 1), Merluccius spp (Mer mr 1, Mer pa 1, Mer po 1, Mer pr 1, Mer se 1), Meriones spp (Mer un 23 kD), Metarhizium spp (Met a 30), Metapenaeopsis spp (Met ba 1), Metapenaeus spp (Mete 1, Mete 1.0101, Met e 2), Metasequoia spp (Met gl 2), Metapenaeus spp (Met j 1, Met j 2), Metanephrops spp (Met ja 1), Metapenaeopsis spp (Met la 1), Metanephrops spp (Met t 2), Micromesistius spp (Mic po 1), Micropogonias spp (Mic un 1), Mimachlamys spp (Mim n 1), Momordica spp (Mom c RIP), Morus spp (Mor a 17 kD, Mor a 4), Morone spp (Mor am 1), Morus spp (Mor n 3, Mor n 3.0101), Morone spp (Mor sa 1, Mor sc 1), Mugil spp (Mug c 1), Muraenolepis spp (Mur mi 1), Musa spp (Mus a 1, Mus a 1.0101, Mus a 2, Mus a 2.0101, Mus a 3, Mus a 3.0101, Mus a 4, Mus a 4.0101, Mus a 5, Mus a 5.0101, Musa 5.0102), Mus spp (Mus m 1, Mus m 1.0101, Mus m 1.0102, Mus m 2, Mus m Gelatin, Mus m IgG, Mus m MSA, Mus m Muromonab, Mus m Phosvitin), Mustela spp (Mus p 17 kD), Musa spp (Mus xp 1, Mus xp 2, Mus xp 5), Mycteroperca spp (Myc bo 1, Myc mi 1, Myc ph 1), Myceliophthora spp (Myc sp Laccase), Myrmecia spp (Myr p 1, Myr p 1.0101, Myr p 2, Myr p 2.0101, Myr p 2.0102, Myr p 3, Myr p 3.0101), Mytilus spp (Myt e 1, Myt g 1, Myt g PM), Myzus spp (Myz p 7, Myz p 7.0101), Nemorhedus spp (Nae go Hya), Necator spp (Nec a Calreticulin), Nemipterus spp (Nem vi 1), Neosartorya spp (Neo fi 1, Neo fi 22), Neochen spp (Neo ju 1), Neoscona spp (Neo n 7, Neo n 7.0101), Nephelium spp (Nep I GAPDH), Nephrops spp (Nep n 1, Nep n DF9), Neptunea spp (Nep po 1, Nep po 1.0101), Nicotiana spp (Nic t 8, Nic t Osmotin, Nic t Villin), Nimbya spp (Nim c 1, Nim s 1), Nippostrongylus spp (Nip b Agl), Nycticebus spp (Nyc c 1), Octopus spp (Oct f 1, Oct 11, Oct v 1, Oct v 1.0101, Oct v PM), Ocyurus spp (Ocy ch 1), Olea spp (Ole e 1, Ole e 1.0101, Ole e 1.0102, Ole e 1.0103, Ole e 1.0104, Ole e 1.0105, Ole e 1.0106, Ole e 1.0107, Ole e 10, Ole e 10.0101, Ole e 11, Ole e 11.0101, Ole e 11.0102, Ole e 12, Ole e 13, Ole e 2, Ole e 2.0101, Ole e 3, Ole e 3.0101, Ole e 36 kD, Ole e 4, Ole e 4.0101, Ole e 5, Ole e 5.0101, Ole e 6, Ole e 6.0101, Ole e 7, Ole e 7.0101, Ole e 8, Ole e 8.0101, Ole e 9, Ole e 9.0101), Ommastrephes spp (Omm b 1, Omm b 1.0101), Oncorhynchus spp (Onc ke 1, Onc ke 18 kD, Onc ke alpha2l, Onc ke Vitellogenin, Onc m 1, Onc m 1.0101, Onc m 1.0201, Onc m alpha2l, Onc m Protamine, Onc m Vitellogenin, Onc ma 1, Onc ma FPA, Onc ma FSA, Onc ma TPI, Onc n 1), Onchocerca spp (Onc o 3, Onc o 3.0101), Oncorhynchus spp (Onc is 1), Onchocerca spp (Onc v 3, Onc v 3.0101), Oratosquilla spp (Ora o 1, Ora o 1.0101), Oreochromis spp (Ore a 1, Ore mo 1, Ore mo 2, Ore mo FPA, Ore mo SCAF7145, Ore ni 1, Ore ni 18 kD, Ore ni 45 kD), Ornithonyssus spp (Orn sy 10, Orn sy 10.0101, Orn sy 10.0102), Oryctolagus spp (Ory c 1, Ory c 1.0101, Ory c 2, Ory c Casein, Ory c Phosvitin, Ory c RSA), Oryza spp (Ory s 1, Ory s 1.0101, Ory s 11, Ory s 12, Ory s 12.0101, Ory s 13, Ory s 14, Ory s 17 kD, Ory s 19 kD, Ory s 2, Ory s 23, Ory s 3, Ory s 7, Ory s akTI, Ory s GLP52, Ory s GLP63, Ory s Glyoxalase I, Ory s NRA), Ostrya spp (Ost c 1, Ost c 1.0101), Ovis spp (Ovi a ALA, Ovi a BLG, Ovi a Casein, Ovi a Casein alphaS1, Ovi a Casein alphaS2, Ovi a Casein beta, Ovi a Casein kappa, Ovi a Phosvitin, Ovi a SSA), Pachycondyla spp (Pac c 3), Pagrus spp (Pag m 1, Pag pa 1), Pampus spp (Pam ar 1, Pam c 1), Pandalus spp (Pan b 1, Pan b 1.0101), Pangasius spp (Pan bo 1), Pandalus spp (Pan e 1, Pan e 1.0101, Pan e 4), Panulirus spp (Pan h 1, Pan hy 1), Pangasius spp (Pan hy 18 kD, Pan hy 45 kD), Panulirus spp (Pan j 1), Panthera spp (Pan 11, Pan o 1, Pan p 1), Panulirus spp (Pan s 1, Pan s 1.0101), Panthera spp (Pan t 1), Pan spp (Pan tr TCTP), Papaver spp (Pap s 17 kD, Pap s 2, Pap s 34 kD), Papilio spp (Pap xu 7, Pap xu 7.0101, Pap xu 7.0102), Paralichthys spp (Par a 1), Parasilurus spp (Par as 1, Par c 1), Paralithodes spp (Par c 1.0101, Par c 1.0102, Par f 1), Parthenium spp (Par h 1), Parietaria spp (Par j 1, Par j 1.0101, Par j 1.0102, Par j 1.0103, Par j 1.0201, Par j 2, Par j 2.0101, Par j 2.0102, Par j 3, Par j 3.0101, Par j 3.0102, Par j 4, Par j 4.0101, Par j J1-J2), Paralichthys spp (Par le 1), Parietaria spp (Par m 1, Par o 1, Par o 1.0101), Paralichthys spp (Par of 1, Par of alpha2l), Parahucho spp (Par pe Vitellogenin), Passiflora spp (Pas e Chitinase, Pas e Hevein), Paspalum spp (Pas n 1, Pas n 1.0101, Pas n 13), Patinopecten spp (Pat y 1), Pediculus spp (Ped h 7, Ped h 7.0101), Penaeus spp (Pen a 1, Pen a 1.0101, Pen a 1.0102, Pen a 1.0102 (103-117), Pen a 1.0102 (109-123), Pen a 1.0102 (1-15), Pen a 1.0102 (115-129), Pen a 1.0102 (121-135), Pen a 1.0102 (127-141), Pen a 1.0102 (13-27), Pen a 1.0102 (133-147), Pen a 1.0102 (139-153), Pen a 1.0102 (145-159)), Farfantepenaeus spp (Pen a 1.0102 (151-165)), Penaeus spp (Pen a 1.0102 (157-171), Pen a 1.0102 (163-177), Pen a 1.0102 (169-183), Pen a 1.0102 (175-189), Pen a 1.0102 (181-195), Pen a 1.0102 (187-201), Pen a 1.0102 (193-207), Pen a 1.0102 (19-33), Pen a 1.0102 (199-213), Pen a 1.0102 (205-219), Pena 1.0102 (211-225), Pena 1.0102 (217-231), Pena 1.0102 (223-237), Pen a 1.0102 (229-243)), Farfantepenaeus spp (Pen a 1.0102 (235-249)), Penaeus spp (Pen a 1.0102 (241-255), Pen a 1.0102 (247-261), Pen a 1.0102 (253-267), Pen a 1.0102 (25-39), Pen a 1.0102 (259-273), Pen a 1.0102 (265-279), Pen a 1.0102 (270-284), Pen a 1.0102 (31-45), Pen a 1.0102 (37-51), Pen a 1.0102 (43-57), Pen a 1.0102 (49-63)), Farfantepenaeus spp (Pen a 1.0102 (55-69)), Penaeus spp (Pen a 1.0102 (61-75), Pen a 1.0102 (67-81), Pen a 1.0102 (7-21), Pen a 1.0102 (73-87), Pen a 1.0102 (79-93), Pen a 1.0102 (85-99), Pen a 1.0102 (91-105), Pen a 1.0102 (97-111), Pen a 1.0103), Penicillium spp (Pen b 13, Pen b 13.0101, Pen b 26, Pen b 26.0101, Pen c 1, Pen c 13, Pen c 13.0101, Pen c 18, Pen c 19, Pen c 19.0101, Pen c 2, Pen c 22, Pen c 22.0101, Pen c 24, Pen c 24.0101, Pen c 3, Pen c 3.0101, Pen c 30, Pen c 30.0101, Pen c 32, Pen c 32.0101, Pen c MnSOD, Pen ch 13, Pen ch 13.0101, Pen ch 18, Pen ch 18.0101, Pen ch 20, Pen ch 20.0101, Pen ch 31, Pen ch 31.0101, Pen ch 33, Pen ch 33.0101, Pen ch 35, Pen ch 35.0101, Pen ch MnSOD), Penaeus spp (Pen i 1, Pen i 1.0101, Pen m 1, Pen m 1.0101, Pen m 1.0102, Pen m 2, Pen m 2.0101, Pen m 3, Pen m 3.0101, Pen m 4, Pen m 4.0101, Pen m 6, Pen m 6.0101), Penicillium spp (Pen o 18, Pen o 18.0101), Penaeus spp (Pena o 1, Pena o 1.0101), Periplaneta spp (Per a 1, Per a 1.0101, Per a 1.0102, Per a 1.0103, Per a 1.0104, Per a 1.0105, Per a 1.0201, Per a 10, Per a 10.0101, Per a 2, Per a 3, Per a 3.0101, Per a 3.0201, Per a 3.0202, Per a 3.0203, Per a 4, Per a 5, Per a 6, Per a 6.0101, Per a 7, Per a 7.0101, Per a 7.0102, Per a 7.0103, Per a 9, Per a 9.0101, Per a Cathepsin, Per a FABP, Per a Trypsin, Per f 1, Per f 7, Per f 7.0101), Perna spp (Per v 1), Persea spp (Pers a 1, Pers a 1.0101, Pers a 4), Petroselinum spp (Pet c 1, Pet c 2, Pet c 3), Phalaris spp (Pha a 1, Pha a 1.0101, Pha a 5, Pha a 5.0101, Pha a 5.02, Pha a 5.03, Pha a 5.04), Phaseolus spp (Pha v 3, Pha v 3.0101, Pha v 3.0201, Pha v aAI, Pha v aAI.0101, Pha v Chitinase, Pha v PHA, Pha v Phaseolin), Phleum spp (Phl p 1, Phl p 1.0101, Phl p 1.0102, Phl p 11, Phl p 11.0101, Phl p 12, Phl p 12.0101, Phl p 12.0102, Phl p 12.0103, Phl p 13, Phl p 13.0101, Phl p 2, Phl p 2.0101, Phl p 3, Phl p 3.0101, Phl p 3.0102, Phl p 4, Phl p 4.0101, Phl p 4.0102, Phl p 4.0201, Phl p 4.0202, Phl p 4.0203, Phl p 4.0204, Phl p 5, Phl p 5.0101, Phl p 5.0102, Phl p 5.0103, Phl p 5.0104, Phl p 5.0105, Phl p 5.0106, Phl p 5.0107, Phl p 5.0108, Phl p 5.0109, Phl p 5.0201, Phl p 5.0202, Phl p 5.0203, Phl p 5.0204, Phl p 5.0205, Phl p 5.0206, Phl p 5.0207, Phl p 6, Phl p 6.0101, Phl p 6.0102, Phl p 7, Phl p 7.0101, Phl p P1-P2-P5-P6, Phl p P2-P6, Phl p P5-P1, Phl p P6-P2), Phoenix spp (Pho d 2, Pho d 2.0101, Pho d 40 kD, Pho d 90 kD), Phodopus spp (Pho s 21 kD), Phoma spp (Pho t 1), Phragmites spp (Phr a 1, Phr a 12, Phr a 13, Phr a 4, Phr a 5), Phytolacca spp (Phy a RIP), Pimpinella spp (Pim a 1, Pim a 2), Pinna spp (Pin a 1), Piper spp (Pip n 14 kD, Pip n 28 kD), Pisum spp (Pis s 1, Pis s 1.0101, Pis s 1.0102, Pis s 2, Pis s 2.0101, Pis s 5, Pis s Agglutinin, Pis s Albumin), Pistacia spp (Pis v 1, Pis v 1.0101, Pis v 2, Pis v 2.0101, Pis v 2.0201, Pis v 3, Pis v 3.0101, Pis v 4, Pis v 4.0101, Pis v 5, Pis v 5.0101), Platanus spp (Pla a 1, Pla a 1.0101, Pla a 2, Pla a 2.0101, Pla a 3, Pla a 3.0101, Pla a 8), Platichthys spp (Pla f 1), Plantago spp (Pla 11, Pla 11.0101, Pla I 1.0102, Pla 11.0103, Pla I Cytochrome C), Platanus spp (Pla oc 1, Pla or 1, Pla or 1.0101, Pla or 2, Pla or 2.0101, Pla or 3, Pla or 3.0101, Pla or 4, Pla or CyP, Pla r 1), Plectropomus spp (Ple ar 1), Pleospora spp (Ple h 1), Plectropomus spp (Ple le 1), Plodia spp (Plo i 1, Plo i 1.0101, Plo i 2, Plo i 2.0101), Poa spp (Poa p 1, Poa p 1.0101, Poa p 10, Poa p 12, Poa p 13, Poa p 2, Poa p 4, Poa p 5, Poa p 5.0101, Poa p 6, Poa p 7), Polistes spp (Pol a 1, Pol a 1.0101, Pol a 2, Pol a 2.0101, Pol a 5, Pol a 5.0101, Pol d 1, Pol d 1.0101, Pol d 1.0102, Pol d 1.0103, Pol d 1.0104, Pol d 4, Pol d 4.0101, Pol d 5, Pol d 5.0101, Pol e 1, Pol e 1.0101, Pol e 2, Pole 4, Pol e 4.0101, Pol e 5, Pol e 5.0101, Pol f 5, Pol f 5.0101, Pol g 1, Pol g 1.0101, Pol g 2, Pol g 4, Pol g 5, Pol g 5.0101, Pol he MLT, Pol m 5, Pol m 5.0101), Polypedilum spp (Pol n 1), Pollicipes spp (Pol po 1), Pollachius spp (Pol vi 1), Polybia spp (Poly p 1, Poly p 1.0101, Poly p 2, Poly p 5, Poly s 5, Poly s 5.0101), Pomatomus spp (Porn sa 1), Pongo spp (Pon ab HSA), Pontastacus spp (Pon I 4, Pon I 4.0101, Pon I 7, Pon I 7.0101), Portunus spp (Por s 1, Por s 1.0101, Por s 1.0102, Por tr 1, Por tr 1.0101), Protortonia spp (Pro ca 38 kD), Procumbarus spp (Pro cl 1, Pro cl 1.0101, Pro cl 21 kD), Prosopis spp (Pro j 20 kD), Prunus spp (Pm ar 1, Pm ar 1.0101, Pm ar 3, Pm ar 3.0101, Pm av 1, Pm av 1.0101, Pm av 1.0201, Pm av 1.0202, Pm av 1.0203, Pm av 2, Pm av 2.0101, Pm av 3, Pm av 3.0101, Pm av 4, Pm av 4.0101, Pm c 1, Pm d 1, Pru d 2, Pru d 3, Pru d 3.0101, Pru d 4, Pru du 1, Pru du 2, Pru du 2S Albumin, Pru du 3, Pru du 3.0101, Pm du 4, Pm du 4.0101, Pm du 4.0102, Pm du 5, Pm du 5.0101, Pm du 6, Pm du 6.0101, Pm du 6.0201, Pm du Conglutin, Pm p 1, Pm p 1.0101, Pm p 2, Pm p 2.0101, Pm p 2.0201, Pm p 2.0301, Pm p 3, Pm p 3.0101, Pm p 3.0102, Pm p 4, Pm p 4.0101, Pm p 4.0201, Pm sa 3), Psilocybe spp (Psi c 1, Psi c 1.0101, Psi c 2, Psi c 2.0101), Psoroptes spp (Pso o 1, Pso o 10, Pso o 10.0101, Pso o 11, Pso o 13, Pso o 14, Pso o 2, Pso o 21, Pso o 3, Pso o 5, Pso o 7), Puma spp (Pum c 1), Punica spp (Pun g 3), Pyrus spp (Pyr c 1, Pyr c 1.0101, Pyr c 3, Pyr c 3.0101, Pyr c 4, Pyr c 4.0101, Pyr c 5, Pyr c 5.0101, Pyr py 2), Quercus spp (Que a 1, Que a 1.0101, Que a 1.0201, Que a 1.0301, Que a 1.0401, Que a 2, Que a 4), Rachycentron spp (Rac ca 1), Rana spp (Ran e 1, Ran e 1.0101, Ran e 2, Ran e 2.0101), Ranina spp (Ran ra 1), Rangifer spp (Ran t BLG), Rattus spp (Rat n 1, Rat n 1.0101, Rat n Casein, Rat n Gelatin, Rat n IgG, Rat n Phosvitin, Rat n RSA, Rat n Transferrin), Rhizomucor spp (Rhi m AP), Rhizopus spp (Rhi nv Lipase, Rhi o Lipase), Rhomboplites spp (Rho au 1), Rhodotorula spp (Rho m 1, Rho m 1.0101, Rho m 2, Rho m 2.0101), Ricinus spp (Ric c 1, Ric c 1.0101, Ric c 2, Ric c 3, Ric c 8, Ric c RIP), Rivulus spp (Riv ma 1), Robinia spp (Rob p 2, Rob p 4, Rob p Glucanase), Rosa spp (Ros r 3), Roystonea spp (Roy e 2), Rubus spp (Rub i 1, Rub i 1.0101, Rub i 3, Rub i 3.0101, Rub i Chitinase, Rub i CyP), Saccharomyces spp (Sac c Carboxypeptidase Y, Sac c CyP, Sac c Enolase, Sac c Glucosidase, Sac c Invertase, Sac c MnSOD, Sac c P2, Sac c Profilin), Salvelinus spp (Sal f 1), Salsola spp (Sal k 1, Sal k 1.0101, Sal k 1.0201, Sal k 1.0301, Sal k 1.0302, Sal k 2, Sal k 2.0101, Sal k 3, Sal k 3.0101, Sal k 4, Sal k 4.0101, Sal k 4.0201, Sal k 5, Sal k 5.0101), Salvelinus spp (Sal le Vitellogenin), Salmo spp (Sal s 1, Sal s 1.0101, Sal s 1.0201, Sal s 2, Sal s 2.0101, Sal s Gelatin), Sambucus spp (Sam n 1), Sander spp (San lu 1), Saponaria spp (Sap o RIP), Sardinops spp (Sar m 1), Sarkidiornis spp (Sar ml 1), Sardina spp (Sar p 1), Sarcoptes spp (Sar s 1, Sar s 14, Sar s 3, Sar s GST, Sar s PM), Sardinops spp (Sar sa 1, Sar sa 1.0101), Schistosoma spp (Sch j GST, Sch j PM, Sch j Sj22, Sch j Sj67, Sch ma Sm20, Sch ma Sm21, Sch ma Sm22, Sch ma Sm31), Sciaenops spp (Sci oc 1), Scomber spp (Sco a 1), Scombermorus spp (Sco ca 1), Scomberomorus spp (Sco g 1), Scomber spp (Sco j 1, Sco ma 1, Sco s 1), Scolopendra spp (Sco y 7, Sco y 7.0101), Scylla spp (Scy o 1, Scy o 1.0101, Scy o 2, Scy pa 1, Scy pa 2, Scy s 1, Scy s 1.0101, Scy s 2), Sebastes spp (Seb fa 1, Seb in 1, Seb m 1, Seb m 1.0101, Seb m 1.0201), Secale spp (Sec c 1, Sec c 12, Sec c 13, Sec c 2, Sec c 20, Sec c 20.0101, Sec c 20.0201, Sec c 28, Sec c 3, Sec c 4, Sec c 4.0101, Sec c 4.0201, Sec c 5, Sec c 5.0101, Sec c akTI, Sec c akTI.0101), Senecio spp (Sen j MDH, Sen j PL), Sepia spp (Sep e 1, Sep e 1.0101), Sepioteuthis spp (Sep 11, Sep I 1.0101), Sepia spp (Sep m 1), Seriola spp (Ser d 1, Ser la 1), Sergestes spp (Ser lu 1), Seriola spp (Ser q 1, Ser ri 1), Sesamum spp (Ses i 1, Ses i 1.0101, Ses i 2, Ses i 2.0101, Ses i 3, Ses i 3.0101, Ses i 4, Ses i 4.0101, Ses i 5, Ses i 5.0101, Ses i 6, Ses i 6.0101, Ses i 7, Ses i 7.0101, Ses i 8), Shigella spp (Shi bo GST, Shi dy GST), Simulia spp (Sim vi 1, Sim vi 2, Sim vi 3, Sim vi 4, Sim vi 70 kD), Sinapis spp (Sin a 1, Sin a 1.0101, Sin a 1.0104, Sin a 1.0105, Sin a 1.0106, Sin a 1.0107, Sin a 1.0108, Sin a 2, Sin a 2.0101, Sin a 3, Sin a 3.0101, Sin a 4, Sin a 4.0101), Sinonovacula spp (Sin c 1, Sin c 1.0101), Solenopsis spp (Sol g 2, Sol g 2.0101, Sol g 3, Sol g 3.0101, Sol g 4, Sol g 4.0101, Sol g 4.0201, Sol i 1, Sol i 1.0101, Sol i 2, Sol i 2.0101, Sol i 3, Sol i 3.0101, Sol i 4, Sol i 4.0101), Solenocera spp (Sol me 1), Solenopsis spp (Sol r 1, Sol r 2, Sol r 2.0101, Sol r 3, Sol r 3.0101, Sol s 2, Sol s 2.0101, Sol s 3, Sol s 3.0101, Sol s 4), Solea spp (Sol so 1, Sol so TPI), Solanum spp (Sola t 1, Sola t 1.0101, Sola t 2, Sola t 2.0101, Sola t 3, Sola t 3.0101, Sola t 3.0102, Sola t 4, Sola t 4.0101, Sola t 8, Sola t Glucanase), Sorghum spp (Sorb 1, Sor h 1, Sor h 1.0101, Sor h 12, Sor h 7), Sparus spp (Spa a 1), Sphyrna spp (Sph ti 1), Spirulina spp (Spi mx beta_Phycocyanin), Spinacia spp (Spi o 2, Spi o RuBisCO), Squilla spp (Squ ac 1, Squ ac 1.0101, Squ o 1, Squ o 1.0101), Staphylococcus spp (Sta a FBP, Sta a SEA, Sta a SEB, Sta a SEC, Sta a SED, Sta a SEE, Sta a TSST), Stachybotrys spp (Sta c 3, Sta c 3.0101, Sta c Cellulase, Sta c Hemolysin, Sta c SchS34, Sta c Stachyrase A), Stemphylium spp (Ste b 1, Ste c 1, Ste v 1), Stolephorus spp (Sto i 1), Struthio spp (Str c 1, Str c 2, Str c 3), Streptococcus spp (Str dy Streptokinase), Streptomyces spp (Str g Pronase), Streptococcus spp (Str pn PspC), Strongylocentrotus spp (Str pu 18 kD, Str pu Vitellogenin), Streptococcus spp (Str py SPEA, Str py SPEC, Str py Streptokinase), Strongyloides spp (Str st 45 kD), Streptomyces spp (Str v PAT), Styela spp (Sty p 1), Suidasia spp (Sui m 1, Sui m 13, Sui m 2, Sui m 3, Sui m 5, Sui m 5.01, Sui m 5.02, Sui m 5.03, Sui m 6, Sui m 7, Sui m 8, Sui m 9), Sus spp (Sus s ACTH, Sus s ALA, Sus s Amylase, Sus s BLG, Sus s Casein, Sus s Casein alphaS1, Sus s Casein alphaS2, Sus s Casein beta, Sus s Casein kappa, Sus s Gelatin, Sus s HG, Sus s Insulin, Sus s Lipase, Sus s Pepsin, Sus s Phosvitin, Sus s PRVB, Sus s PSA, Sus s TCTP), Syntelopodeuma spp (Syn y 7, Syn y 7.0101), Syringa spp (Syr v 1, Syr v 1.0101, Syr v 1.0102, Syr v 1.0103, Syr v 2, Syr v 3, Syr v 3.0101), Tabanus spp (Tab y 1, Tab y 1.0101, Tab y 2, Tab y 2.0101, Tab y 5, Tab y 5.0101), Tadorna spp (Tad ra 1), Talaromyces spp (Tal st 22, Tal st 3, Tal st 8), Taraxacum spp (Tar o 18 kD), Taxodium spp (Tax d 2), Tegenaria spp (Teg d Hemocyanin), Teladorsagia spp (Tel ci 3), Thaumetopoea spp (Tha p 1, Tha p 1.0101, Tha p 2, Tha p 2.0101), Theragra spp (The c 1), Thermomyces spp (The I Lipase, The sp Lipase, The sp Xylanase), Thunnus spp (Thu a 1, Thu a 1.0101, Thu a Collagen, Thu al 1, Thu at 1, Thu o 1, Thu o Collagen), Thuja spp (Thu oc 3, Thu p 1), Thunnus spp (Thu t 1, Thu to 1), Thyr sites spp (Thy at 1), Thyrophygus spp (Thy y 7, Thy y 7.0101), Todarodes spp (Tod p 1, Tod p 1.0101, Tod p 1.0102), Toxoptera spp (Tox c 7, Tox c 7.0101), Toxocara spp (Tox ca TES120, Tox ca TES26, Tox ca TES30), Toxoplasma spp (Tox g HSP70), Trachypenaeus spp (Tra c 1), Trachinotus spp (Tra ca 1), Trachurus spp (Tra j 1, Tra j Gelatin, Tra tr Gelatin), Triticum spp (Tri a 1, Tri a 10 kD, Tri a 12, Tri a 12.0101, Tri a 12.0102, Tri a 12.0103, Tri a 12.0104, Tri a 13, Tri a 14, Tri a 14.0101, Tri a 14.0201, Tri a 15, Tri a 15.0101, Tri a 18, Tri a 18.0101, Tri a 19, Tri a 19.0101, Tri a 2, Tri a 21, Tri a 21.0101, Tri a 23kd, Tri a 25, Tri a 25.0101, Tri a 26, Tri a 26.0101, Tri a 27, Tri a 27.0101, Tri a 28, Tri a 28.0101, Tri a 29, Tri a 29.0101, Tri a 29.0201, Tri a 3, Tri a 30, Tri a 30.0101, Tri a 31, Tri a 31.0101, Tri a 32, Tri a 32.0101, Tri a 33, Tri a 33.0101, Tri a 34, Tri a 34.0101, Tri a 35, Tri a 35.0101, Tri a 36, Tri a 36.0101, Tri a 37, Tri a 37.0101, Tri a 4, Tri a 4.0101, Tri a 4.0201, Tri a 5, Tri a 7, Tri a aA_SI, Tri a alpha_Gliadin, Tri a bA, Tri a Bd36K, Tri a beta_Gliadin, Tri a Chitinase, Tri a CM16, Tri a DH, Tri a Endochitinase, Tri a gamma_Gliadin, Tri a Germin, Tri a Gliadin, Tri a GST, Tri a LMW Glu, Tri a LMW-GS B16, Tri a LMW-GS P42, Tri a LMW-GS P73, Tri a LTP2, Tri a omega2_Gliadin, Tri a Peroxidase, Tri a Peroxidase 1, Tri a SPI, Tri a TLP, Tri a Tritin, Tri a XI), Tritirachium spp (Tri al Proteinase K), Tribolium spp (Tri ca 17, Tri ca 17.0101, Tri ca 7, Tri ca 7.0101), Trichostrongylus spp (Tri co 3, Tri co 3.0101), Trichophyton spp (Tri eq 4), Trigonella spp (Tri fg 1, Tri fg 2, Tri fg 3, Tri fg 4), Trichosanthes spp (Tri k RIP), Trichiurus spp (Tri le 1), Triticum spp (Tri m Peroxidase), Trichophyton spp (Tri me 2, Tri me 4), Trisetum spp (Tri p 1, Tri p 5), Trichinella spp (Tri ps 3, Tri ps 3.0101), Trichophyton spp (Tri r 2, Tri r 2.0101, Tri r 4, Tri r 4.0101), Trichoderma spp (Tri rs Cellulase), Triticum spp (Tri s 14), Trichophyton spp (Tri sc 2, Tri sc 4, Tri so 2), Trichinella spp (Tri sp 3, Tri sp 3.0101, Tri sp 3.0102, Tri sp 3.0103, Tri sp 3.0104, Tri sp 3.0105, Tri sp 3.0106), Trichophyton spp (Tri t 1, Tri t 1.0101, Tri t 4, Tri t 4.0101), Triticum spp (Tri td 14, Tri td akTI), Trichoderma spp (Tri v Cellulase), Trichophyton spp (Tri ye 4), Triatoma spp (Tria p 1, Tria p 1.0101), Triplochiton spp (Trip s 1), Turbo spp (Tur c 1, Tur c PM), Tyrophagus spp (Tyr p 1, Tyr p 10, Tyr p 10.0101, Tyr p 10.0102, Tyr p 13, Tyr p 13.0101, Tyr p 2, Tyr p 2.0101, Tyr p 24, Tyr p 24.0101, Tyr p 3, Tyr p 3.0101, Tyr p 4, Tyr p 5, Tyr p 5.01, Tyr p 5.02, Tyr p 5.03, Tyr p 7, Tyr p alpha Tubulin), Ulocladium spp (Ulo a 1, Ulo at 1, Ulo b 1, Ulo c 1, Ulo co 1, Ulo cu 1, Ulo mu 1, Ulo ob 1, Ulo se 1, Ulo su 1, Ulo to 1), Uncia spp (Unc u 1), Urophycis spp (Uro to 1), Vaccinium spp (Vac m 3), Varroa spp (Var j 13 kD), Venerupis spp (Ven ph 1, Ven ph 1.0101), Vespula spp (Ves f 1, Ves f 2, Ves f 5, Ves f 5.0101, Ves g 1, Ves g 2, Ves g 5, Ves g 5.0101, Ves m 1, Ves m 1.0101, Ves m 2, Ves m 2.0101, Ves m 5, Ves m 5.0101, Ves m MLT, Ves p 1, Ves p 2, Ves p 5, Ves p 5.0101, Ves s 1, Ves s 1.0101, Ves s 2, Ves s 5, Ves s 5.0101, Ves v 1, Ves v 1.0101, Ves v 2, Ves v 2.0101, Ves v 2.0201, Ves v 3, Ves v 3.0101, Ves v 5, Ves v 5.0101, Ves v 5-Pol a 5, Ves vi 5, Ves vi 5.0101), Vespa spp (Vesp c 1, Vesp c 1.0101, Vesp c 2, Vesp c 5, Vesp c 5.0101, Vesp c 5.0102, Vesp m 1, Vesp m 1.0101, Vesp m 5, Vesp m 5.0101, Vesp ma 1, Vesp ma 2, Vesp ma 5, Vesp ma MLT, Vesp v MLT), Vigna spp (Vig r 1, Vig r 1.0101, Vig r 17 kD, Vig r 5, Vig r 8S Globulin, Vig r Albumin, Vig r beta-Conglycinin), Vitis spp (Vit v 1, Vit v 1.0101, Vit v 4, Vit v 5, Vit v Glucanase, Vit v TLP), Xiphias spp (Xip g 1, Xip g 1.0101, Xip g 25 kD), Zea spp (Zea m 1, Zea m 1.0101, Zea m 11, Zea m 12, Zea m 12.0101, Zea m 12.0102, Zea m 12.0103, Zea m 12.0104, Zea m 12.0105, Zea m 13, Zea m 14, Zea m 14.0101, Zea m 14.0102, Zea m 2, Zea m 20S, Zeam 22, Zeam 25, Zeam 25.0101, Zeam 27 kD Zein, Zeam 3, Zeam 4, Zeam 5, Zeam 50 kD Zein, Zea m 7, Zea m Chitinase, Zea m G1, Zea m G2, Zea m PAO, Zea m Zm13), Zeus spp (Zeu fa 1), Ziziphus spp (Ziz m 1, Ziz m 1.0101), Zoarces spp (Zoa a ISP III), and Zygophyllum spp (Zyg f 2).

Heterologous Antigens

The compositions herein described can comprise a plurality of heterologous antigens (e.g, a plurality of disparate AP). In some embodiments, “heterologous antigens” are antigens that are of different origins, such as derived from pathogens of different taxonomic groups such as different strains, species, subgenera, genera, subfamilies or families and/or from antigenically divergent pathogens (e.g., variants thereof). Classification of viruses into various taxonomic groups is well understood by those skilled in the art. Each of the disparate AP of the plurality of disparate AP can differ with respect to each other.

At least two of the fusion proteins of the plurality of fusion proteins can be different from each other with respect to the AP, and wherein the population of ENPs thereby display a plurality of disparate AP. The population of ENPs can comprise one or more homotypic ENPs, wherein the plurality of fusion proteins of a homotypic ENP can be the same as each other with respect to the AP, and wherein a homotypic ENP thereby does not display a plurality of disparate AP. The population of ENPs can comprise one or more heterotypic ENPs, wherein at least two of the fusion proteins of a heterotypic ENP can be different from each other with respect to the AP, and wherein a heterotypic ENP thereby displays a plurality of disparate AP. The population of ENPs can comprise a mixture of two or more homotypic ENPs that differ from each other with respect to the AP of the plurality of fusion proteins present in said two or more homotypic ENPs, and wherein the population of ENPs thereby displays a plurality of disparate AP. The population of ENPs can comprise a mixture of two or more heterotypic ENPs that differ from each other with respect to the AP of the plurality of fusion proteins of said two or more heterotypic ENPs. Heterotypic ENPs can be capable of eliciting heterologous antibody responses against an additional infectious agent, and wherein said heterotypic ENPs do not display AP derived from said additional infectious agent.

In some embodiments, the plurality of disparate AP comprises: between about 2 and about 500 (e.g., 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 128, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, or a number or a range between any two of these values) antigenic polypeptides that differ from each other; AP of a same protein type; and/or AP of different protein types.

The plurality of AP can be of a same protein type or corresponding proteins. AP of a same protein type may or may not have identical amino acid sequences, but generally share some sequence homology. For example, the coronavirus S proteins of different coronaviruses are of a same protein type or corresponding proteins. As another example, envelope proteins from different coronaviruses are considered the same protein type or corresponding proteins. In some embodiments, proteins of different coronavirus taxonomic groups having the same function are considered the same protein type or corresponding proteins. In some embodiments, coronavirus antigens of a same protein type have at least 50% sequence identity, for example at least 65%, 70%, 80%, 90%, 95%, 98%, 99%, or more sequence identity.

Alternatively, in some embodiments the plurality of AP can comprise coronavirus proteins of different protein types. AP of different protein types typically have different functions. For example, the plurality of AP can comprise coronavirus S proteins or portions thereof as well as other coronavirus proteins such as a coronavirus N protein or a portion thereof, a coronavirus HE protein or a portion thereof, a coronavirus papain-like protease or a portion thereof, a coronavirus 3CL protease or a portion thereof, and/or a coronavirus M protein or a portion thereof.

The same ENP can comprise the AP derived from two or more strains of the same family, same genus, and/or same species, of infectious agent. The plurality of disparate AP can have a sequence identity of about, at least, or at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values, with one another.

The plurality of disparate AP can comprise a plurality of coronavirus (CoV) antigens, and the plurality of CoV antigens can comprise a first CoV antigen of a first CoV and a second CoV antigen of a second CoV that is different from the first CoV. The plurality of CoV antigens can comprise a CoV spike protein (S protein) or a portion thereof, a CoV envelope protein (E protein) or a portion thereof, a CoV nucleocapsid protein (N protein) or a portion thereof, a CoV hemagglutinin-esterase protein (HE protein) or a portion thereof, a CoV papain-like protease or a portion thereof, a CoV 3CL protease or a portion thereof, a CoV membrane protein (M protein) or a portion thereof, or a combination thereof. The plurality of CoV antigens can comprise a CoV S protein or a portion thereof. The first CoV antigen, the second CoV antigen, or both can comprise a CoV S protein or a portion thereof. The AP can comprise an amino acid sequence at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values, identical to SEQ ID NO: 24.

The number of the first APs (e.g., first CoV antigen molecules) and the number of the second APs (e.g., second CoV antigen molecules) can be in a ratio from 1:100 to 100:1 (e.g., 1:1, 1:1.1, 1:1.2, 1:1.3, 1:1.4, 1:1.5, 1:1.6, 1:1.7, 1:1.8, 1:1.9, 1:2, 1:2.5, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, 1:50, 1:51, 1:52, 1:53, 1:54, 1:55, 1:56, 1:57, 1:58, 1:59, 1:60, 1:61, 1:62, 1:63, 1:64, 1:65, 1:66, 1:67, 1:68, 1:69, 1:70, 1:71, 1:72, 1:73, 1:74, 1:75, 1:76, 1:77, 1:78, 1:79, 1:80, 1:81, 1:82, 1:83, 1:84, 1:85, 1:86, 1:87, 1:88, 1:89, 1:90, 1:91, 1:92, 1:93, 1:94, 1:95, 1:96, 1:97, 1:98, 1:99, 1:100 to 1:1, 1.1:1, 1.2:1, 1.3:1, 1.4:1, 1.5:1, 1.6:1, 1.7:1, 1.8:1, 1.9:1, 2:1, 2.5:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, or a number or a range between any of these values). The plurality of CoV antigens can comprise three, four, five, size seven, or eight CoV antigens, each of a CoV different from one another. The plurality of CoV antigens can comprise at least a third CoV antigen of a third CoV and a fourth CoV antigen of a fourth CoV, and the first, second, third and fourth CoVs can be different from one another.

The plurality of disparate (e.g., heterologous) AP can comprise at least m pathogenic antigens of an mth infectious agent, wherein m is an integer greater than 2 (e.g., 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 128, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, or a number or a range between any two of these values), and wherein each mth pathogenic antigen is different from one another (e.g., heterologous). In some embodiments, m is an integer greater than 50. The plurality of disparate AP can comprise two or more of a 1st pathogenic antigen (PA) of a 1st infectious agent (IA), a 2nd PA of a 2nd IA, a 3rd PA of a 3rd IA, a 4th PA of a 4th IA, a 5th PA of a 5th IA, a 6th PA of a 6th IA, a 7th PA of a 7th IA, a 8th PA of a 8th IA, a 9th PA of a 9th IA, a 10th PA of a 10th IA, a 11th PA of a 11th IA, a 12th PA of a 12th IA, a 13th PA of a 13th IA, a 14th PA of a 14th IA, a 15th PA of a 15th IA, a 16th PA of a 16th IA, a 17th PA of a 17th IA, a 18th PA of a 18th IA, a 19th PA of a 19th IA, a 20th PA of a 20th IA, a 21st PA of a 21st IA, a 22nd PA of a 22nd IA, a 23rd PA of a 23rd IA, a 24th PA of a 24th IA, a 25th PA of a 25th IA, a 26th PA of a 26th IA, a 27th PA of a 27th IA, a 28th PA of a 28th IA, a 29th PA of a 29th IA, a 30th PA of a 30th IA, a 31st PA of a 31st IA, a 32nd PA of a 32nd IA, a 33rd PA of a 33rd IA, a 34th PA of a 34th IA, a 35th PA of a 35th IA, a 36th PA of a 36th IA, a 37th PA of a 37th IA, a 38th PA of a 38th IA, a 39th PA of a 39th IA, a 40th PA of a 40th IA, a 41st PA of a 41st IA, a 42nd PA of a 42nd IA, a 43rd PA of a 43rd IA, a 44th PA of a 44th IA, a 45th PA of a 45th IA, a 46th PA of a 46th IA, a 47th PA of a 47th IA, a 48th PA of a 48th IA, a 49th PA of a 49th IA, and a 50th PA of a 50th IA. The 1st, 2nd, 3rd, 4th, 5th, 6th, 7th, 8th, 9th, 10th, 11th, 12th, 13th, 14th, 15th, 16th, 17th, 18th, 19th, 20th, 21st, 22nd, 23rd, 24th, 25th, 26th, 27th, 28th, 29th, 30th, 31st, 32nd, 33rd, 34th, 35th, 36th, 37th, 38th, 39th, 40th, 41st, 42nd, 43rd, 44th, 45th, 46th, 47th, 48th, 49th, and 50th pathogenic antigens can be the same or different from one another. The 1st, 2nd, 3rd, 4th, 5th, 6th, 7th, 8th, 9th, 10th, 11th, 12th, 13th, 14th, 15th, 16th, 17th, 18th, 19th, 20th, 21st, 22nd, 23rd, 24th, 25th, 26th, 27th, 28th, 29th, 30th, 31st, 32nd, 33rd, 34th, 35th, 36th, 37th, 38th, 39th, 40th, 41st, 42nd, 43rd, 44th, 45th, 46th, 47th, 48th, 49th, and 50th infectious agents can be the same or different from one another. The plurality of disparate (e.g., heterologous) AP can comprise a plurality of CoV antigens, a plurality of influenza antigens, and/or a plurality of HIV antigens.

The compositions provided herein can induce broadly protective anti-infectious agent responses by eliciting broadly neutralizing antibodies. As an example, broadly neutralizing antibodies are antibodies that can neutralize coronaviruses from a taxonomic group that is not only the same as but also differs from the taxonomic groups of the coronaviruses from which the coronavirus antigens used to elicit the antibodies are derived. Broadly neutralizing response can also be referred to as heterologously neutralizing response. In some embodiments, the compositions herein described can elicit broadly neutralizing antibodies that neutralize one or more infectious agents from a subfamily, genus, subgenus, species, and/or strain that differ from the subfamily, genus, subgenus, species, and/or strain of the infectious agents from which AP are derived to produce the fusion proteins provided herein.

Enveloped Nanoparticles

The ENPs can comprise at least about 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) more of the AP and/or can be at least as immunogenic (e.g., 1-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) as compared to a multi-component nanoparticle approach, optionally as compared to a SpyCatcher-based nanoparticle approach or a lentiviral Gag-based approach, further optionally the multi-component nanoparticle approach comprises two or more separate polypeptides.

The ENPs can comprise at least about 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) more of the AP, an at least about 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) higher density of the AP, and/or can be at least as immunogenic (e.g., 1-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values), as compared to a nanoparticle approach that does not comprise the ERD, optionally as compared to a SpyCatcher-based or Gag-based nanoparticle approach.

The ENPs can have one or more dimensions of a eukaryotic virus. In some embodiments, less than about 1%, 2%, 3%, 4%, 5%, 6%, 7%, 8%, 9%, or 10%, of the ENPs of the population of ENPs have a particle size smaller than about 10 nm. In some embodiments, less than about 1%, 2%, 3%, 4%, 5%, 6%, 7%, 8%, 9%, or 10%, of the ENPs of the population of ENPs have a particle size exceeding about 80 nm. In some embodiments, the average diameter of the ENPs of the population of ENPs range from about 5 nm to about 80 nm, from about 15 nm to about 50 nm, or from about 20 nm to about 40 nm. The average diameter of the ENPs of the population of ENPs can be about 10 nm, about 12 nm, about 14 nm, about 16 nm, about 18 nm, about 20 nm, about 22 nm, about 24 nm, about 26 nm, about 28 nm, about 30 nm, about 32 nm, about 34 nm, about 36 nm, about 38 nm, about 40 nm, about 42 nm, about 44 nm, about 46 nm, about 48 nm, or about 50 nm, optionally the average is the mean, median or mode, optionally the mean is the arithmetic mean, geometric mean, and/or harmonic mean.

In some embodiments, the ENPs have a minimum diameter of about 10 nm, about 12 nm, about 14 nm, about 16 nm, about 18 nm, about 20 nm, about 22 nm, about 24 nm, about 26 nm, about 28 nm, about 30 nm, about 32 nm, about 34 nm, about 36 nm, about 38 nm, about 40 nm, about 42 nm, about 44 nm, about 46 nm, about 48 nm, or about 50 nm. In some embodiments, the ENPs have a maximum diameter of about 10 nm, about 12 nm, about 14 nm, about 16 nm, about 18 nm, about 20 nm, about 22 nm, about 24 nm, about 26 nm, about 28 nm, about 30 nm, about 32 nm, about 34 nm, about 36 nm, about 38 nm, about 40 nm, about 42 nm, about 44 nm, about 46 nm, about 48 nm, about 50 nm, about 52 nm, about 54 nm, about 56 nm, about 58 nm, about 60 nm, about 62 nm, about 64 nm, about 66 nm, about 68 nm, about 70 nm, about 72 nm, about 74 nm, about 76 nm, about 78 nm, or about 80 nm.

In some embodiments, storage of the ENPs at 4° C. for at least three months reduces immunogenicity less than about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50%. The composition can be stable for at least about 2 weeks, about 1 month, about 2 months, about 3 months, about 4 months, about 5 months, about 6 months, or about 1 year, after storage as a liquid at a temperature of about 4° C. At least about at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values, of the ENPs can be immunogenic at least 1 month after storage as a liquid at a temperature of about 5° C.

Vectors and Carriers

The nucleic acid composition (e.g., n polynucleotides encoding n fusion proteins) can be complexed or associated with one or more lipids or lipid-based carriers, thereby forming liposomes, lipid nanoparticles (LNPs), lipoplexes, and/or nanoliposomes, optionally encapsulating the nucleic acid composition. In some embodiments, the nucleic acid composition (e.g., n polynucleotides encoding n fusion proteins) is, comprises, or further comprises, one or more vectors. At least one of the one or more vectors can be a viral vector, a plasmid, a transposable element, a naked DNA vector, a lipid nanoparticle (LNP), or any combination thereof. The viral vector can be an AAV vector, a lentivirus vector, a retrovirus vector, an adenovirus vector, a herpesvirus vector, a herpes simplex virus vector, a cytomegalovirus vector, a vaccinia virus vector, a MVA vector, a baculovirus vector, a vesicular stomatitis virus vector, a human papillomavirus vector, an avipox virus vector, a Sindbis virus vector, a VEE vector, a Measles virus vector, an influenza virus vector, a hepatitis B virus vector, an integration-deficient lentivirus (IDLV) vector, or any combination thereof. The transposable element can be piggybac transposon or sleeping beauty transposon. The polynucleotide(s) encoding fusion protein(s) can be comprised in the one or more vectors. The polynucleotide(s) encoding fusion protein(s) can be comprised in the same vector and/or different vectors. The polynucleotide(s) encoding fusion protein(s) can be situated on the same nucleic acid and/or different nucleic acids.

The one or more vectors can be a DNA vaccine. The DNA vaccine can be a plasmid-based DNA vaccine, a minicircle-based DNA vaccine, a bacmid-based DNA vaccine, a minigene-based DNA vaccine, a ministring DNA (linear covalently closed DNA vector) vaccine, a closed-ended linear duplex DNA (CELiD or ceDNA) vaccine, a doggybone™ DNA vaccine, a dumbbell shaped DNA vaccine, or a minimalistic immunological-defined gene expression (MIDGE)-vector DNA vaccine. In some embodiments, the DNA vaccine elicits at least 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) higher neutralizing antibody responses against an infectious agent as compared to a DNA vaccine that encodes the AP but not the ERD.

The polynucleotide(s) encoding fusion protein(s) (e.g., n polynucleotides encoding n fusion proteins) can be operably linked to one or more promoters capable of inducing transcription of said polynucleotide(s). The promoter can comprise a ubiquitous promoter, an inducible promoter, a tissue-specific promoter and/or a lineage-specific promoter. The ubiquitous promoter can be selected from the group comprising a cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus, a Ubiquitin C promoter (UBC), a phosphoglycerate kinase-1 (PGK) promoter, 3-phosphoglycerate kinase promoter, a cytomegalovirus enhancer, human β-actin (HBA) promoter, chicken β-actin (CBA) promoter, a CAG promoter, a CASI promoter, a CBH promoter, or any combination thereof.

As used herein, the term “promoter” is a nucleotide sequence that permits binding of RNA polymerase and directs the transcription of a gene. Typically, a promoter is located in the 5′ non-coding region of a gene, proximal to the transcriptional start site of the gene. Sequence elements within promoters that function in the initiation of transcription are often characterized by consensus nucleotide sequences. Examples of promoters include, but are not limited to, promoters from bacteria, yeast, plants, viruses, and mammals (including humans). A promoter can be inducible, repressible, and/or constitutive. Inducible promoters initiate increased levels of transcription from DNA under their control in response to some change in culture conditions, such as a change in temperature.

As used herein, the term “operably linked” is used to describe the connection between regulatory elements and a gene or its coding region. Typically, gene expression is placed under the control of one or more regulatory elements, for example, without limitation, constitutive or inducible promoters, tissue-specific regulatory elements, and enhancers. A gene or coding region is said to be “operably linked to” or “operatively linked to” or “operably associated with” the regulatory elements, meaning that the gene or coding region is controlled or influenced by the regulatory element. For instance, a promoter is operably linked to a coding sequence if the promoter effects transcription or expression of the coding sequence.

The polynucleotide(s) encoding fusion protein(s) can be operably linked to a tandem gene expression element (e.g., an internal ribosomal entry site (IRES), foot-and-mouth disease virus 2A peptide (F2A), equine rhinitis A virus 2A peptide (E2A), porcine teschovirus 2A peptide (P2A) or Thosea asigna virus 2A peptide (T2A), or any combination thereof). The polynucleotide(s) encoding fusion protein(s) can comprise a transcript stabilization element (e.g., woodchuck hepatitis post-translational regulatory element (WPRE), bovine growth hormone polyadenylation (bGH-polyA) signal sequence, human growth hormone polyadenylation (hGH-polyA) signal sequence, or any combination thereof).

The nucleic acid composition can be or can comprise mRNA. The composition (e.g., nucleic acid composition) can be an mRNA vaccine. The mRNA can be formulated in a lipid nanoparticle (LNP). The term “lipid nanoparticle”, also referred to as LNP, refers to a particle having at least one dimension on the order of nanometers (e.g., 1-1,000 nm) which includes one or more lipids. In some embodiments, such lipid nanoparticles comprise a cationic lipid and one or more excipient selected from neutral lipids, charged lipids, steroids and polymer conjugated lipids (e.g., a pegylated lipid). In some embodiments, the mRNA, or a portion thereof, is encapsulated in the lipid portion of the lipid nanoparticle or an aqueous space enveloped by some or all of the lipid portion of the lipid nanoparticle, thereby protecting it from enzymatic degradation or other undesirable effects induced by the mechanisms of the host organism or cells e.g. an adverse immune response. In some embodiments, the mRNA or a portion thereof is associated with the lipid nanoparticles. An LNP may comprise any lipid capable of forming a particle to which the one or more nucleic acid molecules are attached, or in which the one or more nucleic acid molecules are encapsulated. The term “lipid” refers to a group of organic compounds that are derivatives of fatty acids (e.g., esters) and are generally characterized by being insoluble in water but soluble in many organic solvents. Lipids are usually divided in at least three classes: (1) “simple lipids” which include fats and oils as well as waxes; (2) “compound lipids” which include phospholipids and glycolipids; and (3) “derived lipids” such as steroids.

The LNP can comprise one or more of an ionizable cationic lipid, a non-cationic lipid (e.g., a neutral lipid), a sterol, and a PEG-modified lipid. The LNP can comprise 0.5-15 mol % PEG-modified lipid, 5-25 mol % non-cationic lipid, 25-55 mol % sterol, and 20-60 mol % ionizable cationic lipid. The LNP can comprise 40-55 mol % ionizable cationic lipid, 5-15 mol % neutral lipid, 35-45 mol % sterol, and 1-5 mol % PEG-modified lipid. In some embodiments, the RNA (e.g., mRNA) of the disclosure is formulated in a lipid nanoparticle (LNP). Lipid nanoparticles typically comprise ionizable cationic lipid, non-cationic lipid, sterol and PEG lipid components along with the nucleic acid cargo of interest. The lipid nanoparticles of the disclosure can be generated using components, compositions, and methods as are generally known in the art, see for example PCT/US2016/052352; PCT/US2016/068300; PCT/US2017/037551; PCT/US2015/027400; PCT/US2016/047406; PCT/US2016/000129; PCT/US2016/014280; PCT/US2016/014280; PCT/US2017/038426; PCT/US2014/027077; PCT/US2014/055394; PCT/US2016/052117; PCT/US2012/069610; PCT/US2017/027492; PCT/US2016/059575 and PCT/US2016/069491 all of which are incorporated by reference herein in their entirety.

In some embodiments, the LNP comprises: 47 mol % ionizable cationic lipid, 11.5 mol % neutral lipid, 38.5 mol % sterol, and 3.0 mol % PEG-modified lipid; 48 mol % ionizable cationic lipid, 11 mol % neutral lipid, 38.5 mol % sterol, and 2.5 mol % PEG-modified lipid; 49 mol % ionizable cationic lipid, 10.5 mol % neutral lipid, 38.5 mol % sterol, and 2.0 mol % PEG-modified lipid; 50 mol % ionizable cationic lipid, 10 mol % neutral lipid, 38.5 mol % sterol, and 1.5 mol % PEG-modified lipid; or 51 mol % ionizable cationic lipid, 9.5 mol % neutral lipid, 38.5 mol % sterol, and 1.0 mol % PEG-modified lipid.

The ionizable cationic lipid can be heptadecan-9-yl 8 ((2 hydroxyethyl)(6 oxo 6-(undecyloxy)hexyl)amino)octanoate. The neutral lipid can be 1,2 distearoyl-sn-glycero-3 phosphocholine (DSPC). The sterol can be cholesterol. The PEG-modified lipid can be 1-monomethoxypolyethyleneglycol-2,3-dimyristylglycerol with polyethylene glycol of average molecular weight 2000 (PEG2000 DMG).

The wt/wt ratio of lipid to mRNA can be from about 1:100 to about 100:1 (e.g., 1:1, 1:1.1, 1:1.2, 1:1.3, 1:1.4, 1:1.5, 1:1.6, 1:1.7, 1:1.8, 1:1.9, 1:2, 1:2.5, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, 1:50, 1:51, 1:52, 1:53, 1:54, 1:55, 1:56, 1:57, 1:58, 1:59, 1:60, 1:61, 1:62, 1:63, 1:64, 1:65, 1:66, 1:67, 1:68, 1:69, 1:70, 1:71, 1:72, 1:73, 1:74, 1:75, 1:76, 1:77, 1:78, 1:79, 1:80, 1:81, 1:82, 1:83, 1:84, 1:85, 1:86, 1:87, 1:88, 1:89, 1:90, 1:91, 1:92, 1:93, 1:94, 1:95, 1:96, 1:97, 1:98, 1:99, 1:100 to 1:1, 1.1:1, 1.2:1, 1.3:1, 1.4:1, 1.5:1, 1.6:1, 1.7:1, 1.8:1, 1.9:1, 2:1, 2.5:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, or a number or a range between any of these values).

The LNP can comprise a cationic lipid. The cationic lipid is can be cationisable, i.e. it becomes protonated as the pH is lowered below the pKa of the ionizable group of the lipid, but is progressively more neutral at higher pH values. When positively charged, the lipid is then able to associate with negatively charged nucleic acids. In some embodiments, the cationic lipid comprises a zwitterionic lipid that assumes a positive charge on pH decrease. The LNP may comprise any lipid capable of forming a particle to which the one or more nucleic acid molecules are attached, or in which the one or more nucleic acid molecules are encapsulated. In some embodiments, the LNP may comprise any further cationic or cationisable lipid, i.e. any of a number of lipid species which carry a net positive charge at a selective pH, such as physiological pH. Such lipids include, but are not limited to, N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB); N-(2,3dioleoyloxy)propyl)-N,N,N-trimethylammonium chloride (DOTAP); 3-(N—(N′,N′dimethylaminoethane)-carbamoyl)cholesterol (DC-Chol), N-(1-(2,3-dioleoyloxy)propyl)N-2-(sperminecarboxamido)ethyl)-N,N-dimethylammonium trifluoracetate (DOSPA), dioctadecylamidoglycyl carboxyspermine (DOGS), 1,2-dioleoyl-3-dimethylammonium propane (DODAP), N,N-dimethyl-2,3-dioleoyloxy)propylamine (DODMA), and N-(1,2dimyristyloxyprop-3-yl)-N,N-dimethyl-N-hydroxyethyl ammonium bromide (DMRIE).

Additionally, a number of commercial preparations of cationic lipids are available which can be used in embodiments provided herein. These include, for example, LIPOFECTIN® (commercially available cationic liposomes comprising DOTMA and 1,2-dioleoyl-sn-3phosphoethanolamine (DOPE), from GIBCO/BRL, Grand Island, N.Y.); LIPOFECTAMINE® (commercially available cationic liposomes comprising N-(1-(2,3dioleyloxy)propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.). The following lipids are cationic and have a positive charge at below physiological pH: DODAP, DODMA, DMDMA, 1,2-dilinoleyloxy-N,N-dimethylaminopropane (DLinDMA), 1,2-dilinolenyloxy-N,N-dimethylaminopropane (DLenDMA).

Exemplary neutral lipids include, for example, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-lcarboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoylphosphatidyethanol amine (SOPE), and 1,2-dielaidoyl-sn-glycero-3-phophoethanolamine (transDOPE). In some embodiments, the neutral lipid is 1,2-distearoyl-sn-glycero-3phosphocholine (DSPC).

In some embodiments, the cationic lipid is an amino lipid. Suitable amino lipids useful include those described in WO2012/016184, incorporated herein by reference in its entirety. Representative amino lipids include, but are not limited to, 1,2-dilinoleyoxy-3-(dimethylamino)acetoxypropane (DLin-DAC), 1,2-dilinoleyoxy-3 morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino)propane (DLin-MPZ), 3-(N,Ndilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl-[1,3]-dioxolane (DLin-K-DMA).

In some embodiments, a non-cationic lipid comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-0-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine,1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.

In some embodiments, a PEG modified lipid comprises a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is DMG-PEG, PEG-c-DOMG (also referred to as PEG-DOMG), PEG-DSG and/or PEG-DPG. In some embodiments, a sterol comprises cholesterol, fecosterol, sitosterol, ergosterol, campesterol, stigmasterol, brassicasterol, tomatidine, ursolic acid, alpha-tocopherol, and mixtures thereof.

In some embodiments, the nucleic acid composition comprising an mRNA sequence is a modified mRNA sequence. In this context, a modification as defined herein can lead to a stabilization of the mRNA sequence provided herein. In some embodiments, there is thus provided a stabilized mRNA sequence comprising at least one coding region as defined herein (e.g. polynucleotide(s) encoding fusion protein(s)). In some embodiments, the nucleic acid composition comprising an mRNA sequence may thus be provided as a “stabilized mRNA sequence”, that is to say as an mRNA that is essentially resistant to in vivo degradation (e.g. by an exo- or endo-nuclease). Such stabilization can be effected, for example, by a modified phosphate backbone of an mRNA provided herein. A backbone modification can be a modification in which phosphates of the backbone of the nucleotides contained in the mRNA are chemically modified. Nucleotides that can be used in this connection contain e.g. a phosphorothioate-modified phosphate backbone, such as at least one of the phosphate oxygens contained in the phosphate backbone being replaced by a sulfur atom. Stabilized mRNAs may further include, for example: non-ionic phosphate analogues, such as, for example, alkyl and aryl phosphonates, in which the charged phosphonate oxygen is replaced by an alkyl or aryl group, or phosphodiesters and alkylphosphotriesters, in which the charged oxygen residue is present in alkylated form. Such backbone modifications typically include, without implying any limitation, modifications from the group consisting of methylphosphonates, phosphoramidates and phosphorothioates (e.g. cytidine-5′-O-(1-thiophosphate)). The term “mRNA modification” as used herein may refer to chemical modifications comprising backbone modifications as well as sugar modifications or base modifications. In this context, a modified mRNA (sequence) as defined herein may contain nucleotide analogues/modifications, e.g. backbone modifications, sugar modifications or base modifications. A backbone modification can be a modification, in which phosphates of the backbone of the nucleotides contained in an mRNA compound comprising an mRNA sequence as defined herein are chemically modified. A sugar modification can be a chemical modification of the sugar of the nucleotides of the mRNA compound comprising an mRNA sequence as defined herein. Furthermore, a base modification can be a chemical modification of the base moiety of the nucleotides of the mRNA compound comprising an mRNA sequence. In this context, nucleotide analogues or modifications can be selected from nucleotide analogues, which are applicable for transcription and/or translation.

The mRNA provided herein can comprise a 5′ untranslated region (UTR), a 3′ UTR, and/or a cap (e.g., a CAP analogue). A modified mRNA sequence as defined herein, can be modified by the addition of a so-called “5′-CAP structure”, which can stabilize the mRNA as described herein. A 5′-CAP is an entity, typically a modified nucleotide entity, which generally “caps” the 5′-end of a mature mRNA. A 5′-CAP may typically be formed by a modified nucleotide, particularly by a derivative of a guanine nucleotide. In some embodiments, the 5′-CAP is linked to the 5′-terminus via a 5′-5′-triphosphate linkage. A 5′-CAP may be methylated, e.g. m7GpppN, wherein N is the terminal 5′ nucleotide of the nucleic acid carrying the 5′-CAP, typically the 5′-end of an mRNA. m7GpppN is the 5′-CAP structure, which naturally occurs in mRNA transcribed by polymerase II and is therefore in some embodiments is not considered as modification comprised in a modified mRNA in this context. Accordingly, a modified mRNA sequence may comprise a m7GpppN as 5′-cap, but additionally the modified mRNA sequence typically comprises at least one further modification as defined herein. A CAP analogue refers to a non-polymerizable di-nucleotide that has CAP functionality in that it facilitates translation or localization, and/or prevents degradation of the RNA molecule when incorporated at the 5′-end of the RNA molecule. Non-polymerizable means that the CAP analogue will be incorporated only at the 5′-terminus because it does not have a 5′ triphosphate and therefore cannot be extended in the 3′-direction by a template-dependent RNA polymerase. CAP analogues include, but are not limited to, a chemical structure selected from the group consisting of m7GpppG, m7GpppA, m7GpppC; unmethylated CAP analogues (e.g., GpppG); dimethylated CAP analogue (e.g., m2,7GpppG), trimethylated CAP analogue (e.g., m2,2,7GpppG), dimethylated symmetrical CAP analogues (e.g., m7Gpppm7G), or anti reverse CAP analogues (e.g., ARCA; m7,2′OmeGpppG, m7,2′dGpppG, m7,3′OmeGpppG, m7,3′dGpppG and their tetraphosphate derivatives) (Stepinski et al., 2001. RNA 7(10):1486-95). Further CAP analogues have been described previously (U.S. Pat. No. 7,074,596, WO2008/016473, WO2008/157688, WO2009/149253, WO2011/015347, and WO2013/059475).

The mRNA can comprise one or more modified nucleotides selected from the group comprising pseudouridine, N-1-methyl-pseudouridine, 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine. The mRNA can comprise a modified nucleotide in place of one or more uridines. The modified nucleoside can be selected from pseudouridine (ψ), N 1-methyl-pseudouridine (m PP), and 5-methyl-uridine (m5U). In some embodiments, a non-naturally occurring modified nucleotide or nucleoside of the disclosure is one as is generally known or recognized in the art. Non-limiting examples of such non-naturally occurring modified nucleotides and nucleosides can be found, inter alia, in published US application Nos. PCT/US2012/058519; PCT/US2013/075177; PCT/US2014/058897; PCT/US2014/058891; PCT/US2014/070413; PCT/US2015/36773; PCT/US2015/36759; PCT/US2015/36771; or PCT/IB 2017/051367 all of which are incorporated by reference herein.

### Pharmaceutical Compositions and Therapeutic Applications

Vaccine Compositions

Also provided herein include a vaccine composition comprising compositions (e.g., a nucleic acid composition, a population of ENPs) as herein described. Vaccine compositions can comprise the compositions provided herein (e.g., a nucleic acid composition, a population of ENPs) in combination with one or more compatible and pharmaceutically acceptable carriers. For example, a vaccine composition can be or can comprise an mRNA vaccine, a DNA vaccine, and/or a population of ENPs as described herein. A vaccine composition is a pharmaceutical composition that can elicit a prophylactic (e.g., to prevent or delay the onset of a disease, or to prevent the manifestation of clinical or subclinical symptoms thereof) or therapeutic (e.g., suppression or alleviation of symptoms) immune response in a subject.

The phrase “pharmaceutically acceptable” is employed herein to refer to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth: (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

In some embodiments, pharmaceutically acceptable carrier comprise a pharmaceutical acceptable salt. As used herein, a “pharmaceutical acceptable salt” includes a salt of an acid form of one of the components of the compositions herein described. These include organic or inorganic acid salts of the amines. Preferred acid salts are the hydrochlorides, acetates, salicylates, nitrates and phosphates. Other suitable pharmaceutically acceptable salts are well known to those skilled in the art and include basic salts of a variety of inorganic and organic acids.

The vaccine composition can further comprise appropriate adjuvants. Adjuvant refers to any immunomodulating substance capable of being combined with the protein antigens herein described to enhance, improve or otherwise modulate an immune response in a subject, such as, for example, immunostimulatory peptides, oligonucleotide CpG motifs, immunostimulatory carbohydrates and polysaccharides, and immunostimulatory protein or peptide molecules (e.g. cytokines, chemokines, flagellin, and derivatives thereof), Freund's adjuvant, sapanin (e.g., Matrix M1), lecithin, aluminum hydroxide, monophosphoryl lipid A, interleukin-12, STING agonist, Advax, and AS01_{B}, STING agonist (e.g., bis-(3′,5′)-cyclic dimeric guanosine monophosphate (c-di-GMP or cdGMP)). In some embodiments, the nucleic acid composition comprises one or more polynucleotides encoding immunostimulatory agents. In some embodiments, the population of ENPs comprises one or more immunostimulatory agents. The one or more immunostimulatory agents can comprise toll-like receptor (TLR) agonists, cytokine receptor agonists, CD40 agonists, Fc receptor agonists, CpG-containing nucleic acids, complement receptor agonists, or any combination thereof). The TLR agonist can be a TLR-1 agonist, TLR-2 agonist, TLR-3 agonist, TLR-4 agonist, TLR-5 agonist, TLR-6 agonist, TLR-7 agonist, TLR-8 agonist, TLR-9 agonist, and/or TLR-10 agonist. The Fc receptor agonist can be a Fc-gamma receptor agonist. In some embodiments, the complement receptor agonist binds to CD21 or CD35. In some embodiments, the complement receptor agonist induces endogenous complement opsonization of the ENP. The cytokine receptor agonist can be a cytokine. The cytokine receptor agonist can be a small molecule, antibody, fusion protein, or aptamer.

In some embodiments herein described, the vaccine composition comprises one or more adjuvant selected from the group comprising, or consisting of, aluminum salt-based adjuvants (e.g., aluminum hydroxide, alhydrogel), emulsion adjuvants (e.g., AddaVax′, 1V1F59®, AS03, Freund's adjuvant, Montanide ISA51), and toll-like receptor agonists (e.g., CpG, Poly I:C, glucopyranosyl lipid A (GLA), flagellin, and resiquimod (R848)). As a skilled person will understand, both MF58® and AddaVax™ are squalene-based oil-in-water nano-emulsion.

The vaccine composition can be formulated for a variety of modes of administration. Techniques for formulation and administration can be found, for example, in “Remington's Pharmaceutical Sciences”, 18^{th} ed., 1990, Mack Publishing Co., Easton, Pa. In some embodiments, the vaccine compositions of the present disclosure may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension: (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the hydrogel composition. The pharmaceutical compositions can comprise one or more pharmaceutically-acceptable carriers.

Formulations useful in the methods of the present disclosure include those suitable for oral, nasal, topical (including buccal and sublingual), rectal, vaginal, aerosol and/or parenteral administration. The formulations may conveniently be presented in unit dosage form and may be prepared by any methods well known in the art of pharmacy. The amount of active ingredient which can be combined with a pharmaceutically acceptable carrier to produce a single dosage form will vary depending upon the host being treated, the particular mode of administration. The amount of active ingredient, which can be combined with a pharmaceutically acceptable carrier to produce a single dosage form will generally be that amount of the composition (e.g., a nucleic acid composition, a population of ENPs) which produces a therapeutic effect or an immune response. Generally, out of one hundred percent, this amount will range from about 1% to about 99% of active ingredient, optionally from about 5% to about 70%, optionally from about 10% to about 30%.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like.

In solid dosage forms for oral administration (capsules, tablets, pills, dragees, powders, granules and the like), the composition (e.g., a nucleic acid composition, a population of ENPs) is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

The vaccine composition can be formulated for parenteral administration by injection, e.g. by bolus injection or continuous infusion. Formulations for injection can be presented in a unit dosage form, e.g. in ampoules or in multi-dose containers, with an optionally added preservative. The pharmaceutical compositions can further be formulated as suspensions, solutions or emulsions in oily or aqueous vehicles, and may contain other agents including suspending, stabilizing and/or dispersing agents.

Applications

The vaccine compositions disclosed herein (e.g., a nucleic acid composition, a population of ENPs) can be employed in a variety of therapeutic or prophylactic applications to stimulate an immune response in a subject in need, to treat or prevent a coronavirus infection in a subject in need, and/or to treat or prevent a disease or disorder in a subject in need (e.g., a disease or disorder caused by an infectious agent, such as by a coronavirus).

There are provided, in some embodiments, methods of stimulating an immune response in a subject in need thereof. In some embodiments, the method comprises: administering to the subject a pharmaceutically effective amount of a composition disclosed herein (e.g., a nucleic acid composition, a population of ENPs), thereby stimulating an immune response in the subject.

There are provided, in some embodiments, methods of treating or preventing a disease or disorder in a subject in need thereof. In some embodiments, the method comprises: administering to the subject a pharmaceutically effective amount of a composition disclosed herein (e.g., a nucleic acid composition, a population of ENPs), thereby treating or preventing the disease or disorder in the subject. The disease or disorder can be a disease or disorder caused by an infectious agent. The disease or disorder caused by an infectious agent can be a disease or disorder caused by a coronavirus (CoV) infection.

There are provided, in some embodiments, methods for treating or preventing a coronavirus (CoV) infection in a subject in need thereof. In some embodiments, the method comprises: administering to the subject a pharmaceutically effective amount of a composition disclosed herein (e.g., a nucleic acid composition, a population of ENPs), thereby treating or preventing the CoV infection in the subject.

As used herein, the term “treatment” or “treat” refers to an intervention made in response to a disease, disorder or physiological condition (e.g., a coronavirus infection) manifested by a patient. The aim of treatment may include, but is not limited to, one or more of the alleviation or prevention of symptoms, slowing or stopping the progression or worsening of a disease, disorder, or condition and the remission of the disease, disorder or condition. The term “treat” and “treatment” includes, for example, therapeutic treatments, prophylactic treatments, and applications in which one reduces the risk that a subject will develop a disorder or other risk factor. Treatment does not require the complete curing of a disorder and encompasses embodiments in which one reduces symptoms or underlying risk factors. In some embodiments, “treatment” refers to both therapeutic treatment and prophylactic or preventative measures. Those in need of treatment include those already affected by a disease or disorder or undesired physiological condition as well as those in which the disease or disorder or undesired physiological condition is to be prevented. As used herein, the term “prevention” refers to any activity that reduces the burden of the individual later expressing those symptoms. This can take place at primary, secondary and/or tertiary prevention levels, wherein: a) primary prevention avoids the development of symptoms/disorder/condition; b) secondary prevention activities are aimed at early stages of the condition/disorder/symptom treatment, thereby increasing opportunities for interventions to prevent progression of the condition/disorder/symptom and emergence of symptoms; and c) tertiary prevention reduces the negative impact of an already established condition/disorder/symptom by, for example, restoring function and/or reducing any condition/disorder/symptom or related complications. The term “prevent” does not require the 100% elimination of the possibility of an event. Rather, it denotes that the likelihood of the occurrence of the event has been reduced in the presence of the compound or method.

The term “condition” as used herein indicates a physical status of the body of an individual (as a whole or as one or more of its parts), that does not conform to a standard physical status associated with a state of complete physical, mental and social well-being for the individual. Conditions herein described include but are not limited disorders and diseases wherein the term “disorder” indicates a condition of the living individual that is associated to a functional abnormality of the body or of any of its parts, and the term “disease” indicates a condition of the living individual that impairs normal functioning of the body or of any of its parts and is typically manifested by distinguishing signs and symptoms.

Signs and symptoms manifesting a disease or disorder caused by a coronavirus infection can include, but not limited to, fever, cough, tiredness, a loss of taste or smell, shortness of breath or difficulty breathing, muscle aches, chills, sore throat, runny nose, headache, chest pain, pink eye (conjunctivitis), nausea, vomiting, diarrhea, rash, pneumonia and acute respiratory distress syndrome. Diseases or disorders caused by a coronavirus infection may also include severe complications including but not limited to heart disorders including arrhythmias, cardiomyopathy, acute cardiac injury, coagulation disorders including thromboembolism and pulmonary emboli, disseminated intravascular coagulation (DIC), hemorrhage, and arterial clot formation, Guillain-Barré syndrome, sepsis, shock, multiorgan failure, and multi system inflammatory syndrome, and any combination thereof.

The terms “subject”, “subject in need”, and “individual” as used herein refer to an animal and in particular higher animals and in particular vertebrates such as mammals and more particularly human beings. In some embodiments, the subject or individual has been exposed to an infectious agent (e.g., a coronavirus). The term “exposed” indicates the subject has come in contact with a person or an animal that is known to be infected with an infectious agent (e.g., a coronavirus). In some embodiments, a subject in need can be a healthy subject exposed to or at risk of being exposed to an infectious agent (e.g., a coronavirus). In some embodiments, subjects in need include those already suffering from the disease or disorder caused by an infection of the infectious agent or those diagnosed with an infection.

Accordingly, the vaccine composition can be administered in advance of any symptom, for example, in advance of a coronavirus infection. The vaccine composition can also be administered at or after the onset of a symptom of disease or infection, for example, after development of a symptom of infection or after diagnosis of the infection.

The phrase “therapeutically effective amount” as used herein means that amount of a composition disclosed herein (e.g., a vaccine composition) which is effective for producing some desired therapeutic effect and/or generating a desired response, such as reduce or eliminate a sign or symptom of a condition or disease, such as pneumonia, at a reasonable benefit/risk ratio. The therapeutically effective amount also varies depending on the structure and AP of the fusion protein(s), the route of administration utilized, and the specific diseases or disorders to be treated as will be understood to a person skilled in the art. For example, if a given clinical treatment is considered effective when there is at least a 20% reduction in a measurable parameter associated with a disease or disorder, a therapeutically effective amount of the composition for the treatment of that disease or disorder is the amount necessary to achieve at least a 20% reduction in that measurable parameter.

A therapeutically effective amount of the vaccine composition herein described can be estimated from data obtained from cell culture assays and further determined from data obtained in animal studies, followed up by human clinical trials. For example, toxicity and therapeutic efficacy of the vaccine compositions described herein can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions that exhibit large therapeutic indices are preferred.

In some embodiments, the determination of a therapeutically effective amount of the vaccine composition can be measured by measuring the titer of antibodies produced against an infectious agent. Methods of determining antibody titers and methods of performing virus neutralization arrays are known to those skilled in the art as well as exemplified in the example section of the present disclosure (see, Examples).

In some embodiments, the vaccine composition can be used for treating and preventing a broad spectrum of infections or a disease and disorder caused by such infections by inducing broadly protective anti-infectious agent responses. For example, the vaccine composition herein described can elicit broadly neutralizing antibodies that neutralize one or more coronaviruses from a subfamily, genus, subgenus, species, and/or strain that differ from the subfamily, genus, subgenus, species, and/or strain of the infectious agents from which the AP are derived to produce the vaccine composition.

Immunogenic levels of the fusion protein and/or ENP can be produced in serum of the subject at about 1 hour to about 6 months (e.g., 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, or a number or a range between any of these values) post administration of the composition (e.g., vaccine composition).

A neutralizing antibody titer of about 50 to about 100000 half-maximal inhibitory dilutions (ID₅₀s values) can be produced in the serum of the subject at about 1 hour to about 6 months (e.g., 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, or a number or a range between any of these values) post administration of the composition (e.g., vaccine composition).

In some embodiments, the composition (e.g., vaccine composition) elicits at least about 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) less off-target immune responses against undesired epitopes as compared to a non-enveloped NP-based composition. Said undesired epitopes can comprise the NP scaffold of said non-enveloped NP-based composition.

In some embodiments, the composition (e.g., vaccine composition) elicits an at least 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) higher neutralizing antibody titer as compared to an approach comprising administration of (i) a soluble version of the AP, and/or (ii) an mRNA vaccine encoding the AP and not encoding the ERD. In some embodiments, the composition (e.g., vaccine composition) elicits an at least as high neutralizing antibody titer (e.g., 1-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) as compared to an approach comprising administration of a protein-based nanoparticle presenting the AP. In some embodiments, administration of the composition (e.g., vaccine composition) elicits protective and long-lasting immunity against the infectious agent(s) and variants thereof.

In some embodiments, an at least as low dose of the composition (e.g., vaccine composition) can be needed to generate a comparable immune response as compared to an approach comprising administration of a protein-based nanoparticle presenting the AP. In some embodiments, an at least about 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) lower dose of the composition (e.g., vaccine composition) can be needed to generate a comparable immune response as compared to an approach comprising administration of (i) a soluble version of the AP, and/or (ii) an mRNA vaccine encoding the AP and not encoding the ERD.

In some embodiments, following administration of a first dose or second dose of the composition (e.g., vaccine composition), the composition induces: (i) an at least as potent (e.g., 1-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) serum neutralizing titers against the infectious agent or variants thereof as compared to an approach comprising administration of a protein-based nanoparticle presenting the AP, optionally the composition comprises a population of ENPs; (ii) an at least 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) more potent serum neutralizing titers against the infectious agent or variants thereof as compared to an approach comprising administration of a soluble version of the AP, optionally the composition comprises a population of ENPs; and/or (iii) an at least 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) more potent serum neutralizing titers against the infectious agent or variants thereof as compared to an approach comprising administration of an mRNA vaccine encoding the AP and not encoding the ERD, optionally the composition comprises an mRNA vaccine encoding a fusion protein comprising the AP.

In some embodiments, potency of serum neutralizing titers can be measured by geometric means for serum half-maximal inhibitory dilutions (ID₅₀s values) against the infectious agent or variants thereof, optionally about 1 day to about 6 months (e.g., 1 day, 2 days, 3 days, 4 days, 5 days, 6 days, 1 week, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, or a number or a range between any of these values) after administration of a first dose or a second dose of the composition (e.g., vaccine composition).

In some embodiments, the subject: is a human subject; is a newborn or infant of an age of not more than 3 years, of not more than 2 years, of not more than 1.5 years, of not more than 1 year (12 months), of not more than 9 months, 6 months or 3 months, or is between 6 months and 2 years; is immunocompromised, has a pulmonary disease, and/or is 65 years of age or older; has a chronic pulmonary disease, optionally chronic obstructive pulmonary disease (COPD) or asthma; and/or has an underlying comorbid condition, optionally selected from heart disease, diabetes, and lung disease.

The composition (e.g., vaccine composition) can be administered in an effective amount to: (i) induce a robust antibody response against the AP in the subject, optionally a robust antibody response comprises a neutralizing antibody response, further optionally a robust antibody response comprises Fc domain effector functions that recruit immune cells to infected cells, optionally said immune cells are macrophages, neutrophils, and/or natural killer cells, further optionally said recruitment induces antibody-dependent cellular cytotoxicity (ADCC) and/or antibody-dependent cellular phagocytosis (ADCP); (ii) elicit a robust CD4 and/or CD8 T cell response against the AP in the subject; and/or (iii) elicit a balanced Th1/Th2 response against the AP in the subject.

The method can comprise administering to the subject at least two doses (e.g., 2, 3, 4, 5, 6, 7, 8, 9, 10, 11, 12, 13, 14, 15, 16, 17, 18, 19, 20, or a number or a range between any of these values) of the composition (e.g., vaccine composition). The second dose of the composition (e.g., vaccine composition) can be administered to the subject at least 1 day (e.g., 1 day, 2 days, 3 days, 4 days, 5 days, 6 days, 1 week, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, or a number or a range between any of these values) after a first dose of the composition (e.g., vaccine composition) is administered to the subject.

The vaccine compositions herein described can be administered using techniques well known to those skilled in the art, such as injection, inhalation or insulation or by oral, parenteral or rectal administration. The vaccine composition can be administered by means including, but not limited to, traditional syringes and needleless injection devices. Suitable routes of administration include, but are not limited to, parenteral delivery, such as intramuscular, intradermal, subcutaneous, intramedullary injections, as well as, intrathecal, direct intraventricular, intravenous, intraperitoneal, intranasal, or intraocular injections. For injection, the vaccine composition herein described can be formulated in aqueous solutions, optionally in physiologically compatible buffers such as Hanks' solution, Ringer's solution, or physiological saline buffer. In some embodiments, administering can comprise aerosol delivery, nasal delivery, vaginal delivery, rectal delivery, buccal delivery, ocular delivery, local delivery, topical delivery, intracisternal delivery, intraperitoneal delivery, oral delivery, intramuscular injection, intravenous injection, subcutaneous injection, intranodal injection, intratumoral injection, intraperitoneal injection, intradermal injection, or any combination thereof. The composition can be administered intramuscularly (e.g., into a deltoid region of an arm). In some embodiments, the composition is: (i) co-administered with an adjuvant; or (ii) not co-administered with an adjuvant.

In some embodiments, the compositions provided herein can be administered to a subject systematically. The wording “systemic administration” as used herein indicates any route of administration by which a vaccine composition is brought in contact with the body of the individual, so that the resulting composition location in the body is systemic (i.e. non limited to a specific tissue, organ or other body part where the vaccine is administered). Systemic administration includes enteral and parenteral administration. Enteral administration is a systemic route of administration where the substance is given via the digestive tract, and includes but is not limited to oral administration, administration by gastric feeding tube, administration by duodenal feeding tube, gastrostomy, enteral nutrition, and rectal administration. Parenteral administration is a systemic route of administration where the substance is given by route other than the digestive tract and includes but is not limited to intravenous administration, intra-arterial administration, intramuscular administration, subcutaneous administration, intradermal, administration, intraperitoneal administration, and intravesical infusion.

The vaccine composition herein disclosed can be administered to a subject using a prime/boost protocol. In such protocol, a first vaccine composition is administered to the subject (prime) and then after a period of time, a second vaccine composition can be administered to the subject (boost). Administration of the second composition (boost composition) can occur days, weeks or months after administration of the first composition (prime composition). For example, the boost composition can be administered about three days, 1 week, 2 weeks, 3 weeks, 4 weeks, 5 weeks, 6 weeks, 7 weeks, 8 weeks, 12 weeks, 16 weeks, 20 weeks, 24 weeks, or 28 weeks, or a number or a range between any two of these values, after the prime composition is administered. In some embodiments, the boost composition can be administered about 4 weeks after administration of the prime composition.

Therefore, the vaccine composition can be administered to the subject in need two or more times. For example, the methods herein described can comprise administering to the subject a first vaccine composition, and after a period of time, administering to the subject a second vaccine composition. The prime vaccine composition and the boost vaccine composition can be, but need not be, the same composition. In some embodiments, the prime vaccine composition and the boost vaccine composition can contain same or different fusion proteins.

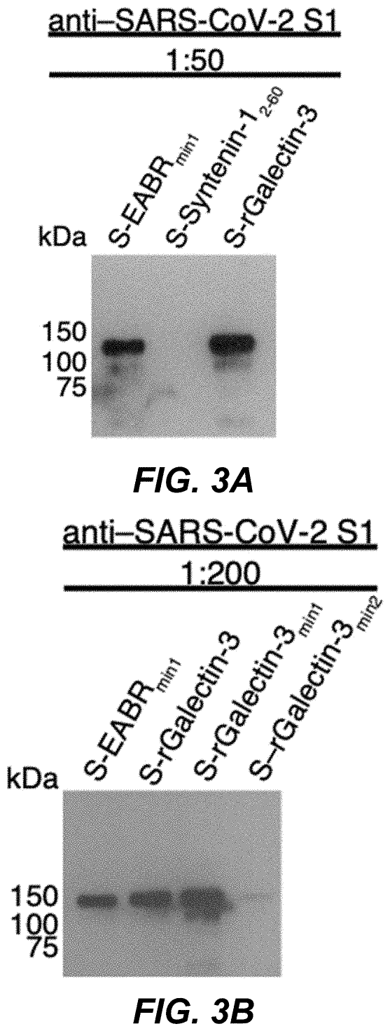

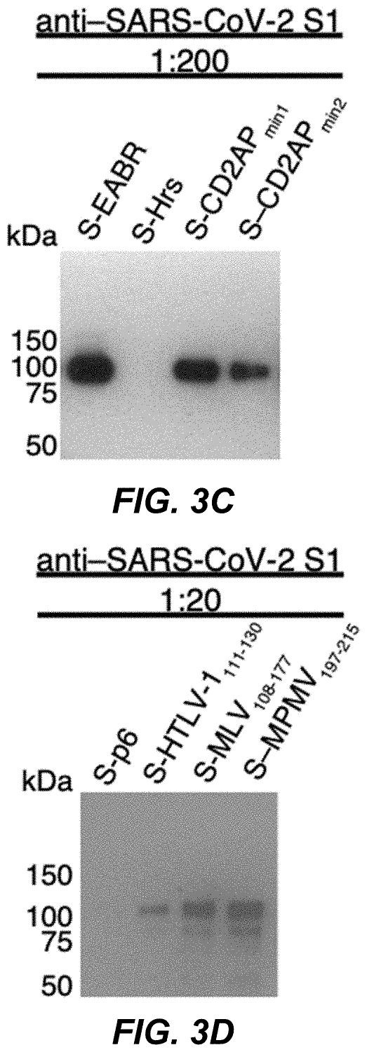

The vaccine compositions provided herein can be used to protect a subject against infection by heterologous infectious agents (e.g., infectious agents different from those that the AP(s) are derived from). For example, a vaccine composition made using coronavirus antigens of a first coronavirus and a second coronavirus is capable of protecting an individual against infection by not only the first and second coronaviruses (i.e., the matched strains), but also coronaviruses from different taxonomic groups (i.e., mismatched strains or coronavirus strains different from the first and second coronaviruses). For example, a vaccine composition made using coronavirus antigens from WIV1, Rf1, RmYN02 and pang17 can elicit broadly neutralizing antibodies, thereby protecting the subject against infection by not only WIV1, Rf1, RmYN02 and pang17 at a comparable magnitude, but also coronavirus SARS-CoV2, SHC014, SARS-CoV, Yun 11, BM-4831 and BtKY72 (see e.g., FIGS. 3C-F).

In some embodiments, the compositions provided herein thereof can protect an individual against infection by an antigenically divergent infectious agents. For example, in some embodiments, a vaccine composition made using coronavirus antigens of a first coronavirus and a second coronavirus is also capable of protecting an individual against infection by emerging coronavirus variants of the first and second coronaviruses. For example, a vaccine composition made using coronavirus antigens of SARS-CoV2 and SHC014 can protect an individual against infection by antigenically divergent coronavirus strains of Sarbecovirus and by diverging coronavirus strains of the future.

In some embodiments, administering the composition (e.g., vaccine composition) induces neutralizing responses against: (i) the infectious agent(s) from which the antigenic polypeptide(s) are derived; and/or (ii) additional infectious agent(s) from which the antigenic polypeptide(s) are not derived, optionally different from the 1st, 2nd, 3rd, 4th, 5th, 6th, 7th, 8th, 9th, 10th, 11th, 12th, 13th, 14th, 15th, 16th, 17th, 18th, 19th, 20th, 21st, 22nd, 23rd, 24th, 25th, 26th, 27th, 28th, 29th, 30th, 31st, 32nd, 33rd, 34th, 35th, 36th, 37th, 38th, 39th, 40th, 41st, 42nd, 43rd, 44th, 45th, 46th, 47th, 48th, 49th, and/or 50th infectious agent. In some embodiments, administering the composition (e.g., vaccine composition) induces neutralizing responses against: the coronaviruses the plurality of coronavirus antigens are of; coronaviruses different from the 1st, 2nd, 3rd, 4th, 5th, 6th, 7th, 8th, 9th, 10th, 11th, 12th, 13th, 14th, 15th, 16th, 17th, 18th, 19th, 20th, 21st, 22nd, 23rd, 24th, 25th, 26th, 27th, 28th, 29th, 30th, 31st, 32nd, 33rd, 34th, 35th, 36th, 37th, 38th, 39th, 40th, 41st, 42nd, 43rd, 44th, 45th, 46th, 47th, 48th, 49th, and/or 50th CoV; and/or additional coronaviruses different from the coronaviruses the plurality of coronavirus antigens are of.

In some embodiments, administering the composition (e.g., vaccine composition) results in treating or preventing: infection caused by a coronavirus different from the first coronavirus and the second coronavirus; infection caused by additional coronaviruses different from the coronaviruses the plurality of coronavirus antigens are of; infection caused by the coronaviruses the plurality of coronavirus antigens are of; the disease or disorder caused by a coronavirus different from the first coronavirus and the second coronavirus; the disease or disorder caused by additional coronaviruses different from the coronaviruses the plurality of coronavirus antigens are of; and/or the disease or disorder caused by the coronaviruses the plurality of coronavirus antigens are of.

The disease or disorder can be a blood disease, an immune disease, a neurological disease or disorder, a cancer, an infectious disease, a genetic disease, a disorder caused by aberrant mtDNA, a metabolic disease, a disorder caused by aberrant cell cycle, a disorder caused by aberrant angiogenesis, a solid tumor, a disorder cause by aberrant DNA damage repair, or any combination thereof.

The disease or disorder can be an infectious disease selected from the group consisting of an Acute Flaccid Myelitis (AFM), Anaplasmosis, Anthrax, Babesiosis, Botulism, Brucellosis, Campylobacteriosis, Carbapenem-resistant Infection, Chancroid, Chikungunya Virus Infection, Chlamydia, Ciguatera, Difficile Infection, Perfringens, Coccidioidomycosis fungal infection, coronavirus infection, Covid-19 (SARS-CoV-2), Creutzfeldt-Jacob Disease/transmissible spongiform encephalopathy, Cryptosporidiosis (Crypto), Cyclosporiasis, Dengue 1,2,3 or 4, Diphtheria, E. coli infection/Shiga toxin-producing (STEC), Eastern Equine Encephalitis, Hemorrhagic Fever (Ebola), Ehrlichiosis, Encephalitis, Arboviral or parainfectious, Non-Polio Enterovirus, D68 Enteroviru (EV-D68), Giardiasis, Glanders, Gonococcal Infection, Granuloma inguinale, Haemophilus Influenza disease Type B (Hib or H-flu), Hantavirus Pulmonary Syndrome (HPS), Hemolytic Uremic Syndrome (HUS), Hepatitis A (Hep A), Hepatitis B (Hep B), Hepatitis C (Hep C), Hepatitis D (Hep D), Hepatitis E (Hep E), Herpes, Herpes Zoster (Shingles), Histoplasmosis infection, Human Immunodeficiency Virus/AIDS (HIV/AIDS), Human Papillomavirus (HPV), Influenza (Flu), Legionellosis (Legionnaires Disease), Leprosy (Hansens Disease), Leptospirosis, Listeriosis (Listeria), Lyme Disease, Lymphogranuloma venereum infection (LGV), Malaria, Measles, Melioidosis, Meningitis (Viral), Meningococcal Disease (Meningitis (Bacterial)), Middle East Respiratory Syndrome Coronavirus (MERS-CoV), Mumps, Norovirus, Pediculosis, Pelvic Inflammatory Disease (PID), Pertussis (Whooping Cough), Plague (Bubonic, Septicemic, Pneumonic), Pneumococcal Disease (Pneumonia), Poliomyelitis (Polio), Powassan, Psittacosis, Pthiriasis, Pustular Rash diseases (Small pox, monkeypox, cowpox), Q-Fever, Rabies, Rickettsiosis (Rocky Mountain Spotted Fever), Rubella (German Measles), Salmonellosis gastroenteritis (Salmonella), Scabies, Scombroid, Sepsis, Severe Acute Respiratory Syndrome (SARS), Shigellosis gastroenteritis (Shigella), Smallpox, Staphyloccal Infection Methicillin-resistant (MRSA), Staphylococcal Food Poisoning Enterotoxin B Poisoning (Staph Food Poisoning), Saphylococcal Infection Vancomycin Intermediate (VISA), Staphylococcal Infection Vancomycin Resistant (VRSA), Streptococcal Disease Group A (invasive) (Strep A (invasive), Streptococcal Disease, Group B (Strep-B), Streptococcal Toxic-Shock Syndrome STSS Toxic Shock, Syphilis (primary, secondary, early latent, late latent, congenital), Tetanus Infection, Trichomoniasis, Trichonosis Infection, Tuberculosis (TB), Tuberculosis Latent (LTBI), Tularemia, Typhoid Fever Group D, Vaginosis, Varicella (Chickenpox), Vibrio cholerae (Cholera), Vibriosis (Vibrio), Ebola Virus Hemorrhagic Fever, Lasa Virus Hemorrhagic Fever, Marburg Virus Hemorrhagic Fever, West Nile Virus, Yellow Fever, Yersenia, and Zika Virus Infection.

The disease can be associated with expression of a tumor-associated antigen (e.g., a proliferative disease, a precancerous condition, a cancer, and a non-cancer related indication associated with expression of the tumor antigen). The cancer can be selected from the group consisting of colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of said cancers, and metastatic lesions of said cancers. The cancer can be a hematologic cancer chosen from one or more of chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CIVIL), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or pre-leukemia.

### Kits

The compositions disclosed herein (e.g., a nucleic acid composition, a population of ENPs) can be provided as components of a kit.

Kits can include compositions disclosed herein (e.g., a nucleic acid composition, a population of ENPs, a vaccine composition) as well components for making such compositions. As such, kits can include, for example, primers, nucleic acid molecules, expression vectors, nucleic acid constructs encoding protein antigens and/or particle-forming subunits described herein, cells, buffers, substrates, reagents, administration means (e.g., syringes), and instructions for using any of said components. It should be appreciated that a kit may comprise more than one container comprising any of the aforementioned, or related, components. For example, certain parts of the kit may require refrigeration, whereas other parts can be stored at room temperature. Thus, as used herein, a kit can comprise components sold in separate containers by one or more entity, with the intention that the components contained therein be used together.

The composition (e.g., a nucleic acid composition, a population of ENPs, a vaccine composition) can comprise Tris buffer, sucrose, and/or sodium acetate. The composition can comprise an adjuvant (e.g., aluminum hydroxide, alhydrogel, AddaVax, MF59, AS03, Freund's adjuvant, Montanide ISA51, CpG, Poly I:C, glucopyranosyl lipid A, flagellin, resiquimod, or any combination thereof). The composition can be a lyophilized composition. In some embodiments, the lyophilized composition has a water content of less than about 10%. The nucleic acid composition and the LNP-forming components can be in separate vials. The composition can be a pharmaceutical composition, and the pharmaceutical composition can comprise one or more pharmaceutically acceptable carriers, diluents and/or excipients. The composition can comprise instructions for use of the composition for: stimulating an immune response in a subject in need thereof treating or preventing a disease or disorder caused by an infectious agent in a subject in need thereof and/or treating or preventing a CoV infection in a subject in need thereof.

The composition can be formulated or to be formulated: as a liquid, a solid, or a combination thereof; for injection; for intramuscular administration, intranasal administration, transdermal administration, aerosol delivery, nasal delivery, vaginal delivery, rectal delivery, buccal delivery, ocular delivery, local delivery, topical delivery, intracisternal delivery, intraperitoneal delivery, oral delivery, intramuscular injection, intravenous injection, subcutaneous injection, intranodal injection, intratumoral injection, intraperitoneal injection, intradermal injection; and/or as particles (e.g., iron oxide particles, liposomes, micelles, polymer complexes, cationic peptide nanoemulsions, virus-like particles (VLPs), LNPs and/or lipoplex (LPX) particles).

### EXAMPLES

Some aspects of the embodiments discussed above are disclosed in further detail in the following examples, which are not in any way intended to limit the scope of the present disclosure.

### Example 1

### Self-Assembling Viral Spike-EABR Nanoparticles as a Vaccine Platform Technology

The COVID-19 pandemic represents the 3^{rd} outbreak caused by zoonotic transmission of a beta-coronavirus (beta-CoV) in the last 20 years. Hence there is an urgent need for new vaccine strategies to control the ongoing pandemic and prevent future CoV outbreaks. mRNA vaccines have emerged as an ideal platform for the development of rapid-response vaccines, but clinical studies have shown that neutralizing antibody titers elicited by mRNA vaccines are ˜10-fold lower than titers elicited by protein nanoparticle (NP) vaccines. This is a concern with regards to the emergence of SARS-CoV-2 variants of concern (VOCs) that are less sensitive to vaccine-induced antibodies. Thus, rapid-response vaccine technologies are needed that elicit potent antibody responses with a single injection and/or lower doses, to ensure lasting protection against VOCs, reduce costs, and accelerate global distribution. Moreover, prevention of future CoV pandemics requires the development of a universal CoV vaccine that elicits cross-reactive immune responses against a broad spectrum of CoV strains by focusing responses to conserved epitopes. Disclosed herein is EABR NP (e.g., ENP) technology, which modifies membrane proteins such as CoV spike (S) proteins to self-assemble into virus-resembling NPs that bud from the cell surface. NP assembly is induced by inserting a short amino acid sequence derived from the CEP55 protein (EABR domain) into the cytoplasmic tail designed to recruit proteins from the endosomal sorting complex required for transport (ESCRT) pathway. EABR NP assembly can be further enhanced by introducing an endocytosis-preventing motif derived from the murine low-affinity gamma Fc region receptor II FcRII-B1 isoform. EABR NPs presenting the SARS-CoV-2 S protein (S-EABR NPs) are 20-40 nm in diameter, surrounded by a lipid bilayer, and densely-coated with spikes. S-EABR NPs incorporate >10-fold more S protein compared to conventional NP approaches such as co-expression of S with HIV-1 Gag or the SARS-CoV-2 structural proteins M, N, and E. Studies in mice showed that intramuscular injections of S-EABR NPs presenting the SARS-CoV-2 S protein elicited 10-fold higher neutralizing antibody titers than soluble S protein and protein-based NPs that displayed the receptor-binding domain (RBD) of the S protein. S-EABR NPs also elicited more potent serum neutralizing titers against the Omicron VOC than an mRNA vaccine encoding the S protein. An advantage of the EABR technology is that S-EABR constructs can be delivered as mRNA vaccines since NP assembly only requires expression of a single genetically-encoded component. The EABR NP technology can be used to develop a hybrid mRNA vaccine approach that combines attributes of mRNA and protein-based NP vaccines to elicit protective and long-lasting immunity against SARS-CoV-2 and VOCs. While the S protein encoded by conventional mRNA vaccines is only presented on the cell surface, fusion proteins generated by engineered S-EABR constructs are presented on cell surfaces and also self-assemble into virus-like NPs that are secreted from cells and widely distribute inside the body, thereby mimicking a natural infection. Studies in mice demonstrated that compared to a conventional mRNA vaccine, the hybrid mRNA vaccine approach elicited 21- and 7-fold higher neutralizing antibody responses against SARS-CoV-2 and the Delta variant, respectively. Increased immune responses can ensure lasting protection against SARS-CoV-2 VOCs, enabling, e.g., lower vaccine doses than existing vaccines, which can reduce costs and expedite global distribution.

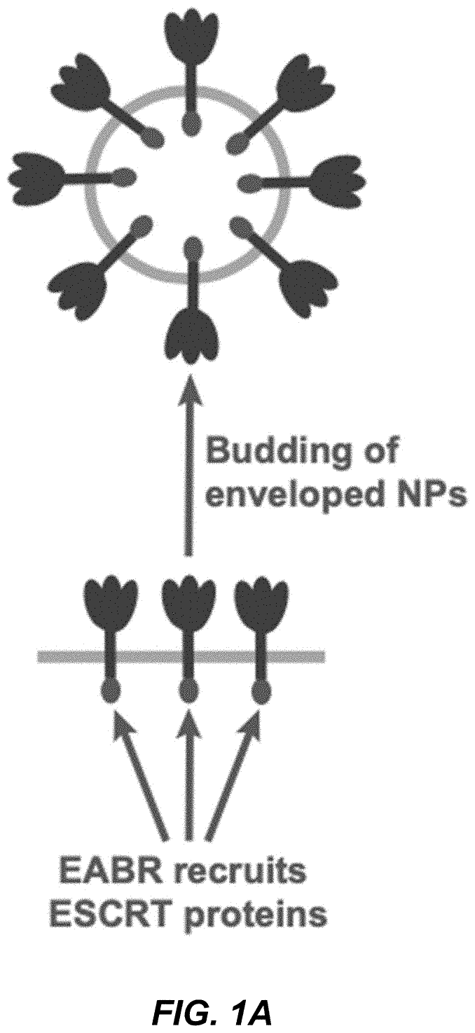

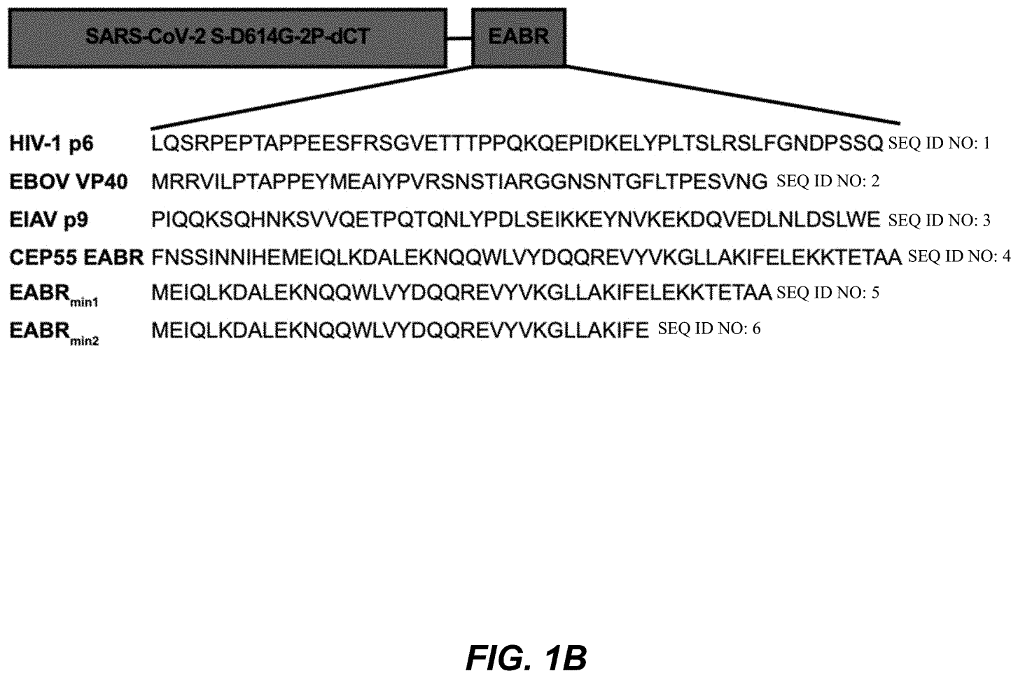

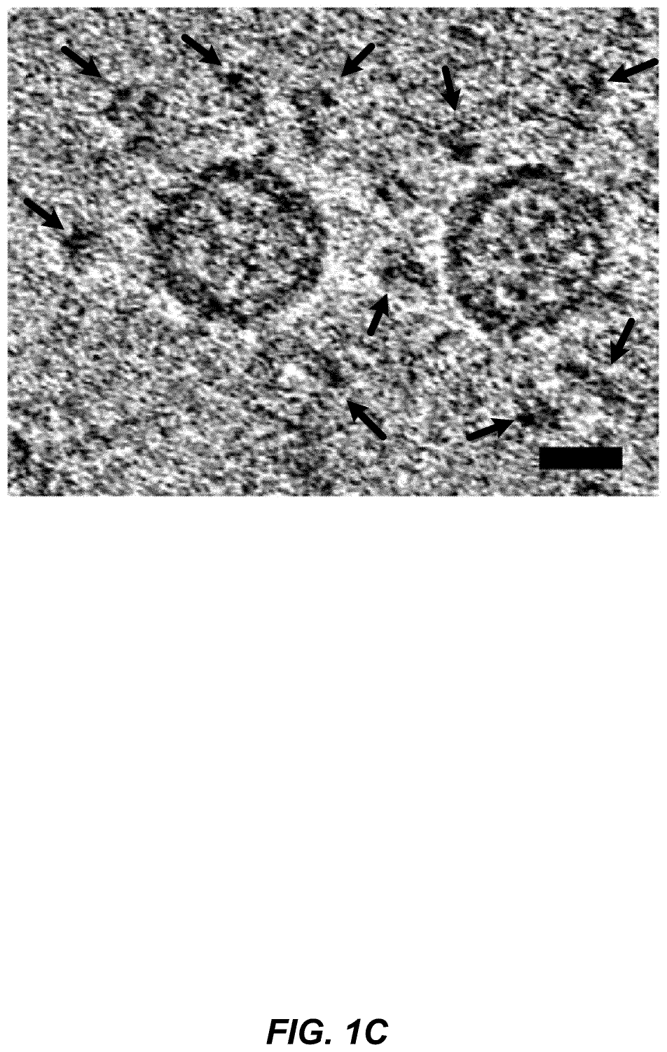

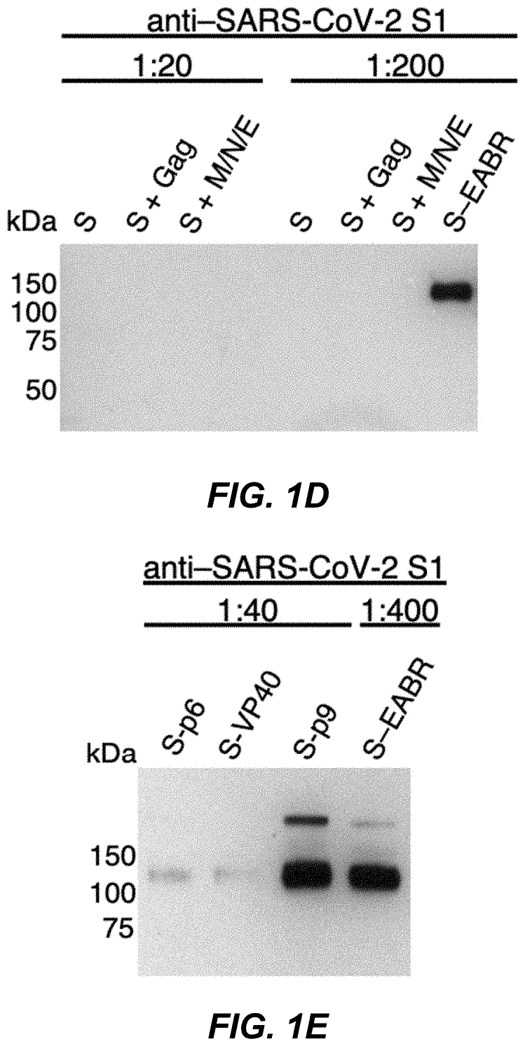

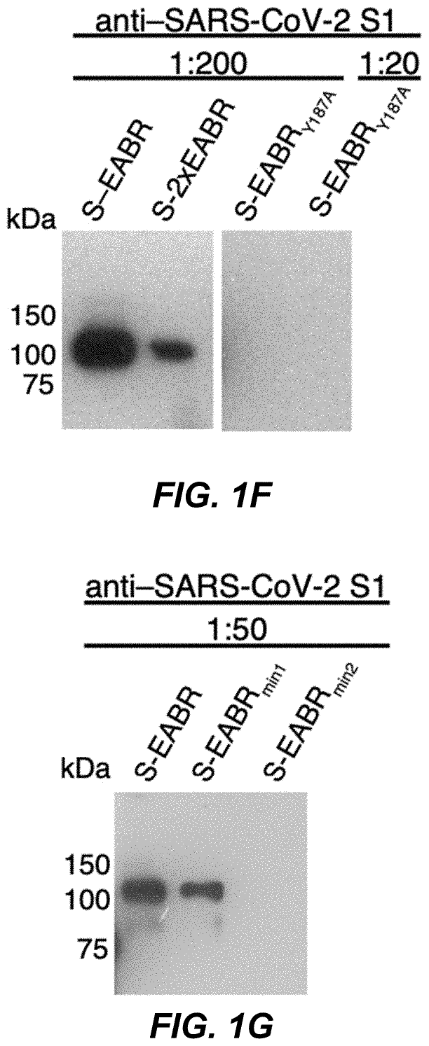

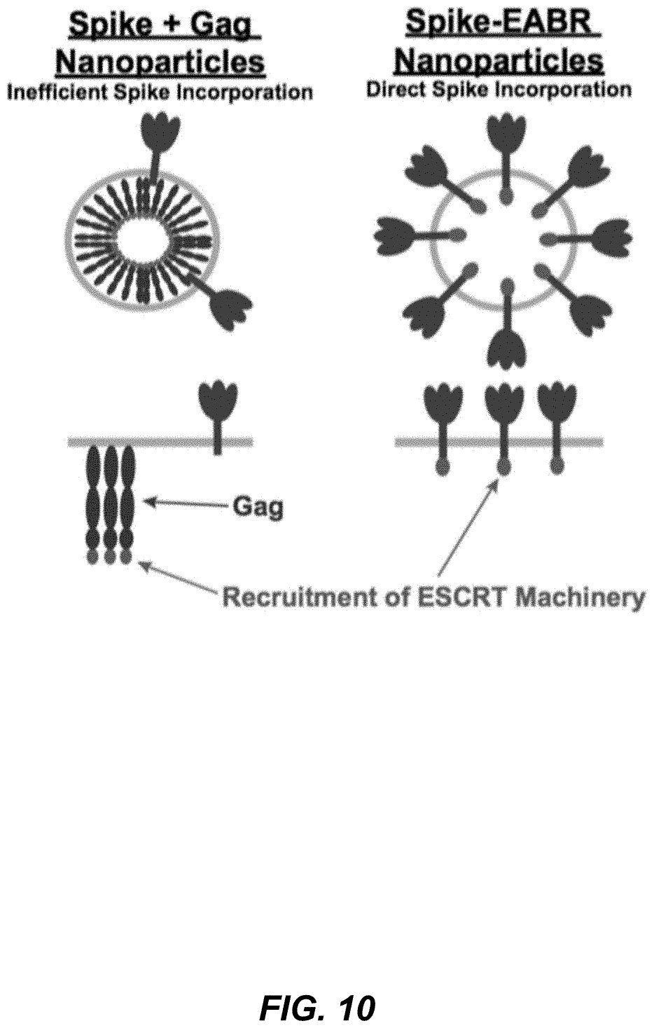

Multivalent display of viral surface proteins on NPs is widely known to enhance antibody responses. Conventional protein-NP vaccines require two components: i) the surface protein itself; and ii) a structural scaffold protein that self-assembles to form the NP such as the Gag protein from lentiviruses. Presented herein are methods to engineer the surface protein itself to advantageously self-assemble into densely-coated NPs without the need for additional proteins. NP assembly is achieved by inserting a short amino acid sequence at the end of the cytoplasmic tail of the surface protein, which recruits host proteins from the endosomal sorting complex required for transport (ESCRT) pathway that has been shown to drive the viral budding process for a number of enveloped viruses (FIG. 1A, FIG. 10). The inserted ESCRT-recruiting domain is derived from the ESCRT and ALIX binding region (EABR) of the human CEP55 protein (residues 170-213) FIG. 1B), which interacts with the ESCRT proteins TSG101 and ALIX during cytokinesis.

This technology was evaluated by fusing the EABR domain to the C-terminus of the SARS-CoV-2 S protein, separated by a short Gly-Ser linker (FIG. 1B). This version of the S protein contained a D614G mutation that has been shown to increase infectivity of SARS-CoV-2. Two previously described proline substitutions (2P) were introduced into the S2 subunit to stabilize the prefusion conformation. To ensure efficient cell surface expression, the C-terminal 21 residues were truncated from the cytoplasmic tail of S as it contains an endoplasmic reticulum (ER)-retention signal.

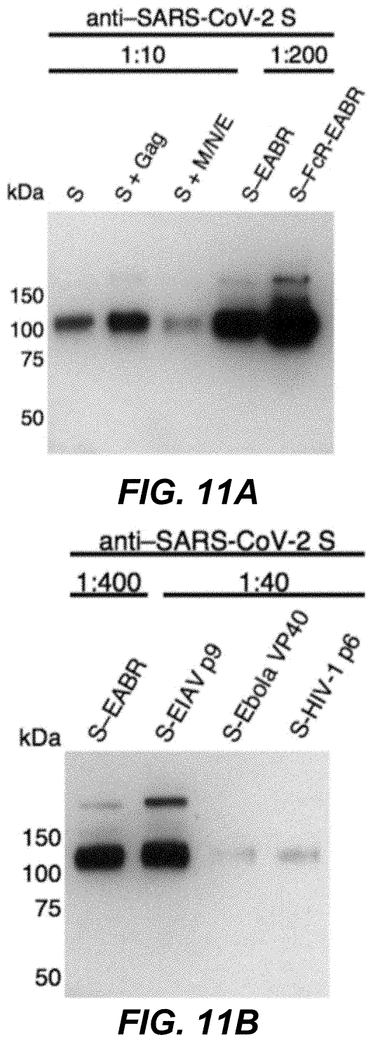

Expi293 cells were transiently-transfected to generate S-EABR NPs. After 48 hours, supernatants were collected and NPs were purified by ultracentrifugation on a 20% sucrose cushion. Cryo-electron tomograms showed that S-EABR NPs are 20-40 nm in diameter, surrounded by a lipid bilayer, and densely-coated with spikes (FIG. 1C). Western blot analysis demonstrated that purified S-EABR NPs contained >20-fold more S protein than NPs produced by co-expression of S and HIV-1 Gag or S and the SARS-CoV-2 structural proteins, M, N, and E (FIG. 1D, FIG. 11A) suggesting that S-EABR NPs incorporate S more efficiently than conventional NP approaches.

The EABR domain was >10-fold more effective at generating S-containing NPs than viral ESCRT-interacting proteins such as EIAV p9, EBOV VP40, and HIV-1 p6 (FIG. 1E, FIG. 11B). However, addition of a second EABR domain reduced S-EABR NP production (FIG. 1F). To verify that S-EABR NP production is dependent on recruitment of ESCRT proteins, a Y187A mutation was introduced in the EABR domain, which has been shown to abolish interactions with TSG101 and ALIX and consequently impaired S-EABR NP production (FIG. 1F). To identify the minimal EABR sequence required for NP self-assembly, SARS-CoV-2 S constructs fused to the complete EABR domain (CEP55₁₇₀₋₂₁₃), EABRₘᵢₙ₁ (CEP55₁₈₀₋₂₁₃), and EABRₘᵢₙ₂ (CEP55₁₈₀₋₂₀₄) (FIG. 1B) were generated. While S-EABR NP yields were diminished for EABRₘᵢₙ₂ (FIG. 1G) and for EABRₘᵢₙ₃ (not shown), production efficiency was retained for EABRₘᵢₙ₁ (FIG. 1G).

TABLE 1
AMINO ACID SEQUENCES OF EABR DOMAINS

| NAME | SEQUENCE | SEQ ID NO |
|---|---|---|
| EABR | FNSSINNIHEMEIQLKDALEKNQQWLVYDQQREVYVKGLLAKIFELEKKTETAA | 4 |
| EABRmin1 | MEIQLKDALEKNQQWLVYDQQREVYVKGLLAKIFELEKKTETAA | 5 |
| EABRmin2 | MEIQLKDALEKNQQWLVYDQQREVYVKGLLAKIFE | 6 |
| EABRmin3 | MEIQLKDALEKNQQWLVYDQQREVYVKG | 23 |
| muEABRmut1 | KEMQLRDALDKNHQWLLYDQQREAYVRGLLARIFELEKRTETAAA | 7 |
| chicEABRmut1 | VEKQLRDALEKNHQWLLYDQQREAYVRGLLGRIFELEQRSEIVSQQQP | 8 |
| chicEABRmut2 | VEKQLKDALDKNQQWLLYDQQREAYVRGLLGRIFELEQRSEIVSQQQP | 9 |
| chamEABRmut1 | IGMQLRDALDKNQQWLLYDQQREAYVQGLLARIFELEQQAKEAKPEE | 10 |
| goldEABRmut1 | LQEQLRDALEKNHNWLLYDQQREAYVQGVLTRTKELETQLNEANQAL | 11 |

(EABR), EABRₘᵢₙ₁, EABRₘᵢₙ₂, EABRₘᵢₙ₃, ₘᵤEABRₘᵤₜ₁, _{chic}EABRₘᵤₜ₁, _{chic}EABRₘᵤₜ₂, _{cham}EABRₘᵤₜ₁, and
_{gold}EABRₘᵤₜ₁, Residues that differ from the human EABR sequence are in bold.

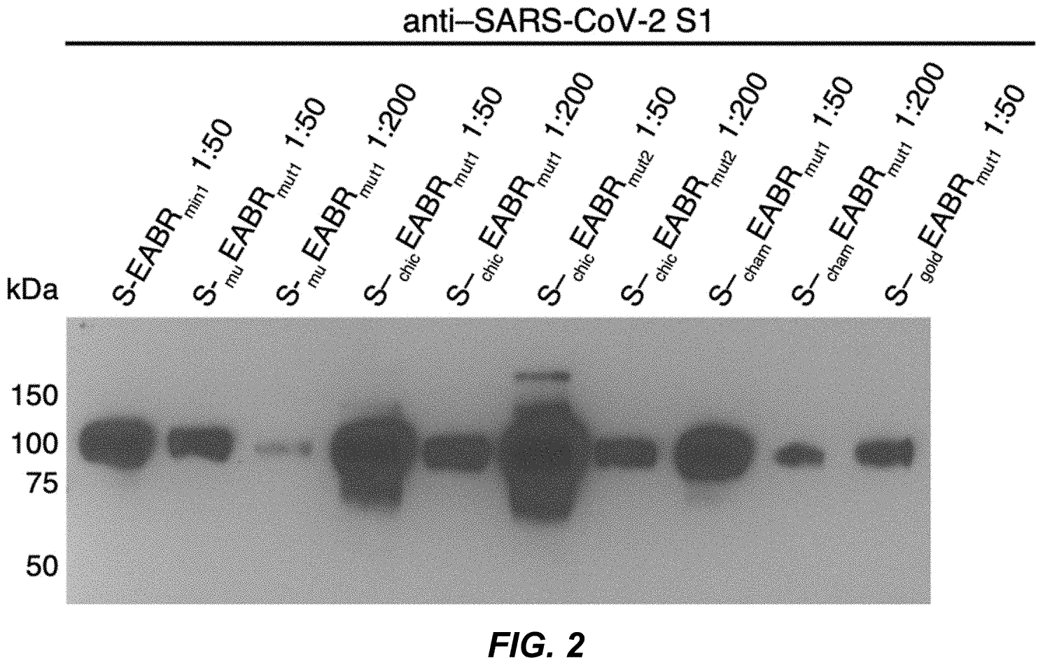

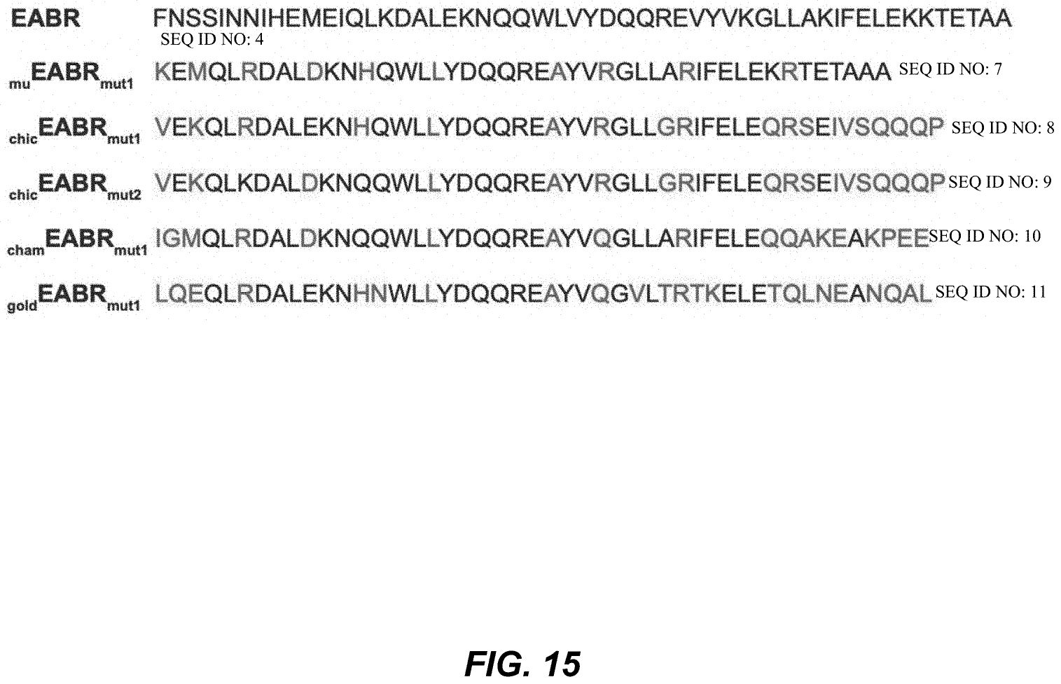

To prevent potential T-cell responses against the human EABR domain, EABR domains were evaluated from different species and additional mutations were introduced into regions that were identical to the human EABR (Table 1, FIG. 15). All constructs (e.g., fusion protein constructs) generated S-EABR NPs, and the two constructs based on the chicken EABR domain, chicEABRmut1 and chicEABRmut2, improved S-EABR NP production compared to the human EABRmin1 construct (FIG. 2). In some embodiments, the EABR sequence can be optimized to further increase NP production.

TABLE 2
AMINO ACID SEQUENCES OF THE ESCRT-BINDING DOMAINS

| NAME | SEQUENCE | SEQ ID NO |
|---|---|---|
| Syntenin-1₂₋₆₀ | SLYPSLEDLKVDKVIQAQTAFSANPANPAILSEASAPIPHDGNLYPRLYPELSQYMGLS | 12 |
| rGalectin-3 | MADGFSLNDALAGSGNPNPQGWPGAWGNQPGAGGYPGASYPGAYPGQAPPGGYPGQAPP | 13 |
| | SAYPGPTGPSAYPGPTAPGAYPGPTAPGAFPGQPGGPGAYPSAPGAYPSAPGAYPATGP | |
| | FGAPTGPLTVPYDMPLPGGVMPRMLITIIGTVKPNANSITLNFKKGNDIAFHFNPRFNE | |
| | NNRRVIVCNTKQDNNWGREERQSAFPFESGKPFKIQVLVEADHFKVAVNDVHLLQYNHR | |
| | MKNLREISQLGIIGDITLTSASHAMI | |
| rGalectin-3ₘᵢₙ₁ | MADGFSLNDALAGSGNPNPQGWPGAWGNQPGAGGYPGASYPGAYPGQAPPGGYPGQAPP | 14 |
| | SAYPGPTGPSAYPGPTAPGAYPGPTAPGAFPGQPGGPGAYPSAPGAYPSAPGAYPATGP | |
| | FG | |
| rGalectin-3ₘᵢₙ₂ | GPTGPSAYPGPTAPGAYPGPTAPGAFPGQPGGPGAYPSAPGAYPSAPGAYPATGPFGMV | 15 |
| CD2APₘᵢₙ₁ | DYIVEYDYDAVHDDELTIRVGEIIRNVKKLQEEGWLEGELNGRRGMFPDNFVKEIKRET | 16 |
| | EFKDDSLPIKRERHGNVASLVQRISTYGLPAGGIQPHPQTKNIKKKTKKRQCKVLFEYI | |
| | PQNEDELELKVGDIIDINEEVEEGWWSGTLNNKLGLFPSNFVKELEV | |
| CD2APₘᵢₙ₂ | MVDYIVEYDYDAVHDDELTIRVGEIIRNVKKLQEEGWLEGELNGRRGMFPDNFVKEIKR | 17 |
| HTLV-1 | PDSDPQIPPPYVEPTAPQVL | 18 |
| Gag₁₁₁₋₁₃₀ | | |
| MLV | PLPPSAPSLPLEPPRSTPPRSSLYPALTPSLGAKPKPQVLSDSGGPLIDLLTEDPPPYR | 19 |
| Gag₁₀₈₋₁₇₇ | DPRPPPSDRDG | |
| MPMV | PFLTRPPPYNKATPSAPTV | 20 |
| Gag₁₉₇₋₂₁₅ | | |

### Various ESCRT-Recruiting Domains Induce Efficient NP Assembly

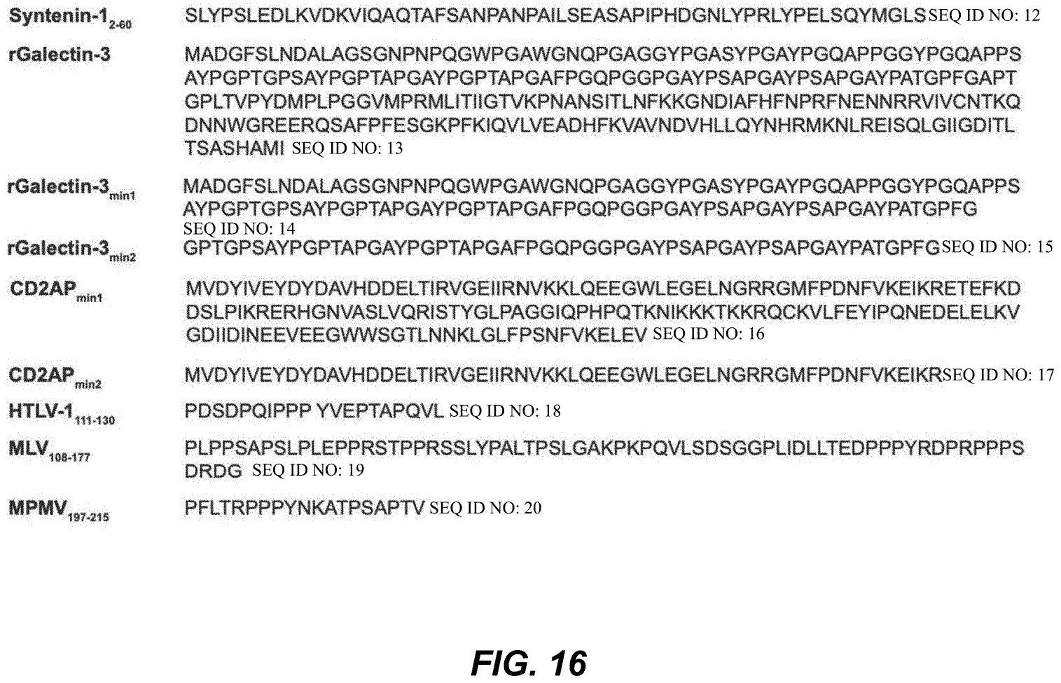

It was investigated whether NP assembly can also be achieved by fusing other ESCRT-binding domains to the cytoplasmic tail of the SARS-CoV-2 S protein. As the CEP55 EABR binds the ESCRT proteins TSG101 and ALIX with high affinity, ESCRT-binding domains from various mammalian and viral proteins that have been shown to interact with these proteins, including Syntenin-1, rat Galectin-3 (rGalectin-3), Hrs, CD2AP, as well as fragments of the HTLV-1, MLV, and MPMV Gag proteins (Table 2, FIG. 16) were evaluated. While NP assembly was inefficient for S-Syntenin-1₂₋₆₀ and S-Hrs, S-rGalectin-3 and S-CD2APₘᵢₙ₁ induced efficient NP assembly with similar yields compared to the original S-EABR construct (FIG. 3A-FIG. 3C). A minimal version of S-rGalectin-3 (S-rGalectin-3ₘᵢₙ₁) was, in some embodiments, even slightly more efficient than S-EABR (FIG. 3B). Further minimization of S-CD2APₘᵢₙ₁ (S-CD2APₘᵢₙ₂) reduced NP yields (FIG. 3C). As previously observed for other viral ESCRT-binding domains (FIG. 1E), NP assembly was >10-fold less efficient for ESCRT-binding domains derived from the HTLV-1, MLV, and MPMV Gag proteins as the purified NP-containing samples needed to be diluted 1:20 for Western blot analysis compared to 1:200 dilutions for the S-EABR or S-rGalectin-3ₘᵢₙ₁ samples (FIG. 3D). The most effective viral ESCRT-binding domains were derived from the EIAV and MPMV Gag proteins (FIG. 1E, FIG. 3D). These results support the overall concept that recruitment of ESCRT proteins to the cytoplasmic tail of membrane proteins induces efficient NP self-assembly, which can be achieved by fusing various ESCRT-binding domains to the C-terminus of the cytoplasmic tail.

### Endocytosis-Preventing Motif (EPM) Enhances NP Assembly

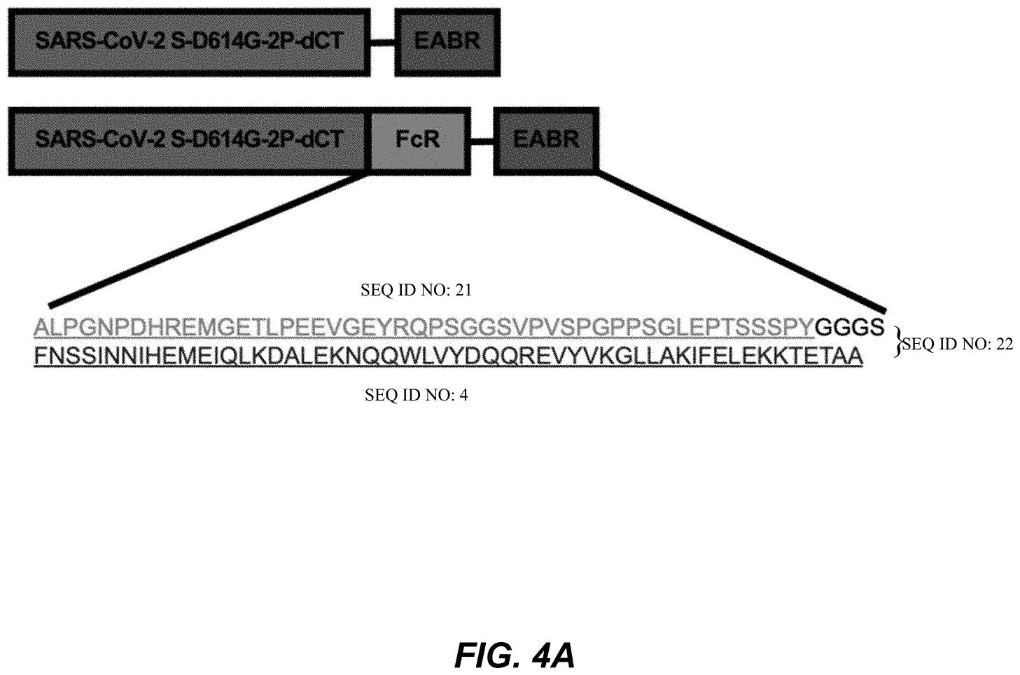

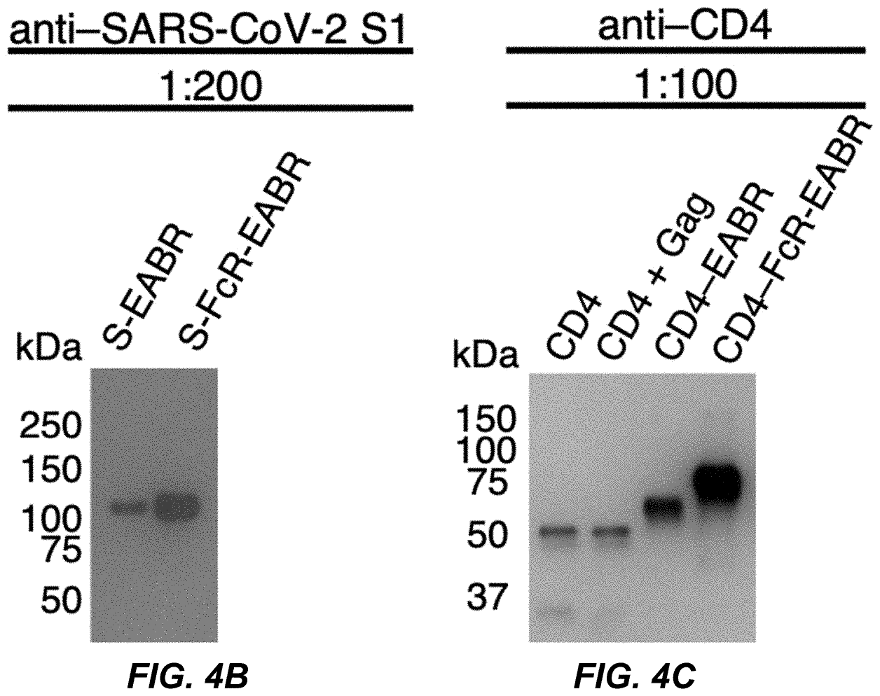

It was hypothesized that S-EABR NP production would be further enhanced by preventing endocytosis of the S-EABR fusion protein. Without being bound by any particular theory, this can result in extending the time S-EABR remains at the plasma membrane to interact with ESCRT proteins. The murine low-affinity gamma Fc region receptor II is expressed in macrophages and B-lymphocytes as two major isoforms, FcRII-B1 and FcRII-B2. The isoforms are generated by alternative mRNA splicing and FcRII-B1 contains a 47 amino acid cytoplasmic tail insertion that has been shown to tether the receptor to the cytoskeleton, thereby preventing localization to coated pits and endocytosis. The S-FcR-EABR construct was generated by inserting this 47-residue sequence upstream of the Gly-Ser linker and the EABR domain (FIG. 4A). Western blot analysis demonstrated that purified NP fractions for S-FcR-EABR NPs contained increased amounts of S protein compared to S-EABR NPs, showing that preventing endocytosis of S improves NP production yields (FIG. 4B). All subsequent S-EABR constructs therefore contained the FcR domain but for simplicity are referred to as “S-EABR”.

The effectiveness of the FcR domain for the human CD4 receptor that gets rapidly endocytosed in the absence of Lck was also evaluated. Insertion of the FcR domain enhanced CD4-EABR NP production (FIG. 4C) demonstrating that the FcR domain effectively prevents endocytosis and enhances EABR NP assembly.

### EABR NP Technology can be Applied to a Wide Range of Membrane Proteins

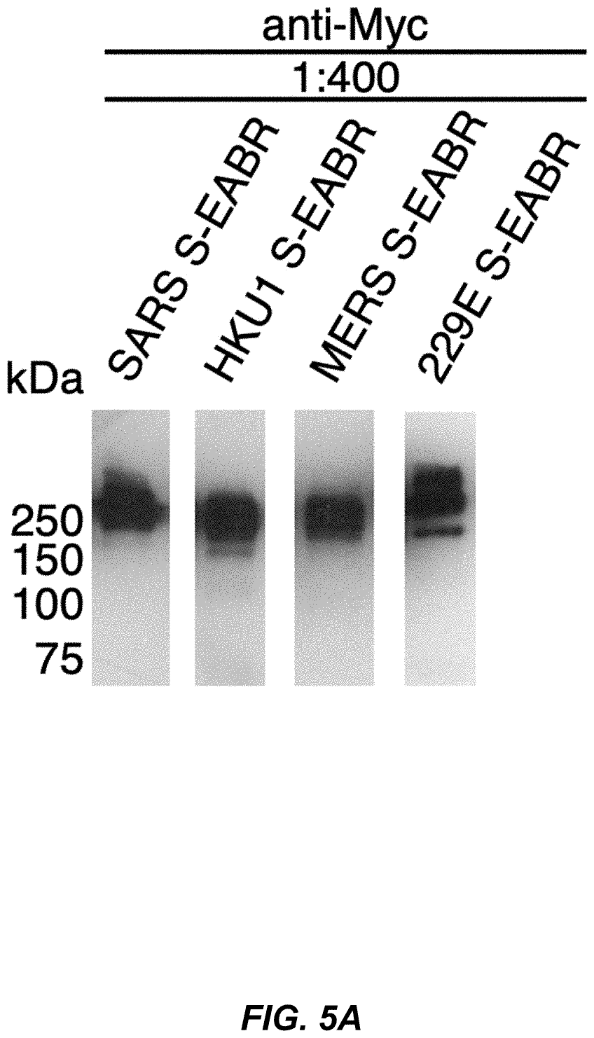

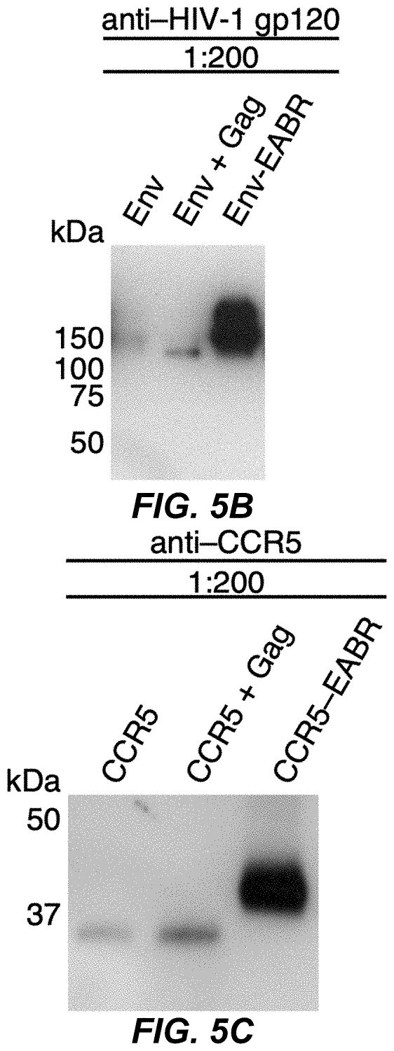

To further assess the modularity of the EABR NP technology, S-EABR NPs for S proteins from other CoV strains that infect humans were generated, including SARS-CoV, MERS-CoV, HKU-1, and 229E, with similar NP yields for all strains (FIG. 5A). Efficient NP self-assembly was also achieved for HIV-1 Env-EABR (YU2 strain), which produced NPs with markedly higher Env content than co-expression of Env and HIV-1 Gag (FIG. 5B). Finally, EABR NPs were also generated for the multi-span transmembrane protein CCR5 (FIG. 5C) suggesting that the EABR NP technology can be applied to a wide range of membrane proteins.

### Purified S-EABR NPs Elicit Potent Immune Responses In Vivo

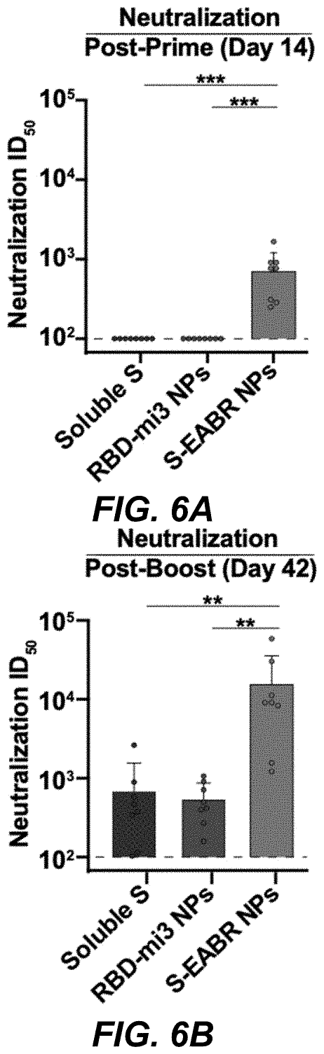

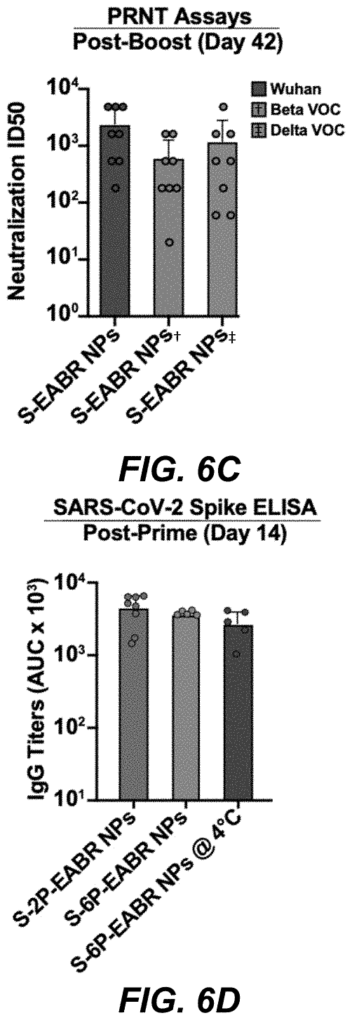

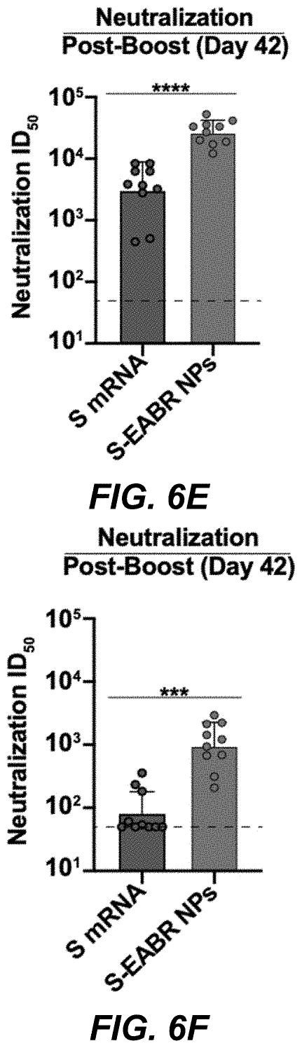

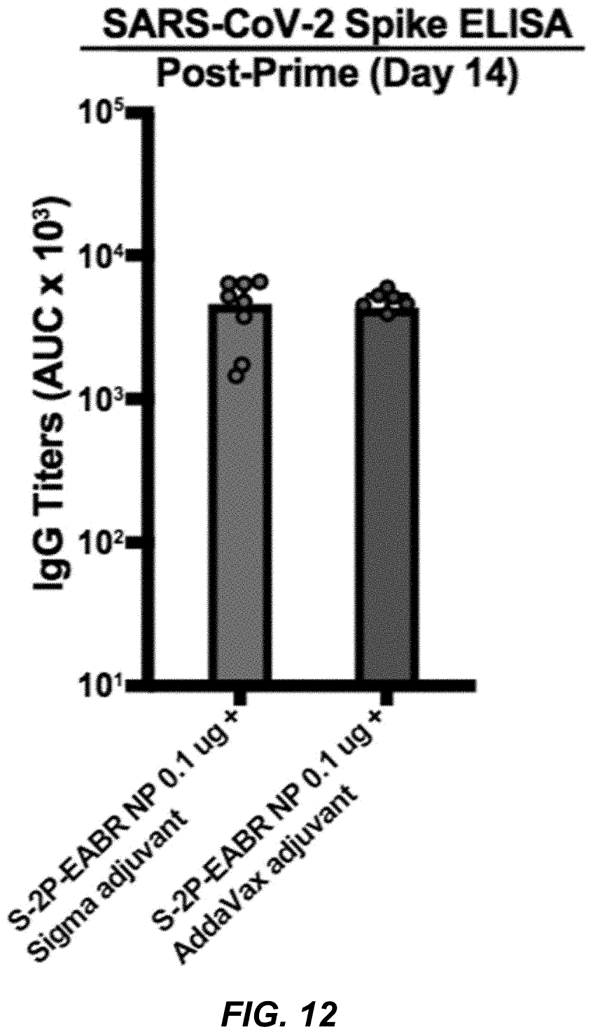

The efficacy of S-EABR NPs as a vaccine candidate against SARS-CoV-2 was evaluated in C57BL/6 mice. S-EABR NPs were purified by ultracentrifugation on a 20% sucrose cushion followed by size exclusion chromatography. S-2P-EABR NPs elicited similar antibody responses when administered in the presence of Sigma or AddaVax adjuvants (FIG. 12). All immunogens in experiments below were administered by subcutaneous injection in the presence of adjuvant (Sigma adjuvant). A single injection of 0.1 μg of S-EABR NPs elicited robust neutralizing antibody titers against lentivirus-based SARS-CoV-2 pseudovirus (assays that correlate with results from authentic virus neutralization) (FIG. 6A). In contrast, no neutralizing antibody responses were detected for the soluble S-2P protein and SpyCatcher-mi3 NPs that displayed the SARS-CoV-2 RBD (FIG. 6A). This dose was 10-50-fold lower than doses used in previous protein-based SARS-CoV-2 immunization studies, highlighting the high potency of the S-EABR NPs. After the second injection, neutralizing antibody titers increased by >10-fold and were 23- and 29-fold higher than titers measured for soluble S-2P and RBD-mi3 NPs, respectively (FIG. 6B). Serum responses were also evaluated against authentic SARS-CoV-2 virus by plaque reduction neutralization tests (PRNT), showing robust neutralizing activity against the early pandemic Wuhan variant (FIG. 6C). As expected, neutralization titers dropped 4-fold and 2-fold against the Beta and Delta variants, respectively.

The effect of 3 months storage at 4° C. on the immunogenicity of S-EABR NPs was also studied. Serum responses decreased by ˜30% for NPs that displayed the S-2P protein (FIG. 6D). However, introduction of additional stabilizing proline mutations into the S2 subunit (S-6P) enhanced the stability of S-EABR NPs and their immunogenicity was retained. Overall, these results demonstrate that purified S-EABR NPs elicit potent immune responses in vivo and represent a new platform technology for NP-based vaccine design.

In a follow-up study, it was demonstrated that two intramuscular injections (days 0 and 28) of 0.5 μg of purified S-EABR NPs in the presence of Addavax adjuvant elicited potent neutralizing responses in BALB/c mice against the Omicron VOC. Compared to an mRNA vaccine encoding the SARS-CoV-2 S protein (similar to the Pfizer/Moderna vaccines), serum neutralizing titers were 8.7- and 11.6-fold higher against the early pandemic Wuhan (FIG. 6E) and the later Omicron VOC (FIG. 6F). Serum neutralizing responses against the Omicron VOC dropped by almost 30-fold compared to titers against the Wuhan variant (FIG. 6E-FIG. 6F), which is consistent with previous reports.

Development of Hybrid mRNA Vaccines that Deliver Self-Assembling EABR NPs

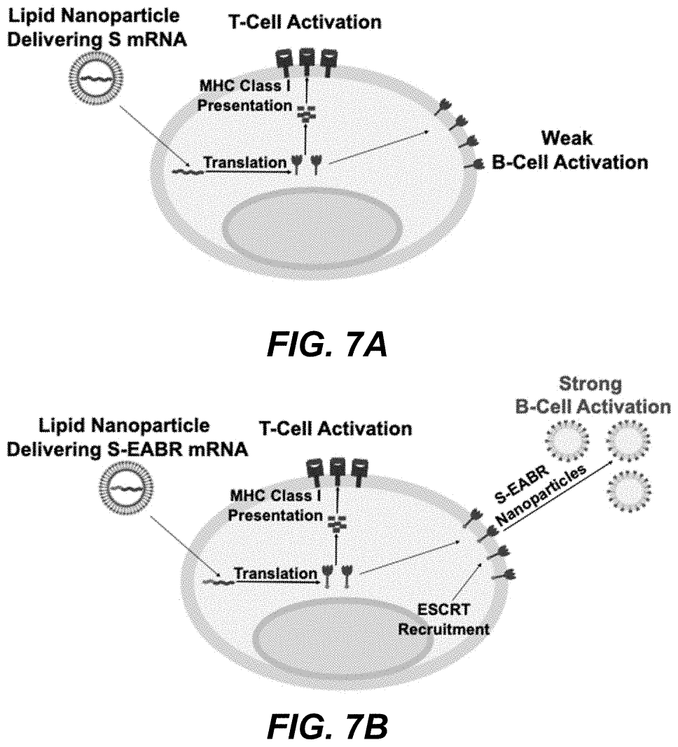

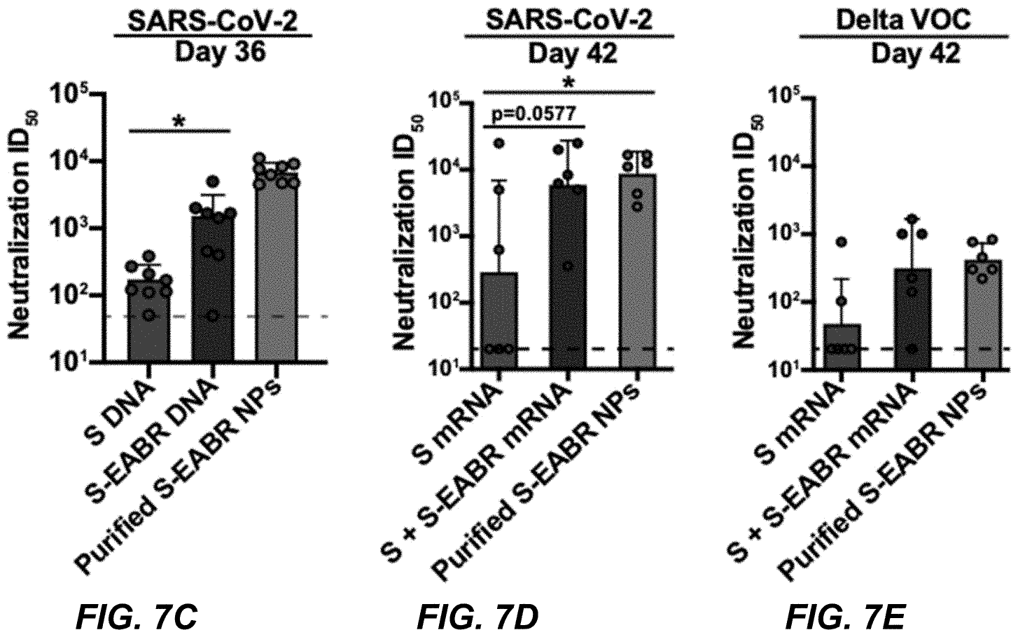

An advantage of the presently disclosed method over existing NP approaches is that S-EABR constructs can be delivered as mRNA vaccines since NP assembly only requires expression of a single genetically-encoded component. The EABR NP technology was applied to develop a hybrid mRNA vaccine approach that combines attributes of mRNA and protein-based NP vaccines to elicit protective and long-lasting immunity against SARS-CoV-2 and VOCs. In contrast to conventional NP technologies, engineered EABR constructs can be delivered as mRNA vaccines since NP assembly only requires a single genetically-encoded component. In comparison to the conventional mRNA vaccines (e.g., Pfizer/Moderna vaccines), mRNA-mediated delivery of the presently disclosed engineered S-EABR construct can, in some embodiments, greatly enhance activation of B-cells, the cells responsible for secreting antibodies, because S-EABR proteins are expressed at the cell surface and self-assemble into virus-resembling NPs that are secreted from cells (FIG. 7A-FIG. 7B). Both S (FIG. 7A) and S-EABR mRNAs (FIG. 7B) can be expressed inside host cells and localize to the cell surface, which elicits potent T-cell activation but only moderate B-cell responses. However, formation and secretion of self-assembling S-EABR NPs by the S-EABR mRNA (FIG. 7B), but not by the S mRNA, can enhance B-cell activation because the NPs would widely distribute inside the body to engage a large number of immune cells, thereby mimicking a natural infection.

To test this hypothesis, a pilot study was performed with DNA vaccines in mice. In some embodiments, plasmid-based DNA vaccines are not as effective as mRNA vaccines, but are easy to produce, cheap, and can be injected directly into animals without LNP packaging. DNA vaccines have also been shown to protect non-human primates (NHPs) from SARS-CoV-2 challenges. This study showed that the S-EABR DNA vaccine elicited 9-fold higher neutralizing antibody responses against lentivirus-based SARS-CoV-2 pseudovirus (assays that correlate with results from authentic virus neutralization) compared to the conventional S DNA vaccine (FIG. 7C). Although neutralizing responses were still 4-fold lower than titers elicited by protein-based S-EABR NPs, these results indicated that a hybrid vaccine approach can enhance the potency of nucleic acid-based vaccines.

To further evaluate hybrid vaccine technology with mRNA, mRNA constructs encoding the SARS-CoV-2 S and S-EABR constructs were synthesized at the Houston Methodist Research Institute and were encapsulated using a standard lipid nanoparticle (LNP) formulation (Precision NanoSystems). Groups of six mice received two intramuscular (IM) injections in weeks 0 and 4 of either 1 μg of the unmodified S mRNA vaccine alone (similar to the Pfizer/Moderna vaccine) or 1 μg of a 1:1 combination of the S and S-EABR mRNAs (0.5 μg S mRNA+0.5 μg S-EABR mRNA). Both mRNA vaccines were compared to 1 μg doses of purified S-EABR NPs that were administered in weeks 0 and 4 in the presence of adjuvant.

Two weeks post-boost injections, serum neutralizing antibody responses were measured by in vitro neutralization assays against lentivirus-based SARS-CoV-2 pseudoviruses for the original Wuhan and Delta variants. The conventional S mRNA vaccine elicited neutralizing antibody responses against the Wuhan and Delta variants in only 3 of 6 and 2 of 6 mice, respectively (FIG. 7D-FIG. 7E). In contrast, potent neutralizing antibody responses against the Wuhan strain were detected for all mice that received the S+S-EABR mRNA combination vaccine (FIG. 7D). Moreover, 5 of 6 serum samples neutralized the Delta variant (FIG. 7E). Geometric means for serum half-maximal inhibitory dilutions (ID₅₀s) against the Wuhan and Delta variants were 21−(p=0.0577) and 7-fold higher, respectively, for the S+S-EABR mRNA combination compared to the conventional S mRNA vaccine. Neutralizing antibody titers elicited by the S+S-EABR mRNA combination were comparable to responses observed for the purified S-EABR NPs showing that hybrid mRNA vaccines can induce similarly potent antibody responses in the absence of adjuvants.

### Mosaic S-EABR NPs as Pan-CoV Vaccine Candidates

Another advantage of the EABR NP technology is that viral surface proteins are maintained in their natural membrane-associated conformation without the need for extensive protein engineering. The EABR technology is therefore the ideal approach for developing mosaic NP-based pan-CoV vaccines that present full-length S proteins from a wide range of CoV strains. The presentation of full-length S proteins on S-EABR NPs ensures that highly conserved regions in the S2 subunit are displayed to the immune system to elicit cross-reactive antibodies that neutralize a broad spectrum of CoV strains. The production of mosaic NPs presenting full-length S proteins would be difficult to achieve using conventional mosaic NP approaches as they require viral surface proteins to be modified into soluble proteins. Full-length CoV S proteins are membrane proteins, and soluble versions of S protein trimers are unstable in the prefusion conformation in the absence of stabilizing mutations, which, in some embodiments, only work for a subset of CoV strains to express stable, soluble S protein trimers. Large-scale expression of eight or more different soluble CoV S proteins to generate mosaic NPs might therefore be unrealistic since effective stabilizing mutations for many CoV S proteins have not been identified. In addition, multiple production steps would be required as all components, including the eight or more CoV S proteins and the NP-assembling proteins need to be individually expressed and purified prior to mosaic NP assembly, followed by a final purification step. Hence NP vaccine technologies are needed that can display a wide range of full-length CoV S proteins without the need for extensive protein engineering and numerous manufacturing steps. A need which is met with the methods and compositions provided herein.

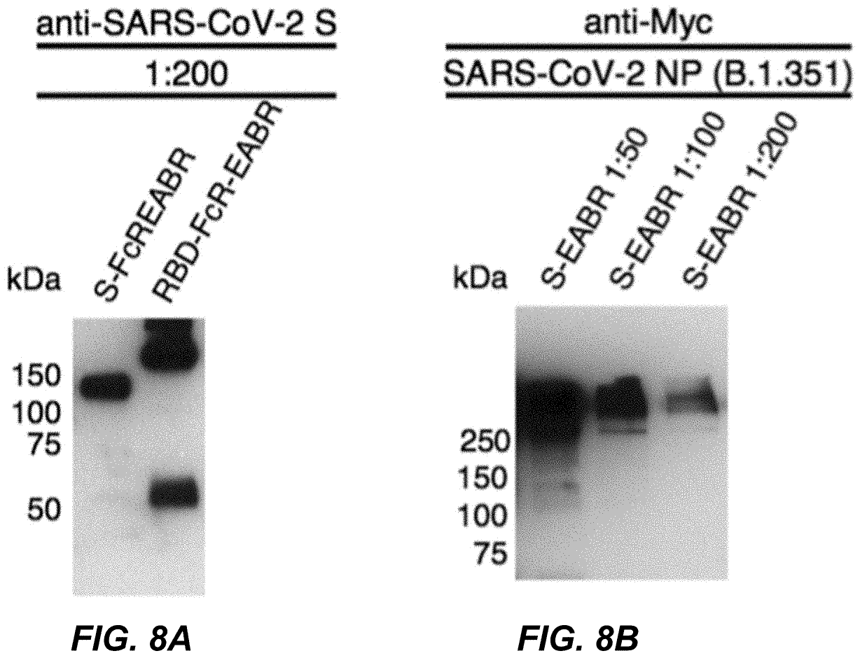

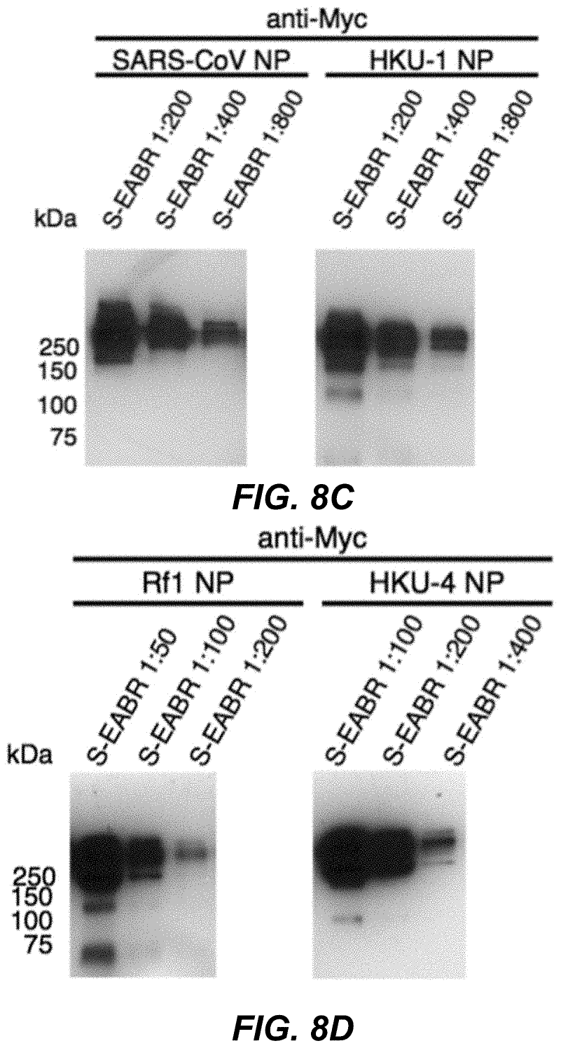

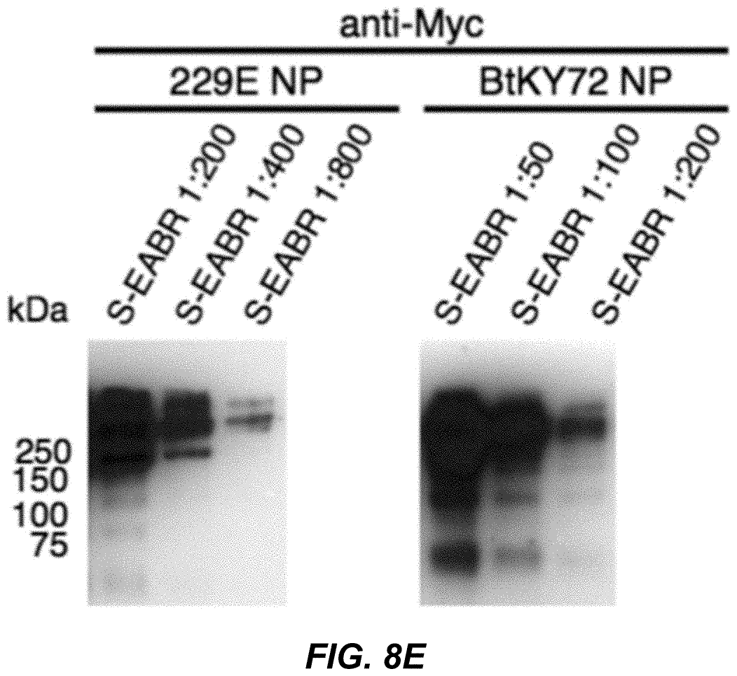

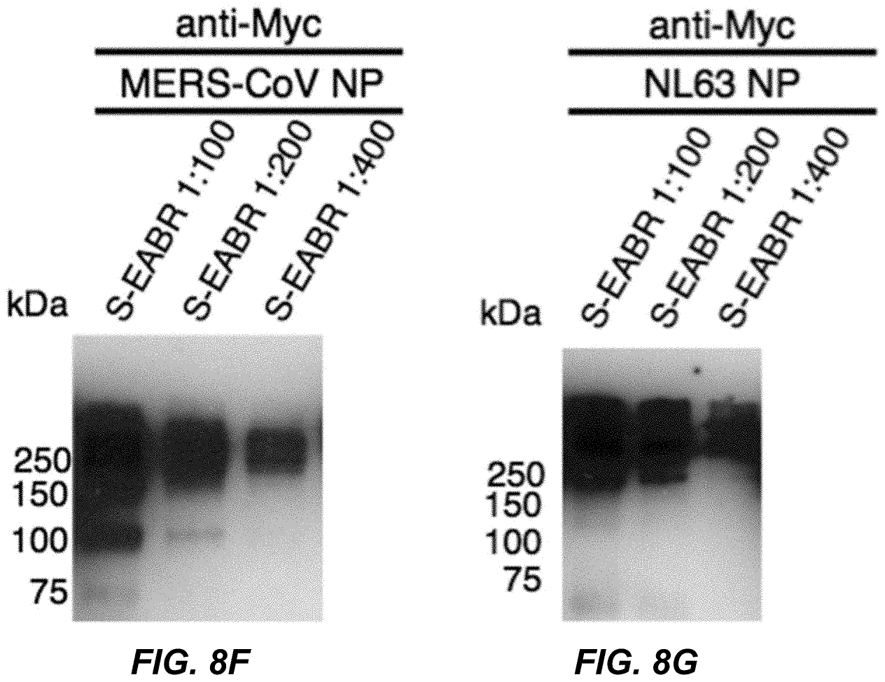

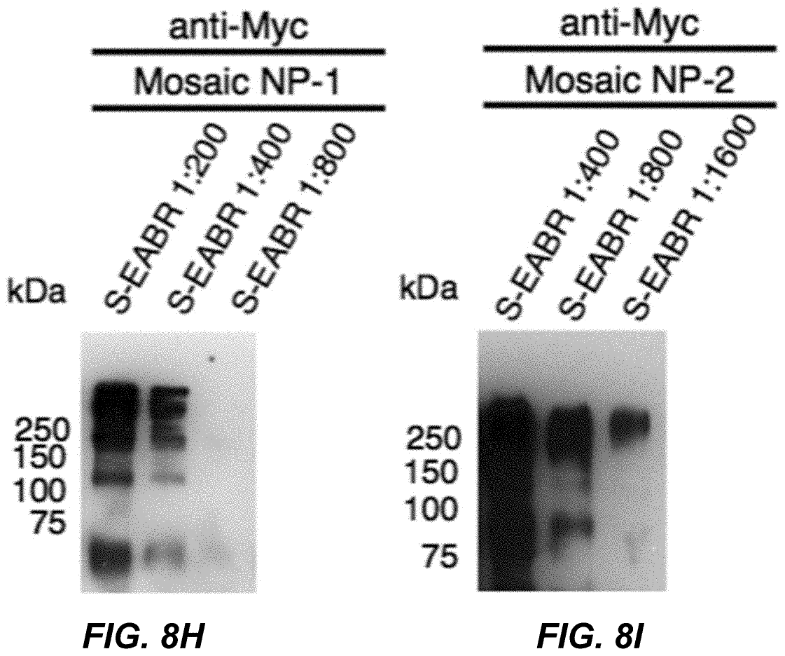

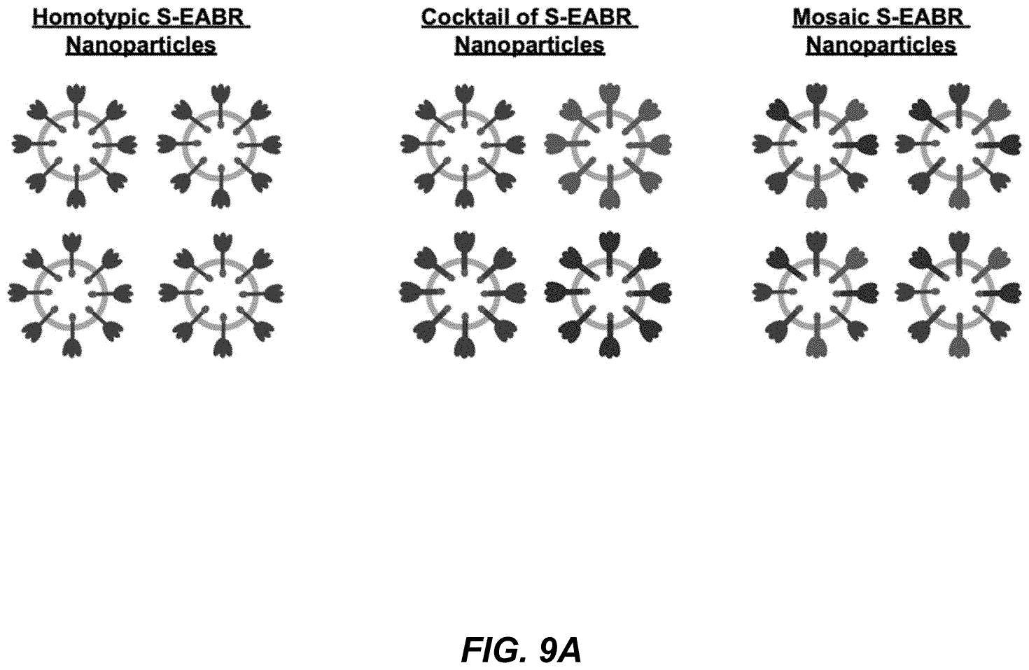

Although RBD-EABR NPs can also be generated with this technology (FIG. 8A), other parts of the S protein are more conserved among CoV strains than the RBD. S-EABR NPs can be generated for a wide range of CoV strains, including the SARS-CoV-2 variant B.1.351 (FIG. 8B), SARS, HKU-1 (FIG. 8C), Rf1, HKU-4 (FIG. 8D), 229E, BtKY72 (FIG. 8E), MERS (FIG. 8F), and NL63 (FIG. 8G). Additionally, two mosaic S-EABR NPs that displayed S proteins from multiple CoV strains on the same NP were made by co-expressing S-EABR constructs from 6 different CoV strains: Mosaic NP-1 (SARS, Rf1, BtKY72, HKU-1, HKU-4, 229E) (FIG. 8H) and mosaic NP-2 (SHC014, HKU-3, HKU-5, HKU-8, HKU-24, BM48-31) (FIG. 8I).

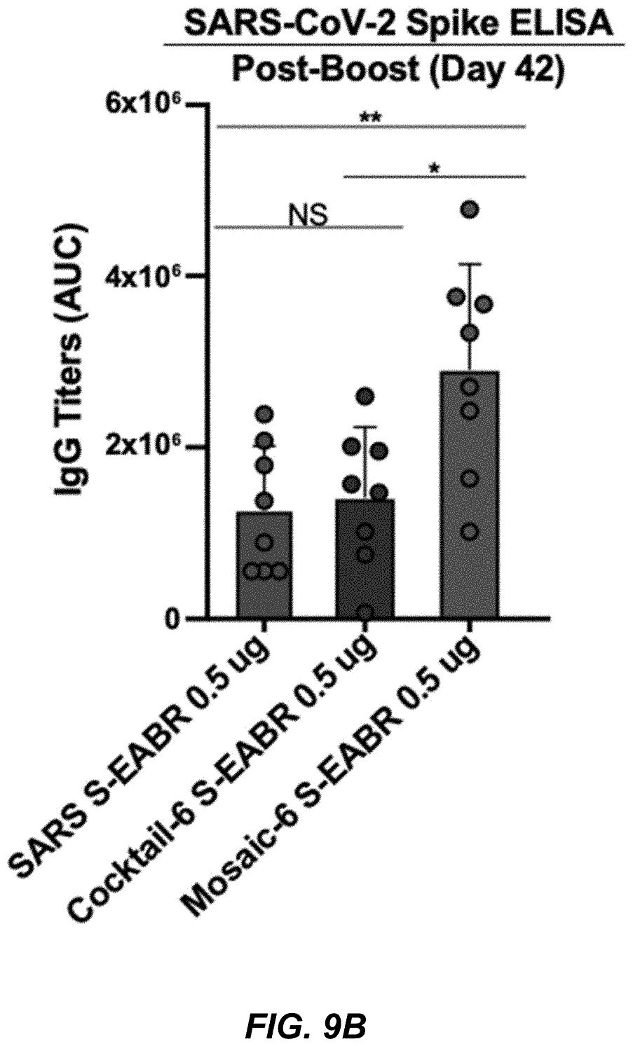

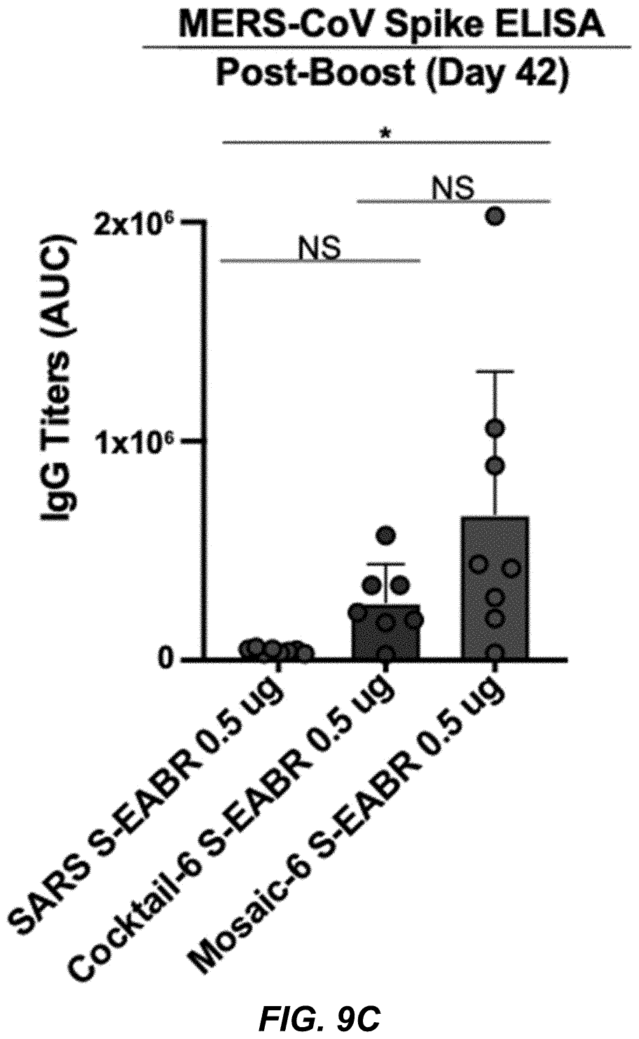

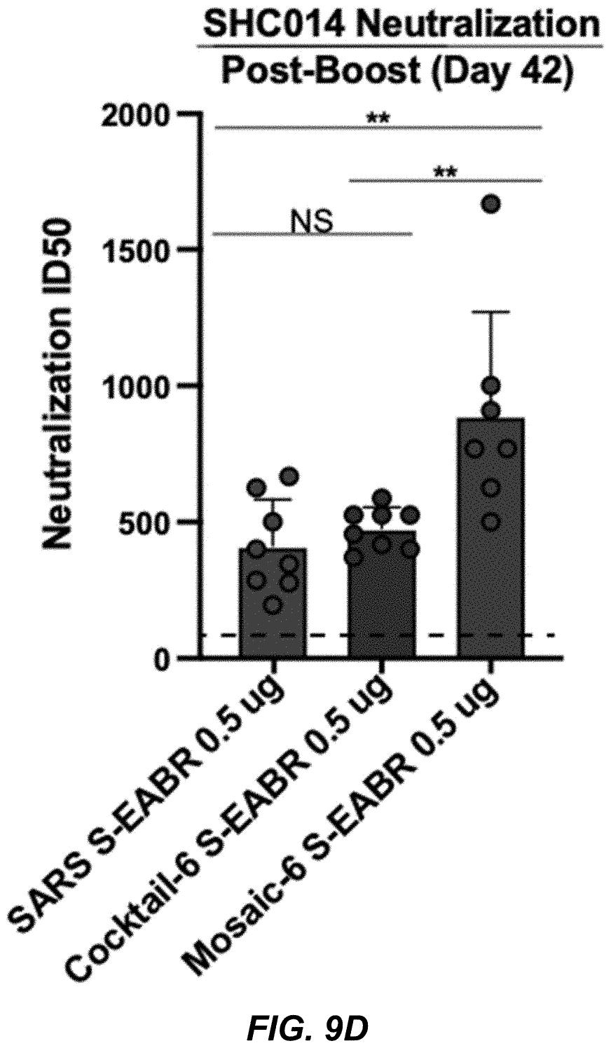

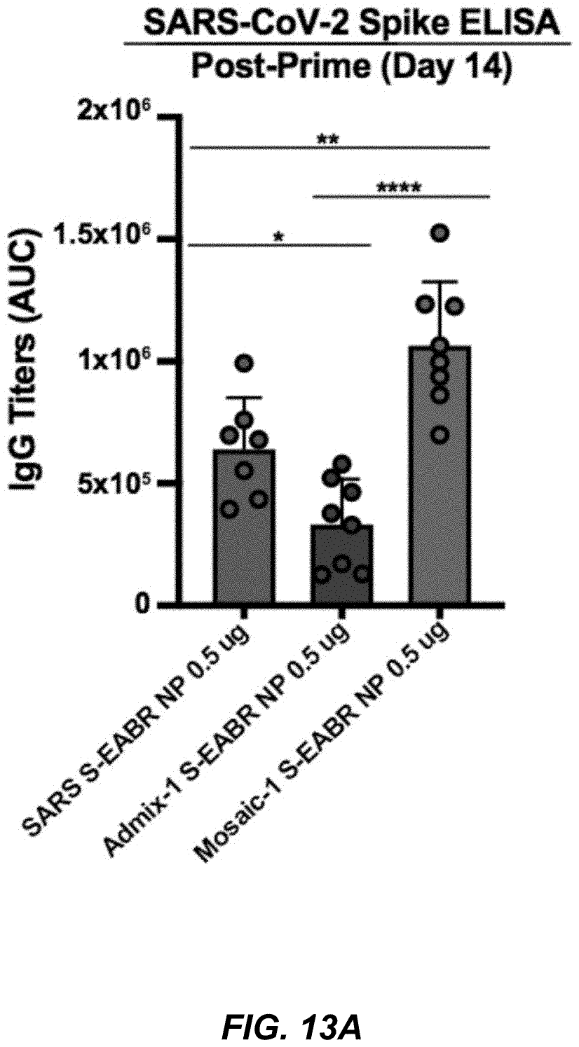

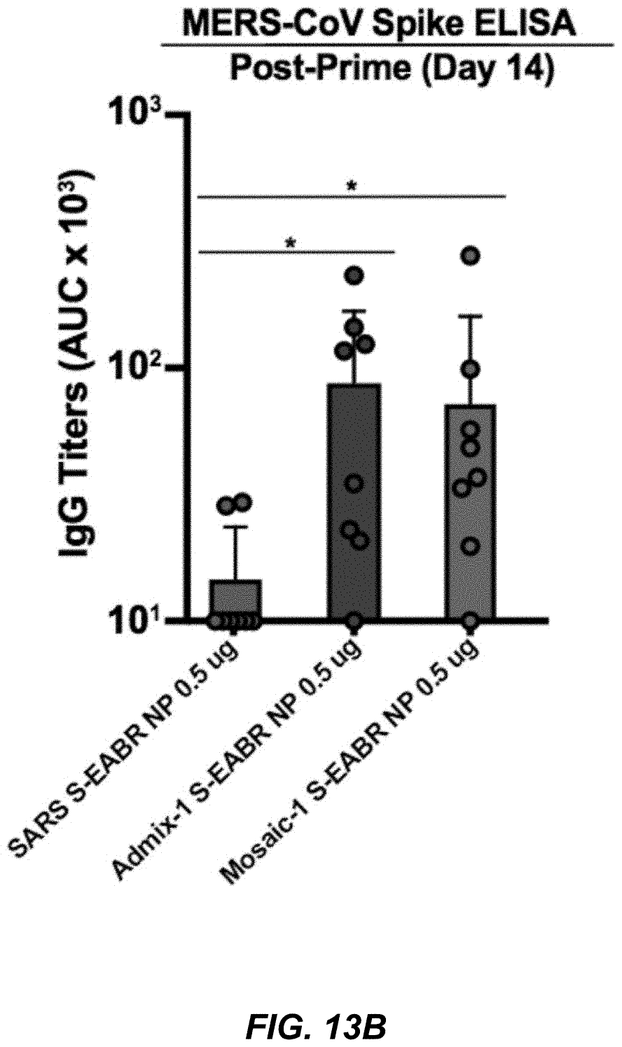

The 1^{st} generation Mosaic NP-1 displayed S proteins from three beta-CoV strains from the sarbecovirus family (SARS, Rf1, BtKY72), one beta-CoV strain from the embecovirus family (HKU1), one beta-CoV strain from the merbecovirus family (HKU4), and one alpha-CoV strain (229E) (FIG. 9A). Whereas mosaic NP production requires expression and purification of individual components for conventional NP technologies, mosaic S-EABR NPs can be harvested directly from culture supernatants and purified in a single step. Compared to homotypic SARS S-EABR NPs and the corresponding cocktail of six individual S-EABR NPs (FIG. 9A), mosaic S-EABR NPs elicited increased heterologous antibody responses in mice against CoV strains that were not displayed on mosaic NPs, including SARS-CoV-2, MERS, and SHC014 (FIG. 9B-FIG. 9D). A single injection of mosaic S-EABR NP-1 elicited significantly higher heterologous antibody responses against SARS-CoV-2 S and MERS-CoV S, which were both not displayed on mosaic S-EABR NP-1, as compared to homotypic NP or the cocktail of individual NPs (FIG. 13A-FIG. 13B).

### Advantages of EABR NP (ENP) Technology

Taken together, the EABR NP technology provided herein exhibits a number of key advantages over existing vaccine NP approaches that make it ideally-suited for the design of rapid-response and universal vaccines against CoVs, as well as potentially other pathogens.

Conventional approaches to generate enveloped NPs such as co-expression of lentiviral Gag proteins and viral surface proteins result in inefficient incorporation of surface proteins into NPs as the two proteins do not interact (FIG. 1A, FIG. 10). In contrast, the EABR-modified surface protein (e.g., fusion protein) itself drives NP assembly and is directly incorporated into the NP (FIG. 1A, FIG. 10) leading to more efficient production of densely-coated and highly immunogenic NPs (FIG. 1C-FIG. 1D, FIG. 11A, FIG. 6A-FIG. 6F).

While non-enveloped NP technologies normally require expression and purification of multiple components to generate NPs for vaccine applications, the EABR NP technology advantageously only requires expression of a single component and the self-assembling NPs can be purified directly from culture supernatants.

Enveloped EABR NPs are also ideally-suited for repeated immunizations to focus immune responses to desired epitopes on viral surface proteins, which can be challenging with non-enveloped NP-based vaccines as repeated immunizations elicit off-target immune responses against undesired epitopes such as the NP scaffold.

Another non-limiting advantage of the EABR NP technology is that viral surface proteins are maintained in their natural membrane-associated conformation without the need for extensive protein engineering. Other NP technologies require surface proteins to be modified into soluble proteins, which can be unstable and express poorly in the absence of extensive stabilizing mutations. As a result, NP-based vaccine approaches against SARS-CoV-2 have focused on displaying the RBD subunit, which is more stable than the full-length S protein. However, other parts of the S protein are more conserved among CoV strains than the RBD. The EABR NP technology is therefore the ideal approach for developing a universal CoV vaccine.

Unlike other NP technologies that require expression and purification of multiple components, the engineered SARS-CoV-2 S-EABR construct can be delivered as an mRNA vaccine as it only requires expression of a single component. In comparison to the SARS-CoV-2 S constructs used in the Pfizer and Moderna vaccines (FIG. 7A), mRNA-mediated delivery of the S-EABR construct can greatly enhance activation of B-cells, because S-EABR proteins will be expressed at the cell surface and self-assemble into S-EABR NPs that bud from the plasma membrane (FIG. 7B). Both S and S-EABR mRNAs can be expressed inside host cells and localize to the cell surface, eliciting potent T-cell activation but only moderate B-cell responses, respectively (FIG. 7A-FIG. 7B). However, formation and secretion of self-assembling S-EABR NPs can potentiate B-cell activation because the NPs would widely distribute inside the body to engage a large number of immune cells, thereby mimicking a natural infection (FIG. 7B). In some embodiments, more potent activation of B-cells would result in higher antibody titers and potentially greater efficacy against future SARS-CoV-2 variants. This hybrid approach retains the excellent manufacturability of mRNA vaccines and the enhanced potency can have commercial advantages as protective immune responses can be achieved with, e.g., lower mRNA doses or even just a single injection, resulting in reduced costs and faster distribution, which are critical attributes for rapid-response vaccines.

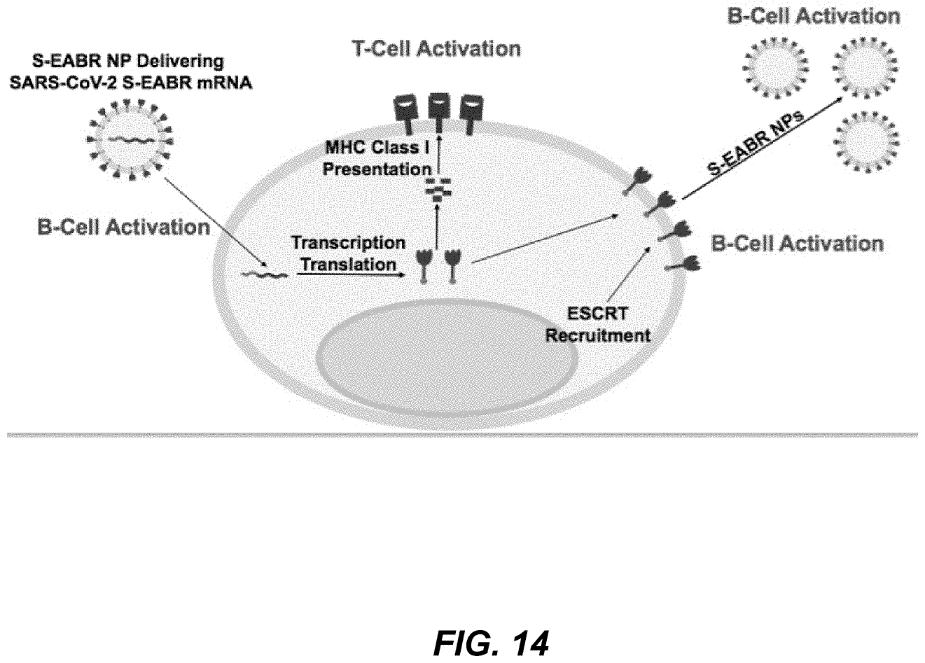

S-EABR NPs can also be engineered to transport and deliver their own mRNA to cells, to, in some embodiments, further potentiate immune responses as the injected S-EABR NPs already elicit potent antibody responses (FIG. 14). Incorporation into S-EABR NPs can be achieved by introducing a specific interaction between the cytoplasmic domain of the S-EABR protein and the mRNA encoding either S or S-EABR. In some embodiments, the bacteriophage MS2 capsid protein (MCP) can be inserted upstream of the EABR domain, which interacts with specific MS2 hairpins are inserted into the 3′ UTR of the S or S-EABR mRNAs. Alternatively, dCas13 can be programmed to recognize a nucleotide sequence within the mRNA and be inserted upstream of the EABR domain. This approach can substantially simplify the production process and reduce manufacturing costs as mRNA normally needs to be generated by in vitro transcription, then purified by HPLC, and finally encapsulated into synthetic lipid NPs.

In addition to the development of fast-response and universal vaccines against CoVs, the EABR NP technology can also be applicable to the design of protein- and/or nucleic acid-based vaccines against a wide range of viral pathogens. This includes but is not limited to HIV, influenza, flaviviruses (e.g., zika, dengue, yellow fever, hepatitis C), filoviruses (EBOV, Marburg), and emerging viral pathogens such as Hantavirus and Nipah virus. As described herein, HIV-1 Env-EABR NPs can be generated, and the purified Env-EABR NPs contained a greater amount of Env protein than NPs produced by co-expression of HIV-1 Gag and Env, suggesting that HIV-1 Env was incorporated more efficiently into Env-EABR NPs (FIG. 5B). The EABR NP technology can also be used to develop vaccines against non-viral infectious diseases such as Malaria and Tuberculosis.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A nucleic acid composition, comprising:
a polynucleotide encoding a fusion protein,
wherein the fusion protein comprises an antigenic polypeptide (AP) and an endosomal sorting complex required for transport (ESCRT)-recruiting domain (ERD),
and wherein a plurality of fusion proteins are capable of self-assembling into an enveloped nanoparticle (ENP) secreted from a cell in which the fusion proteins are expressed, thereby generating a population of ENPs.

2. The nucleic acid composition of claim 1, wherein the ERD recruits one or more ESCRT proteins to the cytoplasmic tail of the fusion protein, and wherein the recruitment of ESCRT proteins via the ERD induces the self-assembly and budding of ENPs.

3. The nucleic acid composition of claim 1, wherein the ERD comprises or is derived from EBOV VP40, Syntenin-1, rat Galectin-3 (rGalectin-3), Hrs, CD2AP, EIAV p9, HIV-1 p6, a Gag protein, and/or the ESCRT and ALIX binding region (EABR) of the human CEP55 protein.

4. The nucleic acid composition of claim 1, wherein the ERD comprises an amino acid sequence having at least 70% sequence identity to an amino acid sequence selected from the group consisting of SEQ ID NOs: 1-20 and 23.

5. The nucleic acid composition of claim 1, wherein the fusion protein comprises an endocytosis-preventing motif (EPM) capable of preventing endocytosis of the fusion protein.

6. The nucleic acid composition of claim 5, wherein the EPM:
tethers the fusion protein to the cytoskeleton, thereby preventing localization to coated pits and endocytosis;
enhances ENP assembly, ENP production, and/or ENP secretion; and/or
prevents endocytosis of the fusion protein, thereby extending the time a fusion protein remains at the plasma membrane to interact with ESCRT proteins.

7. The nucleic acid composition of claim 5, wherein the EPM comprises an amino acid sequence having at least 70% sequence identity to the amino acid sequence of SEQ ID NO: 21.

8. The nucleic acid composition of claim 1, wherein the AP, or a portion thereof, is displayed on the surface of the ENP.

9. The nucleic acid composition of claim 1, wherein the AP comprises or is derived from at least a portion of an antigenic protein associated with a disease or disorder.

10. The nucleic acid composition of claim 9, wherein the disease or disorder is an infectious disease or disorder caused by an infectious agent, wherein the AP comprises or is derived from an antigenic protein of said infectious agent, and wherein the antigenic protein of said infectious agent is a pathogenic antigen.

11. The nucleic acid composition of claim 9,
wherein the disease or disorder is a disease associated with expression of a tumor-associated antigen, and wherein the antigenic protein is a tumor-associated antigen;
wherein the disease or disorder is an autoimmune disease or disorder, and wherein the antigenic protein is an autoimmune antigen; and/or
wherein the disease or disorder is an allergic disease or disorder, and wherein the antigenic protein is an allergenic antigen.

12. The nucleic acid composition of claim 1, comprising:
n polynucleotides each encoding an nth fusion protein,
wherein n is an integer from 2 to 500, and
wherein at least two of the fusion proteins differ with respect to the AP, and wherein the population of ENPs thereby display a plurality of disparate AP.

13. The nucleic acid composition of claim 12, wherein the population of ENPs comprise:
one or more heterotypic ENPs, wherein at least two of the fusion proteins of a heterotypic ENP are different from each other with respect to the AP, and wherein a heterotypic ENP thereby displays a plurality of disparate AP;
a mixture of two or more homotypic ENPs that differ from each other with respect to the AP of the plurality of fusion proteins present in said two or more homotypic ENPs, and wherein the population of ENPs thereby displays a plurality of disparate AP; and/or
a mixture of two or more heterotypic ENPs that differ from each other with respect to the AP of the plurality of fusion proteins of said two or more heterotypic ENPs.

14. The nucleic acid composition of claim 12, wherein the plurality of disparate AP has a sequence identity of about, at least, or at least about 70% with one another.

15. The nucleic acid composition of claim 12,
wherein the plurality of disparate AP comprise two or more of a 1st pathogenic antigen (PA) of a 1st infectious agent (IA), a 2nd PA of a 2nd IA, a 3rd PA of a 3rd IA, a 4th PA of a 4th IA, a 5th PA of a 5th IA, a 6th PA of a 6th IA, a 7th PA of a 7th IA, a 8th PA of a 8th IA, a 9th PA of a 9th IA, a 10th PA of a 10th IA, a 11th PA of a 11th IA, a 12th PA of a 12th IA, a 13th PA of a 13th IA, a 14th PA of a 14th IA, a 15th PA of a 15th IA, a 16th PA of a 16th IA, a 17th PA of a 17th IA, a 18th PA of a 18th IA, a 19th PA of a 19th IA, a 20th PA of a 20th IA, a 21st PA of a 21st IA, a 22nd PA of a 22nd IA, a 23rd PA of a 23rd IA, a 24th PA of a 24th IA, a 25th PA of a 25th IA, a 26th PA of a 26th IA, a 27th PA of a 27th IA, a 28th PA of a 28th IA, a 29th PA of a 29th IA, a 30th PA of a 30th IA, a 31st PA of a 31st IA, a 32nd PA of a 32nd IA, a 33rd PA of a 33rd IA, a 34th PA of a 34th IA, a 35th PA of a 35th IA, a 36th PA of a 36th IA, a 37th PA of a 37th IA, a 38th PA of a 38th IA, a 39th PA of a 39th IA, a 40th PA of a 40th IA, a 41st PA of a 41st IA, a 42nd PA of a 42nd IA, a 43rd PA of a 43rd IA, a 44th PA of a 44th IA, a 45th PA of a 45th IA, a 46th PA of a 46th IA, a 47th PA of a 47th IA, a 48th PA of a 48th IA, a 49th PA of a 49th IA, and a 50th PA of a 50th IA, and
wherein the 1st, 2nd, 3rd, 4th, 5th, 6th, 7th, 8th, 9th, 10th, 11th, 12th, 13th, 14th, 15th, 16th, 17th, 18th, 19th, 20th, 21st, 22nd, 23rd, 24th, 25th, 26th, 27th, 28th, 29th, 30th, 31st, 32nd, 33rd, 34th, 35th, 36th, 37th, 38th, 39th, 40th, 41st, 42nd, 43rd, 44th, 45th, 46th, 47th, 48th, 49th, and 50th pathogenic antigens are different from one another.

16. The nucleic acid composition of claim 1, wherein the nucleic acid composition is, comprises, or further comprises, one or more vectors,
wherein the one or more vectors is a viral vector, a plasmid, a transposable element, a naked DNA vector, a lipid nanoparticle (LNP), or any combination thereof,
and wherein the viral vector is an AAV vector, a lentivirus vector, a retrovirus vector, an adenovirus vector, a herpesvirus vector, a herpes simplex virus vector, a cytomegalovirus vector, a vaccinia virus vector, a MVA vector, a baculovirus vector, a vesicular stomatitis virus vector, a human papillomavirus vector, an avipox virus vector, a Sindbis virus vector, a VEE vector, a Measles virus vector, an influenza virus vector, a hepatitis B virus vector, an integration-deficient lentivirus (IDLV) vector, or any combination thereof.

17. The nucleic acid composition of claim 1, wherein the nucleic acid composition is or comprises mRNA.

18. The nucleic acid composition of claim 17, the mRNA is formulated in a lipid nanoparticle (LNP) comprising one or more of an ionizable cationic lipid, a non-cationic lipid, a sterol, and a PEG-modified lipid.

19. A population of enveloped nanoparticles (ENPs),
wherein each of the ENPs comprises a plurality of fusion proteins each comprising an antigenic polypeptide (AP) and an endosomal sorting complex required for transport (ESCRT)-recruiting domain (ERD).

20. A method of stimulating an immune response in a subject in need thereof, comprising:
administering to the subject a pharmaceutically effective amount of the nucleic acid composition of claim 1, thereby stimulating an immune response in the subject.


SEQUENCE LISTING (24 sequences)

SEQ ID NO: 1 - protein, 52 aa, Artificial Sequence
  HIV-1 p6
LQSRPEPTAPPEESFRSGVETTTPPQKQEPIDKELYPLTSLRSLFGNDPSSQ

SEQ ID NO: 2 - protein, 44 aa, Artificial Sequence
  EBOV VP40
MRRVILPTAPPEYMEAIYPVRSNSTIARGGNSNTGFLTPESVNG

SEQ ID NO: 3 - protein, 51 aa, Artificial Sequence
  EIAV p9
PIQQKSQHNKSVVQETPQTQNLYPDLSEIKKEYNVKEKDQVEDLNLDSLWE

SEQ ID NO: 4 - protein, 54 aa, Artificial Sequence
  EABR
FNSSINNIHEMEIQLKDALEKNQQWLVYDQQREVYVKGLLAKIFELEKKTETAA

SEQ ID NO: 5 - protein, 44 aa, Artificial Sequence
  EABRmin1
MEIQLKDALEKNQQWLVYDQQREVYVKGLLAKIFELEKKTETAA

SEQ ID NO: 6 - protein, 35 aa, Artificial Sequence
  EABRmin2
MEIQLKDALEKNQQWLVYDQQREVYVKGLLAKIFE

SEQ ID NO: 7 - protein, 45 aa, Artificial Sequence
  muEABRmut1
KEMQLRDALDKNHQWLLYDQQREAYVRGLLARIFELEKRTETAAA

SEQ ID NO: 8 - protein, 48 aa, Artificial Sequence
  chicEABRmut1
VEKQLRDALEKNHQWLLYDQQREAYVRGLLGRIFELEQRSEIVSQQQP

SEQ ID NO: 9 - protein, 48 aa, Artificial Sequence
  chicEABRmut2
VEKQLKDALDKNQQWLLYDQQREAYVRGLLGRIFELEQRSEIVSQQQP

SEQ ID NO: 10 - protein, 47 aa, Artificial Sequence
  chamEABRmut1
IGMQLRDALDKNQQWLLYDQQREAYVQGLLARIFELEQQAKEAKPEE

SEQ ID NO: 11 - protein, 47 aa, Artificial Sequence
  goldEABRmut1
LQEQLRDALEKNHNWLLYDQQREAYVQGVLTRTKELETQLNEANQAL

SEQ ID NO: 12 - protein, 59 aa, Artificial Sequence
  Syntenin-1(2-60)
SLYPSLEDLKVDKVIQAQTAFSANPANPAILSEASAPIPHDGNLYPRLYPELSQYMGLS

SEQ ID NO: 13 - protein, 262 aa, Artificial Sequence
  rGalectin-3
MADGFSLNDALAGSGNPNPQGWPGAWGNQPGAGGYPGASYPGAYPGQAPPGGYPGQAPPSAYPGPTGPSAYPGPTAPGAYPGPTAPGAFPGQPGGPGAYPSAPGAYPSAPGAYPATGPFGAPTGPLTVPYDMPLPGGVMPRMLITIIGTVKPNANSITLNFKKGNDIAFHFNPRFNENNRRVIVCNTKQDNNWGREERQSAFPFESGKPFKIQVLVEADHFKVAVNDVHLLQYNHRMKNLREISQLGIIGDITLTSASHAMI

SEQ ID NO: 14 - protein, 120 aa, Artificial Sequence
  rGalectin-3min1
MADGFSLNDALAGSGNPNPQGWPGAWGNQPGAGGYPGASYPGAYPGQAPPGGYPGQAPPSAYPGPTGPSAYPGPTAPGAYPGPTAPGAFPGQPGGPGAYPSAPGAYPSAPGAYPATGPFG

SEQ ID NO: 15 - protein, 57 aa, Artificial Sequence
  rGalectin-3min2
GPTGPSAYPGPTAPGAYPGPTAPGAFPGQPGGPGAYPSAPGAYPSAPGAYPATGPFG

SEQ ID NO: 16 - protein, 167 aa, Artificial Sequence
  CD2APmin1
MVDYIVEYDYDAVHDDELTIRVGEIIRNVKKLQEEGWLEGELNGRRGMFPDNFVKEIKRETEFKDDSLPIKRERHGNVASLVQRISTYGLPAGGIQPHPQTKNIKKKTKKRQCKVLFEYIPQNEDELELKVGDIIDINEEVEEGWWSGTLNNKLGLFPSNFVKELEV

SEQ ID NO: 17 - protein, 59 aa, Artificial Sequence
  CD2APmin2
MVDYIVEYDYDAVHDDELTIRVGEIIRNVKKLQEEGWLEGELNGRRGMFPDNFVKEIKR

SEQ ID NO: 18 - protein, 20 aa, Artificial Sequence
  HTLV-1 (111-130)
PDSDPQIPPPYVEPTAPQVL

SEQ ID NO: 19 - protein, 70 aa, Artificial Sequence
  MLV (108-177)
PLPPSAPSLPLEPPRSTPPRSSLYPALTPSLGAKPKPQVLSDSGGPLIDLLTEDPPPYRDPRPPPSDRDG

SEQ ID NO: 20 - protein, 19 aa, Artificial Sequence
  MPMV (197-215)
PFLTRPPPYNKATPSAPTV

SEQ ID NO: 21 - protein, 48 aa, Artificial Sequence
  FcR
ALPGNPDHREMGETLPEEVGEYRQPSGGSVPVSPGPPSGLEPTSSSPY

SEQ ID NO: 22 - protein, 106 aa, Artificial Sequence
  FcR-EABR sequence
ALPGNPDHREMGETLPEEVGEYRQPSGGSVPVSPGPPSGLEPTSSSPYGGGSFNSSINNIHEMEIQLKDALEKNQQWLVYDQQREVYVKGLLAKIFELEKKTETAA

SEQ ID NO: 23 - protein, 28 aa, Artificial Sequence
  EABRmin3
MEIQLKDALEKNQQWLVYDQQREVYVKG

SEQ ID NO: 24 - protein, 1252 aa, Artificial Sequence
  SARS-CoV-2 S-D614G-2P-dCT
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGS